(19) United States
(12) Patent Application Publication
(10) Pub. No.: US 2021/0059749 A1
(43) Pub. Date: Mar. 4, 2021

(54) Systems and Methods for Ablating Prostate Tissue

(71) Applicant: Santa Anna Tech LLC, Santa Ana, CA (US)

(72) Inventors: Virender K. Sharma, Paradise Valley, AZ (US); George W. Bourne, Boston, MA (US); Jianmin Li, Lexington, MA (US)

(21) Appl. No.: 17/005,982

(22) Filed: Aug. 28, 2020

Related U.S. Application Data
(60) Provisional application No. 63/025,867, filed on May 15, 2020.
(60) Provisional application No. 62/953,116, filed on Dec. 23, 2019.
(60) Provisional application No. 62/893,062, filed on Aug. 28, 2019.

(51) Int. Cl. A61B 18/14

(52) U.S. Cl. CPC A61B 18/1492; A61B 2018/00547

(57) ABSTRACT

Ablation catheters and systems include catheter tips with at least one hollow needle that is extendable at an angle from the catheter body to ablate a target prostate tissue while avoiding structures in regions near the prostate tissue, including the urethra, the ejaculatory duct, and the rectum wall. The vapor ablation system has a pump, a catheter that includes a connection port positioned on a proximal end of the catheter, a lumen in fluid communication with the connection port and configured to receive, via the connection port, saline from the pump, at least one electrode positioned within the lumen, and at least one thermally conductive, elongated element having a lumen and configured to be coupled to the distal tip of the catheter such that a proximal end of the at least one thermally conductive, elongated element is positioned at least 0.1 mm and no more than 60 mm from a distal most electrode of the at least one electrode and such that the lumen of the at least one thermally conductive, elongated element is in fluid communication with the first lumen.

### CROSS-REFERENCE

The present application relies on U.S. Patent Provisional Appl. No. 62/893,062, entitled “Systems and Methods for Prostate and Endometrial Ablation”, filed on Aug. 28, 2019. The present application also relies on U.S. Patent Provisional Appl. No. 62/953,116, entitled “Systems and Methods for Prostate and Endometrial Ablation”, filed on Dec. 23, 2019. The present application also relies on U.S. Patent Provisional Appl. No. 63/025,867, entitled “Systems and Methods for Genitourinary Ablation”, filed on May 15, 2020.

The present application is related to U.S. patent application Ser. No. 15/600,670, entitled “Ablation Catheter with Integrated Cooling” and filed on May 19, 2017, which relies on U.S. Provisional Patent Application No. 62/425,144, entitled “Methods and Systems for Ablation” and filed on Nov. 22, 2016, and U.S. Provisional Patent Application No. 62/338,871, entitled “Cooled Coaxial Ablation Catheter” and filed on May 19, 2016, for priority.

U.S. patent application Ser. No. 15/600,670 is also a continuation-in-part application of U.S. patent application Ser. No. 15/144,768, entitled “Induction-Based Micro-Volume Heating System”, filed on May 2, 2016, and issued as U.S. Pat. No. 10,064,697 on Sep. 4, 2018, which is a continuation-in-part application of U.S. patent application Ser. No. 14/594,444, entitled “Method and Apparatus for Tissue Ablation”, filed on Jan. 12, 2015, and issued as U.S. Pat. No. 9,561,068 on Feb. 7, 2017, which is a continuation-in-part application of U.S. patent application Ser. No. 14/158,687, of the same title, filed on Jan. 17, 2014, and issued as U.S. Pat. No. 9,561,067 on Feb. 7, 2017, which, in turn, relies on U.S. Provisional Patent Application No. 61/753,831, of the same title and filed on Jan. 17, 2013, for priority.

U.S. patent application Ser. No. 14/158,687 is also a continuation-in-part application of U.S. patent application Ser. No. 13/486,980, entitled “Method and Apparatus for Tissue Ablation”, filed on Jun. 1, 2012, and issued as U.S. Pat. No. 9,561,066 on Feb. 7, 2017, which, in turn, relies on U.S. Provisional Patent Application No. 61/493,344, of the same title and filed on Jun. 3, 2011, for priority.

U.S. patent application Ser. No. 13/486,980 is also a continuation-in-part application of U.S. patent application Ser. No. 12/573,939, entitled “Method and Apparatus for Tissue Ablation” and filed on Oct. 6, 2009, which, in turn, relies on U.S. Provisional Patent Application No. 61/102,885, of the same title and filed on Oct. 6, 2008, for priority.

All of the above referenced applications are herein incorporated by reference in their entirety.

### FIELD

The present specification relates to systems and methods configured to generate and deliver vapor for ablation therapy. More particularly, the present specification relates to systems and methods comprising a vapor ablation catheter and vapor generation for delivering ablation therapy to specific areas in the prostate, the endometrium, and the urinary bladder.

### BACKGROUND

Benign Prostatic Hyperplasia (BPH) refers to enlargement of the prostate gland. The enlargement may be non-cancerous and is common in men as they grow older. However, the enlargement of the prostate gland, due to BPH, may result in compressing of the urethra, thereby impeding the flow of urine from the bladder through the urethra. Anatomically, the median and lateral lobes are usually enlarged, due to their highly glandular composition. The anterior lobe has little in the way of glandular tissue and is seldom enlarged. Carcinoma of the prostate typically occurs in the posterior lobe—hence the ability to discern an irregular outline per rectal examination.

The earliest microscopic signs of BPH usually begin between the age of 30 and 50 years old men in the peri-urethral zone (PuZ), which is posterior to the proximal urethra. In BPH, most of the growth occurs in the transition zone (TZ) of the prostate. In addition to these two classic areas, the peripheral zone (PZ) is also involved to a lesser extent. Prostatic cancer typically occurs in the PZ. However, BPH nodules, usually from the TZ, are often biopsied anyway to rule out cancer in the TZ. BPH is nodular hyperplasia and not diffuse hyperplasia, affecting the TZ and PuZs of the prostate. Adenoma from the TZ form the lateral lobes while adenoma from the PuZ form the middle lobe in clinical diseases.

Transurethral needle ablation (TUNA) is a procedure used to treat the symptoms caused by BPH. The ablation procedure is used to treat the extra prostate tissue that causes the symptoms of BPH.

Prostate cancer is diagnosed in approximately 8% of men between the ages of 50 and 70 and tends to occur in men as they grow older. Men experiencing symptoms with prostate cancer often exhibit symptoms similar to those encountered with BPH and can also suffer from sexual problems caused by the disease. Typically, men diagnosed with prostate cancer when the cancer is at an early stage have a very good prognosis. Therapy ranges from active surveillance to surgery and radiation and chemotherapy depending on the severity of the disease and the age of the patient.

Dysfunctional uterine bleeding (DUB), or menorrhagia, affects 30% of women in reproductive age. The associated symptoms have considerable impact on a woman's health and quality of life. The condition is typically treated with endometrial ablation or a hysterectomy. The rates of surgical intervention in these women are high. Almost 30% of women in the US will undergo hysterectomy by the age of 60, with menorrhagia or DUB being the cause for surgery in 50-70% of these women. Endometrial ablation techniques have been FDA approved for women with abnormal uterine bleeding and with intramural fibroids less than 2 cm in size. The presence of submucosal uterine fibroids and a large uterus size have been shown to decrease the efficacy of standard endometrial ablation. Of the five FDA approved global ablation devices (namely, Thermachoice, hydrothermal ablation, Novasure, Her Option, and microwave ablation (MEA)) only microwave ablation has been approved for use where the submucosal fibroids are less than 3 cm in size and are not occluding the endometrial cavity and, additionally, for large uteri up to 14 cm in width.

Bladder cancer is a rare form of cancer occurring as a result of an abnormal growth of cells within the bladder. The abnormal cells form a tumor. FIG. 22A illustrates multiple stages of cancer of a bladder 2200, as known in the medical field. Referring to the figure, at a first stage (Tis), a bladder tumor 2202 is above a mucosa 2204 layer within the bladder 2200. At a second stage (Ta), a tumor 2206 spreads to the mucosa 2204. At a third stage (T1), a tumor 2208 spreads to a submucosa 2210 layer beneath the mucosa 2204. At a fourth stage (T2), a tumor 2212 spreads to a superficial muscle 2214 beneath the submucosa 2210. At a fifth stage (T3a), a tumor 2216 spreads to a deep muscle 2218 beneath the superficial muscle 2214. At a sixth stage (T3b), a tumor 2220 spreads to a perivesical fat layer 2222 beyond the deep muscle 2218. At a seventh stage (T4b), a tumor 2224 spreads to areas outside the perivesical fat layer 2222. At an eighth stage (T4a), a tumor 2226 spreads to extravesical structures 2228 outside the bladder 2200. Ablation techniques may be used to treat cancer of stages from first to fourth, which are non-muscle invasive or superficial bladder cancers. Further, ablation techniques may be used to palliate cancer of stages from fifth onwards, which are invasive bladder cancers.

A bladder's function is to hold urine that is made in the kidneys and travels down to the bladder through tubes called ureters. Urine exits the bladder into the urethra which in turn ports the urine out of the body. Some individuals suffer from an over-active bladder (OAB) that leads to an urge to pass urine several times in a day, even when the bladder is not full. Ablation techniques may be used to treat patients with OAB.

As the bladder is meant to hold urine, vapors from ablation methods may not be effective in the presence of urine over the tissue that needs to be ablated. It is therefore desirable to provide a way to ablate bladder tissues after complete removal of fluids, water, and/or urine away from the target tissue.

Ablation, as it pertains to the present specification, relates to the removal or destruction of a body tissue, via the introduction of a destructive agent, such as radiofrequency energy, laser energy, ultrasonic energy, cyroagents, or steam. Ablation is commonly used to eliminate diseased or unwanted tissues, such as, but not limited to cysts, polyps, tumors, hemorrhoids, and other similar lesions. Ablation techniques may be used in combination with chemotherapy, radiation, surgery, and Bacillus Calmette-Guérin (BCG) vaccine therapy, among others.

Steam-based ablation systems, such as the ones disclosed in U.S. Pat. Nos. 9,615,875, 9,433,457, 9,376,497, 9,561,068, 9,561,067, and 9,561,066, disclose ablation systems that controllably deliver steam through one or more lumens toward a tissue target. One problem that all such steam-based ablation systems have is the potential overheating or burning of healthy tissue. Steam passing through a channel within a body cavity heats up surfaces of the channel and may cause exterior surfaces of the medical tool, other than the operational tool end itself, to become excessively hot. As a result, physicians may unintentionally burn healthy tissue when external portions of the device, other than the distal operational end of the tool, accidentally contacts healthy tissue. U.S. Pat. Nos. 9,561,068, 9,561,067, and 9,561,066 are hereby incorporated herein by reference.

Furthermore, it is often desirable to rapidly cool a treatment area after the application of steam or some other ablative agent. Current systems largely rely, however, on a natural cooling process that prolongs treatment time. Alternatively, current medical treatment methods may flush an area with fluid, but that requires implementing a separate medical tool, thereby complicating the procedure and also prolonging treatment times.

It is therefore desirable to have steam-based ablation devices that integrate into the device itself safety mechanisms which prevent unwanted ablation during use. It is further desirable to be able to provide a way to augment the natural cooling process to thereby decrease total treatment time as well as be able to increase the vapor delivery time. Finally, it is desirable to provide an easy to implement cooling mechanism that does not rely on a separate medical tool to deliver fluid to cool the treatment area.

### SUMMARY

The present specification discloses a vapor ablation system for ablating prostate tissue of a patient, wherein the system comprises: at least one pump; a catheter having a length extending between a proximal end and a distal tip, wherein the catheter comprises: a connection port positioned on the proximal end of the catheter, wherein, through the connection port, the catheter is in fluid communication with the at least one pump; a first lumen in fluid communication with the connection port and configured to receive, via the connection port, saline from the at least one pump; at least one electrode positioned within the first lumen; and at least one thermally conductive, elongated element having a lumen and configured to be coupled to the distal tip of the catheter such that a proximal end of the at least one thermally conductive, elongated element is positioned at least 0.1 mm and no more than 60 mm from a distal most electrode of the at least one electrode and such that the lumen of the at least one thermally conductive, elongated element is in fluid communication with the first lumen; and a controller having at least one processor in data communication with the at least one pump, wherein, upon activating, the controller is configured to: control a delivery of saline into the first lumen; and control a delivery of an electrical current to the at least one electrode positioned within the first lumen.

Optionally, the at least one thermally conductive, elongated element comprises a needle and a needle attachment component. The needle may have a tapered distal tip. The needle and the needle attachment component may be made of the same material and the same material may be stainless steel. A proximal portion of the needle may be configured to be threaded onto a distal end of the needle attachment component

Optionally, the vapor ablation system further comprises a needle chamber coupled to the distal tip of the catheter and configured to be retractable along a length of the catheter. The needle chamber may have an exterior surface and an internal lumen that defines an internal surface, wherein the exterior surface comprises a first material, wherein the internal surface comprises a second material, and wherein the first material is different from the second material. The first material may be a polymer and the second material may be metal. The needle chamber may have an internal lumen that defines an internal surface, wherein the internal lumen is curved to receive a curved needle. The at least one thermally conductive, elongated element may comprise a needle, wherein, in a pre-deployment state, the needle chamber is configured to be positioned over the needle and wherein, in a post-deployment state, the needle chamber is configured to be retracted toward a proximal end of the catheter and the needle is positioned outside the needle chamber. Optionally, the needle is further adapted to have a pre-needle chamber state, wherein, in the pre-needle chamber state, the needle has a first degree of curvature, wherein, in the pre-deployment state, the needle has a second degree of curvature, wherein, in the post-deployment state, the needle has a third degree of curvature, wherein the first degree of curvature is different from both the second degree of curvature and third degree of curvature, and wherein the second degree of curvature is different from the third degree of curvature. Optionally, the needle is further adapted to have a pre-needle chamber state, wherein, in the pre-needle chamber state, the needle has a first degree of curvature, wherein, in the pre-deployment state, the needle has a second degree of curvature, wherein, in the post-deployment state, the needle has a third degree of curvature, wherein the first degree of curvature is greater than both the second degree of curvature and third degree of curvature, and wherein the third degree of curvature is greater than the second degree of curvature. Optionally, in a post-deployment state, the needle is configured to extend outward at an angle between 30° and 90° from an external surface of the catheter.

Optionally, the at least one thermally conductive, elongated element comprises a needle and a needle attachment component wherein the needle comprises an internal channel in fluid communication with the first lumen and a port to allow a passage of vapor to an external environment from the internal channel.

Optionally, the at least one thermally conductive, elongated element comprises more than one needle.

Optionally, the at least one thermally conductive, elongated element comprises a needle having a length extending from a proximal end to a tapered, distal end and further comprises insulation positioned over the length of needle. The insulation may be adapted to cover at least 5% of the length of the needle, beginning from the proximal end wherein the insulation is adapted to no more than 90% of the length of the needle, beginning from the proximal end.

Optionally, the controller is adapted to control the delivery of saline into the first lumen and control the delivery of the electrical current to the at least one electrode such that greater than 0% and less than 75% of a contiguous circumference of a prostatic urethra of the patient is ablated.

Optionally, the controller is adapted to control the delivery of saline into the first lumen and control the delivery of the electrical current to the at least one electrode such that greater than 0% and less than 75% of a contiguous circumference of an ejaculatory duct of the patient is ablated.

Optionally, the controller is adapted to control the delivery of saline into the first lumen and control the delivery of the electrical current to the at least one electrode such that greater than 0% and less than 75% of a thickness of the rectal wall is ablated.

Optionally, the controller is adapted to control the delivery of saline into the first lumen and control the delivery of the electrical current to the at least one electrode such that greater than 0% and less than 75% of one of a contiguous circumference of an ejaculatory duct and a central zone of the prostate is ablated.

Optionally, the controller is adapted to control the delivery of saline into the first lumen and control the delivery of the electrical current to the at least one electrode such that a transitional zone of a prostate of the patient is ablated and greater than 0% and less than 75% of an anterior fibromuscular strauma of the patient is ablated.

The present specification also discloses a vapor ablation system for treating diseases, wherein the system comprises: at least one pump; a catheter in fluid communication through a catheter connection port with the at least one pump, wherein a proximal end of the catheter is connected to the catheter connection port to place the catheter in fluid communication with the at least one pump, wherein the catheter comprises: at least one lumen to transport saline delivered from the at least one pump; at least one electrode positioned within the at least one lumen; a plurality of openings proximate to a distal end of the catheter; a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to: control the delivery of saline into the at least one lumen in the catheter; control the delivery of an electrical current to the at least one electrode positioned within the at least one lumen of the first catheter; and control vapor generated from the saline.

Optionally, the plurality of thermally conductive elements are needles.

Optionally, the plurality of thermally conductive elements are extended at an angle between 30° and 90° from the catheter.

Optionally, the system is used for ablating prostate tissue of a patient through the urethra of the patient, wherein greater than 0% and less than 75% of a contiguous circumference of a prostatic urethra of the patient is ablated.

Optionally, the system is used for ablating prostate tissue of a patient through the urethra of the patient, wherein greater than 0% and less than 75% of a contiguous circumference of an ejaculatory duct of the patient is ablated.

Optionally, the system is used for ablating prostate tissue of a patient through the rectal wall of the patient, wherein greater than 0% and less than 75% of a thickness of the rectal wall is ablated.

Optionally, the system is configured to ablate at least one of a central zone or a transitional zone of a prostate while ablating greater than 0% and less than 75% of a contiguous circumference of a prostatic urethra.

Optionally, the system is configured to ablate at least one of a central zone or a transitional zone of a prostate while ablating greater than 0% and less than 75% of a contiguous circumference of an ejaculatory duct.

Optionally, the system is configured to ablate a median lobe of a prostate while ablating greater than 0% and less than 75% of one of a contiguous circumference of an ejaculatory duct and a central zone of the prostate.

Optionally, the system is configured to ablate a transitional zone of a prostate while ablating greater than 0% and less than 75% of an Anterior Fibromuscular Strauma (AFS).

The present specification also discloses a method of ablating a prostatic tissue of a patient, comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one electrode positioned within the at least one lumen; a plurality of openings proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; inserting said catheter into a urethra of said patient; extending said thermally conductive elements through said plurality of openings and into said prostatic tissue; and programming said controller to control a delivery of said vapor such that greater than 0% and less than 75% of a circumference of a prostatic tissue or proximate tissue is ablated.

Optionally, the thermally conductive elements comprise needles.

Optionally, said prostatic tissue or proximate tissue is a prostatic urethra.

Optionally, said prostatic tissue or proximate tissue is an ejaculatory duct.

Optionally, said prostatic tissue or proximate tissue is a rectal wall.

The present specification also discloses a vapor ablation system for treating diseases, wherein the system comprises: at least one pump; a coaxial catheter for inserting into a vagina of a patient towards the cervix, the coaxial catheter comprising: an outer catheter for advancing to the internal os of the cervix of the patient; an inner catheter for advancing into the uterus of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport saline delivered from the at least one pump; at least one electrode positioned within the at least one lumen; at least two positioning elements separated along a length of the inner catheter, wherein a distal positioning element is advanced until the distal end of the distal positioning element contacts fundus of the uterus, and a proximal positioning element is advanced for positioning proximate an internal os of the patient and for creating a partial seal or contact with the internal os; and at least one openings proximate to the distal positioning element of the inner catheter; a controller having at least one processor in data communication with the at least one pump, wherein, upon activating, the controller is configured to: control the delivery of saline into the at least one lumen in the coaxial catheter; control the delivery of an electrical current to the at least one electrode positioned within the at least one lumen of the inner catheter; and control vapor generated from the saline.

Optionally, the inner catheter is used to measure a length of the uterine cavity of the patient. Optionally, the measured length is used to determine an amount of the vapor to be used for ablating.

Optionally, the partial seal is a temperature dependent seal and breaks once the temperature inside the sealed portion of the uterus exceeds 90° C.

Optionally, the partial seal is a pressure dependent seal and breaks once the temperature inside the sealed portion of the uterus exceeds 101° C. and the pressure exceeds 0.5 psi. Optionally, the partial seal is a pressure dependent seal and breaks once the temperature inside the sealed portion of the uterus exceeds 102° C. and the pressure exceeds 1.0 psi. Optionally, the partial seal is a pressure dependent seal and breaks once the temperature inside the sealed portion of the uterus exceeds 103° C. and the pressure exceeds 1.5 psi.

Optionally, the controller controls the vapor to an amount to keep endometrial pressure below at least one of 50 mm Hg and 10% above atmospheric pressure. Optionally, the controller controls the vapor to an amount to keep endometrial pressure below at least one of 30 mm Hg and 10% above atmospheric pressure. Optionally, the controller controls the vapor to an amount to keep endometrial pressure below at least one of 15 mm Hg and 10% above atmospheric pressure.

Optionally, at least one of the inner and the outer catheter comprise a venting element to allow venting of the uterus. Optionally, the venting element comprises grooves.

Optionally, the proximal positioning element comprises at least one opening to allow venting of the uterus.

Optionally, the inner catheter includes a pressure sensor to allow maintaining a pressure of vapor within the uterus to less than 50 mm Hg. Optionally, the inner catheter includes a pressure sensor to allow maintaining a pressure of vapor within the uterus to less than 30 mm Hg. Optionally, the inner catheter includes a pressure sensor to allow maintaining a pressure of vapor within the uterus to less than 15 mm Hg.

Optionally, each positioning element comprises an uncovered wire mesh.

The present specification also discloses a method of ablating a prostatic tissue of a patient, comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one positioning element on a distal end of the at least one lumen; at least one electrode positioned within the at least one lumen; an outer sheath covering the at least one lumen; a plurality of openings on the outer sheath proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; inserting the distal end of the catheter into a urethra of said patient; extending the distal end of the catheter into a bladder of said patient; retracting the outer sheath to expose the at least one lumen and the positioning element; expanding the positioning element; extending said thermally conductive elements through said plurality of openings and into said prostatic tissue; and programming said controller to control a delivery of said vapor such that greater than 0% and less than 75% of a circumference of a prostatic tissue or proximate tissue is ablated.

Optionally, the thermally conductive elements comprise needles.

Optionally, said prostatic tissue or proximate tissue is a prostatic urethra.

Optionally, said prostatic tissue or proximate tissue is an ejaculatory duct.

Optionally, said prostatic tissue or proximate tissue is a rectal wall.

Optionally, expanding the positioning element comprises positioning the positioning element proximate the bladder neck.

Optionally, expanding the positioning element comprises positioning the positioning element within the prostatic urethra.

The present specification also discloses a method of ablating an endometrial tissue of a patient, comprising: providing an ablation system comprising: at least one pump; a coaxial catheter for inserting into a vagina of a patient towards the cervix, the coaxial catheter comprising: an outer catheter for advancing to the internal os of the cervix of the patient; an inner catheter for advancing into the uterus of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport saline delivered from the at least one pump; at least one electrode positioned within the at least one lumen; at least two positioning elements separated along a length of the inner catheter, wherein a distal positioning element is advanced until the distal end of the distal positioning element contacts a fundus of the uterus, and a proximal positioning element is advanced for positioning proximate an internal os of the patient and for creating a partial seal with the internal os; and a plurality of openings positioned on said inner catheter and between said distal positioning element and said proximal positioning element for the delivery of vapor; a controller having at least one processor in data communication with the at least one pump, wherein, upon activating, the controller is configured to control the delivery of saline into the at least one lumen in the coaxial catheter, and control vapor generated from the saline; inserting the distal end of the catheter until the distal end of the distal positioning element contacts fundus of the uterus and a proximal positioning element is advanced for positioning proximate an internal os of the patient; expanding the distal positioning element; expanding the proximal positioning element for creating a partial seal within the internal os; and programming said controller to control a delivery of said vapor for ablating the endometrial tissue.

Optionally, the distal positioning element and the proximal positioning element each have a funnel shape.

The present specification also discloses a method of ablating a median lobe of a prostate in a patient with median lobe hyperplasia, the method comprising: passing a catheter with at least one needle into the patient's spongy urethra and through a prostatic urethra such that a distal end of the catheter is positioned within the patient's bladder; extending the at least one needle from the distal end of the catheter and passing the needle through a bladder or bladder neck wall and into the median lobe; delivering ablative agent through the at least one needle and into the median lobe to ablate prostatic tissue; and using a controller to control a flow of ablative agent to maintain a pressure in the bladder and median lobe below 5 atm.

Optionally, the catheter further comprises at least one positioning element and the method further comprises, prior to extending the at least one needle, deploying the at least one positioning element to position the catheter in the bladder and stabilize the at least one needle.

The present specification also discloses a method of ablating a median lobe of a prostate in a patient with median lobe hyperplasia, the method comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one electrode positioned within the at least one lumen; an outer sheath covering the at least one lumen; a plurality of openings on the outer sheath proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; inserting the catheter into the patient's spongy urethra and through a prostatic urethra such that a distal end of the catheter is positioned within the patient's bladder; extending the plurality of thermally conductive elements from the distal end of the catheter, through a bladder wall and into the median lobe; delivering ablative agent through the plurality of thermally conductive elements and into the median lobe to ablate prostatic tissue; and programming the controller to control to control a flow of ablative agent to maintain a pressure in the bladder and median lobe below 5 atm.

Optionally, the catheter further comprises at least one positioning element and the method further comprises, prior to extending the extending the plurality of thermally conductive elements, deploying the at least one positioning element to position the catheter in the bladder and stabilize the plurality of thermally conductive elements.

The present specification also discloses a method for ablating at least one of a target area within or proximate a urinary bladder of a patient, the method comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one electrode positioned within the at least one lumen; a plurality of openings proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; draining fluid within the urinary bladder from vicinity of the target area; inserting the catheter into a ureter of the patient; extending the thermally conductive elements through the plurality of openings and into or proximate the target area; and programming the controller to control a delivery of the vapor such that the target area is ablated.

Optionally, the target area is at least one of a tissue, a tumor, or a nerve. Optionally, target area is a tissue inside the urinary bladder. Optionally, the target area is within an adventitial space beneath a trigone of the patient. Optionally, the target area is within one of a bladder neck, an internal urinary sphincter (IUS), and nerves supplying the IUS and bladder neck, of the patient.

Optionally, draining the fluid comprises draining urine from the urinary bladder.

Optionally, draining the fluid further comprises performing at least one of: removing urine from the urinary bladder; insufflating air into the urinary bladder; and positioning the patient so as to have the target area positioned away from a dependent part of the urinary bladder, allowing for urine to drain away from the urinary bladder.

Optionally, the thermally conductive elements comprise needles.

Optionally, the method further comprises applying a positioning element proximate the target area and enclosing at least a portion of the target area.

Optionally, the method further comprises maintaining a pressure within the urinary bladder at below 5 atm.

The present specification also discloses a method for ablating at least one of a target area within or proximate a urinary bladder of a patient, the method comprising: providing an ablation system comprising: at least one pump; a coaxial catheter for inserting into a ureter the patient, the coaxial catheter comprising: an outer catheter for advancing to the ureter of the patient; an inner catheter for advancing into the ureter of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport saline delivered from the at least one pump; at least one electrode positioned within the at least one lumen; at least one positioning element along a length of the inner catheter, wherein the at least one positioning element is advanced until the distal end of the positioning element encloses the target area; and at least one opening proximate to the positioning element of the inner catheter; a controller having at least one processor in data communication with the at least one pump, wherein, upon activating, the controller is configured to: control the delivery of saline into the at least one lumen in the coaxial catheter; control the delivery of an electrical current to the at least one electrode positioned within the at least one lumen of the inner catheter; and control vapor generated from the saline; draining fluid within the urinary bladder from vicinity of the target area; inserting the coaxial catheter into a ureter of the patient; applying the positioning element proximate the target area enclosing at least a portion of the target area; and programming the controller to control a delivery of the vapor such that the target area is ablated.

Optionally, the target area is at least one of a tissue, a tumor, or a nerve. Optionally, the target area is a tissue inside the urinary bladder. Optionally, draining the fluid comprises draining urine from the urinary bladder.

Optionally, draining the fluid further comprises performing at least one of: removing urine from the urinary bladder; insufflating air into the urinary bladder; positioning the patient so as to have the target area positioned away from a dependent part of the urinary bladder, allowing for urine to drain away from the urinary bladder.

Optionally, the method further comprises maintaining a pressure within the urinary bladder at below 5 atm.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1A illustrates an ablation system, in accordance with embodiments of the present specification;

FIG. 1B is a transverse cross-section view of a flexible heating chamber, in accordance with an embodiment of the present specification;

FIG. 1C illustrates transverse and longitudinal cross-section views of first and second arrays of electrodes of a flexible heating chamber, in accordance with an embodiment of the present specification;

FIG. 1D is a transverse cross-section view of the heating chamber of FIG. 1B, including assembled first and second arrays of electrodes, in accordance with an embodiment of the present specification;

FIG. 1E is a longitudinal cross-section view of the heating chamber of FIG. 1B, including assembled first and second arrays of electrodes, in accordance with an embodiment of the present specification;

FIG. 1F is a first longitudinal view of two heating chambers of FIG. 1B arranged in series in a catheter tip, in accordance with an embodiment of the present specification;

FIG. 1G is a second longitudinal view of two heating chambers of FIG. 1B arranged in series in a catheter tip, in accordance with an embodiment of the present specification;

FIG. 1H illustrates a multiple lumen balloon catheter incorporating one heating chamber of FIG. 1B, in accordance with an embodiment of the present specification;

FIG. 1I illustrates a multiple lumen balloon catheter incorporating two heating chambers of FIG. 1B, in accordance with an embodiment of the present specification;

FIG. 1J illustrates a catheter with proximal and distal positioning elements and an electrode heating chamber, in accordance with embodiments of the present specification;

FIG. 1K illustrates an ablation system for the ablation of prostatic tissue, in accordance with embodiments of the present specification;

FIG. 1L illustrates a catheter for use in the ablation of prostatic tissue, in accordance with embodiments of the present specification;

FIG. 1M illustrates a system for use in the ablation of prostatic tissue, in accordance with another embodiment of the present specification;

FIG. 1N illustrates an ablation system for the ablation of endometrial tissue, in accordance with embodiments of the present specification;

FIG. 1O illustrates a catheter for use in the ablation of endometrial tissue, in accordance with embodiments of the present specification;

FIG. 1P illustrates a system for use in the ablation of endometrial tissue, in accordance with another embodiment of the present specification;

FIG. 1Q illustrates a controller for use with an ablation system, in accordance with an embodiment of the present specification;

FIG. 1R illustrates a system for use in the ablation of prostatic tissue, in accordance with another embodiment of the present specification;

FIG. 1S illustrates a needle attachment component of a system for use in the ablation of prostatic tissue, in accordance with some embodiments of the present specification;

FIG. 1T illustrates a needle chamber of a system for use in the ablation of prostatic tissue, in accordance with some embodiments of the present specification;

FIG. 2A illustrates a single lumen double balloon catheter comprising an in-line heating element, in accordance with an embodiment of the present specification;

FIG. 2B illustrates a coaxial lumen double balloon catheter comprising an in-line heating element, in accordance with an embodiment of the present specification;

FIG. 3A illustrates a typical anatomy of a prostatic region for descriptive purposes;

FIG. 3B illustrates an exemplary transparent view of prostate anatomy, highlighting a peripheral zone, in addition to other zones in the periphery of the prostate;

FIG. 3C illustrates an oblique top-down transparent view of a prostate, showing the various zones and prostatic urethra;

FIG. 4A is an illustration of a water cooled catheter, in accordance with another embodiment of the present specification;

FIG. 4B is a cross-section view of a tip section of the water cooled catheter of FIG. 4A;

FIG. 4C illustrates embodiments of a distal end of a catheter for use with the system of FIG. 1M;

FIG. 4D illustrates other embodiments of a distal end of a catheter for use with the system of FIG. 1M;

FIG. 4E illustrates an embodiment of a slit flap used to cover openings of FIGS. 4C and 4D, in accordance with some embodiments of the present specification;

FIG. 4F illustrates an embodiment of a positioning element to be positioned at a distal end of an ablation catheter, to position the ablation catheter in the prostatic urethra, in accordance with the present specification;

FIG. 4G illustrates a distal end of an ablation catheter advanced through a prostatic urethra, in accordance with an exemplary embodiment of the present specification;

FIG. 4H illustrates a distal end of an ablation catheter advanced into a bladder, in accordance with an exemplary embodiment of the present specification;

FIG. 4I illustrates a distal end of an ablation catheter advanced further into a bladder, in accordance with an exemplary embodiment of the present specification;

FIG. 4J illustrates a positioning element expanded at a distal end of an ablation catheter and being retracted to position the positioning element proximate a bladder neck or urethra, in accordance with an exemplary embodiment of the present specification;

FIG. 4K illustrates at least one needle extended from a distal end of an ablation catheter and into prostatic tissue, in accordance with an exemplary embodiment of the present specification;

FIG. 4L illustrates an ablative agent being delivered through one or more needles and into prostatic tissue, in accordance with an exemplary embodiment of the present specification;

FIG. 4M illustrates an ablation catheter advanced into a prostatic urethra and having a positioning element at a position proximal to needles positioned at the distal end of the catheter, in accordance with an alternative embodiment of the present specification;

FIG. 4N illustrates needles at a distal end of an ablation catheter deployed into prostatic tissue, in accordance with the alternative embodiment of the present specification;

FIG. 4O is a flow chart illustrating the steps involved in using an ablation catheter to ablate a prostate of a patient, in accordance with embodiments of the present specification;

FIG. 5A illustrates prostate ablation being performed on an enlarged prostrate in a male urinary system by using the device, in accordance with an embodiment of the present specification;

FIG. 5B is an illustration of transurethral prostate ablation being performed on an enlarged prostrate in a male urinary system using an ablation device, in accordance with one embodiment of the present specification;

FIG. 5C is an illustration of transurethral prostate ablation being performed on an enlarged prostrate in a male urinary system using an ablation device, in accordance with another embodiment of the present specification;

FIG. 5D is a flow chart listing the steps involved in a transurethral enlarged prostate ablation process using an ablation catheter, in accordance with one embodiment of the present specification;

FIG. 5E is an illustration of transrectal prostate ablation being performed on an enlarged prostrate in a male urinary system using an ablation device, in accordance with one embodiment of the present specification;

FIG. 5F is an illustration of transrectal prostate ablation being performed on an enlarged prostrate in a male urinary system using a coaxial ablation device having a positioning element, in accordance with another embodiment of the present specification;

FIG. 5G is a close-up illustration of the distal end of the catheter and needle tip of the ablation device;

FIG. 5H is a flow chart listing the steps involved in a transrectal enlarged prostate ablation process using an ablation catheter, in accordance with one embodiment of the present specification;

FIG. 6A is an illustration of an ablation catheter, in accordance with an embodiment of the present specification;

FIG. 6B is a cross-section view of a tip of the ablation catheter of FIG. 6A;

FIG. 6C is an illustration of transurethral prostate ablation being performed using the ablation catheter of FIG. 6A, in accordance with an embodiment;

FIG. 6D is a flow chart listing the steps involved in a transurethral enlarged prostate ablation process, in accordance with an embodiment;

FIG. 7A is an illustration of an ablation catheter, in accordance with another embodiment of the present specification;

FIG. 7B is a cross-section view of a tip of the ablation catheter of FIG. 7A;

FIG. 7C is an illustration of transurethral prostate ablation being performed using the ablation catheter of FIG. 7A, in accordance with an embodiment;

FIG. 7D is a flow chart listing the steps involved in a transurethral enlarged prostate ablation process, in accordance with an embodiment.

FIG. 8A is an illustration of one embodiment of a positioning element of an ablation catheter, depicting a plurality of thermally conducting elements attached thereto;

FIG. 8B is an illustration of one embodiment of a positioning element of an ablation catheter, depicting a plurality of hollow thermally conducting elements attached thereto;

FIG. 9 is a flowchart illustrating one embodiment of a method of ablation of a tissue using a needle catheter device;

FIG. 10 is a flowchart illustrating a method of ablation of a submucosal tissue using a needle catheter device, according to one embodiment of the present specification;

FIG. 11A is an exemplary illustration of shape changing needles, according to one embodiment of the present specification;

FIG. 11B illustrates different embodiments of needles, in accordance with the present specification;

FIG. 11C illustrates an exemplary process of delivery of an ablative agent from hollow openings at the edges of a pair of needles, such as double needles of FIG. 11B, in accordance with some embodiments of the present specification;

FIG. 11D illustrates exemplary depths of needles of different curvatures, in accordance with some embodiments of the present specification;

FIG. 11E illustrates exemplary depths of needles, relative to needles of FIG. 11D, in accordance with some embodiments of the present specification;

FIG. 11F illustrates exemplary lengths of needles of FIG. 11E, extending in a straight line from the port to the farthest distance reached by the body of the needles, in accordance with some embodiments of the present specification;

FIG. 11G illustrates different views of a single needle assembly extending from a port, in accordance with some embodiments of the present specification;

FIG. 11H illustrates one or more holes at the sharp edge of the needle in another horizontal view of the needle, in accordance with some embodiments of the present specification;

FIG. 11I illustrates different views of a double needle assembly extending from a port, in accordance with some embodiments of the present specification;

FIG. 11J illustrates different views of another double needle assembly extending from a port, in accordance with some embodiments of the present specification;

FIG. 11K illustrates an insulation on a single needle configuration and a double needle configuration, in accordance with some embodiments of the present specification;

FIG. 11L illustrates a single needle configuration with insulation, positioned inside a prostatic tissue in accordance with some embodiments of the present specification;

FIG. 11M illustrates a single needle configuration with insulation, positioned inside a uterine fibroid in accordance with some embodiments of the present specification;

FIG. 11N illustrates a double needle configuration where the two needles are inserted into separate prostate lobes, in accordance with some embodiments of the present specification;

FIG. 11O illustrates an exemplary embodiment of a steerable catheter shaft in accordance with some embodiments of the present specification;

FIG. 11P illustrates a needle with an open tip, in accordance with some embodiments of the present specification;

FIG. 11Q illustrates an alternative embodiment of a needle with an occluded tip and comprising holes or openings along an uninsulated length of the needle, in accordance with the present specification;

FIG. 12 is an illustration of transurethral prostate ablation being performed using an ablation device, in accordance with one embodiment of the present specification;

FIG. 13A is an illustration of one embodiment of a positioning element of an ablation catheter with needles attached to the catheter body;

FIG. 13B is an illustration of another embodiment of positioning elements for an ablation catheter;

FIG. 13C illustrates a cross section of the distal tip of a catheter, in accordance with an embodiment of the present specification;

FIG. 14 illustrates one embodiment of a handle mechanism that may be used for deployment and retrieval of ablation needles at variable depths of insertion;

FIG. 15A is a flowchart illustrating a method of ablation of prostatic tissue in accordance with one embodiment of the present specification;

FIG. 15B is a flowchart illustrating a method of ablation of prostatic tissue in accordance with another embodiment of the present specification;

FIG. 15C illustrates a compressed catheter with an expandable element being advanced into a prostatic urethra, in accordance with an embodiment of the present specification;

FIG. 15D illustrates an expanded expandable element of a catheter, pressing on urethral walls which presses on a prostate, and ablative agent being delivered through from inside the expandable member and into prostatic tissue, in accordance with an embodiment of the present specification;

FIG. 15E illustrates a widened prostatic urethra, after removing an expandable catheter, in accordance with an embodiment of the present specification;

FIG. 15F illustrates an expanded expandable element of a catheter and an exemplary use of one or more needles to allow delivery of an ablative agent, such as steam or vapor through a hollow exit at the edge of the needle, in accordance with some embodiments of the present specification;

FIG. 15G illustrates an ablation catheter used to ablate prostatic tissue of a patient with median lobe hyperplasia via a trans-cystic approach, in accordance with one embodiment of the present specification;

FIG. 15H illustrates an ablation catheter used to ablate prostatic tissue of a patient with median lobe hyperplasia via a trans-cystic approach, in accordance with another embodiment of the present specification;

FIG. 15I is a flowchart listing the steps in one method of using an ablation catheter to ablate prostatic tissue of a patient with median lobe hyperplasia via a trans-cystic approach, in accordance with one embodiment of the present specification;

FIG. 16A is an International Prostate Symptom Score (IPSS) Questionnaire;

FIG. 16B is a Benign Prostatic Hypertrophy Impact Index Questionnaire (BPHIIQ);

FIG. 17A illustrates a typical anatomy of a uterus and uterine tubes of a human female;

FIG. 17B illustrates the location of the uterus and surrounding anatomical structures within a female body;

FIG. 18A illustrates an exemplary ablation catheter arrangement for ablating the uterus, in accordance with some embodiments of the present specification;

FIG. 18B illustrates an exemplary embodiment of grooves configured in an inner catheter of FIG. 18A, in accordance with some embodiments of the present specification;

FIG. 18C is a flowchart of a method of using the catheter of FIG. 18A for ablation of endometrial tissue, in accordance with embodiments of the present specification;

FIG. 18D illustrates a catheter for endometrial ablation, in accordance with other embodiments of the present specification;

FIG. 18E illustrates a catheter having an expanded distal positioning element advanced through a cervical canal and into a uterus, in accordance with embodiments of the present specification;

FIG. 18F illustrates catheter having an expanded distal positioning element and an expanded proximal positioning element, further advanced into a uterus, in accordance with embodiments of the present specification;

FIG. 18G illustrates vapor delivered into a uterus through a plurality of ports on a catheter body and positioned between proximal and distal positioning elements, in accordance with embodiments of the present specification;

FIG. 18H is a flow chart illustrating the steps involved in using an ablation catheter to ablate an endometrium of a patient, in accordance with embodiments of the present specification;

FIG. 18I illustrates a side view, cross-section side view, and distal end front-on view of an endometrial ablation catheter, in accordance with some embodiments of the present specification;

FIG. 18J illustrates a perspective side view of the catheter of FIG. 18I with the stent extending over the inner catheter, and extending out from the outer catheter, in accordance with some embodiments of the present specification;

FIG. 18K illustrates a cross-section side view, a perspective side view, and a distal end front-on view of a braided stent, in accordance with some embodiments of the present specification;

FIG. 18L illustrates a side perspective view of a distal end of an inner catheter, in accordance with some embodiments of the present specification;

FIG. 18M illustrates a side front perspective view of a distal end of an inner catheter, in accordance with some embodiments of the present specification;

FIG. 18N illustrates a top perspective view of a distal end of an inner catheter, in accordance with some embodiments of the present specification;

FIG. 18O illustrates different views of a double-positioning element catheter with an atraumatic olive tip end, in accordance with another embodiment of the present specification;

FIG. 18P illustrates distal ends of ablation catheters having distal positioning elements and a plurality of ports along a length of the catheter shaft, in accordance with some embodiments of the present specification;

FIG. 18Q illustrates distal ends of ablation catheters having distal olive tips, positioning elements, and a plurality of ports along a length of the catheter shaft, in accordance with some embodiments of the present specification;

FIG. 18R illustrates a side view a distal end of an ablation catheter having a distal olive tip, two positioning elements, and a plurality of ports along a length of the catheter shaft, in accordance with some embodiments of the present specification;

FIG. 18S illustrates a rear perspective view of the catheter of FIG. 18R;

FIG. 18T illustrates a distal end of an ablation catheter with a half-circle openings at the distal end and a distal positioning element, in accordance with some embodiments of the present specification;

FIG. 18U illustrates a distal end of an ablation catheter having a spherical shaped distal positioning element and a cover extending over the entirety or a portion of the positioning element, in accordance with an exemplary embodiment of the present specification;

FIG. 18V illustrates a distal end of an ablation catheter having a spherical shaped distal positioning element, in accordance with another exemplary embodiment of the present specification;

FIG. 18W illustrates a distal end of an ablation catheter having a conical shaped distal positioning element, in accordance with yet another exemplary embodiment of the present specification;

FIG. 18X illustrates an atraumatic soft tip of a catheter shaft that is used for insertion in to a cervix, in accordance with some embodiments of the present specification;

FIG. 19A illustrates a configuration of a disc for use with the catheter arrangement of FIG. 18A, in accordance with one embodiment of the present specification;

FIG. 19B illustrates a configuration of a disc for use with the catheter arrangement of FIG. 18A, in accordance with another embodiment of the present specification;

FIG. 19C illustrates multiple configurations of discs for use with the catheter arrangement of FIG. 18A, in accordance with yet other embodiments of the present specification;

FIG. 19D illustrates an assembly of catheter with a handle, and a cervical collar, in accordance with some embodiments of the present specification;

FIG. 19E illustrates a position of the cervical collar as it sits at an external os, outside the uterus and cervix, before deployment of the catheter, in accordance with some embodiments of the present specification;

FIG. 19F illustrates an exemplary position of hands to hold the catheter for deploying the proximal positioning element, in accordance with some embodiments of the present specification;

FIG. 19G illustrates expanding of the proximal positioning element while the user pushes the handle of the catheter to extend the inner catheter within the uterus, in accordance with some embodiments of the present specification;

FIG. 19H illustrates deploying of the distal positioning element, which may be uncoated or selectively coated with silicone, in accordance with some embodiments of the present specification;

FIG. 19I illustrates turn of a dial to further retract first positioning element to partially seal cervical os, so as to isolate the uterus, in accordance with some embodiments of the present specification;

FIG. 19J illustrates a distal end of an ablation catheter having two positioning elements and a plurality of ports along a length of the catheter shaft, in accordance with some embodiments of the present specification;

FIG. 19K illustrates a distal end of an ablation catheter having two positioning elements a distal olive tip, and a plurality of ports along a length of the catheter shaft, in accordance with some embodiments of the present specification;

FIG. 19L illustrates a connector for connecting a distal positioning element to a distal end of an ablation catheter, in accordance with some embodiments of the present specification;

FIG. 19M illustrates another connector for connecting a distal positioning element to a distal end of an ablation catheter, in accordance with other embodiments of the present specification;

FIG. 19N illustrates a connector for connecting a proximal positioning element to a distal end of an ablation catheter, in accordance with some embodiments of the present specification;

FIG. 19O illustrates another connector for connecting a proximal positioning element to a distal end of an ablation catheter, in accordance with other embodiments of the present specification;

FIG. 19P illustrates a shaft of an ablation catheter depicting a plurality of ports, in accordance with some embodiments of the present specification;

FIG. 20A illustrates endometrial ablation being performed in a female uterus by using an ablation device, in accordance with an embodiment of the present specification;

FIG. 20B is an illustration of a coaxial catheter used in endometrial tissue ablation, in accordance with one embodiment of the present specification;

FIG. 20C is a flow chart listing the steps involved in an endometrial tissue ablation process using a coaxial ablation catheter, in accordance with one embodiment of the present specification;

FIG. 20D is an illustration of a bifurcating coaxial catheter used in endometrial tissue ablation, in accordance with one embodiment of the present specification;

FIG. 20E is a flowchart listing the steps of a method of using the ablation catheter of FIG. 20D to ablate endometrial tissue, in accordance with one embodiment of the present specification;

FIG. 20F is an illustration of a bifurcating coaxial catheter with expandable elements used in endometrial tissue ablation, in accordance with one embodiment of the present specification;

FIG. 20G is an illustration of the catheter of FIG. 20F inserted into a patient's uterine cavity for endometrial tissue ablation;

FIG. 20H is a flowchart listing the steps of a method of using the ablation catheter of FIG. 20F to ablate endometrial tissue, in accordance with one embodiment of the present specification;

FIG. 20I is an illustration of a bifurcating coaxial catheter used in endometrial tissue ablation, in accordance with another embodiment of the present specification;

FIG. 20J is an illustration of a bifurcating coaxial catheter used in endometrial tissue ablation, in accordance with yet another embodiment of the present specification;

FIG. 20K is an illustration of a water cooled catheter used in endometrial tissue ablation, in accordance with one embodiment of the present specification;

FIG. 20L is an illustration of a water cooled catheter used in endometrial tissue ablation and positioned in a uterus of a patient, in accordance with another embodiment of the present specification;

FIG. 20M is an illustration of a water cooled catheter used in cervical ablation, in accordance with one embodiment of the present specification;

FIG. 20N is an illustration of the catheter of FIG. 20M positioned in a cervix of a patient;

FIG. 20O is a flowchart listing the steps involved in cervical ablation performed using the catheter of FIG. 20M;

FIG. 21A is a flowchart illustrating a method of ablation of endometrial tissue in accordance with one embodiment of the present specification;

FIG. 21B is a flowchart illustrating a method of ablating a uterine fibroid in accordance with one embodiment of the present specification;

FIG. 22A illustrates different stages of cancer of a bladder, as known in the medical field;

FIG. 22B illustrates a system for use in the ablation of bladder tissue, in accordance with an embodiment of the present specification;

FIG. 23 illustrates an exemplary catheter for insertion into a urinary bladder for ablating bladder tissue, in accordance with some embodiments of the present specification;

FIG. 24A illustrates a front end view of a positioning element, in accordance with some embodiments of the present specification;

FIG. 24B illustrates a side view of a distal end of an ablation catheter and positioning element of FIG. 24A;

FIG. 24C illustrates a front side perspective view of the distal end of an ablation catheter and positioning element of FIG. 24B;

FIG. 25A illustrates a close-up view of a connection between a positioning element and a distal end of an ablation catheter, in accordance with some embodiments of the present specification;

FIG. 25B illustrates a side view of the positioning element attached to a distal end of an ablation catheter of FIG. 25A;

FIG. 25C illustrates different types of configurations of positioning elements which may be used in accordance with the various ablation catheters of the embodiments of the present specification;

FIG. 26A illustrates positioning of a needle ablation catheter for delivering vapor to selectively ablate nerve-rich layers of deep detrusor and adventitial space beneath trigone, in accordance with embodiments of the present specification;

FIG. 26B illustrates positioning of a needle ablation device for delivering vapor to selectively ablate the bladder neck, internal urinary sphincter (IUS), and nerves supplying the IUS and bladder neck, in accordance with embodiments of the present specification;

FIG. 27A illustrates different views of a coaxial needle that may be used for ablation for treatment of OAB, in accordance with some embodiments of the present specification;

FIG. 27B illustrates the distal ends of coaxial needles comprising inner and outer tubes with lumens, in accordance with some embodiments of the present specification;

FIG. 28 is a flow chart illustrating an exemplary process of ablating the urinary bladder and/or its peripheral areas, in accordance with some embodiments of the present specification;

FIG. 29 illustrates a system for use in the ablation and imaging of prostatic tissue, in accordance with an embodiment of the present specification;

FIG. 30 illustrates a system for use in the ablation and imaging of endometrial tissue, in accordance with an embodiment of the present specification;

FIG. 31 illustrates a system for use in the ablation and imaging of bladder tissue, in accordance with an embodiment of the present specification;

FIG. 32 illustrates various components of an optical/viewing system for direct visualization of ablation in accordance with the embodiments of the present specification;

FIG. 33 illustrates components of a distal end of an ablation system that may be used in treatment of benign prostatic hyperplasia (BPH), and abnormal uterine bleeding (AUB), for use in accordance with the embodiments of the present specification;

FIG. 34 illustrates an image of a distal end of an ablation catheter viewed on a display device, in accordance with some embodiments of the present specification;

FIG. 35A depicts a cross-sectional view of an embodiment of a combination catheter comprising lumens for an optical/electrical catheter alongside a lumen for an ablation catheter, in accordance with some embodiments of the present specification;

FIG. 35B depicts a cross-sectional view of another embodiment of a combination catheter comprising lumens for an optical/electrical catheter alongside a lumen for an ablation catheter, in accordance with some embodiments of the present specification; and

FIG. 35C depicts a cross-sectional view of yet another embodiment of a combination catheter comprising a lumen for an optical/electrical catheter alongside a lumen for an ablation catheter, in accordance with some embodiments of the present specification.

### DETAILED DESCRIPTION

In various embodiments, the ablation devices and catheters described in the present specification are used in conjunction with any one or more of the heating systems described in U.S. patent application Ser. No. 14/594,444, entitled “Method and Apparatus for Tissue Ablation”, filed on Jan. 12, 2015 and issued as U.S. Pat. No. 9,561,068 on Feb. 7, 2017, which is herein incorporated by reference in its entirety. U.S. patent application Ser. No. 15/600,670, entitled “Ablation Catheter with Integrated Cooling” and filed on May 19, 2017; Ser. No. 15/144,768, entitled “Induction-Based Micro-Volume Heating System” filed on May 2, 2016, and issued as U.S. Pat. No. 10,064,697 on Sep. 4, 2018; Ser. No. 14/158,687, entitled “Method and Apparatus for Tissue Ablation”, filed on Jan. 17, 2014, and issued as U.S. Pat. No. 9,561,067 on Feb. 7, 2017; Ser. No. 13/486,980, entitled “Method and Apparatus for Tissue Ablation”, filed on Jun. 1, 2012, and issued as U.S. Pat. No. 9,561,066 on Feb. 7, 2017; and, Ser. No. 12/573,939, entitled “Method and Apparatus for Tissue Ablation” and filed on Oct. 6, 2009, are all herein incorporated by reference in their entirety.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the heater or induction-based approaches to generating steam from water described in this application.

Embodiments of the present specification are useful in the treatment of genitourinary structures, where the term “genitourinary” includes all genital and urinary structures, including, but not limited to, the prostate, uterus, and urinary bladder, and any conditions associated therewith, including, but not limited to, benign prostatic hyperplasia (BPH), prostate cancer, uterine fibroids, abnormal uterine bleeding (AUB), overactive bladder (OAB), strictures, and tumors.

Any and all of the needles and needle configurations disclosed in the specification with regards to a particular embodiment, such as including but not limited to, single needles, double needles, multiple needles and insulated needles, are not exclusive to that embodiment and may be used with any other of the embodiments disclosed in the specification in any of the organ systems for any condition related to the organ system such as and not limited to ablation of prostate, uterus, and bladder.

For purposes of the present specification, ‘completely ablating’ is defined as ablating more than 55% of a surface area or a volume around an anatomical structure.

All of the methods and systems for treating the prostate, uterus, and bladder may include optics or visualization as described in the specification to assist with direct visualization during ablation procedures.

All ablation catheters disclosed in the specification, in some embodiments, include insulation at the location of the electrode(s) to prevent ablation of tissue proximate the location of the electrode within the catheter.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The devices and methods of the present specification can be used to cause controlled focal or circumferential ablation of targeted tissue to varying depth in a manner in which complete healing with re-epithelialization can occur. Additionally, the vapor could be used to treat/ablate benign and malignant tissue growths resulting in destruction, liquefaction and absorption of the ablated tissue. The dose and manner of treatment can be adjusted based on the type of tissue and the depth of ablation needed. The ablation devices can be used for the prostate and endometrial ablation and for the treatment of any mucosal, submucosal or circumferential lesion, such as inflammatory lesions, tumors, polyps and vascular lesions. The ablation devices can also be used for the urinary bladder ablation, and for treating an over-active bladder (OAB). The ablation device can also be used for the treatment of focal or circumferential mucosal or submucosal lesions of the genitourinary tract. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radio-opaque or sonolucent material can be incorporated into the body of the catheter for radiological localization. Ferromagnetic materials can be incorporated into the catheter to help with magnetic navigation.

Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor may use temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

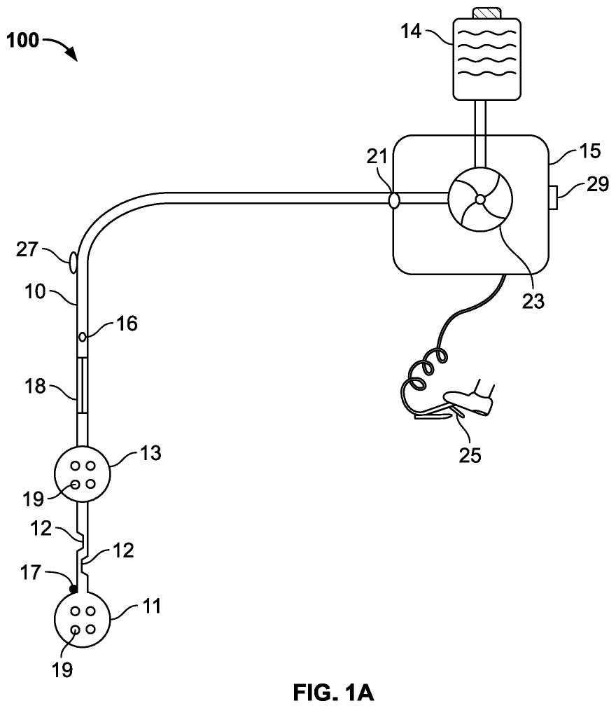

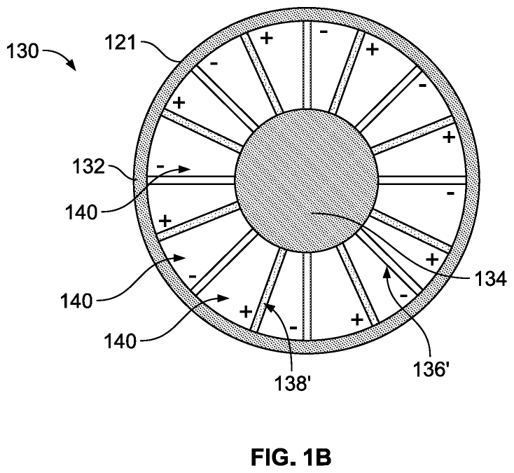

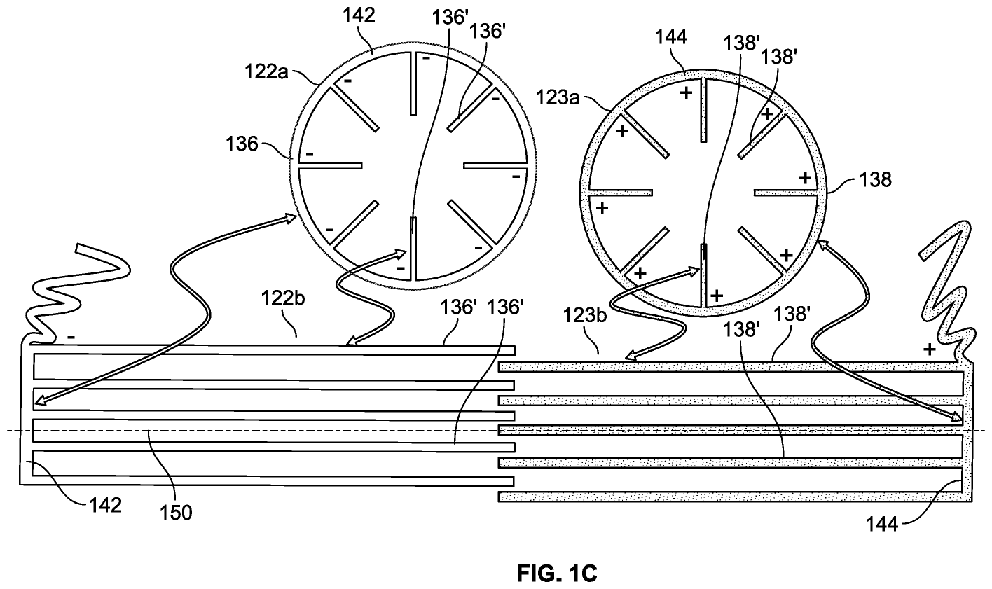

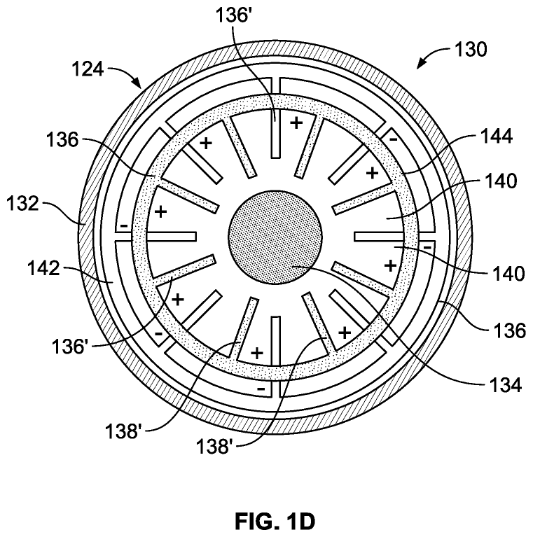

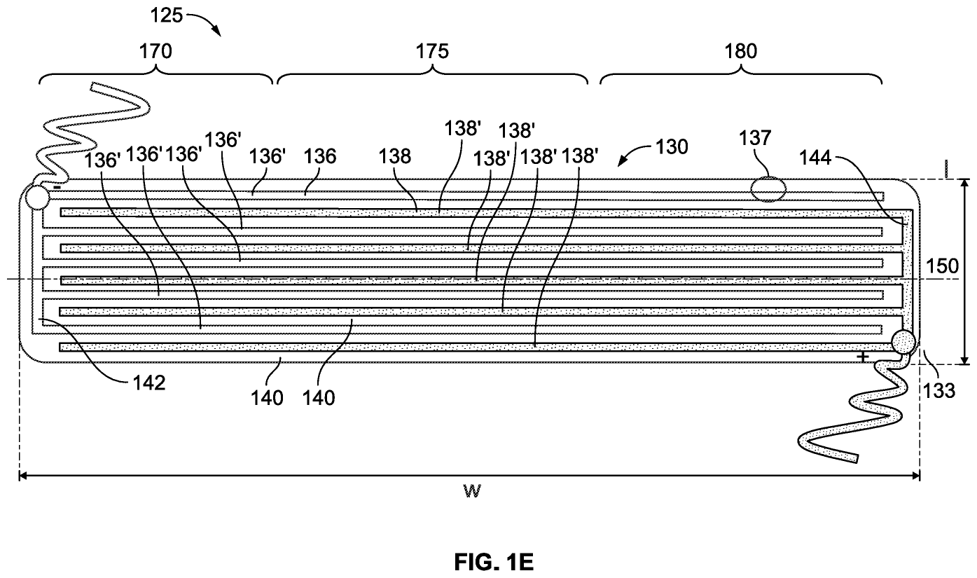

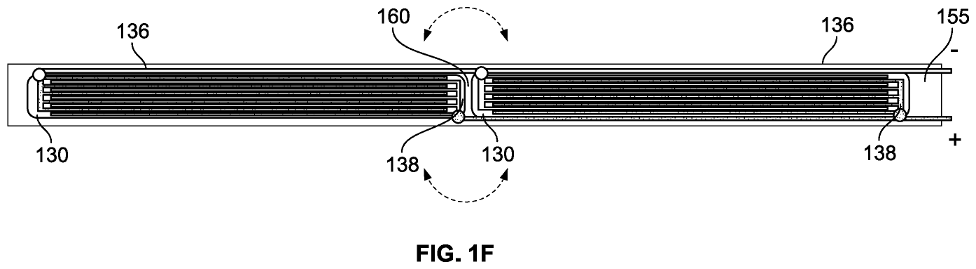

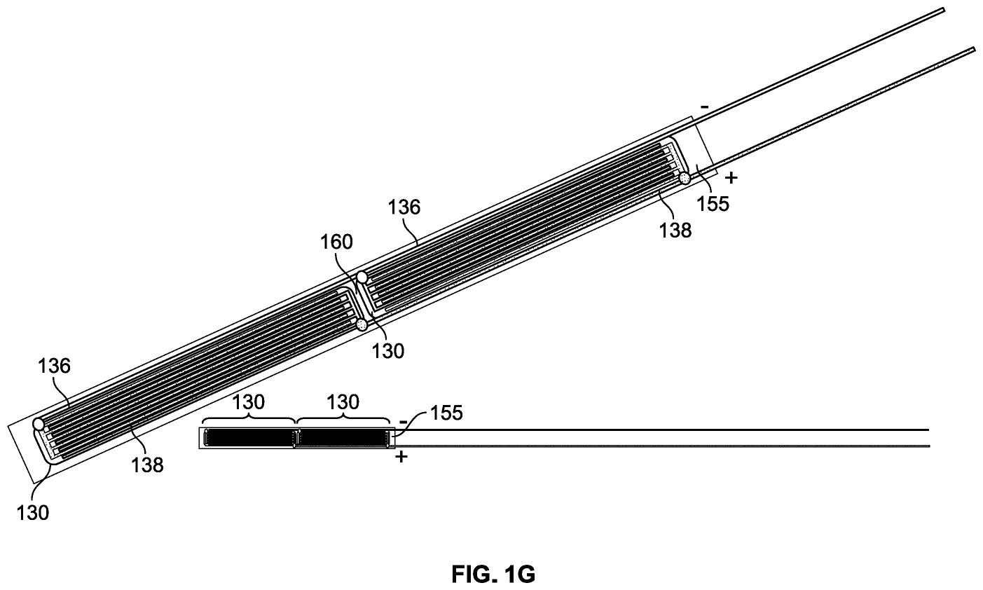

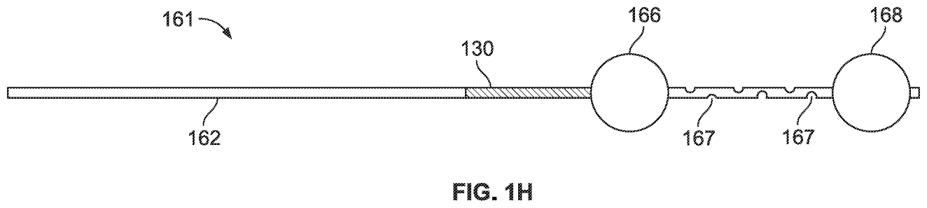

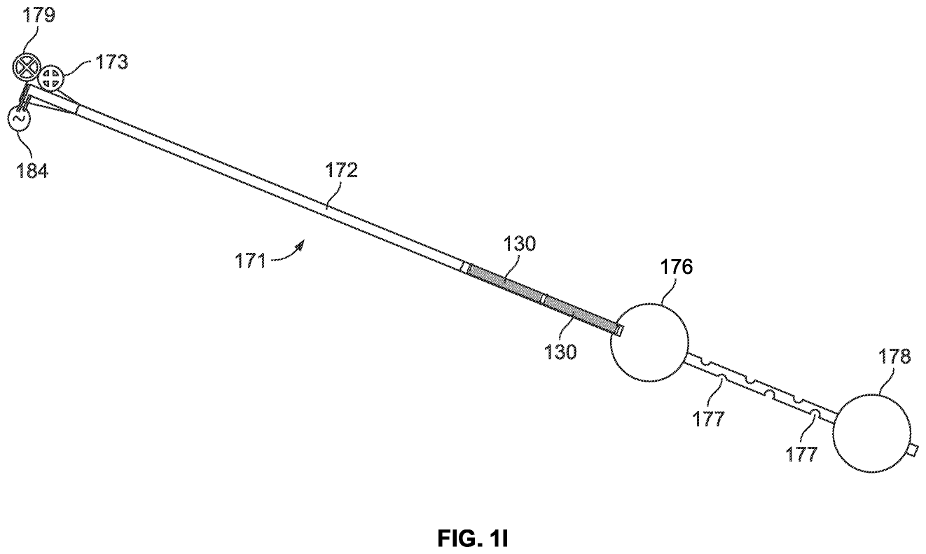

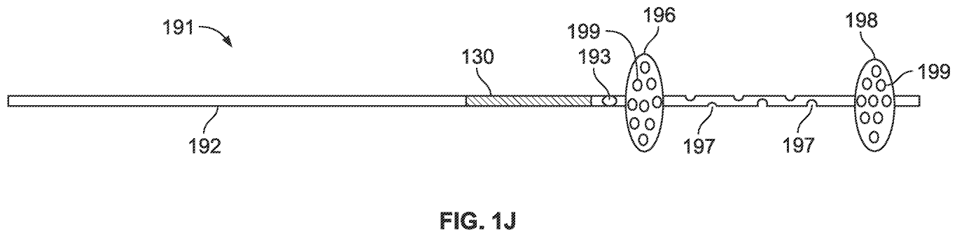

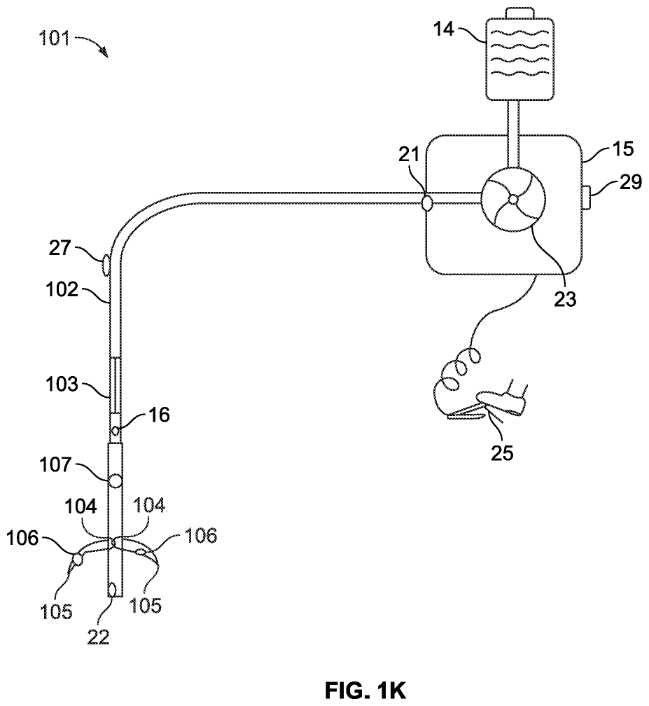

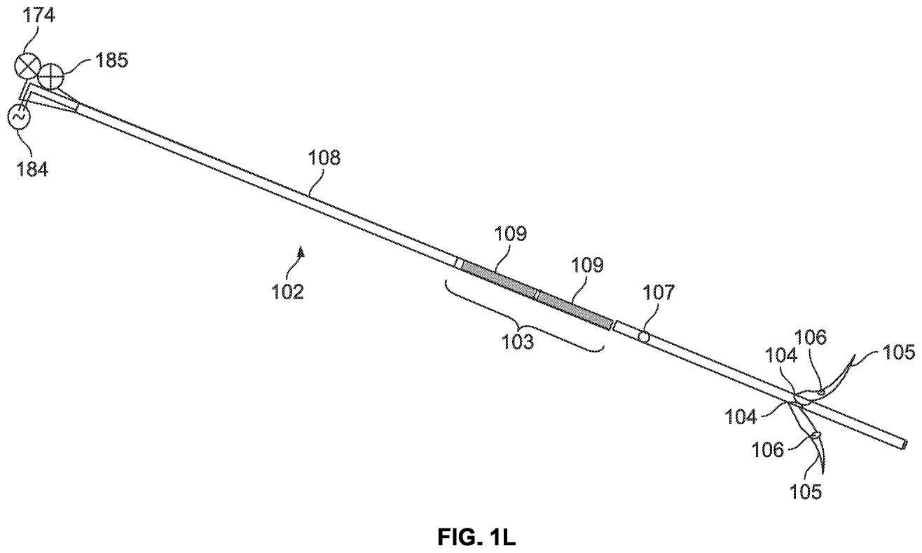

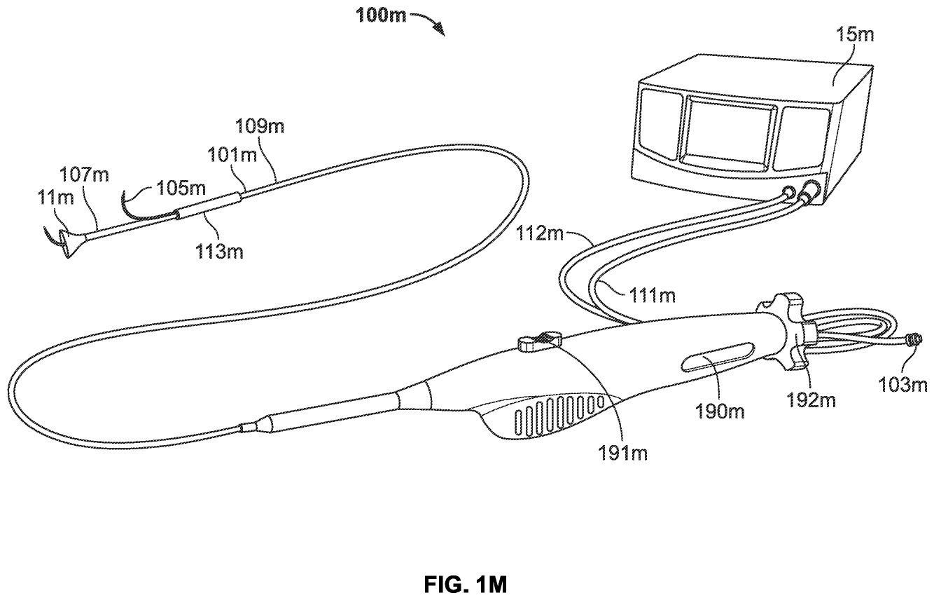

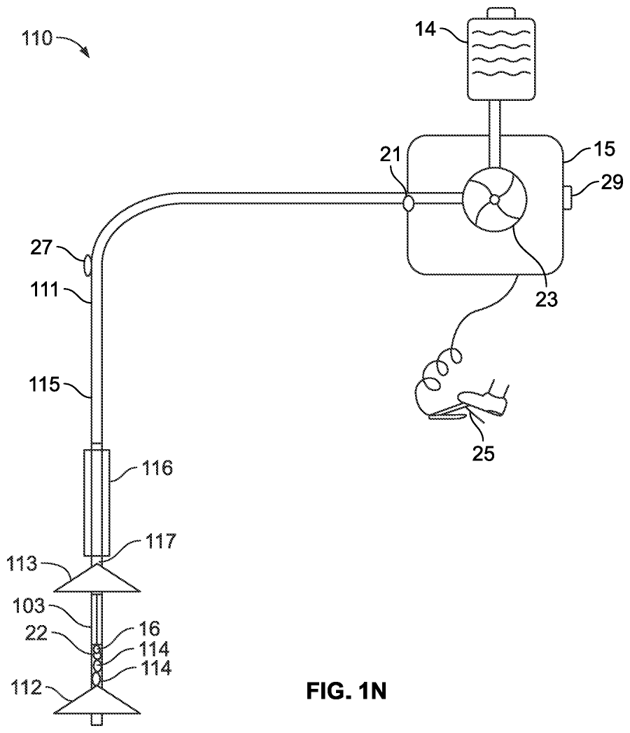

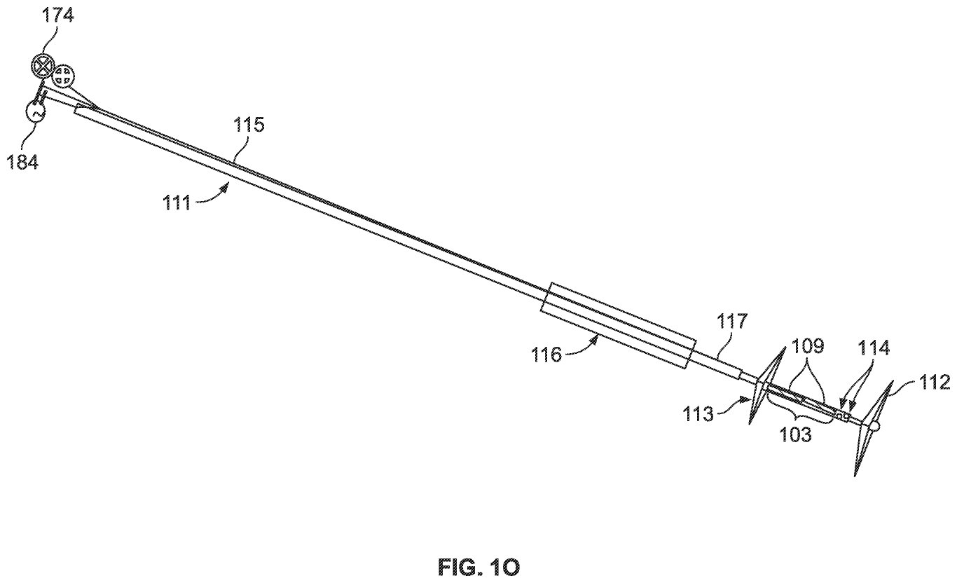

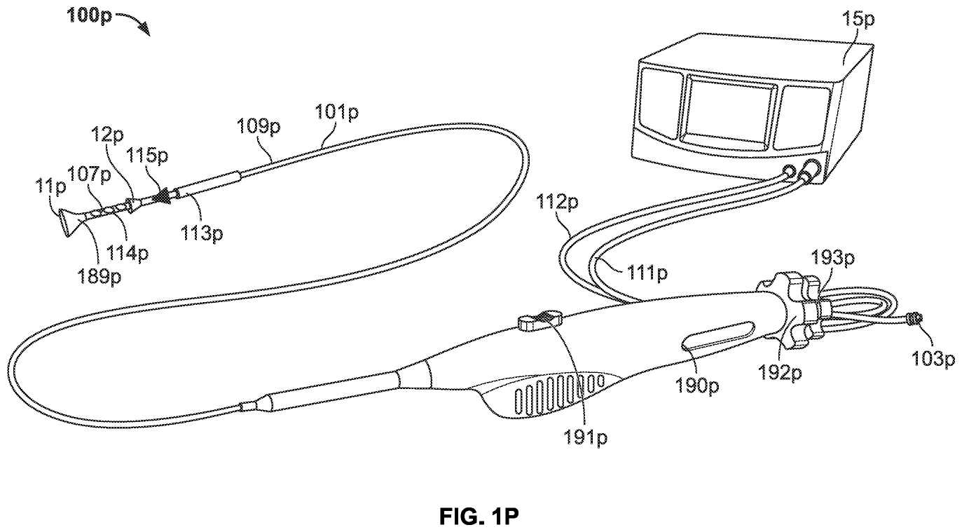

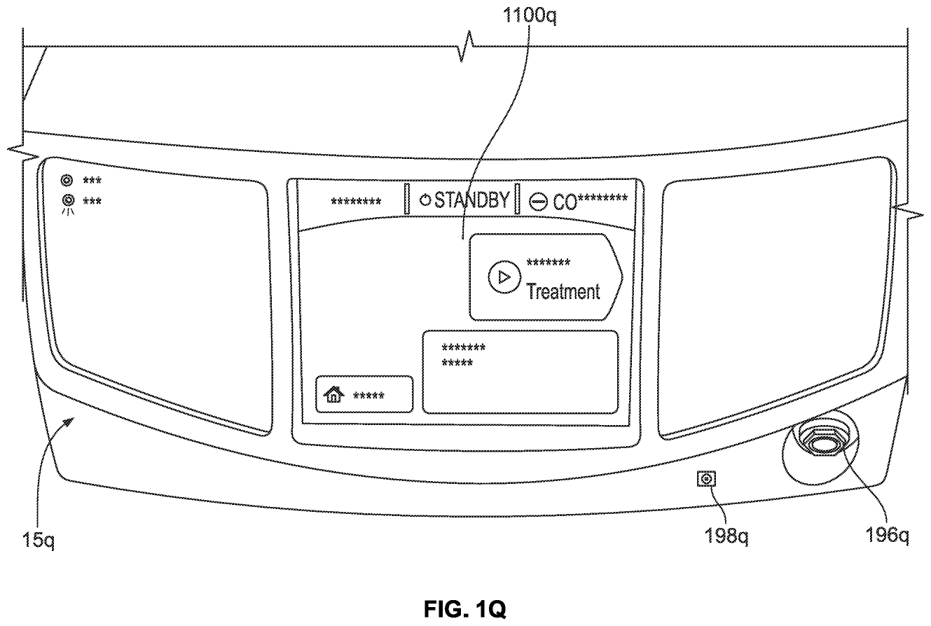

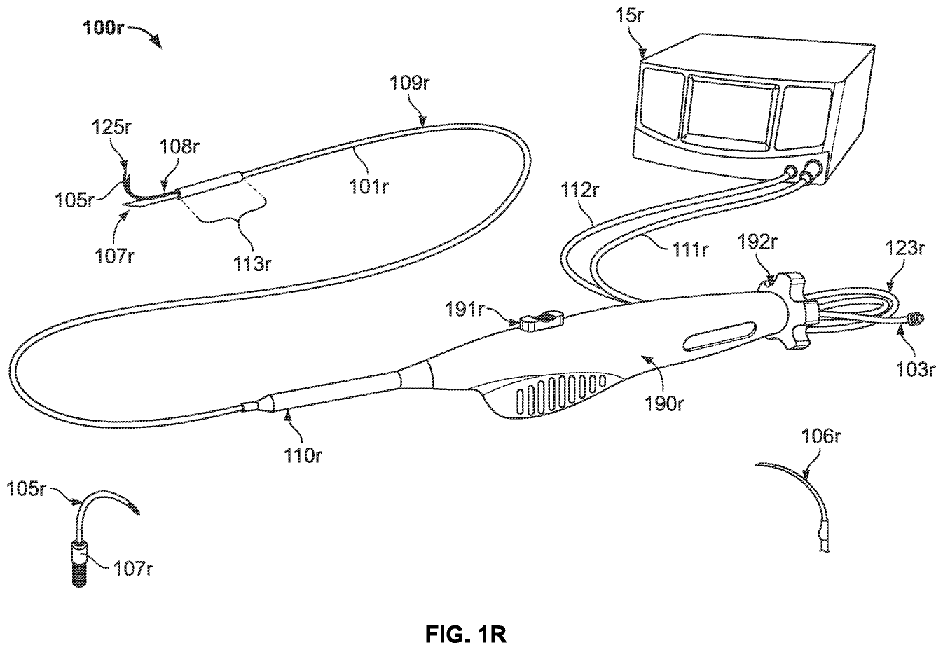

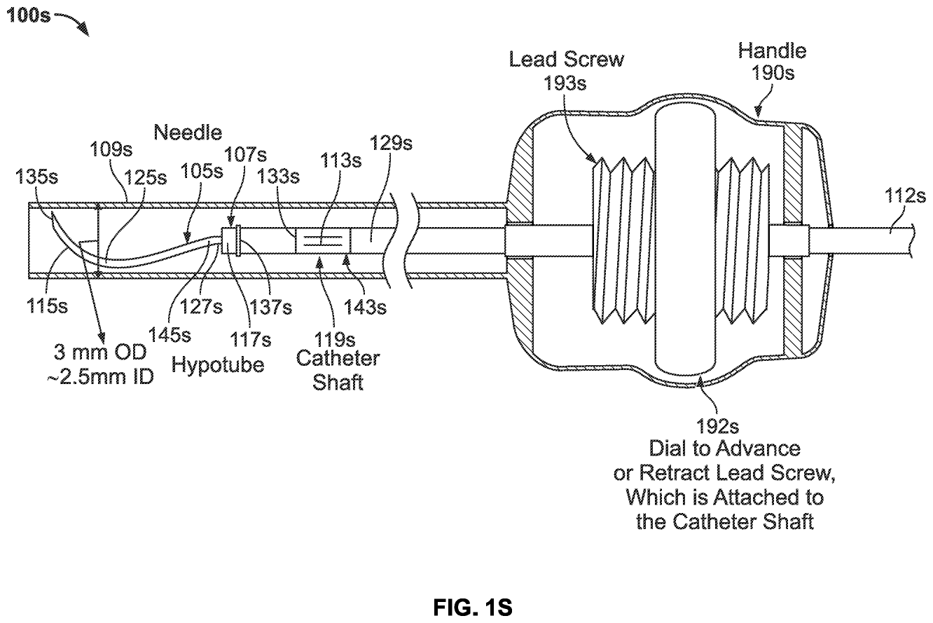

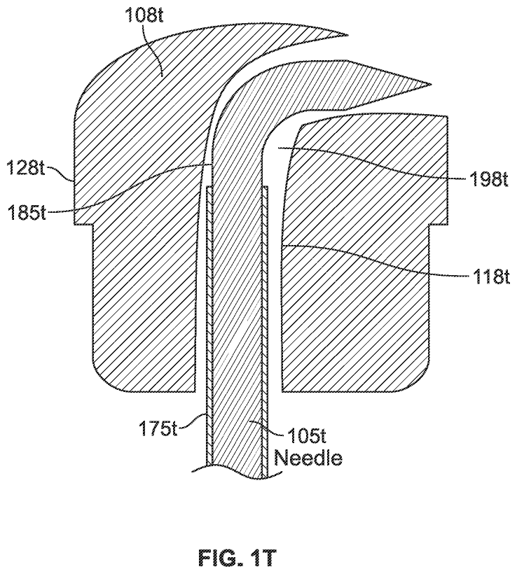

FIG. 1A illustrates an ablation system 100, in accordance with embodiments of the present specification. The ablation system comprises a catheter 10 having at least one first distal attachment or positioning element 11 and an internal heating chamber 18, disposed within a lumen of the catheter 10 and configured to heat a fluid provided to the catheter 10 to change said fluid to a vapor for ablation therapy. The internal heating chamber 18 comprises an electrode or an array of electrodes that are separated from thermally conductive element by a segment of the catheter 10 which is electrically non-conductive. In some embodiments, the catheter 10 is made of or covered with an insulated material to prevent the escape of ablative energy from the catheter body. The catheter 10 comprises one or more infusion ports 12 for the infusion of ablative agent, such as steam. In some embodiments, the one or more infusion ports 12 comprises a single infusion port at the distal end of a needle. In some embodiments, the catheter includes a second positioning element 13 proximal to the infusion ports 12. In various embodiments, the first distal attachment or positioning element 11 and second positioning element 13 may be any one of a disc, hood, cap, or inflatable balloon. In some embodiments the distal attachment or positioning element has a wire mesh structure with or without a covering membrane. In some embodiments, the first distal attachment or positioning element 11 and second positioning element 13 include pores 19 for the escape of air or ablative agent. A fluid, such as saline, is stored in a reservoir, such as a saline pump 14, connected to the catheter 10. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 17 monitors changes in an ablation area to guide flow of ablative agent. In some embodiments, optional sensor 17 comprises at least one of a temperature sensor or pressure sensor. In some embodiments, the catheter 10 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 10, or a switch 29 on the controller 15, for controlling vapor flow. In various embodiments, the switch 29 is positioned on the generator or the catheter handle.

In one embodiment, a user interface included with the controller 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

FIG. 1B is a transverse cross-section view 121 of a flexible heating chamber 130 configured to be incorporated at or into a distal portion or tip of a catheter, in accordance with an embodiment of the present specification. FIG. 1C illustrates a transverse cross-section view 122a and a longitudinal cross-section view 122b of a first array of electrodes 136 along with a transverse cross-section view 123a and a longitudinal cross-section view 123b of a second array of electrodes 138 of a flexible heating chamber for a catheter, in accordance with an embodiment of the present specification. FIGS. 1D and 1E are, respectively, transverse and longitudinal cross-section views 124, 125 of the heating chamber 130 including assembled first and second electrodes 136, 138.

Referring now to FIGS. 1B, 1C, 1D, and 1E simultaneously, the heating chamber 130 comprises an outer covering 132 and a coaxial inner core, channel, or lumen 134. A plurality of electrodes, configured as first and second arrays of electrodes 136, 138, is disposed between the outer covering 132 and the inner lumen 134. In some embodiments, the first and second array of electrodes 136, 138 respectively comprise metal rings 142, 144 from which a plurality of electrode fins or elements 136′, 138′ extend radially into the space between the outer covering 132 and insulative inner core/lumen 134 (see 122a, 123a). The electrode fins or elements 136′, 138′ also extend longitudinally along a longitudinal axis 150 of the heating chamber 130 (see 122b, 123b). In other words, each of the electrode fins 136′, 138′ have a first dimension along a radius of the heating chamber 130 and a second dimension along a longitudinal axis 150 of the heating chamber 130. The electrode fins or elements 136′, 138′ define a plurality of segmental spaces 140 there-between through which saline/water flows and is vaporized into steam. Electrical current is directed from the controller, into the catheter, through a lumen, and to the electrodes 136, 138 which causes the fins or elements 136′, 138′ to generate electrical charge which is then conducted through the saline in order to heat the saline and convert the saline to steam. The first and second dimensions enable the electrodes 136, 138 to have increased surface area for heating the saline/water flowing in the spaces 140. In accordance with an embodiment, the first electrodes 136 have a first polarity and the second electrodes 138 have a second polarity opposite said first polarity. In an embodiment, the first polarity is negative (cathode) while the second polarity is positive (anode).

In embodiments, the outer covering 132 and the inner core/lumen 134 are comprised of silicone, Teflon, ceramic or any other suitable thermoplastic/electrically insulative elastomer known to those of ordinary skill in the art. The inner core/lumen 134, outer covering 132, electrodes 136, 138 (including rings 142, 144 and fins or elements 136′, 138′) are all flexible to allow for bending of the distal portion or tip of the catheter to provide better positioning of the catheter during ablation procedures. In embodiments, the inner core/lumen 134 stabilizes the electrodes 136, 138 and maintains the separation or spacing 140 between the electrodes 136, 138 while the tip of the catheter flexes or bends during use preventing the electrodes from physically contacting one another and shorting out.

As shown in FIGS. 1D and 1E, when the heating chamber 130 is assembled, the electrode fins or elements 136′, 138′ interdigitate or interlock with each other (similar to fingers of two clasped hands) such that a cathode element is followed by an anode element which in turn is followed by a cathode element that is again followed by an anode element and so on, with a space 140 separating each cathode and anode element. In various embodiments, each space 140 has a distance from a cathode element to an anode element ranging from 0.01 mm to 2 mm. In some embodiments, the first array of electrodes 136 has a range of 1 to 50 electrode fins 136′, with a preferred number of 4 electrode fins 136′, while the second array of electrodes 138 has a range of 1 to 50 electrode fins 138′, with a preferred number of 4 electrode fins 138′. In various embodiments, the heating chamber 130 has a width w in a range of 1 to 5 mm and a length/in a range of 5 to 500 mm.

In accordance with an aspect of the present specification, multiple heating chambers 130 can be arranged in the catheter tip. FIGS. 1F and 1G are longitudinal cross-section views of a catheter tip 155 wherein two heating chambers 130 are arranged in series, in accordance with an embodiment of the present specification. Referring to FIGS. 1F and 1G, the two heating chambers 130 are arranged in series such that a space 160 between the two heating chambers 130 acts as a hinge to impart added flexibility to the catheter tip 155 to allow it to bend. The two heating chambers 130 respectively comprise interdigitated first and second arrays of electrodes 136, 138. Use of multiple, such as two or more, heating chambers 130 enables a further increase in the surface area of the electrodes 136, 138 while maintaining flexibility of the catheter tip 155.

Referring now to FIGS. 1B through 1G, for generating steam, fluid is delivered from a reservoir, such as a syringe, to the heating chamber 130 by a pump or any other pressurization means. In embodiments, the fluid is sterile saline or water that is delivered at a constant or variable fluid flow rate. An RF generator, connected to the heating chamber 130, provides power to the first and second arrays of electrodes 136, 138. As shown in FIG. 1E, during vapor generation, as the fluid flows through spaces 140 in the heating chamber 130 and power is applied to the electrodes 136, 138 causing the electrodes to charge which is conducted through the saline, resistively heating the saline and vaporizing the water in the saline. In other embodiments, conductive heating, convection heating, microwave heating, or inductive heating are used to convert the saline to vapor. The fluid is warmed in a first proximal region 170 of the heating chamber 130. When the fluid is heated to a sufficient temperature, such as 100 degrees Centigrade at atmospheric pressure, the fluid begins to transform into a vapor or steam in a second middle region 175. All of the fluid is transformed into vapor by the time it reaches a third distal region 180, after which it can exit a distal end 133 of the heating chamber 130 and exit the catheter tip 155. If the pressure in the heating chamber is greater than atmospheric pressure, higher temperatures will be required and if it is lower than atmospheric pressure, lower temperatures will generate vapor. When there is no saline flow through the chamber, the flow of the current through the chamber will be interrupted (dry electrode) and no heat will be generated. Measurement of the electrode impedance can be used to measure the flow of the saline and dry versus wet electrode.

In one embodiment, a sensor probe may be positioned at the distal end of the heating chambers within the catheter. During vapor generation, the sensor probe communicates a signal to the controller. The controller may use the signal to determine if the fluid has fully developed into vapor before exiting the distal end of the heating chamber. Sensing whether the saline has been fully converted into vapor may be particularly useful for many surgical applications, such as in the ablation of various tissues, where delivering high quality (low water content) steam results in more effective treatment. In some embodiments, the heating chamber includes at least one sensor 137. In various embodiments, said at least one sensor 137 comprises an impedance, temperature, pressure or flow sensor, with the pressure sensor being less preferred. In one embodiment, the electrical impedance of the electrode arrays 136, 138 can be sensed.

In other embodiments, the temperature of the fluid, temperature of the electrode arrays, fluid flow rate, pressure, or similar parameters can be sensed.

FIG. 1H and FIG. 1I illustrate multiple lumen balloon catheters 161 and 171 respectively, in accordance with embodiments of the present specification. The catheters 161, 171 each include an elongate body 162, 172 with a proximal end and a distal end. The catheters 161, 171 include at least one positioning element proximate their distal ends. In various embodiments, the positioning element is a balloon. In some embodiments, the catheters include more than one positioning element.

In the embodiments depicted in FIGS. 1H and 1I, the catheters 161, 171 each include a proximal balloon 166, 176 and a distal balloon 168, 178 positioned proximate the distal end of the body 162, 172 with a plurality of infusion ports 167, 177 located on the body 162, 172 between the two balloons 166, 176, and 168, 178. The body 162, 172 also includes at least one heating chamber 130 proximate and just proximal to the proximal balloon 166, 176. The embodiment of FIG. 1H illustrates one heating chamber 130 included in the body 165 proximate and just proximal to the proximal balloon 166. In some embodiments, multiple heating chambers are arranged in series in the body of the catheter.

In the embodiment of FIG. 1I, two heating chambers 130 are arranged in the body 172 proximate and just proximal to the proximal balloon 176. Referring to FIG. 1I, for inflating the balloons 176, 178 and providing electrical current and liquid to the catheter 171, a fluid pump 179, an air pump 173 and an RF generator 184 are coupled to the proximal end of the body 172. The air pump 173 pumps air via a first port through a first lumen (extending along a length of the body 172) to inflate the balloons 176, 178 so that the catheter 171 is held in position for an ablation treatment. In another embodiment, the catheter 171 includes an additional air-port and an additional air lumen so that the balloons 176, 178 may be inflated individually. The fluid pump 179 pumps the fluid through a second lumen (extending along the length of the body 172) to the heating chambers 130. The RF generator 184 supplies electrical current to the electrodes 136, 138 (FIGS. 1G, 1H), causing the electrodes 136, 138 to generate heat and thereby converting the fluid flowing through the heating chambers 130 into vapor. The generated vapor flows through the second lumen and exits the ports 177. The flexible heating chambers 130 impart improved flexibility and maneuverability to the catheters 161, 171, allowing a physician to better position the catheters 161, 171 when performing ablation procedures, such as ablating Barrett's esophagus tissue in an esophagus of a patient.

FIG. 1J illustrates a catheter 191 with proximal and distal positioning elements 196, 198 and an electrode heating chamber 130, in accordance with embodiments of the present specification. The catheter 191 includes an elongate body 192 with a proximal end and a distal end. The catheter 191 includes a proximal positioning element 196 and a distal positioning element 198 positioned proximate the distal end of the body 192 with a plurality of infusion ports 197 located on the body 192 between the two positioning elements 196, 198. The body 192 also includes at least one heating chamber 130 within a central lumen. In some embodiments, the proximal positioning element 196 and distal positioning element 198 comprises compressible discs which expand on deployment. In some embodiments, the proximal positioning element 196 and distal positioning element 198 are comprised of a shape memory metal and are transformable from a first, compressed configuration for delivery through a lumen of an endoscope and a second, expanded configuration for treatment. In embodiments, the discs include a plurality of pores 199 to allow for the escape of air at the start of an ablation procedure and for the escape of steam once the pressure and/or temperature within an enclosed treatment volume created between the two positioning elements 196, 198 reaches a predefined limit, as described above. In some embodiments, the catheter 191 includes a filter 193 with micro-pores which provides back pressure to the delivered steam, thereby pressurizing the steam. The predetermined size of micro-pores in the filter determine the backpressure and hence the temperature of the steam being generated.

It should be appreciated that the filter 193 may be any structure that permits the flow of vapor out of a port and restricts the flow of vapor back into, or upstream within, the catheter. Preferably, the filter is a thin porous metal or plastic structure, positioned in the catheter lumen and proximate one or more ports. Alternatively, a one-way valve may be used which permits vapor to flow out of a port but not back into the catheter. In one embodiment, this structure 193, which may be a filter, valve or porous structure, is positioned within 5 cm of a port, preferably in a range of 0.1 cm to 5 cm from a port, and more preferably within less than 1 cm from the port, which is defined as the actual opening through which vapor may flow out of the catheter and into the patient.

FIG. 1K illustrates an ablation system 101 suitable for use in ablating prostate tissue, in accordance with some embodiments of the present specification. The ablation system 101 comprises a catheter 102 having an internal heating chamber 103, disposed within a lumen of the catheter 102 and configured to heat a fluid provided to the catheter 102 to change said fluid to a vapor for ablation therapy. In one embodiment the fluid is electrically conductive saline and is converted into electrically non-conductive or poorly conductive vapor. In one embodiment, there is at least a 25% decrease in the conductivity, preferably a 50% decrease and more preferably a 90% decrease in the conductivity, of the fluid as determined by comparing the conductivity of the fluid, such as saline, prior to passing through the heating chamber to the conductivity of the ablative agent, such as steam, after passing through the heating chamber. It should further be appreciated that, for each of the embodiments disclosed in this specification, the term ablative agent preferably refers solely to the heated vapor, or steam, and the inherent heat energy stored therein, without any augmentation from any other energy source, including a radio frequency, electrical, ultrasonic, optical, or other energy modality.

In some embodiments, the catheter 102 is made of or covered with an insulated material to prevent the escape of ablative energy from the catheter body. A plurality of openings 104 are located proximate the distal end of the catheter 102 for enabling a plurality of associated thermally conductive elements, such as needles 105, to be extended (at an angle from the catheter 102, wherein the angle ranges between 30 to 90 degrees) and deployed or retracted through the plurality of openings 104. In accordance with an aspect, the plurality of retractable needles 105 are hollow and include at least one infusion port 106 to allow delivery of an ablative agent, such as steam or vapor, through the needles 105 when the needles 105 are extended and deployed through the plurality of openings 104 on the elongated body of the catheter 102. In some embodiments, the infusion ports are positioned along a length of the needles 105. In some embodiments, the infusion ports 106 are positioned at a distal tip of the needles 105. During use, cooling fluid such as water, air, or CO₂ is circulated through an optional port 107 to cool the catheter 102. Vapor for ablation and cooling fluid for cooling are supplied to the catheter 102 at its proximal end. A fluid, such as saline, is stored in a reservoir, such as a saline pump 14, connected to the catheter 102. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 22 monitors changes in an ablation area to guide flow of ablative agent. In some embodiments, the optional sensor comprises at least one of a temperature sensor or pressure sensor. In some embodiments, the catheter 102 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 102, or a switch 29 on the controller 15, for controlling vapor flow. In some embodiment, the needles have attached mechanism to change their direction from being relatively parallel to the catheter to being at an angle between 30°-90° to the catheter. In one embodiment, the aforementioned mechanism is a pull wire. In some embodiments, the openings in the catheter are shaped to change the direction of the needle from being relatively parallel to the catheter to being at an angle between 30°-90° to the catheter.

In one embodiment, a user interface included with the microprocessor 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

FIG. 1L illustrates another view of a catheter 102 of FIG. 1K, in accordance with some embodiments of the present specification. The catheter 102 includes an elongate body 108 with a proximal end and a distal end. A plurality of openings 104 are located proximate the distal end of the catheter 102 for enabling a plurality of associated thermally conductive elements, such as needles 105, to be extended (at an angle from the catheter 102, wherein the angle ranges between 10 to 90 degrees) and deployed or retracted through the plurality of openings 104. In accordance with an aspect, the plurality of retractable needles 105 are hollow and include at least one infusion port 106 to allow delivery of an ablative agent, such as steam or vapor, through the needles 105 when the needles 105 are extended and deployed through the plurality of openings 104 on the elongated body of the catheter 102. In some embodiments, the infusion ports are positioned along a length of the needles 105. In some embodiments, the infusion ports 106 are positioned at a distal tip of the needles 105. Optionally, during use, cooling fluid, such as water, air, or CO₂ is circulated through an optional port 107 to cool the catheter 102. The body 108 includes at least one heating chamber 103 proximate and just proximal to the optional port 107 or openings 104. In embodiments, the heating chamber 103 comprises two electrodes 109 configured to receive RF current, heat, and convert supplied fluid, such as saline, to vapor or steam, for ablation.

Referring to FIG. 1L, for providing electrical current, fluid for ablation, and optional cooling fluid to the catheter 102, an RF generator 184, a first fluid pump 174, and a second fluid pump 185 are coupled to the proximal end of the body 108. The first fluid pump 174 pumps a first fluid, such as saline, through a first lumen (extending along the length of the body 108) to the heating chamber 103. The RF generator 184 supplies electrical current to the electrodes 109, causing the electrodes 109 to generate heat and thereby converting the fluid flowing through the heating chamber 103 into vapor. The generated vapor flows through the first lumen, openings 104, needles 105, and exits the infusion ports 106 to ablate prostatic tissue. Optionally, in some embodiments, a second fluid pump 185 pumps a second fluid, such as water, through a second lumen (extending along a length of the body 108) to optional port 107, where the second fluid exits the catheter 102 to circulate in and cool the area of ablation. The flexible heating chamber 103 imparts improved flexibility and maneuverability to the catheter 102, allowing a physician to better position the catheter 102 when performing ablation procedures, such as ablating prostate tissue of a patient.

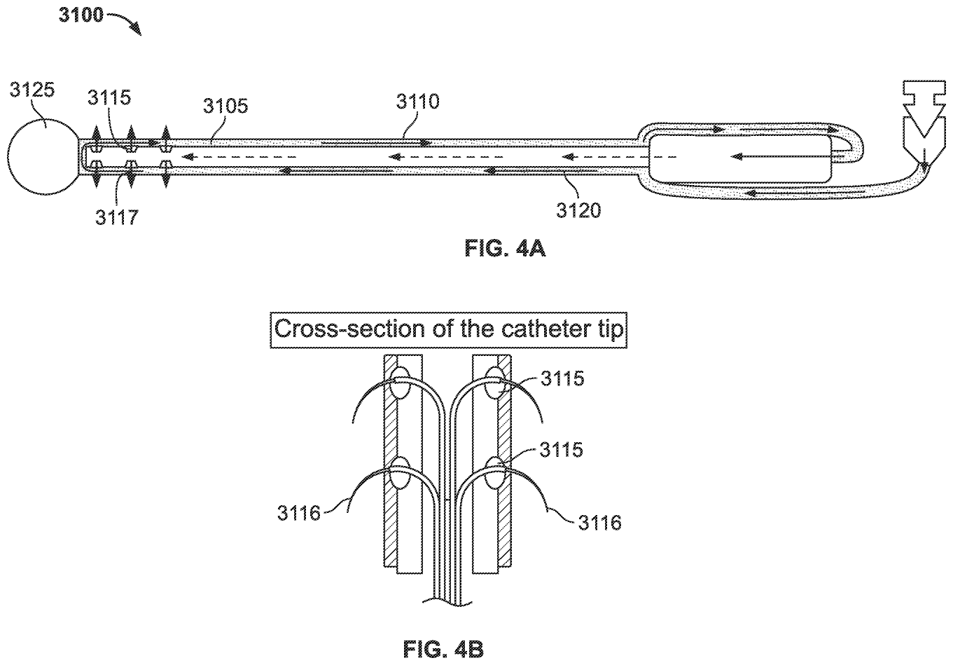

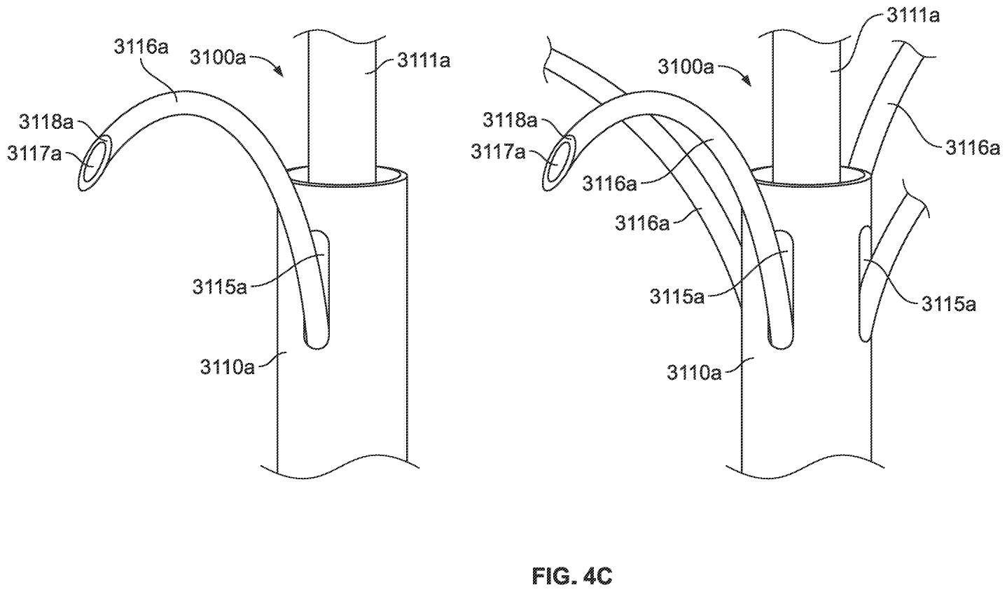

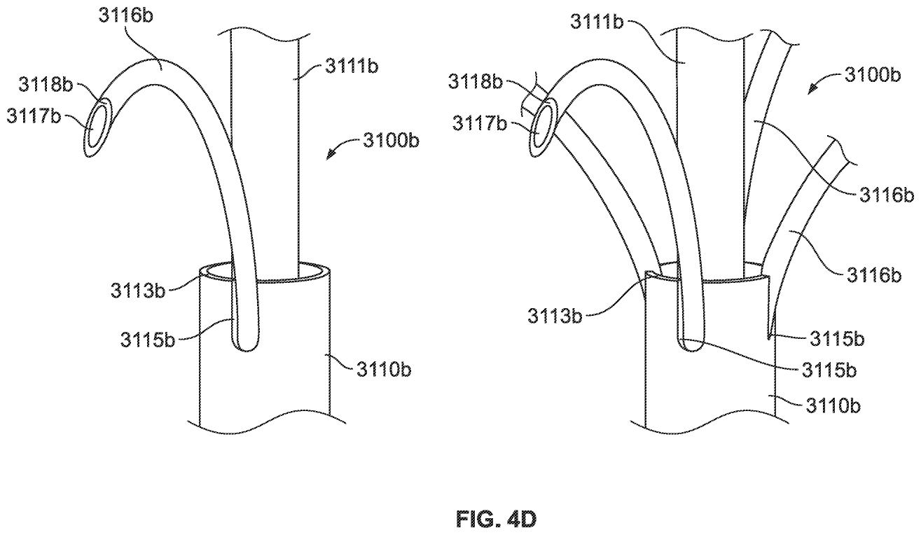

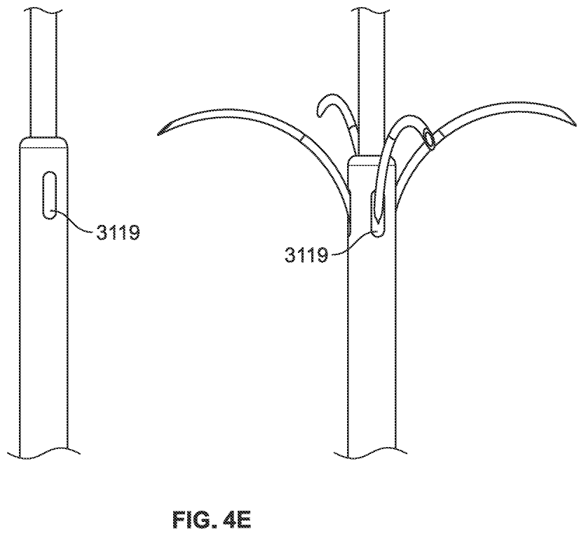

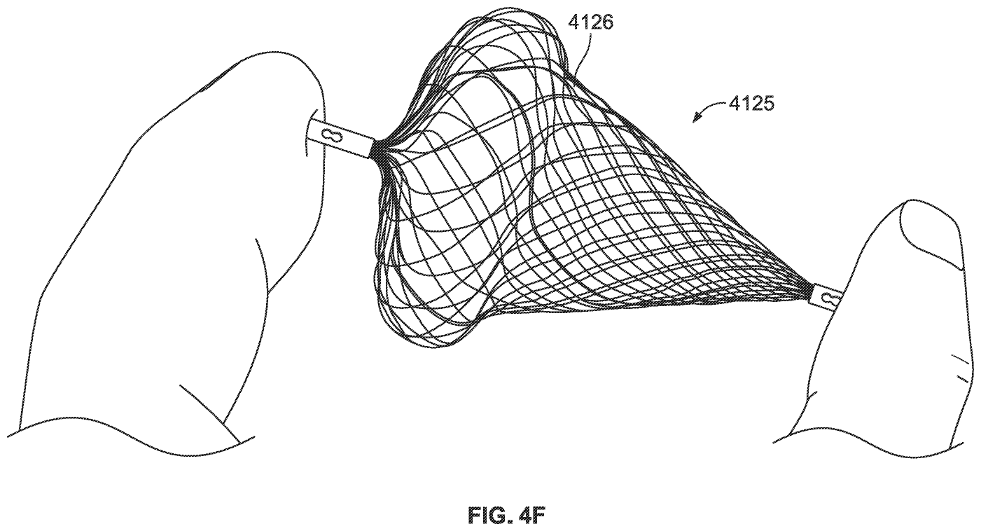

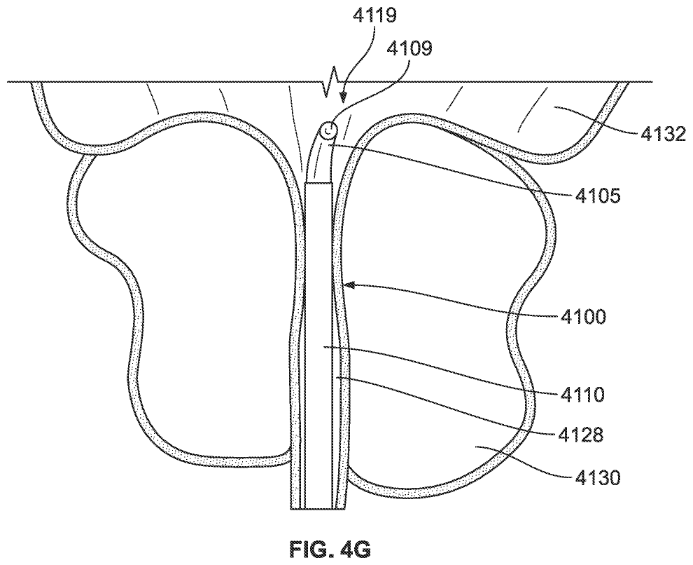

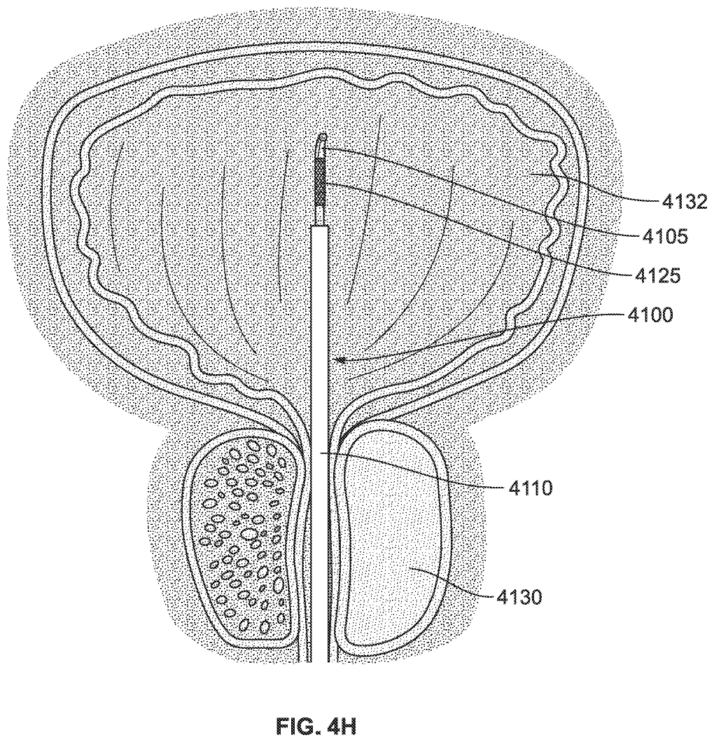

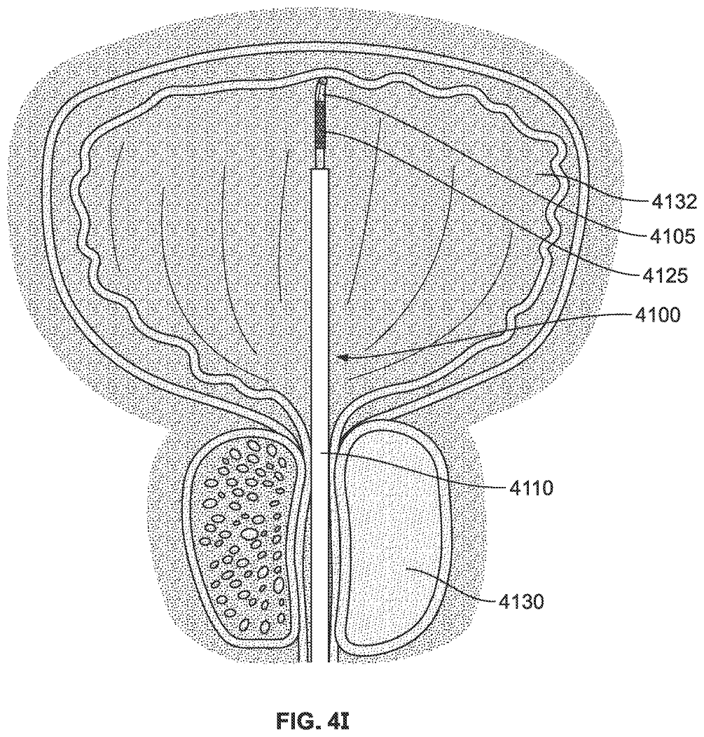

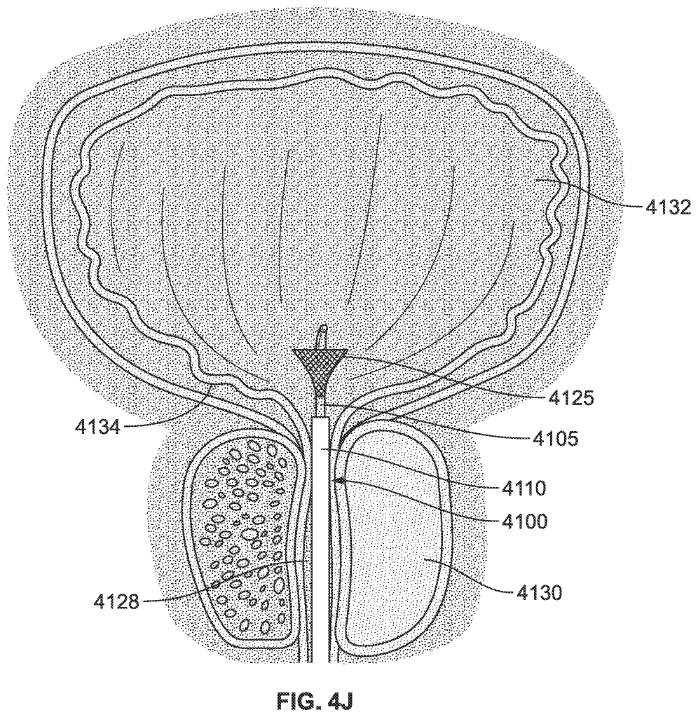

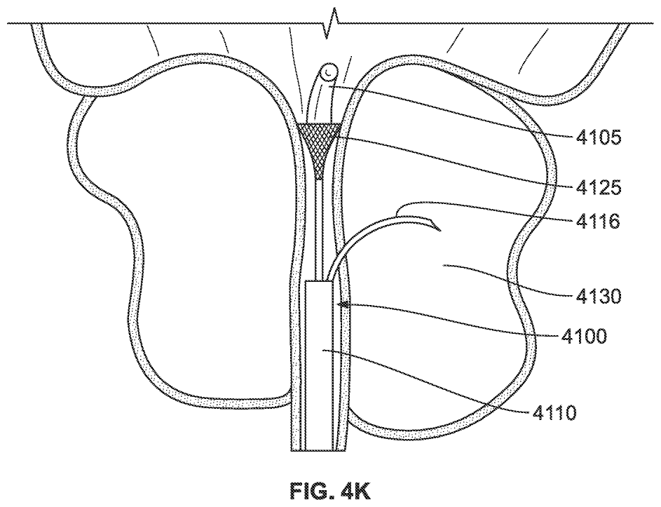

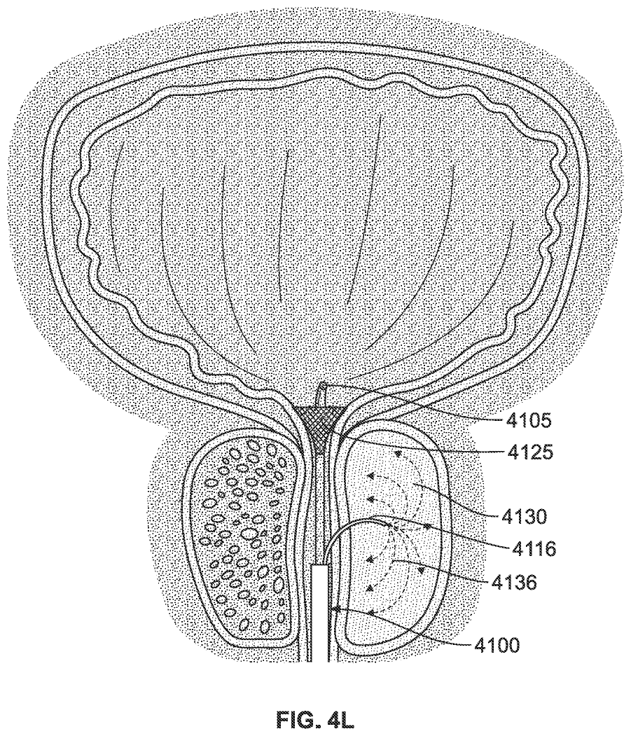

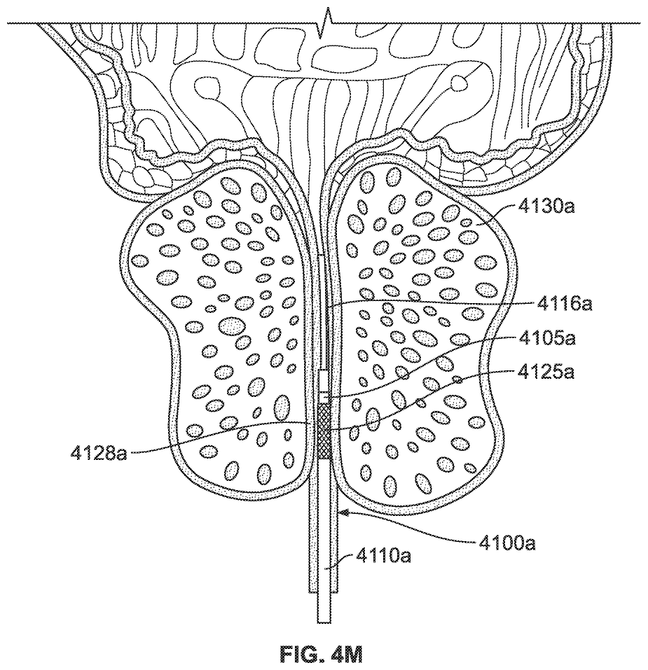

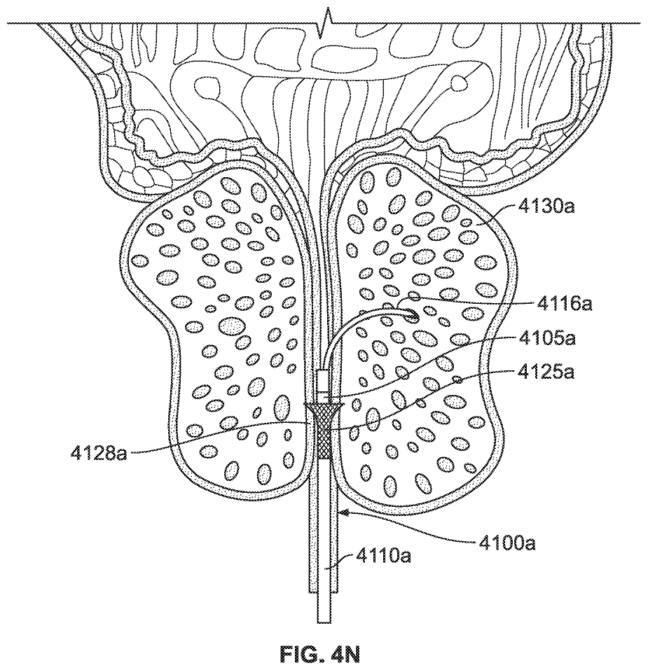

FIG. 1M illustrates a system 100m for use in the ablation of prostatic tissue, in accordance with another embodiment of the present specification. The system 100m comprises a catheter 101m which, in some embodiments, includes a handle 190m having actuators 191m, 192m for extending at least one needle 105m or a plurality of needles from a distal end of the catheter 101m and expanding a positioning element 11m at a distal end of the catheter 101m. In some embodiments, actuators 191m and 192m may be one of a knob or a slide or any other type of switch or button to enable extending of the at least one needle 105m or plurality of needles. Delivery of vapor via the catheter 101m is controlled by a controller 15m. In embodiments, the catheter 101m comprises an outer sheath 109m and an inner catheter 107m. The needle 105m extends from the inner catheter 107m at the distal end of the sheath 109m or, in some embodiments, through openings proximate the distal end of the sheath 109m. In embodiments, the positioning element 11m is expandable, positioned at the distal end of the inner catheter 107m, and may be compressed within the outer sheath 109m for delivery. In some embodiments, actuator 191m comprises a knob which is turned by a first extent, for example, by a quarter turn, to pull back the outer sheath 109m. As the outer sheath 109m retracts, the positioning element 11m is revealed. In embodiments, the positioning element 11m comprises a disc or cone configured as a bladder anchor. In embodiments, actuator/knob is turned by a second extend, for example, by a second quarter turn, to pull back the outer sheath further 109m to deploy the needle 105m. In some embodiments, the number of needles that is deployed is two or more than two. In some embodiments, referring to FIGS. 1M, 4C and 4E simultaneously, the needle or needles 105m, 3116a are deployed out of an internal lumen of the inner catheter 107m, 3111a through slots or openings 3115a in the outer sheath 109m, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

Referring again to FIG. 1M, in some embodiments, the catheter 101m includes a port 103m for the delivery of fluid, for example cooling fluid, during ablation. In some embodiments, port 103m is also configured to provide for fluid collection, provide vacuum, and provide CO₂ for an integrity test. In some embodiments, the port 103m is positioned on the handle 190m. In some embodiments, at least one electrode 113m is positioned at a distal end of the catheter 101m proximal to the needles 105m. The electrode 113m is configured to receive electrical current, supplied by a connecting wire 111m extending from the controller 15m to the catheter 101m, to heat and convert a fluid, such as saline supplied via tubing 112m extending from the controller 15m to the catheter 101m. Heated fluid or saline is converted to vapor or steam to be delivered by needle 105m for ablation.

FIG. 1R illustrates a system 100r for use in the ablation of prostatic tissue, in accordance with another embodiment of the present specification. The system 100r comprises a catheter 101r which, in some embodiments, includes a handle 190r having actuators 191r, 192r for extending at least one needle 105r or a plurality of needles from a distal end of the catheter 101r. A drive mechanism configured within the handle 190r deploys and retracts the needle 105r in and out of a tip of the catheter shaft 101r. In some embodiments, actuators 191r and 192r may be one of a knob or a slide or any other type of switch or button to enable extending of the at least one needle 105r or plurality of needles. In some embodiments, actuator 191r is a button or a switch that allows a physician to activate treatment using system 100r from the handle 190r as well as a foot pedal (not shown). In some embodiments, a strain relief mechanism 110r is configured at a distal end of the handle 190r that connects the handle 190r to the catheter 101r. The strain relief mechanism 110r provides support to the catheter shaft 101r. Delivery of vapor via the catheter 101r is controlled by a controller 15r. A cable sub-assembly 123r including an electrical cable, in the handle 190r, connects the catheter 101r to the controller 15r. In embodiments, the catheter 101r comprises an outer sheath 109r and an inner catheter (not shown).

In various embodiments, the controller 15r (and 15, 15m, 15p, 15q, and 2252 of FIGS. 1A, 1K, & 1N, 1M, 1P, 1Q, and 22B respectively) of the systems of the present specification comprises a computing device having one or more processors or central processing units, one or more computer-readable storage media such as RAM, hard disk, or any other optical or magnetic media, a controller such as an input/output controller, at least one communication interface and a system memory. The system memory includes at least one random access memory (RAM) and at least one read-only memory (ROM). In embodiments, the memory includes a database for storing raw data, images, and data related to these images. The plurality of functional and operational elements is in communication with the central processing unit (CPU) to enable operation of the computing device. In various embodiments, the computing device may be a conventional standalone computer or alternatively, the functions of the computing device may be distributed across a network of multiple computer systems and architectures and/or a cloud computing system. In some embodiments, execution of a plurality of sequences of programmatic instructions or code, which are stored in one or more non-volatile memories, enable or cause the CPU of the computing device to perform various functions and processes as described in the present specification. In alternate embodiments, hard-wired circuitry may be used in place of, or in combination with, software instructions for implementation of the processes of systems and methods described in this application. Thus, the systems and methods described are not limited to any specific combination of hardware and software.

A needle tip assembly 125r is positioned within a needle chamber 108r, within the outer sheath 109r. The needle chamber 108r may be a metal or plastic sleeve that is configured to house the needle 105r during delivery to assist in needle deployment and retraction and is further described with reference to FIG. 1T. The needle tip assembly 125r, including the needle 105r, extends from the inner catheter when pushed out of its chamber 108r, at the distal end of the sheath 109r or, in some embodiments, through openings proximate the distal end of the sheath 109r. In embodiments, a positioning element is also provided at the distal end of the inner catheter. The positioning element may be expandable, and may be compressed within the outer sheath 109r for delivery. In some embodiments, actuator 192r comprises a knob which is turned by a first extent, for example, by a quarter turn, to pull back the outer sheath 109r. As the outer sheath 109r retracts, the positioning element is revealed. In embodiments, actuator/knob 192r is turned by a second extend, for example, by a second quarter turn, to pull back the outer sheath 109r further to deploy the needle 105r. In some embodiments, the number of needles that is deployed is two or more than two. In some embodiments, referring to FIGS. 1R, 4C and 4E simultaneously, the needle or needles 105r, 3116a are deployed out of an internal lumen of the inner catheter 3111a through slots or openings 3115a in the outer sheath 109r, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

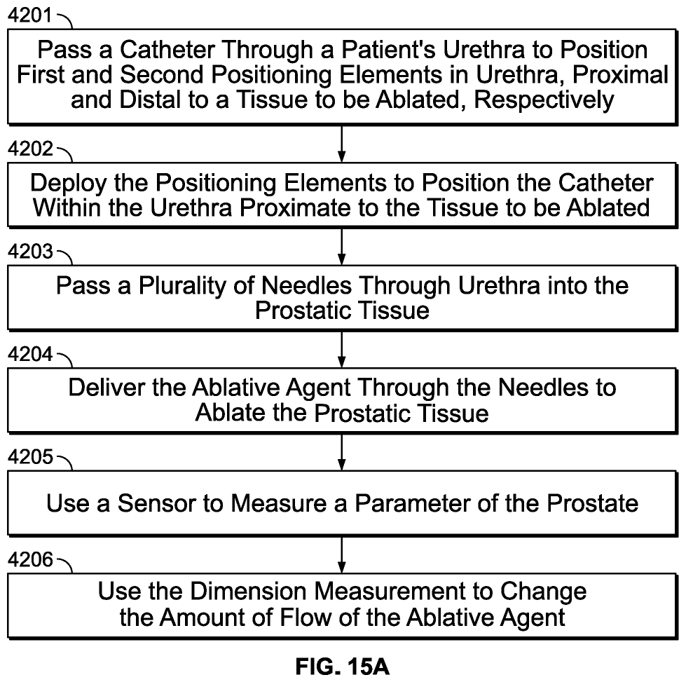

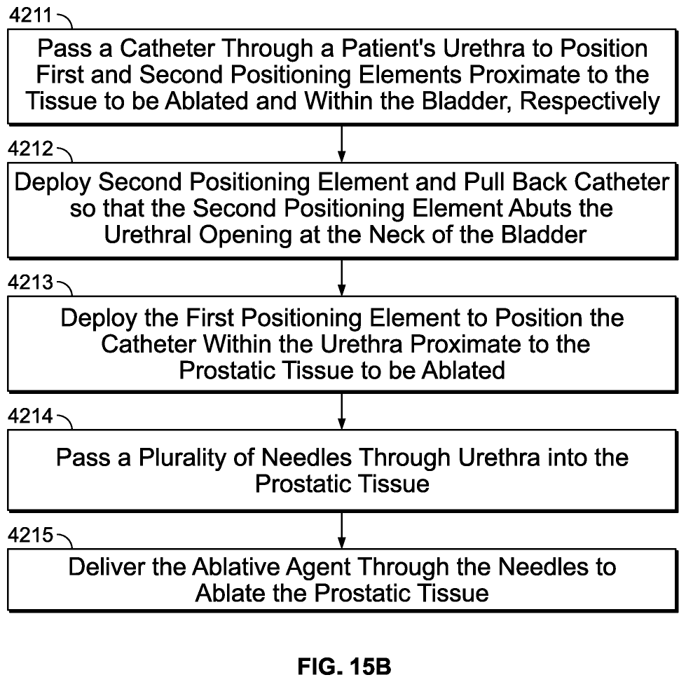

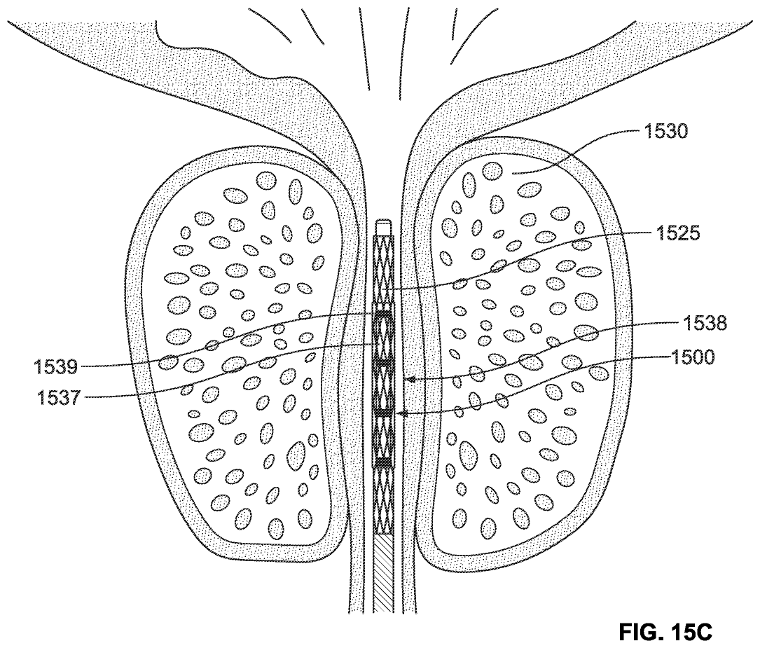

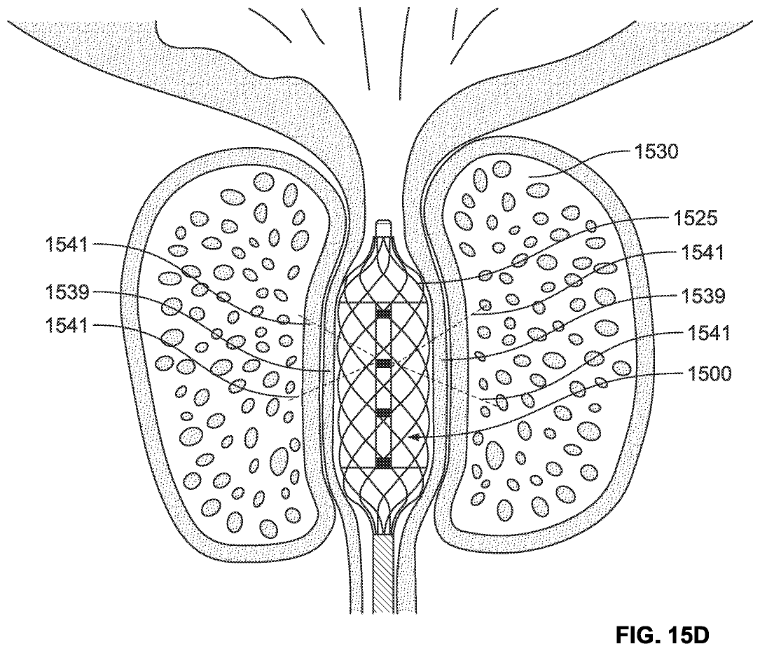

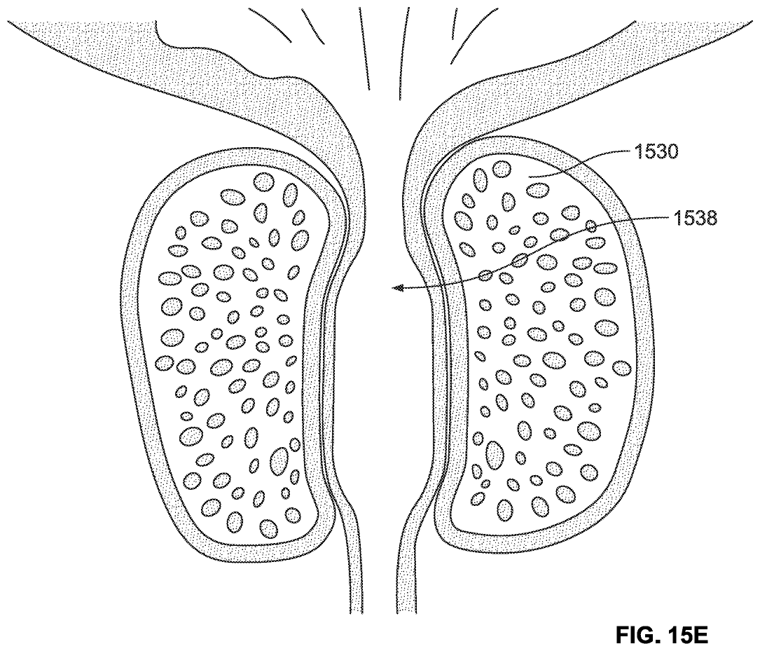

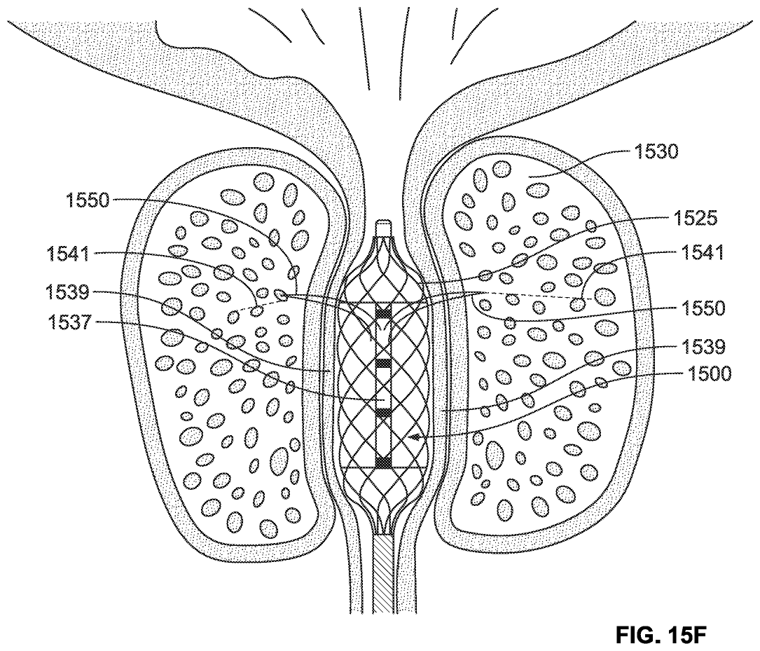

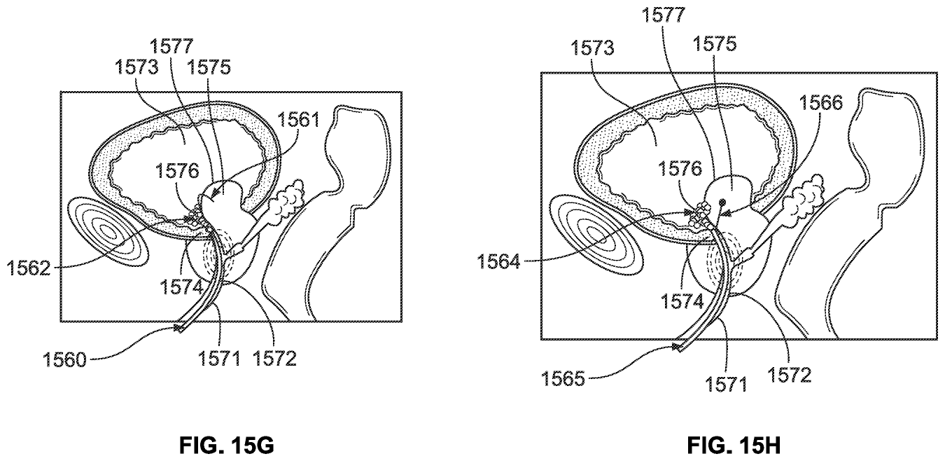

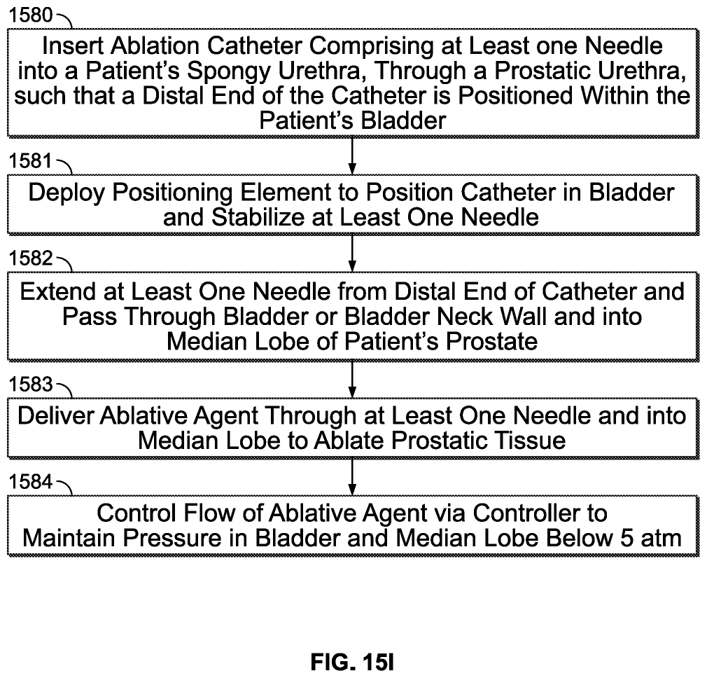

FIG. 1R illustrates an expanded view of a needle tip assembly 125r, which includes a needle 105r attached to a needle attachment component 107r which, in some embodiments, comprises a metal threaded fitting, and is described in further detail with reference to FIG. 15. The needle attachment component, or threaded fitting, 107r connects the needle 105r to the catheter 101r. In embodiments, the needle attachment component 107r comprises a threaded surface fixedly attached to a tip of the catheter 101r and configured to have a needle 105r screwed thereto. In some embodiments, the needle 105r is a 22 to 25 G needle. In some embodiments, needle 105r has a gradient of coating for insulation or echogenicity. An insulation coating 106r may be ceramic, polymer, or any other material suitable for coating the needle 105r and providing insulation and/or echogenicity to the needle 105r. The coatings are provided at the base of the needle 105r to varying lengths to the needle tip.

Referring again to FIG. 1R, in some embodiments, the catheter 101r includes a tubing and connector sub-assembly (port) 103r for the delivery of fluid, for example cooling fluid, during ablation. In some embodiments, port 103r is also configured to provide for fluid collection, provide vacuum, and provide CO₂ for an integrity test. In some embodiments, the port 103r is positioned on the handle 190r. In some embodiments, one or more electrodes 113r is positioned at a distal end of the catheter 101r proximal to the one or more needles 105r. The one or more electrodes 113r is configured to receive electrical current, supplied by a connecting wire 111r extending from the controller 15r to the catheter 101r, to heat and convert a fluid, such as saline, supplied via tubing 112r extending from the controller 15r to the catheter 101r. Heated fluid or saline is converted to vapor or steam to be delivered by needle 105r for ablation.

FIG. 15 illustrates a needle attachment component 107s of a system 100s for use in the ablation of prostatic tissue, in accordance with some embodiments of the present specification. In a preferred embodiment, a needle attachment component 107s, having a lumen 117s defining an internal cavity, is fixedly attached to the end of the inner catheter 119s such that the lumen 129s of the inner catheter 119s is in fluid communication with the lumen 117s of the needle attachment component 107s. Preferably the needle attachment component 107s has, on its distal external surface 127s, a plurality of threads on to which a needle 105s may be screwed. Also preferably, the needle attachment component 107s is made of the same material as the needle 105s, preferably metal and more preferably stainless steel.

It is important for the proximal portion 137s of the needle attachment component 107s to be spaced in a very specific range from the one or more electrodes 113s. Too close and the electricity from the electrode(s) 113s may flow into the needle attachment component 107s, into the needle 105s, and to tissue of the patient. Too far and the vapor generated by the electrode(s) 113s may heat the length of the inner catheter 119s and outer catheter 109s between the electrode 113s and the needle attachment component 107s, exposing tissue that should not be ablated to excessive heat, possibly causing strictures, and also causing vapor to prematurely condense before passing through the needle 105s, therefore resulting in an insufficient amount of vapor reaching the tissue to be ablated. Therefore, in a preferred embodiment, the most distal electrode 133s of the plurality of electrodes 113s positioned within the catheter lumen 129s is separated by the most proximal part 137s of the needle attachment component 107s by a distance of at least 0.1 mm to a distance of no more than 60 mm. These distance ranges insure that a) no electricity will be communicated to the tissue along with, or independent of, the vapor, b) that sufficient amounts of vapor will be communicated to the tissue to be ablated, and c) that the distance separating the point of generation of vapor and the needle attachment component 107s is small, thereby insuring the associated catheter length is not excessively heated and that the tissue contacting the catheter length is not unduly ablated.

The needle 105s is defined by a metal casing 115s, a lumen passing therethrough 125s, a sharp, and preferably tapered, tip 135s, and a proximal base 145s configured to thread, or otherwise attach to, the needle attachment component 107s. The needle 105s is also curved in a first direction that extends away from the axial length of the catheter 109s. In one embodiment, the needle 105s has a bending capacity that changes depending on the direction of bending. For example, the needle 150s may be more easily bent in a plane parallel to the first direction as opposed to a plane perpendicular to the first direction. Optionally, the needle 105s may be more easily bent in a plane perpendicular to the first direction as opposed to a plane parallel to the first direction. Optionally, a housing 143s of the electrode 113s may also be more easily bent in one direction versus another direction. For example, the electrode housing 143s may also be more easily bent in a plane perpendicular to the first direction as opposed to a plane parallel to the first direction. Optionally, the electrode housing 143s may also be more easily bent in a plane parallel to the first direction as opposed to a plane perpendicular to the first direction. In embodiments, a length of tubing 112s at a proximal end of the catheter handle 190s provides saline to the catheter for conversion to vapor. In embodiments, a dial 192s on the handle 190s may be turned by a user to advance or retract a lead screw 193s attached to the inner catheter 119s to expose or retract the needle 105s from the outer catheter 109s. In some embodiments, the outer catheter 109s comprises a hypo-tube having an outer diameter of 3 mm and an inner diameter of approximately 2.5 mm. In some embodiments, the needle 105s is a 25 gauge needle.

FIG. 1T illustrates a needle chamber 108t of a system for use in the ablation of prostatic tissue, in accordance with some embodiments of the present specification. In embodiments, the catheter further comprises a retractable needle chamber 108t configured to be positioned over the needle 105t and needle attachment component (107s in FIG. 1S). The needle chamber 108t may be retracted using a control on the handle and, once retracted, will expose the needle 105t. To ensure the needle 105t maintains the right radius, degree or extent of curvature, in operation, preferably the needle 105t, pre-deployment and before being positioned in the needle chamber 108t, has a first radius, degree or extent of curvature. Before being positioned in a patient, the needle 105t, having the first radius, degree or extent of curvature, is encased within, and covered by, the needle chamber 108t, resulting in the needle 105t adopting a second radius, degree or extent of curvature. Finally, in use and when inside the patient, the needle chamber 108t may be retracted to expose the needle. Upon doing so, the needle 105t would adopt a third radius, degree or extent of curvature. In this embodiment, the first radius, degree or extent of curvature is greater than the third radius, degree or extent of curvature which is greater than the second radius, degree or extent of curvature. Stated differently, the first radius, degree or extent of curvature is the largest, the third radius, degree or extent of curvature is the smallest and the second radius, degree or extent of curvature is in between the two.

The needle chamber 108t is preferably cylindrical having an internal surface 118t with a higher degree of hardness or stiffness relative to its outside surface 128t. Preferably, the outside surface 128t is made of a polymer while the inside surface 118t comprises a metal.

This permits the outside needle chamber surface 128t to be atraumatic, and decrease the possibility of injuring the patient, while the inside needle chamber surface 118t protects from inadvertent puncturing or damage by the needle 105t itself.

In another embodiment, the needle chamber 108t may be configured to receive the needle 105t such that it conforms to the curvature of the needle 105t. Accordingly, in one embodiment, the internal lumen 138t of the needle chamber 108t is curved, reflecting, at least to some degree, the curvature of the needle 105t.

Finally, insulation 175t is positioned along the length of the needle 105t and on the external surface 185t of the needle 105t. A sufficient amount of insulation 175t serves to protect tissue that should not be ablated and improves the dynamics of vapor distribution. Measured from the proximal end of the needle 105t, it is preferred to have the insulation extend at least 5% along the length of the needle 105t and no more than 90% along the length of the needle 105t, and more preferably at least 5% along the length of the needle 105t and no more than 75% along the length of the needle 105t.

FIG. 1N illustrates an ablation system 110 suitable for use in ablating an endometrial tissue, in accordance with embodiments of the present specification. The ablation system 110 comprises a catheter 111 having a catheter body 115 comprising an outer catheter 116 with an inner catheter 117 concentrically positioned inside and extendable outside from a distal end of the outer catheter 116. The inner catheter 117 includes at least one first distal attachment or positioning element 112 and a second proximal attachment or positioning element 113. The inner catheter 117 is positioned within the outer catheter 116 during positioning of the catheter 111 within a cervix or uterus of a patient. The first and second positioning elements 112, 113, in first, compressed configurations, are constrained by, and positioned within, the outer catheter 116 during positioning of the catheter 111. Once the distal end of the outer catheter 111 has been positioned within a cervix of a patient, the inner catheter 117 is extended distally from the distal end of the outer catheter 116 and into a uterus of the patient. The first and second positioning elements 112, 113 expand and become deployed within the uterus. In embodiments, the first and second positioning elements 112, 113 comprise shape memory properties, allowing them to expand once deployed. In some embodiments, the first and second positioning elements 112, 113 are comprised of Nitinol. In some embodiments, once deployed, the first, distal positioning element 112 is configured to contact a uterine wall, positioning the inner catheter 117 within the uterus, and the second, proximal positioning element 113, once deployed, is configured to abut a distal portion of the cervix just within the uterus, blocking passage of ablative vapor back into the cervical os. An internal heating chamber 103 is disposed within a lumen of the inner catheter 117 and configured to heat a fluid provided to the catheter 111 to change said fluid to a vapor for ablation therapy. In some embodiments, the internal heating chamber is positioned just distal to the second positioning element 113. In some embodiments, the catheter 111 is made of or covered with an insulated material to prevent the escape of ablative energy from the catheter body. The inner catheter 117 comprises one or more infusion ports 114 for the infusion of an ablative agent, such as steam. In some embodiments, the one or more infusion ports 114 are positioned on the catheter 111 between the first and second positioning elements 112 and 113. In various embodiments, the first distal attachment or positioning element 112 and second positioning element 113 comprise discs. A fluid, such as saline, is stored in a reservoir, such as a saline pump 14, connected to the catheter 111. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 22 monitors changes in an ablation area to guide flow of ablative agent. In some embodiments, the optional sensor comprises at least one of a temperature sensor or pressure sensor. In some embodiments, the catheter 111 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 111, or a switch 29 on the controller 15, for controlling vapor flow.

In one embodiment, a user interface included with the microprocessor 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels. In another embodiment, the outer catheter 116 abuts a cervical canal mucosa without blocking the cervix and the outflow from the uterine cavity. A space between the outer catheter 116 and inner catheter 117 allows for venting via a channel for heated air, vapor or fluid to escape out of the uterine cavity without contacting and damaging the cervical canal.

The ablation device also includes safety mechanisms to prevent users from being burned while manipulating the catheter, including insulation, and optionally, cool air flush, cool water flush, and alarms/tones to indicate start and stop of treatment.

FIG. 1O illustrates another view of a catheter 111 of FIG. 1N, in accordance with some embodiments of the present specification. The catheter 111 includes an elongate body 115 with a proximal end and a distal end. At the distal end, the catheter body 115 includes an outer catheter 116 with an inner catheter 117 concentrically positioned inside and extendable outside from a distal end of the outer catheter 116. The inner catheter 117 includes a distal positioning element 112, proximate its distal end, and a proximal positioning element 113 proximal to the distal positioning element 112. In various embodiments, the positioning elements are discs. The outer catheter 116 is configured to receive the inner catheter 117 and constrain the positioning elements 112, 113 before positioning, as described above. A plurality of infusion ports 114 are located on the inner catheter 117 between the two positioning elements 112, 113. The inner catheter 117 also includes at least one heating chamber 103 just distal to the proximal disc 113. In some embodiments, the heating chamber 103 includes two electrodes 109 configured to receive RF current, heat, and convert supplied fluid, such as saline, to vapor, or steam, for ablation.

Referring to FIG. 1O, for providing electrical current and liquid to the catheter 111, a fluid pump 174 and an RF generator 184 are coupled to the proximal end of the body 115. The fluid pump 174 pumps the fluid, such as saline, through a first lumen (extending along the length of the body 115) to the heating chamber 103. The RF generator 184 supplies electrical current to the electrodes 109, causing the electrodes 109 to generate heat and thereby converting the fluid flowing through the heating chamber 103 into vapor. The generated vapor flows through the first lumen and exits the ports 114 to ablate endometrial tissue. The flexible heating chamber 103 imparts improved flexibility and maneuverability to the catheter 111, allowing a physician to better position the catheter 111 when performing ablation procedures, such as ablating endometrial tissue of a patient.

In various embodiments, the heating electrode 109 is proximal to the proximal positioning element 113, extends beyond the distal end of the proximal positioning element 113, or is completely distal to the distal end of the proximal positioning element 113 but does not substantially extend beyond a proximal end of the distal positioning element 112.

FIG. 1P illustrates a system 100p for use in the ablation of endometrial tissue, in accordance with another embodiment of the present specification. The ablation system 100p comprises a catheter 101p which, in some embodiments, includes a handle 190p having actuators 191p, 192p, 193p for pushing forward a distal bulbous tip 189p of the catheter 101p and for deploying a first distal positioning element 101p and a second proximal positioning element 12p at the distal end of the catheter 101p. In embodiments, the catheter 101p comprises an outer sheath 109p and an inner catheter 107p. In embodiments, the catheter 101p includes a cervical collar 115p configured to rest against an external os once the catheter 101p has been inserted into a uterus of a patient. In embodiments, the distal first positioning element 11p and proximal second positioning element 12p are expandable, positioned at the distal end of the inner catheter 107p, and may be compressed within the outer sheath 109p for delivery. In some embodiments, actuators 192p and 193p comprise knobs. In some embodiments, actuator/knob 192b is used to deploy the distal first positioning element 11p. For example, in embodiments, actuator/knob 192p is turned one quarter turn to deploy the distal first positioning element 11p. In some embodiments, actuator/knob 193b is used to deploy the proximal second positioning element 12p. For example, in embodiments, actuator/knob 193p is turned one quarter turn to deploy the proximal second positioning element 12p. In some embodiments, the handle 190p includes only one actuator/knob 192p which is turned a first quarter turn to deploy the first distal positioning element 11p and then a second quarter turn to deploy the second proximal positioning element 12p. In other embodiments, other combinations of actuators/knobs are used to deploy one or both of the first distal positioning element 11p and second proximal positioning element 12p. In some embodiments, the catheter 101p includes a port 103p for the delivery of fluid, for example cooling fluid, during ablation. In some embodiments, port 103p is also configured to provide for fluid collection, provide vacuum, and provide CO₂ for an integrity test. In some embodiments, the port 103p is positioned on the handle 190p. In some embodiments, at least one electrode 113p is positioned at a distal end of the catheter 101p proximal to the proximal second positioning element 12p. The electrode 113p is configured to receive electrical current, supplied by a connecting wire 111p extending from the controller 15p to the catheter 101p, to heat and convert a fluid, such as saline supplied via tubing 112p extending from the controller 15p to the catheter 101p. Heated fluid or saline is converted to vapor or steam to be delivered by ports 114p ablation. In some embodiments, the catheter 101p is made of or covered with an insulated material to prevent the escape of ablative energy from the catheter body. A plurality of small delivery ports 114p is positioned on the inner catheter 107p between the distal first positioning element 11p and the second proximal positioning element 12p. Ports 114p are used for the infusion of an ablative agent, such as steam. Delivery of the ablative agent is controlled by the controller 15p and treatment is controlled by a treating physician via the controller 15p.

FIG. 1Q illustrates a controller 15q for use with an ablation system, in accordance with an embodiment of the present specification. Controller 15q, similar to controllers 15m, 15r, and 15p, controls the delivery of the ablative agent to the ablation system. The controller 15q therefore provides a control interface to a physician for controlling the ablation treatment. An input port 196q on the controller 15q provides a port to connect the controller 15q to the catheter and provide electrical signal to the catheter. A fluid port 198q on the controller 15q provides a port for connecting a supply to fluid such as saline through a tubing to the catheter. In embodiments, a graphical user interface (GUI) 1100q on the controller 15q shows the settings for operating the ablation system, which may be in use and/or modified by the physician during use. In some embodiments, the GUI is a touchscreen allowing for control of the system by a user.

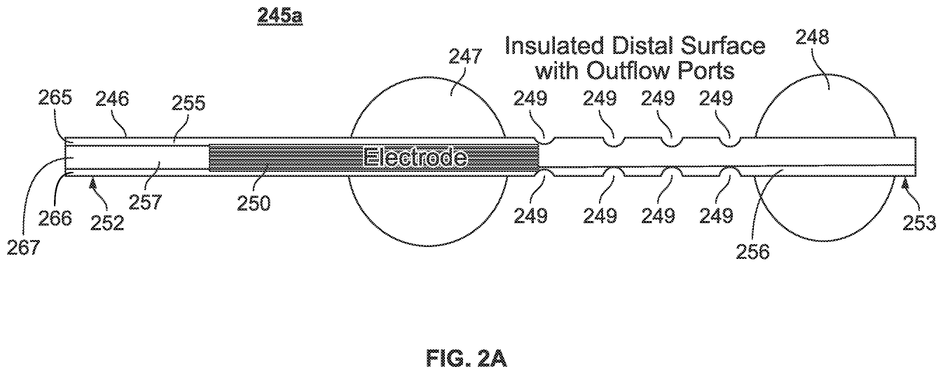

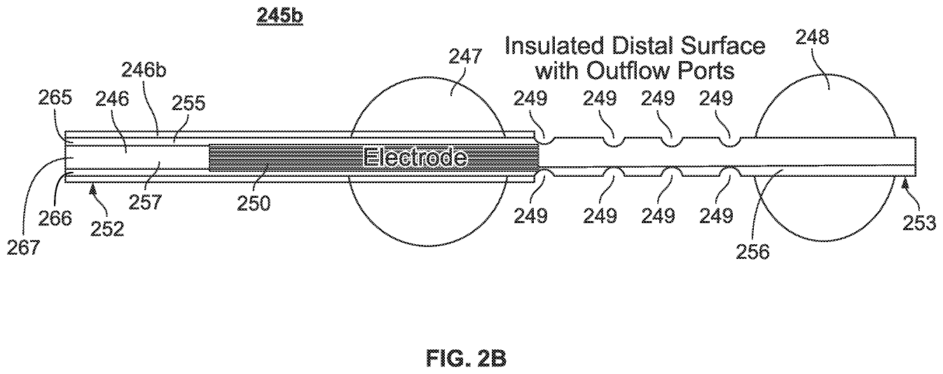

FIGS. 2A and 2B illustrate single and coaxial double balloon catheters 245a, 245b in accordance with embodiments of the present specification. The catheters 245a, 245b include an elongate body 246 with a proximal end 252 and a distal end 253 and a first lumen 255, a second lumen 256, and a third lumen 257 within. In an embodiment, the elongate body 246 is insulated. The catheters 245a, 245b include at least one positioning element 248 proximate their distal end 253. In various embodiments, the positioning element is an inflatable balloon. In some embodiments, the catheters include more than one positioning element. As shown in FIG. 2B, the coaxial catheter 245b includes an outer catheter 246b that accommodates the elongate body 246.

In the embodiments depicted in FIGS. 2A, 2B, the catheters 245a, 245b include a proximal first inflatable balloon 247 and a distal second inflatable balloon 248 positioned proximate the distal end of the body 246 with a plurality of infusion ports 249 located on the body 246 between the two balloons 247, 248. It should be appreciated that, while balloons are preferred, other positioning elements, as previously described, may be used.

The body 246 includes a first lumen 255 (extending along a portion of the entire length of the body 246) in fluid communication with a first input port 265 at the proximal end 252 of the catheter body 246 and with said proximal first balloon 247 to inflate or deflate the proximal first balloons 247, 248 by supplying or suctioning air through the first lumen 255. In an embodiment, use of a two-balloon catheter as shown in FIGS. 2A and 2B results in the creation of a seal and formation of a treatment area having a radius of 3 cm, a length of 9 cm, a surface area of 169.56 cm² and a treatment volume of 254.34 cm³. The body 246 includes a second lumen 256 (extending along the entire length of the body 246) in fluid communication with a second input port 266 at the proximal end 252 of the catheter body 246 and with said distal second balloon 248 to inflate or deflate the distal second balloon 248 by supplying or suctioning air through the second lumen 256. In another embodiment, the body includes only a first lumen for in fluid communication with the proximal end of the catheters and the first and second balloons for inflating and deflating said balloons. The body 246 also includes an in-line heating element 250 placed within a second third lumen 257 (extending along the length of the body 246) in fluid communication with a third input port 267 at the proximal end 252 of the catheter body 246 and with said infusion ports 249. In one embodiment, the heating element 250 is positioned within the third lumen 257, proximate and just proximal to the infusion ports 249. In an embodiment, the heating element 250 comprises a plurality of electrodes. In one embodiment, the electrodes of the heating element 250 are folded back and forth to increase a surface contact area of the electrodes with a liquid supplied to the third lumen 257. The second third lumen 257 serves to supply a liquid, such as water/saline, to the heating element 250.

In various embodiments, a distance of the heating element 250 from a nearest port 249 ranges from 1 mm to 50 cm depending upon a type of therapy procedure to be performed.

A fluid pump, an air pump and an RF generator are coupled to the proximate end of the body 246. The air pump propels air via said first and second inputs 265, 266 through the first and second lumens to inflate the balloons 247, 248 so that the catheters 245a, 245b are held in position for an ablation treatment. The fluid pump pumps a liquid, such as water/saline, via said third input 267 through the second third lumen 257 to the heating element 250. The RF generator supplies power and electrical current to the electrodes of the heating element 250, thereby causing the electrodes to heat and converting the liquid (flowing through around the heating element 250) into vapor. In other embodiments, the electrodes heat the fluid using resistive heating or ohmic heating. The generated vapor exits the ports 249 for ablative treatment of target tissue. In embodiments, the supply of liquid and electrical current, and therefore delivery of vapor, is controlled by a microprocessor.

### Prostate Ablation

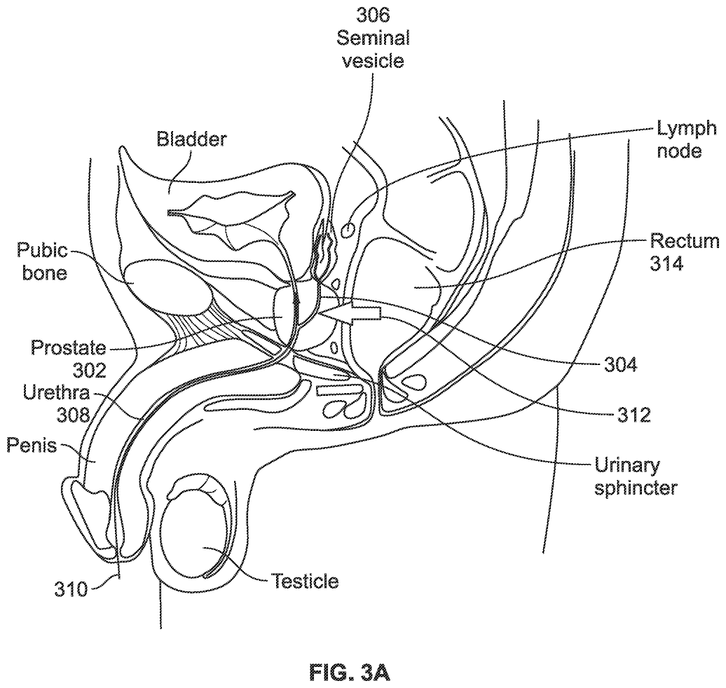

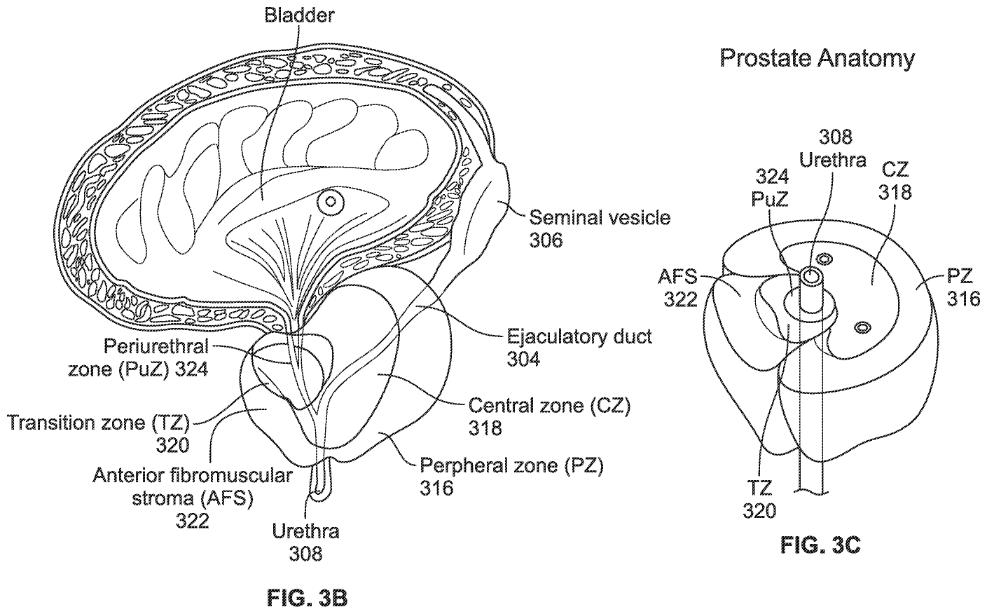

FIG. 3A illustrates a typical anatomy of a prostatic region for descriptive purposes. FIGS. 3B and 3C illustrate exemplary transparent views of prostate 302 anatomy, highlighting the peripheral zone (PZ) 316, in addition to other zones in the periphery of the prostate 302. Referring to the figures, embodiments of the present specification permit the ablation of prostate 302, by ablating PZ 316 prostatic tissue. In accordance with the various embodiments of the present specification, the embodiments enable ablating a prostate 302 tissue without completely ablating a central zone (CZ) 318 prostate tissue so as not to damage an ejaculatory duct 304, emerging from the duct of the seminal vesicle 306, which could cause a stricture of the ejaculatory duct 304. For purposes of the present specification, ‘completely ablating’ is defined as ablating more than 55% of a surface area or a volume around an anatomical structure.

Embodiments of the present specification enable ablating a prostate 302 tissue by ablating one of the numerous anatomical structures along various treatment pathways to treat the prostate 302. FIG. 3A illustrates a pathway 310 along the urethra as an exemplary pathway into the prostatic region for ablation also known as the transurethral approach. An alternative pathway 312 is illustrated through a wall between rectum 314 and prostate 302. In embodiments, prostate 302 tissue is ablated through urethra 308 or through a wall from rectum 314. In either case, the embodiments of the present specification ensure greater than 0% and less than 75% of the circumference of the periurethral zone 324, CZ 318, or any other zone, is ablated during the ablation of the prostate 302. In another embodiment, the prostate is accessed from the base of a bladder around a bladder neck without needing to go through a prostatic urethra, thereby avoiding the risk of ablating and stricturing the prostatic urethra. This approach is best reserved for ablation of benign or malignant obstruction caused by disease in the central zone of the prostate or median lobe hypertrophy.

In one embodiment, the ejaculatory duct 304 is the anatomical structure that is ablated. In another embodiment, the urethra 308 is ablated without completely ablating a circumference of the urethra 308 so as not to cause a stricture of the urethra 308. In other embodiments, the anatomical structures ablated may include the capsule of the prostate, including a rectal wall. In some embodiments, a portion of the prostate 302 or a portion of one or more of the CZ 318, the PZ 316, a transitional zone (TZ) 320, and an Anterior Fibromuscular Strauma (AFS) 322, are ablated. The different anatomical structures are ablated without ablating a contiguous circumference of periurethral zone (PuZ) 324 that surrounds the urethra 308. In some embodiments, greater than 0% and no more than 90% of the contiguous PuZ 324 circumference is ablated. In some embodiments, greater than 0% and less than 75% of the contiguous PuZ 324 circumference is ablated. In some embodiments, greater than 0% and less than 25% of the contiguous PuZ 324 circumference is ablated.

Therefore, in one embodiment, CZ 318 of a prostate 302 is ablated while ablating greater than 0% and less than 75% of a contiguous circumference of prostatic urethra 308. In another embodiment, CZ 318 of a prostate 302 is ablated while ablating greater than 0% and less than 75% of a contiguous circumference of ejaculatory duct 304. In one embodiment, TZ 320 of a prostate 302 is ablated while ablating greater than 0% and less than 75% of a contiguous circumference of prostatic urethra 308. In another embodiment, TZ 320 of a prostate 302 is ablated while ablating greater than 0% and less than 75% of a contiguous circumference of ejaculatory duct 304. In another embodiment, a median lobe of prostate 302 is ablated while ablating greater than 0% and less than 75% of a contiguous circumference of ejaculatory duct 304. In an embodiment a majority of the median lobe or CZ 318, ranging from more than 25% to more than 75%, is ablated without ablating a majority (>75%) of PuZ 324. In an embodiment a majority of TZ 320, ranging from more than 25% to more than 75%, is ablated without ablating a majority (>75%) of AFS 322. In some embodiments, preferably a range of 1% to 25%, and every increment therein, of a contiguous circumference of a prostatic urethra is ablated. In some embodiments, preferably a range of 1% to 25%, and every increment therein, of a contiguous circumference of an ejaculatory duct is ablated. In some embodiments, preferably a range of 1% to 25%, and every increment therein, of a thickness of the rectal wall is ablated. In the various embodiments, a mucosal layer of the rectal wall is not ablated.

FIG. 4A is an illustration of a water-cooled catheter 3100 while FIG. 4B is a cross-section of the tip of the catheter 3100, in accordance with another embodiment of the present specification. Referring now to FIGS. 4A and 4B, the catheter 3100 comprises an elongate body 3105 having a proximal end and a distal end. The distal end includes a positioning element 3125, such as an inflatable balloon. A plurality of openings 3115 are located proximate the distal end for enabling a plurality of associated thermally conductive elements 3116, such as needles, to be extended (at an angle from the catheter 3100, wherein the angle ranges between 10 to 150 degrees) and deployed or retracted through the plurality of openings 3115. In accordance with an aspect, the plurality of retractable needles 3116 are hollow and include at least one opening to allow delivery of an ablative agent, such as steam or vapor 3117, through the needles 3116 when the needles 3116 are extended and deployed through the plurality of openings 3115. This is illustrated in context of FIGS. 1L and 1M. A sheath 3110 extends along the body 3105 of the catheter 3100, including the plurality of openings 3115, to the distal end. The plurality of openings 3115 extend from the body 3105 and through the sheath 3110 to enable the plurality of needles 3116 to be extended beyond the sheath 3110 when deployed. During use, cooling fluid such as water or air 3120 is circulated through the sheath 3110 to cool the catheter 3100. Vapor 3117 for ablation and cooling fluid 3120 for cooling are supplied to the catheter 3100 at its proximal end.

It should be noted that alternate embodiments may include two positioning elements or balloons—one at the distal end and the other proximate the openings 3115 such that the openings 3115 are located between the two balloons.

FIG. 4C illustrates embodiments of a distal end of a catheter 3100a for use with the system 101m of FIG. 1M. In the embodiments shown in FIG. 4C, one or a plurality of openings 3115a is located proximate the distal end of an outer sheath 3110a for enabling one or a plurality of associated thermally conductive elements 3116a, such as needles, to be extended from an inner catheter 3111a (at an angle from the catheter 3100a, wherein the angle ranges between 10 to 90 degrees) and deployed or retracted through the one or plurality of openings 3115a. Each needle 3116a includes a beveled sharp edge 3118a for puncturing a prostatic tissue and an opening 3117a for the delivery of ablative agent. In some embodiments, each needle 3116a has a gradient of coating for insulation or echogenicity. The coating may be ceramic, polymer, or any other material suitable for coating the needles and providing insulation and/or echogenicity to the needles 3116a. The coatings are provided at the base of the needle 3116a to varying lengths to the tips. In some embodiments, each needle 3116a includes a physical gradient in its shape, such as a taper, beveled tip, or any other structural gradient, to regulate and manage steam distribution. In some embodiments, the physical shape of the needle is configured for tissue cutting. The needle edge is configured for puncturing the tissue without causing shearing or damage to the tissue.

In the multi-needle embodiment illustrated in FIG. 4C, the openings 3115a are circumferentially positioned at an equal distance from each other on the outer sheath 3110a. In various embodiments, an opening 3115a may be used to extend one or more needles 3116a. In other embodiments, the openings 3115a and needles 3116a are offset, or circumferentially positioned at an unequal distance from each other on the outer sheath 3110a. FIG. 4D illustrates other embodiments of a distal end of a catheter 3100b for use with the system 101m of FIG. 1M. One or a plurality of openings 3115b are circumferentially positioned around a sheath 3110b at an equal distance from each other and at the distal edge 3113b of sheath 3110b. In some multi-needle embodiments, the plurality of openings 3115b circumferentially positioned around sheath 3110b, are offset, and not always at an equal distance, from each other at the distal edge 3113b of sheath 3110b. The distal end of catheter 3100b can also have gradients of coating for insulation or echogenicity. The coatings may cover the needle surface in a range of 0 to 100% of the needle surface. In embodiments, coatings are concentrated at a proximal end of the needles 3116a to provide insulation to the needles. In some embodiments, coatings are concentrated at a distal end of the needles 3116a to impart the needles 3116a with echogenicity. The coatings may be ceramic, polymer, or any other material that may provide the needles 3116a with insulation and/or echogenicity. The coatings are provided at the base of the needle to varying lengths to the tips. In some embodiments, the needles have a physical gradient (shape, taper, or any other) to regulate and manage steam distribution. In some embodiments, the needle tips are shaped for cutting tissue. One or a plurality of associated thermally conductive elements 3116b, such as needles, is configured to be extended from an inner catheter 3111b (at an angle from the catheter 3100b, wherein the angle ranges between 10 to 90 degrees) and deployed or retracted through the one or plurality of openings 3115b. Each needle 3116b includes a beveled sharp edge 3118b for puncturing a prostatic tissue and an opening 3117b for the delivery of ablative agent. Referring simultaneously to FIGS. 4C and 4D, in accordance with an aspect, each of the retractable needles 3116a, 3116b is hollow and includes at least one opening 3117a, 3117b to allow delivery of an ablative agent, such as steam or vapor, through the one or more needles 3116a, 3116b when the needles 3116a, 3116b are extended and deployed through the one or plurality of openings 3115a, 3115b. This is further illustrated in the context of FIGS. 1L and 1M. Outer sheath 3110a, 3110b extends along a body of the catheter 3100a, 3100b, including the plurality of openings 3115a, 3115b, to the distal end. The plurality of openings 3115a, 3115b extends from the body and through the sheath 3110a, 3110b to enable each of the plurality of needles 3116a, 3116b to be extended beyond the sheath 3110a, 3110b when deployed. In some embodiments, openings 3115a, 3115b are provided with a locking mechanism for locking the needles 3116a, 3116b in their deployed position so that the needles 3116a, 3116b are prevented from being compressed. In some embodiments, the locking mechanisms are operated independently to provide the user with the ability to customize positions of needles 3116a, 3116b for disease location, amount of ablation, and orientation of the needles. The locking mechanism is deployed in all the embodiments of the present specification, for treating various conditions including BPH and AUB. In all of these embodiment, the needles are electrically separated from the vapor generation chamber by a length of the catheter so as to electrically insulate the tissue from the RF electrical current being delivered to the vapor generation chamber.

In different embodiments, size and numbers of openings 3115a, 3115b may vary. Further, in various embodiments, openings 3115a, 3115b that provide exit ports for the steam can be all the same size along the length of the sheath 3110a, 3110b, and may have different patterns such as and not limited to: spiral, circular, or any other pattern. Further, openings 3115a, 3115b may have a gradient of dimensions to force steam distribution into certain regions of the anatomy. In an exemplary embodiment, the diameters of the openings 3115a, 3115b may vary by at least 10% (but not limited to) from top to bottom or from bottom to top. Additionally, the openings 3115a, 3115b may be of different shapes such as round, oval, or any other shape.

FIG. 4E illustrates an embodiment of a slit flap used to cover openings 3115a, 3115b of FIGS. 4C and 4D, in accordance with some embodiments of the present specification. In embodiments, the slit flap 3119 is made from material such as, but not limited to, silicone or polyurethane (PU). A flap 3119 is positioned over each opening 3115a, 3115b. Needles 3116a, 3116b may be extended (at an angle from the catheter 3100a, 3100b, wherein the angle ranges between 30 to 90 degrees) and deployed or retracted through the plurality of flaps 3118.

FIG. 4F illustrates an embodiment of a positioning element 4125 to be positioned at a distal end of an ablation catheter, to position the ablation catheter in the prostatic urethra, in accordance with the present specification. In some embodiments, positioning elements having the same shape as element 4125 are used in the uterus as well for endometrial ablation, as described in embodiments of the present specification. In embodiments, the positioning element comprises a plurality of wires 4126 woven into a pattern, for example a spiral pattern. In embodiments, the wires 4126 are composed a shape memory material to allow for compression of the positioning element 4125 during delivery. In some embodiments, the shape memory material is Nitinol. In various embodiments, the positioning element 4125 has a funnel, bell, spherical, oval, ovoid, or acorn shape and is substantially cylindrical when compressed. The positioning element 4125, when deployed, abuts and rests in the bladder or the bladder neck.

FIGS. 4G to 4L illustrate exemplary steps showing one embodiment of using a catheter 4100, similar to the catheters of FIGS. 4C, 4D, and 4E, to ablate prostate tissue 4130, in accordance with the present specification. An outer catheter or sheath 4110 encompasses an inner catheter 4105. FIG. 4G illustrates advancing a distal end of catheter 4100 through a prostatic urethra 4128. In embodiments, the catheter 4100 includes a coude tip 4109 at its distal end 4119 configured to push through and position against a patient's bladder 4132. In embodiments, the coude tip 4109 is bent or elbow tipped. FIG. 4H illustrates advancing a distal end of catheter 4100 into a bladder 4132, and FIG. 4I illustrates even further advancing a distal end of catheter 4100 into the bladder 4132. As shown in FIGS. 4H and 4I, outer sheath 4110 is retracted slightly to expose a distal end of inner catheter 4105 with a positioning element 4125 in a compressed configuration. Referring to FIG. 4J, positioning element 4125 is expanded and catheter 4100 is retracted to position positioning element 4125 proximate a bladder neck 4134 or the distal end of the prostatic urethra 4128. Referring to FIG. 4K, a needle 4116 is extended from the catheter 4100 and into the prostatic tissue 4130. In embodiments, needle 4116 refers to at least one, and in some embodiments, more than one needle. In embodiments, the needle 4116 is deployed and extended according to the embodiments illustrated in FIGS. 4A, 4C, and 4D. Referring to FIG. 4L, an ablative agent 4136 is delivered through the needle 4116 into prostate tissue 4130.

In an alternative embodiment, referring to FIG. 4M, a catheter 4100a has a positioning element 4125a positioned on the catheter 4100a proximal to needle 4116a, which in turn is positioned at the distal end of the catheter 4100a. In other embodiments, the catheter includes more than one needle. The catheter includes an outer sheath 4110a and an inner catheter 4105a. The positioning element 4125a and needle 4116a are positioned on the inner catheter 4105a, with the needle 4116a distal to the positioning element 4125a. As shown in FIG. 4M, the catheter 4100a is advanced into a prostatic urethra 4128a with both the needle 4116a and positioning element 4125a in collapsed configurations. Referring to FIG. 4N, the positioning element 4125a is expanded to hold the catheter 4100a within the prostatic urethra 4128a and the needle 4116a is deployed into the prostatic tissue 4130a for delivery of ablative agent. In various embodiments, the positioning element 4125a has a funnel, bell, spherical, oval, ovoid, or acorn shape when deployed and is substantially cylindrical when compressed.

FIG. 4O is a flow chart illustrating the steps involved in using an ablation catheter to ablate a prostate of a patient, in accordance with embodiments of the present specification. At step 4140, a coude tip of the catheter is used to push through a patient's prostatic urethra and position a distal end of the catheter against the patient's bladder. At step 4142, an outer sheath of the catheter is retracted, using an actuator, to reveal a positioning element, or bladder anchor, and the positioning element is positioned, for example in the bladder neck, to position the catheter for ablation. At step 4144, the outer sheath is retracted further to deploy a needle or plurality of needles from the catheter and into prostatic tissue. In some embodiments, the one or more needles are deployed out of an internal lumen of an inner catheter of the catheter and through slots in the outer sheath. In another embodiment, the sleeves naturally fold outward as the outer sheath is retracted. At step 4146 vapor or steam is delivered through the one or more needles to ablate the prostatic tissue.

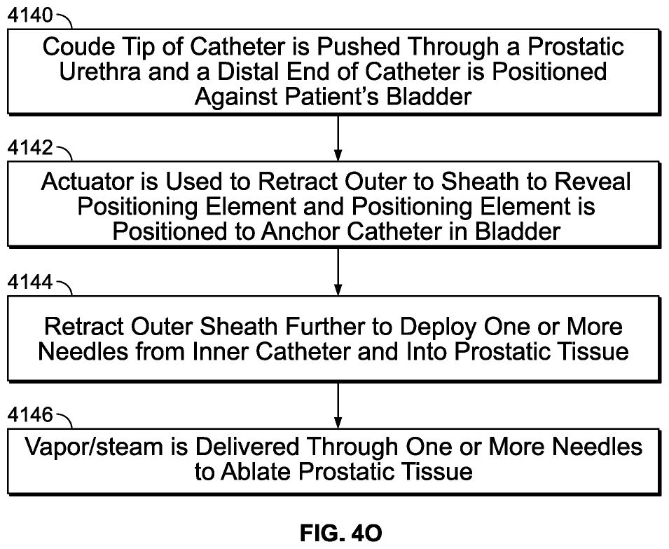

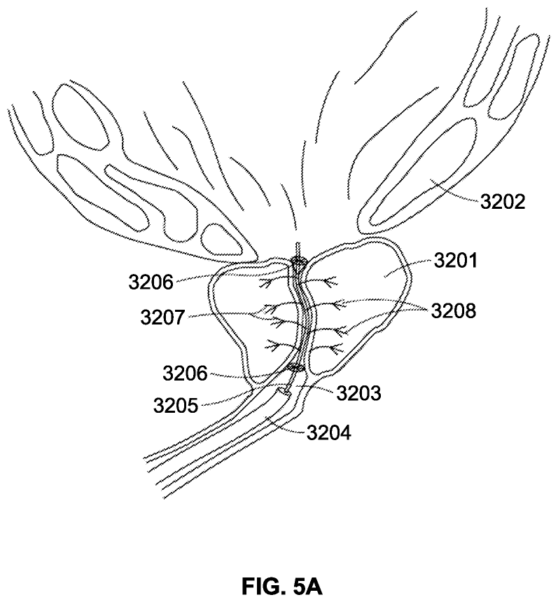

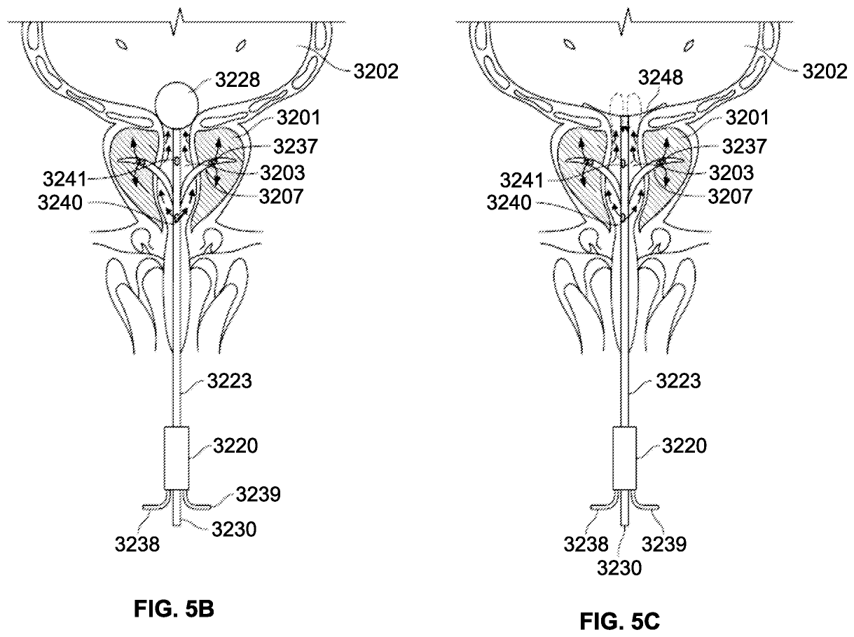

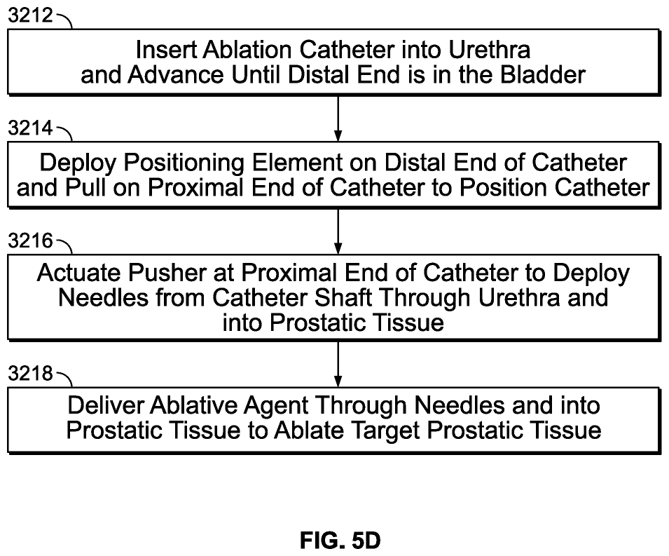

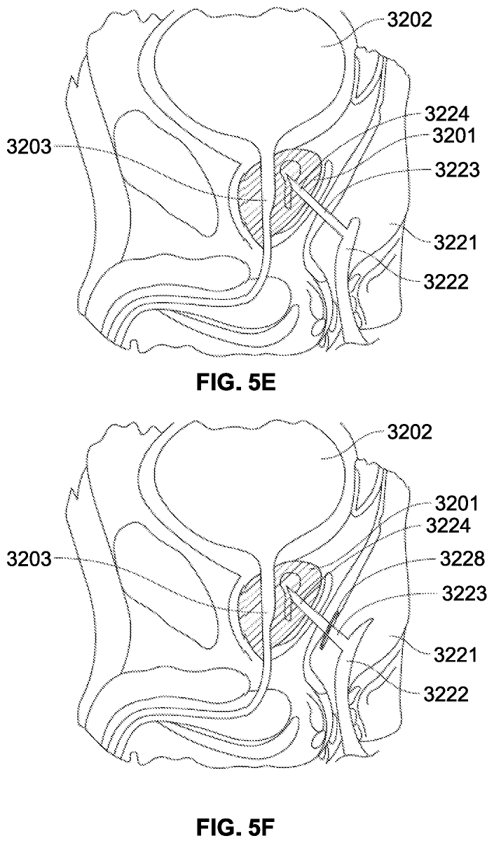

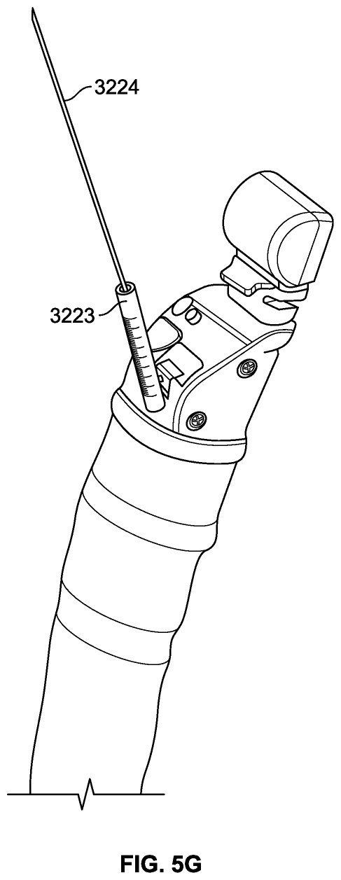

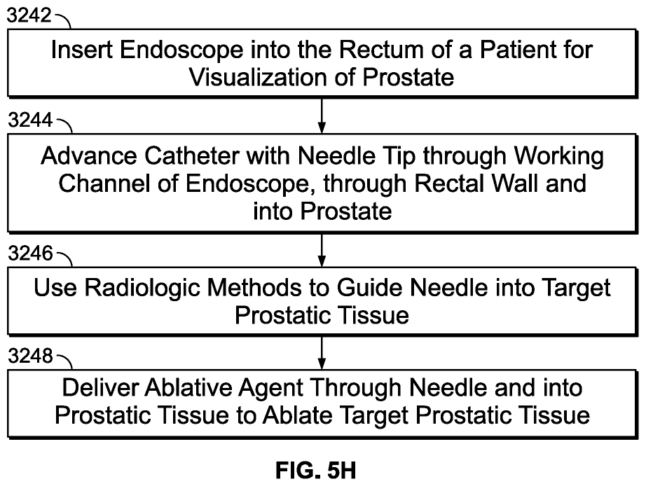

FIG. 5A illustrates prostate ablation being performed on an enlarged prostrate in a male urinary system by using a catheter (such as the catheter 3100 of FIG. 4A—with two positioning elements), in accordance with an embodiment of the present specification. A cross-section of a male genitourinary tract having an enlarged prostate 3201, bladder 3202, and urethra 3203 is illustrated. The urethra 3203 is compressed by the enlarged prostate 3201. The ablation catheter 3205 is passed through the cystoscope 3204 positioned in the urethra 3203 distal to the obstruction. The positioning elements 3206 are deployed to center the catheter in the urethra 3203 and one or more insulated needles 3207 are passed to pierce the prostate 3201. The vapor ablative agent 3208 is passed through the insulated needles 3207 thus causing ablation of the diseased prostatic tissue resulting in shrinkage of the prostate. In one embodiment, only the proximal positioning element is used while in another embodiment, only the distal positioning element is used.

The size of the enlarged prostate could be calculated by using the differential between the extra-prostatic and intra-prostatic urethra. Normative values could be used as baseline. Additional ports for infusion of a cooling fluid into the urethra can be provided to prevent damage to the urethra while the ablative energy is being delivered to the prostrate for ablation, thus preventing complications such as stricture formation.

In one embodiment, the positioning attachment must be separated from the ablation region by a distance of greater than 0.1 mm, preferably 1 mm to 5 mm and no more than 2 cm. In another embodiment, the positioning attachment can be deployed in the bladder and pulled back into the urethral opening/neck of the bladder thus fixing the catheter. In one embodiment, the positioning device is between 0.1 mm and 10 cm in diameter.

FIG. 5B is an illustration of transurethral prostate ablation being performed on an enlarged prostrate 3201 in a male urinary system using an ablation device (such as the catheter 3100 of FIG. 4A—with one positioning element), in accordance with one embodiment of the present specification. Also depicted in FIG. 5B are the urinary bladder 3202 and prostatic urethra 3203. An ablation catheter 3223 with a handle 3220 and a positioning element 3228 is inserted into the urethra 3203 and advanced into the bladder 3202. The positioning element 3228 is inflated and pulled to the junction of the bladder with the urethra, thus positioning needles 3207 at a predetermined distance from the junction. In some embodiments, the positioning element 3228 is inflated to a first volume in the bladder 3202 proximate the junction of the bladder 3202 with the urethra 3203, to position needles 3207 proximate to prostate 3201; and to a second volume, different from the first volume, to position needles 3207 at a different position proximate to prostate 3201. Using a balloon as the positioning element 3228, provides counter-traction while the needles 3207 are being deployed.

Using a pusher 3230, the needles 3207 are then pushed out at an angle between 10 and 90 degrees from the catheter 3223 through the urethra 3203 into the prostate 3201. Vapor is administered through a port 3238 that travels through the shaft of the catheter 3223 and exits from openings 3237 in the needles 3207 into the prostatic tissue, thus ablating the prostatic tissue. In embodiments, the vapor is delivered for a predetermined time, to a predetermined pressure, and to deliver a predetermined amount of energy. In some embodiments, the vapor is delivered for a period that is less than five minutes, and preferably for a period within a range between 2 seconds to 120 seconds, and more preferably for a period of time of 60 to 90 seconds. In embodiments, the vapor is delivered at a pressure that is less than 5 atm and in some cases less than 1 atm, and preferably at a pressure no greater than 10% above atmospheric pressure. In embodiments, the vapor is delivered at an energy in a range of 10 cal to 10,000 cal.

In one embodiment, the needles 3207 are insulated so as to prevent damage to a prostatic urethra 3203 or a periurethral zone. Additionally, in embodiments, the needles are deployed to deliver vapor at a location that is preferentially away from the ejaculatory duct. In some embodiments, a shape of needles 3207 is different during delivery of vapor compared to their shape prior to delivery of vapor.

Optional port 3239 allows for insertion of cool fluid at a temperature<37 degree C. through opening 3240 to cool the prostatic urethra 3203 or the periurethral zone. Optional temperature sensors 3241 can be installed to detect the temperature of the prostatic urethra and modulate the delivery of vapor.

FIG. 5C is an illustration of transurethral prostate ablation being performed on an enlarged prostrate 3201 in a male urinary system using an ablation device, in accordance with another embodiment of the present specification. Also depicted in FIG. 5C are the urinary bladder 3202 and prostatic urethra 3203. An ablation catheter 3223 with a handle 3220 and a positioning element 3248 is inserted into the urethra 3203 and advanced into the bladder 3202. The positioning element 3248 is a compressible disc that is expanded in the bladder 3202 and pulled to the junction of the bladder with the urethra, thus positioning needles 3207 at a predetermined distance from the junction. In some embodiments, the positioning element 3248 is expanded to a first size in the bladder 3202 proximate the junction of the bladder 3202 with the urethra 3203, to position needles 3207 proximate to prostate 3201; and to a second size, different from the first size, to position needles 3207 at a different position proximate to prostate 3201.

Using a pusher 3230, the needles 3207 are then pushed out at an angle between 10 and 90 degree from the catheter 3223 through the urethra 3203 into the prostate 3201. Vapor is administered through a port 3238 that travels through the shaft of the catheter 3223 and exits through openings 3237 in the needles 3207 into the prostatic tissue, thus ablating the prostatic tissue. In embodiments, the vapor is delivered for a predetermined time, to a predetermined pressure, to deliver a predetermined amount of energy. In some embodiments, the vapor is delivered for a period of time that is less than five minutes, and preferably for a period of time within a range of 60 to 90 seconds. In other embodiments, the vapor is delivered for a period of time with a range between 2 seconds and 30 seconds. In another embodiment, the vapor is delivered for a period of time with a range between 30 seconds and 60 seconds. In embodiments, the vapor is delivered at a pressure that is less than 5 atm and in some cases less than 1 atm, and preferably at a pressure no greater than 10% above atmospheric pressure.

In one embodiment, the needles 3207 are insulated so as to prevent damage to a prostatic urethra 3203 or a periurethral zone. Additionally, in embodiments, the needles are deployed to deliver vapor at a location that is preferentially away from the ejaculatory duct. In some embodiments, a shape of needles 3207 is different during delivery of vapor compared to their shape prior to delivery of vapor.

Optional port 3239 allows for insertion of cool fluid at a temperature<37 degree C. through opening 3240 to cool the prostatic urethra 3203 or the periurethral zone. Optional temperature sensors 3241 can be installed to detect the temperature of the prostatic urethra and modulate the delivery of vapor.

FIG. 5D is a flow chart listing the steps involved in a transurethral enlarged prostate ablation process using an ablation catheter, in accordance with one embodiment of the present specification. At step 3212, an ablation catheter (such as the catheter 3100 of FIG. 4A) is inserted into the urethra and advanced until its distal end is in the bladder. A positioning element is then deployed on the distal end of the catheter, at step 3214, and the proximal end of the catheter is pulled so that the positioning element abuts the junction of the bladder with the urethra, thereby positioning the catheter shaft within the urethra. A pusher at the proximal end of the catheter is actuated to deploy needles from the catheter shaft through the urethra and into the prostatic tissue at step 3216. At step 3218, an ablative agent is delivered through the needles and into the prostate to ablate the target prostatic tissue.

FIG. 5E is an illustration of transrectal prostate ablation being performed on an enlarged prostrate in a male urinary system using an ablation device, in accordance with one embodiment of the present specification. Also depicted in FIG. 5E are the urinary bladder 3202 and prostatic urethra 3203. The ablation device comprises a catheter 3223 with a needle tip 3224. An endoscope 3222 is inserted into the rectum 3221 for the visualization of the enlarged prostate 3201. In various embodiments, the endoscope 3222 is an echoendoscope or a transrectal ultrasound such that the endoscope can be visualized using radiographic techniques. The catheter 3223 with needle tip 3224 is passed through a working channel of the endoscope and the needle tip 3224 is passed transrectally into the prostate 3201. A close-up illustration of the distal end of the catheter 3223 and needle tip 3204 is depicted in FIG. 5G. An ablative agent is then delivered through the needle tip 3224 into the prostatic tissue for ablation. In embodiments, the prostatic tissue is ablated without ablating a full thickness of the rectal wall. In some embodiments, no more than 90% of a thickness of the rectal wall is ablated. In some embodiments, greater than 0% and less than 75% of a thickness of the rectal wall is ablated. In some embodiments, preferably a range of 1% to 25%, and every increment therein, of a thickness of the rectal wall is ablated. In some embodiments, a mucosal layer of the rectal wall is not ablated.

In one embodiment, the catheter 3223 and needle tip 3224 are composed of a thermally insulated material. In various embodiments, the needle tip 3224 is an echotip or sonolucent tip that can be observed using radiologic techniques for accurate localization in the prostate tissue. In one embodiment, an optional catheter (not shown) can be placed in the urethra to insert fluid to cool the prostatic urethra 3203. In one embodiment, the inserted fluid has a temperature less than 37° C.

FIG. 5F is an illustration of transrectal prostate ablation being performed on an enlarged prostrate in a male urinary system using a coaxial ablation device having a positioning element, in accordance with another embodiment of the present specification. Also depicted in FIG. 5F are the urinary bladder 3202 and prostatic urethra 3203. The ablation device comprises a coaxial catheter 3223 having an internal catheter with a needle tip 3224 and an external catheter with a positioning element 3228. An endoscope 3222 is inserted into the rectum 3221 for the visualization of the enlarged prostate 3201. In various embodiments, the endoscope 3222 is an echoendoscope or a transrectal ultrasound such that the endoscope can be visualized using radiographic techniques. The coaxial catheter 3223 with needle tip 3224 and positioning element 3228 is passed through a working channel of the endoscope such that the positioning element 3228 comes to rest up against the rectal wall and the internal catheter is advanced transrectally, thereby positioning the needle tip 3224 at a predetermined depth in the prostate 3201. A close-up illustration of the distal end of the catheter 3223 and needle tip 3204 is depicted in FIG. 5G. In one embodiment, the positioning element is a compressible disc that has a first, compressed pre-employment configuration and a second, expanded deployed configuration once it has passed beyond the distal end of the endoscope 3222. An ablative agent is then delivered through the needle tip 3224 into the prostatic tissue for ablation. In embodiments, the prostatic tissue is ablated without ablating a full thickness of the rectal wall. In some embodiments, no more than 90% of a thickness of the rectal wall is ablated. In some embodiments, greater than 0% and less than 75% of a thickness of the rectal wall is ablated. In some embodiments, preferably a range of 1% to 25%, and every increment therein, of a thickness of the rectal wall is ablated. In some embodiments, a mucosal layer of the rectal wall is not ablated.

In one embodiment, the coaxial catheter 3223, needle tip 3224, and positioning element 3228 are composed of a thermally insulated material. In various embodiments, the needle tip 3224 is an echotip or sonolucent tip that can be observed using radiologic techniques for accurate localization in the prostate tissue. In one embodiment, an optional catheter (not shown) can be placed in the urethra to insert fluid to cool the prostatic urethra 3203. In one embodiment, the inserted fluid has a temperature less than 37° C.

FIG. 5H is a flow chart listing the steps involved in a transrectal enlarged prostate ablation process using an ablation catheter, in accordance with one embodiment of the present specification. At step 3242, an endoscope is inserted into the rectum of a patient for visualization of the prostate. A catheter with a needle tip is then advanced, at step 3244, through a working channel of the endoscope and through the rectal wall and into the prostate. Radiologic methods are used to guide the needle into the target prostatic tissue at step 3246. At step 3248, an ablative agent is delivered through the needle and into the prostate to ablate the target prostatic tissue. In embodiments, the prostatic tissue is ablated without ablating a full thickness of the rectal wall. In some embodiments, no more than 90% of a thickness of the rectal wall is ablated. In some embodiments, greater than 0% and less than 75% of a thickness of the rectal wall is ablated. In some embodiments, preferably a range of 1% to 25%, and every increment therein, of a thickness of the rectal wall is ablated. In some embodiments, a mucosal layer of the rectal wall is not ablated.

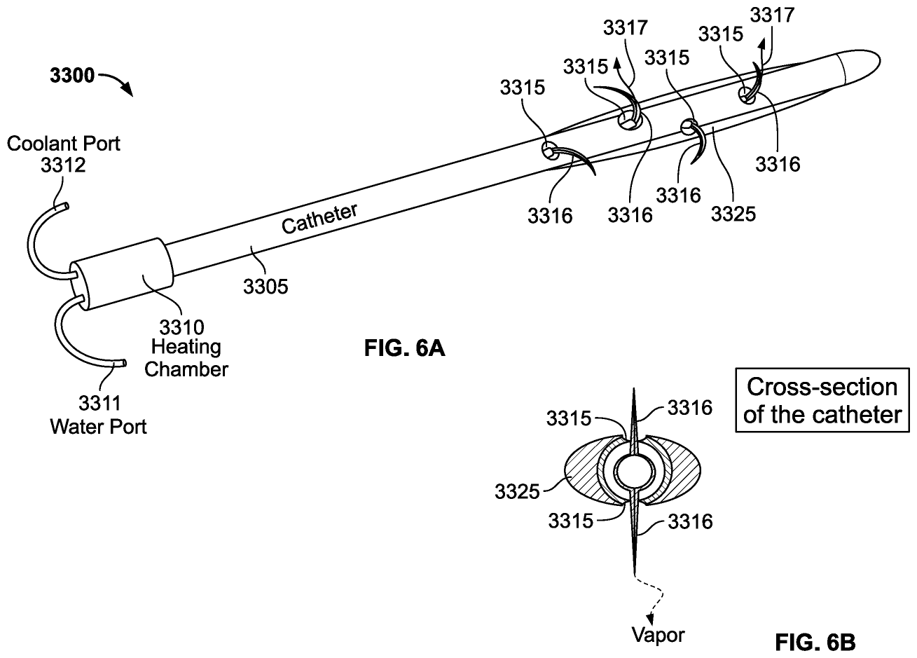

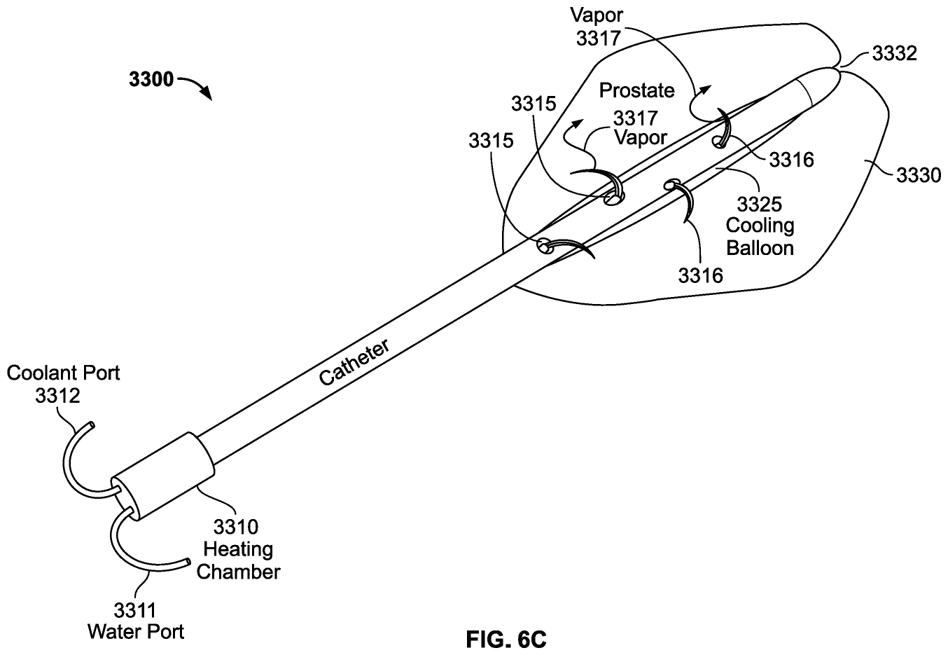

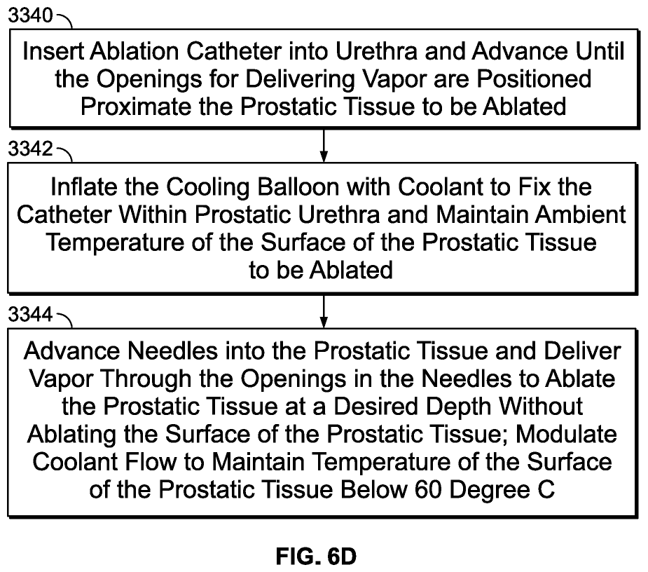

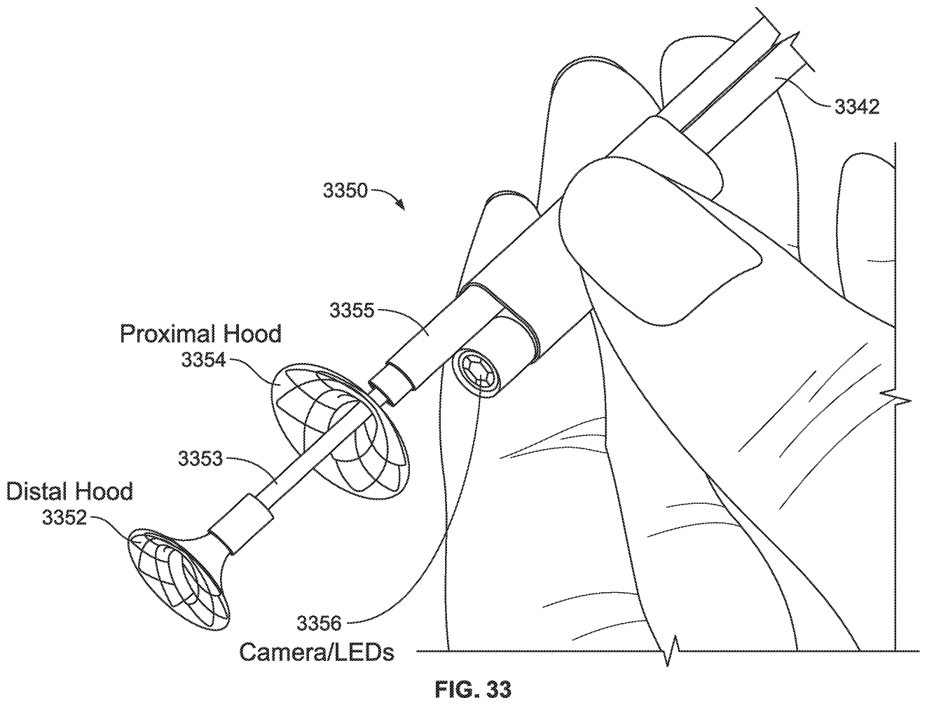

FIG. 6A illustrates an ablation catheter 3300 while FIG. 6B is a cross-section of the tip of the catheter 3300, in accordance with an embodiment of the present specification. Referring now to FIGS. 33A and 33B, the catheter 3300 comprises an elongate body 3305 having a proximal end and a distal end. A plurality of openings 3315 and an inflatable balloon 3325 are located proximate the distal end. The plurality of openings 3315 enable a plurality of associated thermally conductive elements 3316, such as needles, to be extended (at an angle from the catheter 3300, wherein the angle ranges between 30 to 90 degrees) or retracted through the plurality of openings 3315. In accordance with an aspect, the plurality of retractable needles 3316 are hollow and include at least one opening to allow delivery of an ablative agent, such as steam or vapor 3317, through the needles 3316 when the needles are extended and deployed through the plurality of openings 3315. The plurality of openings 3315 extend from the body 3305 and through the balloon 3325 to enable the plurality of needles 3316 to be extended beyond the balloon 3325 when deployed.

A heating chamber 3310 is located at the proximal end of the catheter 3300. The heating chamber 3310 comprises a metal coil wound about a ferromagnetic core. The chamber 3310 is filled with water via a water inlet port 3311 at a proximal end of the chamber 3310. Alternating current is provided to the coil creating a magnetic field that induces electric current flow in the ferromagnetic core thereby heating the chamber 3310 and causing the water within to vaporize. The resulting steam or vapor 3317 exits the needles 3316 to ablate target tissue. The balloon 3325 is inflated by filling it with a coolant that is supplied to the balloon 3325 through a coolant port 3312 at the proximal end of the chamber 3310. During use, the balloon 3325 is inflated with the coolant while vapor or steam 3317, generated in the chamber 3310, is delivered through the plurality of needles 3316. Since the needles 3316 pierce into the target tissue during use, the steam or vapor 3317 delivered through the pierced needles 3316 cause ablation of tissue located deep within the target tissue. The coolant filled inflated balloon 3325 contacts the surface of the non-target tissue and maintains the ambient temperature on the surface of the non-target tissue to a desired level, such as below 60 degrees C. in some embodiments. This enables the vapor 3317 to ablate deeper target tissue without circumferentially ablating the non-target tissue at the surface. In some embodiments, the heating chamber 3310 is at the distal end of the catheter proximal to the most proximal needle 3316 and the plurality of openings 3315, and is configured to use RF energy to generate vapor using resistive or ohmic heating of saline. In all embodiments, the plurality of the needles are electrically isolated from the heating chamber 3310 by a segment of the catheter 3305 so as to prevent the RF electrical current from the electrode passing into the tissues and into the human body. In various embodiments a conductive fluid such as saline is heated to a non-conductive ablative agent such as steam so as to minimize the chances of RF electrical current from passing from the heating chamber into the prostatic tissue and patient's body. It is desirable to isolate the patient from the RF electrical current so as not to interfere with any implanted electromedical devices.

FIG. 6C is an illustration of prostate ablation being performed on an enlarged prostrate in a male urinary system using the ablation catheter 3300 of FIG. 6A, in accordance with an embodiment of the present specification. Also depicted in FIG. 6C are the prostate 3330 and prostatic urethra 3332. Referring now to FIGS. 33A and 33C, the ablation catheter 3300 with the heating chamber 3310 and the inflatable cooling balloon 3325 is inserted into the patient's urethra and advanced into the prostatic urethra 3332 so as to position the plurality of openings 3315 proximate the tissue to be ablated. The cooling balloon 3325 is inflated by filling it with coolant supplied from the coolant port 3312, so that the inflated cool balloon 3325 abuts the surface of the prostatic urethra proximate to the prostatic tissue to be ablated. Using a pusher, the needles 3316 are then pushed out at an angle (ranging between 10 and 90 degrees, in various embodiments) from the catheter 3300 into the prostate 3330. Water (through the water inlet port 3311) is administered into the chamber 3310 where it is converted into steam or vapor 3317. The steam or vapor 3317 travels through the body 3305 of the catheter and exits from openings in the needles 3316 into the prostatic tissue, thus ablating the prostatic tissue. In one embodiment, the needles 3316 are insulated. The coolant filled inflated balloon 3325 maintains the ambient temperature on the surface of the prostatic urethra tissue to a desired level, such as below 60 degree C. in some embodiments. This enables the vapor 3317 to ablate deeper prostatic tissue without circumferentially ablating the prostatic urethra tissue at the surface. Optional temperature sensors can be installed to detect the temperature of the prostatic urethra and modulate the delivery of vapor. In some embodiments, the heating chamber 3310 is at the distal end of the catheter proximal to the most proximal needle 3316 and the plurality of openings 3315, and is configured to use RF energy to generate vapor using resistive or ohmic heating of saline. In embodiments, the needles are separated from the RF electrode by an insulative segment of the catheter to minimize or prevent passage of RF current into the patient's tissue and prevent electrical interference with electromedical implants.

FIG. 6D is a flow chart listing the steps involved in a transurethral enlarged prostate ablation process using the ablation catheter 3300 of FIG. 6A, in accordance with one embodiment of the present specification. Referring now to FIGS. 6A and 6D, at step 3340, the ablation catheter 3300 is inserted into the urethra and advanced until the plurality of openings 3315 are positioned proximate the prostatic tissue to be ablated within the prostatic urethra. At step 3342, the cooling balloon 3325 is inflated, with coolant supplied from the coolant port 3312, to fix the catheter 3300 within the prostatic urethra and maintain ambient temperature on the surface of the tissue to be ablated. Using a pusher, at step 3344, the needles 3316 are then pushed out at an angle (between 30 and 90 degrees, in various embodiments) from the catheter 3300 through the prostatic urethra and into the prostate up to a desirable depth. Vapor is delivered, from openings in the needles 3316, into the prostatic tissue at the desirable depth, thus ablating the prostatic tissue, without ablating the surface of the prostatic urethra. An optional temperature sensor is utilized to monitor the temperature of the surface of the prostatic urethra and control or modulate the flow of the coolant to maintain the temperature of the surface of the prostatic urethra below, say, 60 degrees C.

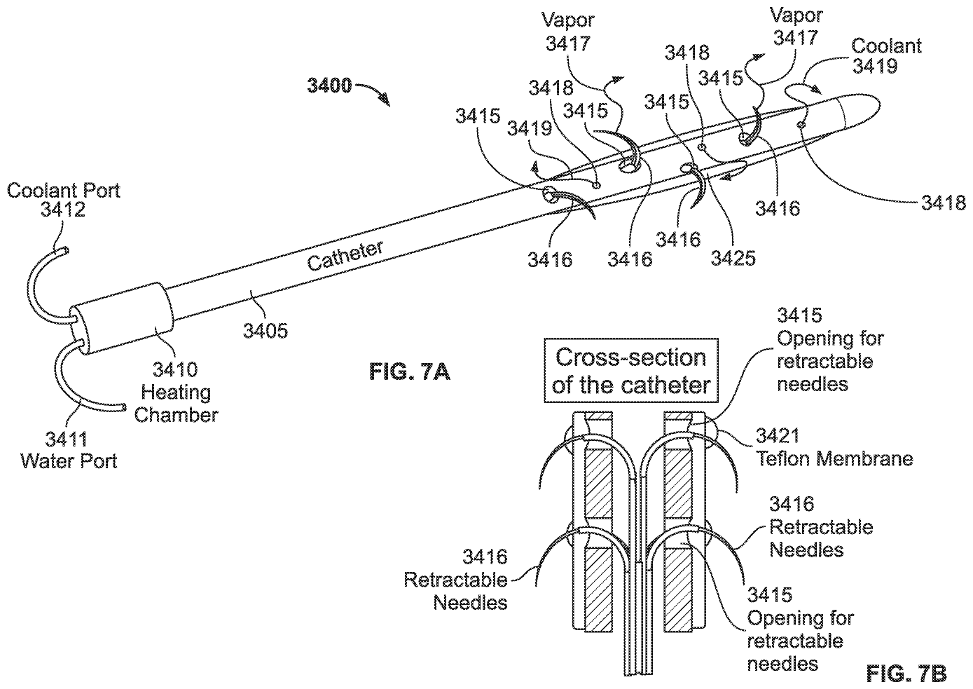

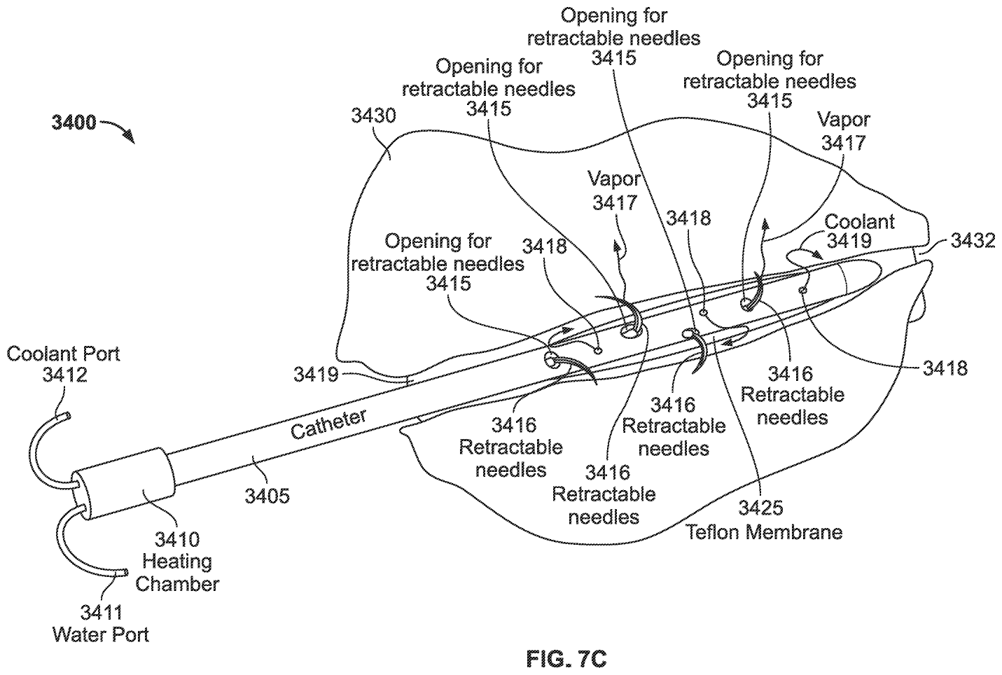

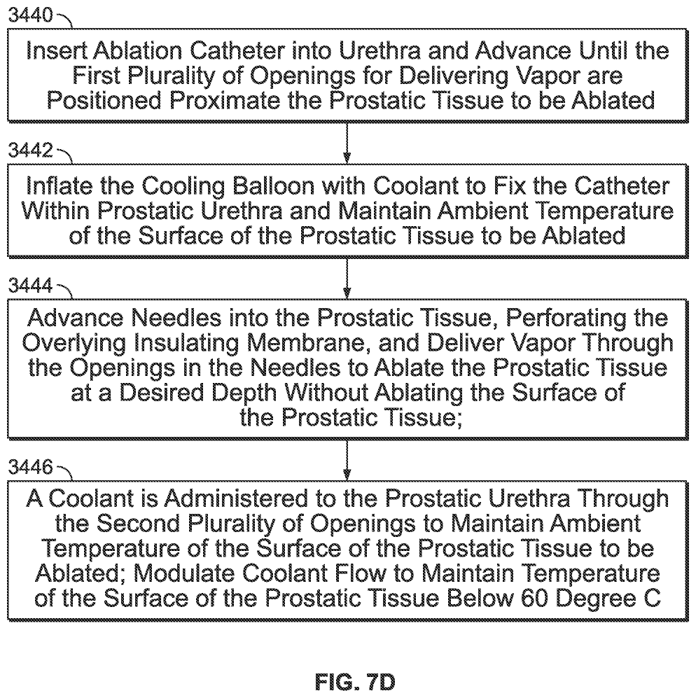

FIG. 7A illustrates an ablation catheter 3400 while FIG. 7B is a cross-section of the tip of the catheter 3400, in accordance with an embodiment of the present specification. Referring now to FIGS. 7A and 7B, the catheter 3400 comprises an elongate body 3405 having a proximal end and a distal end. A first plurality of openings 3415, a second plurality of openings 3418, and a silicone or Teflon membrane 3425, covering the first and second pluralities of openings, are located proximate the distal end. The first plurality of openings 3415 enables a plurality of associated thermally conductive elements 3416, such as needles, to be extended (at an angle from the catheter 3400, wherein the angle ranges between 30 to 90 degrees) or retracted through the plurality of openings 3415. The second plurality of openings 3418 enables a coolant 3419, supplied via coolant port 3412 at the proximal end of the catheter 3400, to be delivered to the ablation zone. In accordance with an aspect, the plurality of retractable needles 3416 are hollow and include at least one opening to allow delivery of an ablative agent, such as steam or vapor 3417, through the needles 3416 when the needles are extended and deployed through the first plurality of openings 3415. The plurality of openings 3415 extend from the body 3405 and through the balloon 3425 to enable the plurality of needles 3416 to be extended beyond the membrane 3425 when deployed. The needles 3416 pierce through the membrane 3425, when deployed, such that the membrane 3425 insulates the needles 3416 as these are being deployed and pierced into a target tissue.

A heating chamber 3410 is located at the proximal end of the catheter 3400. The heating chamber 3410 comprises a metal coil wound about a ferromagnetic core. The chamber 3410 is filled with water via a water inlet port 3411 at a proximal end of the chamber 3410. Alternating current is provided to the coil creating a magnetic field that induces electric current flow in the ferromagnetic core thereby heating the chamber 3410 and causing the water within to vaporize. The resulting steam or vapor 3417 exits the needles 3416 to ablate target tissue. The coolant port 3412, at the proximal end of the chamber 3410, supplies coolant 3419 for delivery through the second plurality of openings 3418 into the prostatic urethra. During use, the coolant 3419 is delivered to the ablation zone through coolant openings 3418, while vapor or steam 3417, generated in the chamber 3410, is delivered through the plurality of needles 3416. In some embodiments, the heating chamber 3410 is located in the catheter body proximate opening 3415 and is configured to use RF resistive heating for generating steam or vapor.

Since the needles 3416 pierce into the target tissue during use, the steam or vapor 3417 delivered through the pierced needles 3416 cause ablation of tissue located deep within the target tissue. The coolant 3419 directly contacts the surface of the non-target urethral tissue and maintains the ambient temperature on the surface of the non-target tissue to a desired level, such as below 60 degrees C. in some embodiments, preventing or diminishing clinically significant or circumferential thermal injury to the non-target tissue. This enables the vapor 3417 to ablate deeper prostatic tissue without circumferentially ablating the urethral tissue at the surface. Also, the membrane 3425 insulates the piercing needles 3416 and prevents the coolant 3419 from significantly cooling the needles 3416. In some embodiments, the heating chamber 3410 is at the distal end of the catheter proximal to the most proximal needle 3416 and plurality of openings 3415, and is configured to use RF energy to generate vapor using resistive or ohmic heating of saline. The catheter is optimized to minimize any leakage of RF electrical current into the tissue. In no situation is leakage sufficient to create a clinically significant ablation lesion.

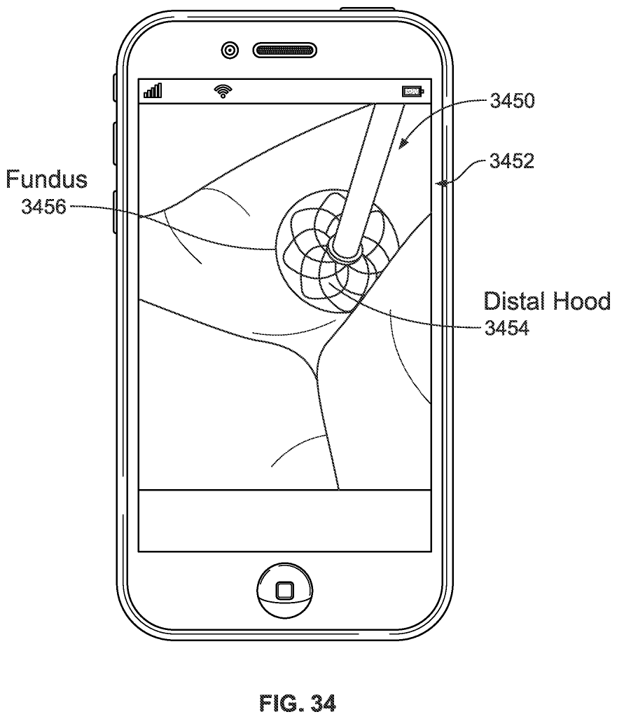

FIG. 7C is an illustration of prostate ablation being performed on an enlarged prostrate in a male urinary system using the ablation catheter 3400 of FIG. 7A, in accordance with an embodiment of the present specification. Also depicted in FIG. 7C are the prostate 3430 and prostatic urethra 3432. Referring now to FIGS. 34A and 34C, the ablation catheter 3400 with the heating chamber 3410 and the inflatable cooling balloon 3425 is inserted into the patient's urethra and advanced into the prostatic urethra 3432 so as to position the first plurality of openings 3415 and second plurality of openings 3418 proximate the prostatic tissue to be ablated. The coolant 3419 is delivered, through the second plurality of openings 3418, to the prostatic urethra 3432. Using a pusher, the needles 3416 are then pushed out at an angle (ranging between 30 and 90 degrees, in various embodiments) from the catheter 3400 into the prostate 3430. The pushed out needles 3416 also perforate or traverse the insulating membrane 3425 covering the openings 3415.

Water or saline (through the water inlet port 3411) is administered into the chamber 3410 where it is converted into steam or vapor 3417. The steam or vapor 3417 travels through the body 3405 of the catheter and exits from openings in the needles 3416 into the prostatic tissue, thus ablating the prostatic tissue. The needles 3416 are insulated by the membrane 3421 while the needles 3416 perforate the membrane 3425. The coolant filled inflated balloon 3425, as well as the coolant 3419 delivered to the prostatic urethra 3432, via the second plurality of openings 3418, maintain the ambient temperature on the surface of the prostatic tissue to a desired level, such as below 60 degree C. in some embodiments and, preferably, below 40 degree C. in other embodiments. This enables the vapor 3417 to ablate deeper prostatic tissue without ablating the prostatic urethra tissue at the surface in clinically significant or circumferential fashion. Optional temperature sensors can be installed to detect the temperature of the prostatic urethra and modulate the delivery of vapor 3417 and/or coolant 3419.

FIG. 7D is a flow chart listing the steps involved in a transurethral enlarged prostate ablation process using the ablation catheter 3400 of FIG. 7A, in accordance with one embodiment of the present specification. Referring now to FIGS. 34A and 34D, at step 3440, the ablation catheter 3400 is inserted into the urethra and advanced until the first plurality of openings 3415 is positioned proximate the prostatic tissue to be ablated within the prostatic urethra. At step 3442, the cooling balloon 3425 is inflated, with coolant supplied from the coolant port 3412, to fix the catheter 3400 within the prostatic urethra and maintain ambient temperature on the surface of the prostatic tissue to be ablated. Using a pusher, at step 3444, the needles 3416 are then pushed out at an angle (between 10 and 90 degrees, in various embodiments) from the catheter 3400 to pierce through the insulating membrane 3421, through the prostatic urethra and into the prostate up to a desirable depth. Vapor 3417 is delivered, from openings in the needles 3416, into the prostatic tissue at the desirable depth, thus ablating the prostatic tissue, without ablating the surface of the prostatic tissue. At step 3446, coolant 3419 is administered into the prostatic urethra, via the second plurality of openings 3418, to maintain ambient temperature on the surface of the prostatic tissue to be ablated. The membrane 3421 insulates the piercing needles 3416 from the coolant 3419 administered into the prostatic urethra. An optional temperature sensor is utilized to monitor the temperature of the surface of the prostatic tissue and control or modulate the flow of the coolant to maintain the temperature of the surface of the prostatic tissue below a specific temperature, which, in some embodiments, is 60 degree C.

Referring back to FIGS. 6A and 7A, in accordance with some embodiments, a pump, such as a syringe pump or a peristaltic pump, is used to control the flow of water to the heating chamber 3310, 3410.

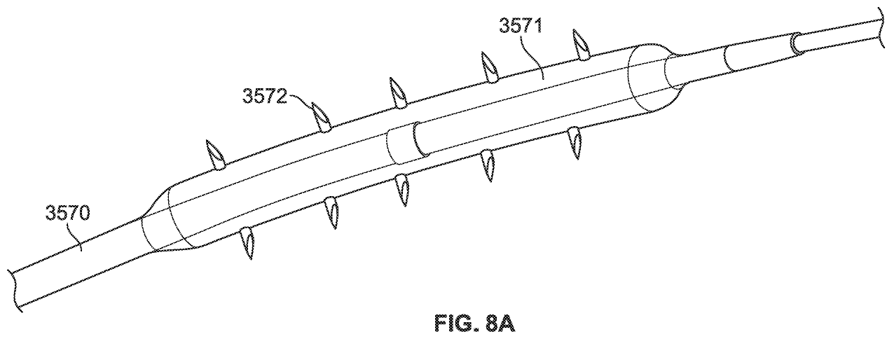

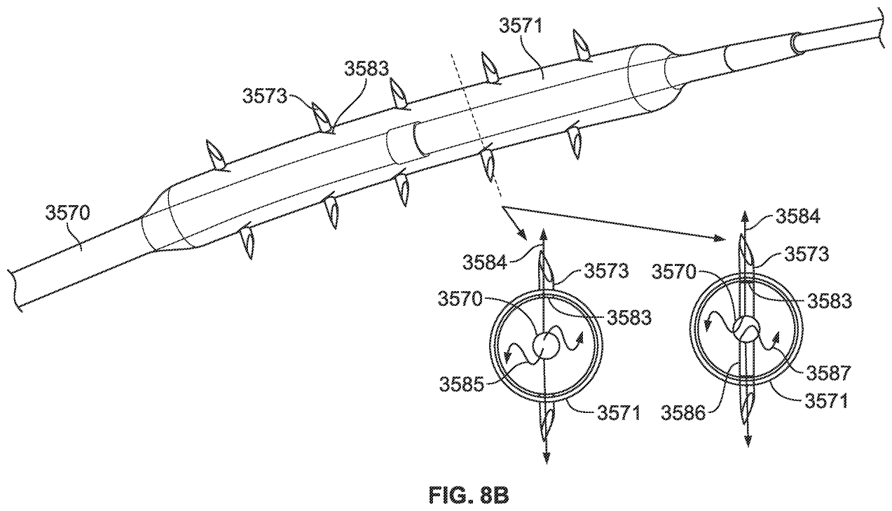

In various embodiments, the catheters of the present specification further include at least one thermally conducting element attached to the positioning element. The at least one thermally conducting element is configured to physically contact, and, in some embodiments, penetrate, a target tissue and enhance the delivery of thermal energy into the target tissue for ablation. FIG. 8A is an illustration of one embodiment of a positioning element 3571 of an ablation catheter 3570, depicting a plurality of thermally conducting elements 3572 attached thereto. In various embodiments, the positioning element 3571 is an inflatable balloon. The positioning element, or balloon 3571, is inflated to a first volume to bring the thermally conducting elements 3572 into contact with a target tissue. An ablative agent is then delivered to the target tissue through the catheter 3570 and out via at least one delivery port at the distal end of the catheter 3570. Thermal energy from the ablative agent is transferred from the lumen of the catheter 3570 into the air in the balloon 3571, further expanding the volume of the balloon 3571 and pushing the thermally conducting elements 3572 further into the target tissue. Thermal energy from the air in the balloon 3571 is transferred to the thermally conducting elements 3572 and is released into the target tissue for ablation. In various embodiments, the thermally conducting elements 3572 comprise solid or hollow metal spikes or needles. In various embodiments, the balloon 3571 is composed of a thermally insulating material so that ablative thermal energy is predominantly transferred from the thermally conducting elements 3572 into the target tissue.

FIG. 8B is an illustration of one embodiment of a positioning element 3571 of an ablation catheter 3570, depicting a plurality of hollow thermally conducting elements 3573 attached thereto. In one embodiment, each hollow thermally conducting element 3573 includes a valve 3583 at the inlet from a lumen of the positioning element 3571 to a lumen of the hollow thermally conducting element 3573. In various embodiments, the positioning element 3571 is an inflatable balloon. The positioning element, or balloon 3571, is inflated to a first volume to bring the thermally conducting elements 3572 into contact with a target tissue. An ablative agent is then delivered to the target tissue through the catheter 3570 and out via at least one delivery port at the distal end of the catheter 3570. Thermal energy from the ablative agent is transferred from the lumen of the catheter 3570 into the air in the balloon 3571, further expanding the volume of the balloon 3571 and pushing the thermally conducting elements 3573 further into the target tissue. Thermal energy from the air in the balloon 3571 is transferred to the thermally conducting elements 3573 and is released into the target tissue for ablation. In various embodiments, the thermally conducting elements 3573 comprise hollow metal spikes or needles. The thermally conducting elements 3573 include at least one opening at their distal ends which are in fluid communication with a lumen of the thermally conducting elements 3573, which, in turn, is in fluid communication with the interior of the balloon 3571. As seen in the cross section of the catheter 3570, vapor follows a first pathway 3584 to pass from the interior of the balloon 3571, through the thermally conducting elements 3573, and out to the target tissue. In one embodiment, each thermally conducting element 3573 includes a valve 3583 positioned at its junction with the balloon 3571 to control the flow of vapor into each hollow thermally conducting element 3573. In one embodiment, the vapor also follows a second pathway 3585 into the interior of the balloon 3571 to transmit thermal energy and assist in balloon expansion 3571. In another embodiment, flexible tubes 3586 connect the lumen of each thermally conducting element 3573 with a lumen of the catheter 3570, bypassing the interior of the balloon 3571. In one embodiment, the tubes 3586 are composed of silicone. In this embodiment, the vapor can only travel via the first pathway 3584 and air 3587 is used to expand the balloon 3571. In various embodiments, the balloon 3571 is composed of a thermally insulating material so that ablative thermal energy is predominantly transferred from the thermally conducting elements 3573 into the target tissue. In various embodiments, the thermally conducting elements 3573 possess shape memory properties such that they change shape from being generally parallel to the catheter 3570 at a temperature below a patient's body temperature to being generally perpendicular to the catheter 3570 at temperatures above the patient's body temperature.

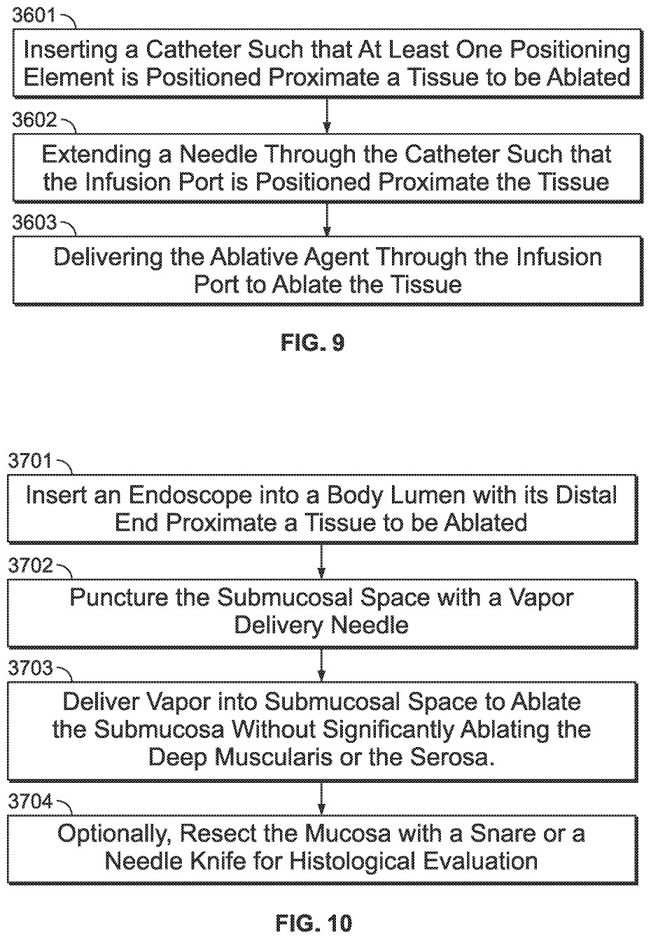

FIG. 9 is a flowchart illustrating one embodiment of a method of ablation of a tissue using a needle catheter device as described above. The device includes a thermally insulated catheter having a hallow shaft and a retractable needle through which an ablative agent can travel, at least one infusion port on the needle for delivery of the ablative agent, at least one positioning element on a distal end of the catheter, and a controller comprising a microprocessor for controlling the delivery of ablative agent. Referring to FIG. 9, in the first step 3601, a catheter is inserted such that a positioning element is positioned proximate to the tissue to be ablated. The next step 3602 involves extending the needle through the catheter such that the infusion port is positioned proximate to the tissue. Finally in step 3603, an ablative agent is delivered through the infusion port to ablate the tissue. In another embodiment, the device does not include a positioning element and the method does not include a step of positioning the positioning element proximate the tissue to be ablated.

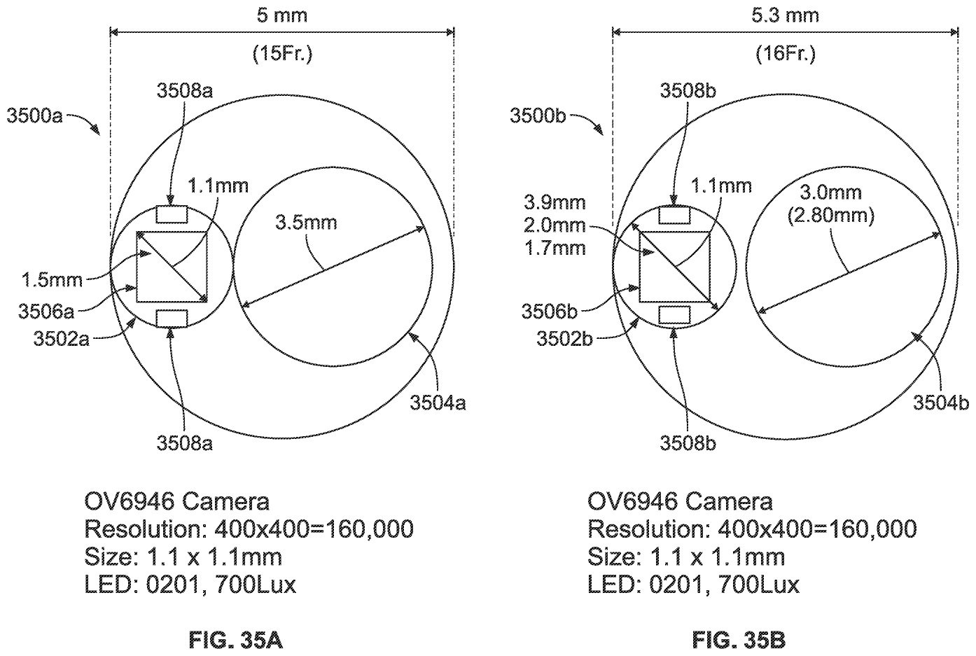

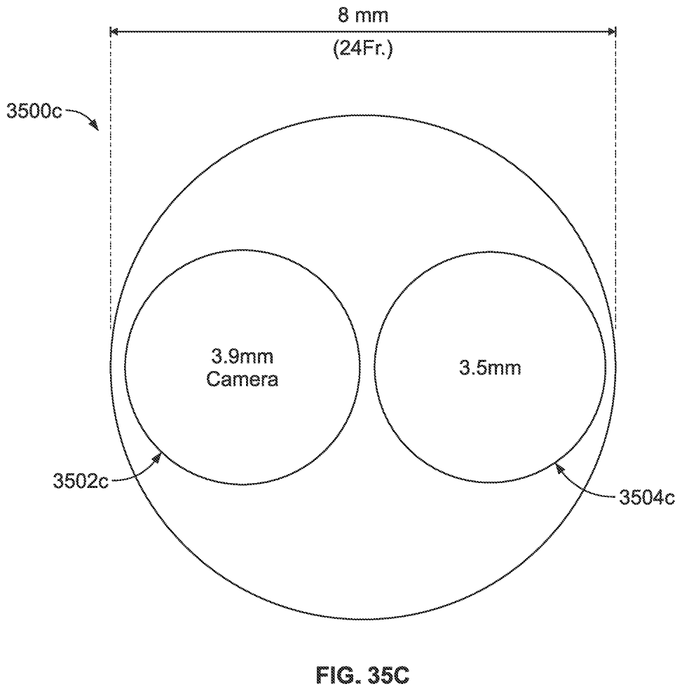

In one embodiment, the needle catheter device described in FIGS. 35A and 35B is also used for vapor ablation of submucosal tissue.

FIG. 10 is a flowchart illustrating a method of ablation of a submucosal tissue using a needle catheter device similar to those as described above. Referring to FIG. 10, in the first step 3701, an endoscope is inserted into a body lumen with its distal end proximate a tissue to be ablated. Next, in step 3702, the submucosal space is punctured using a vapor delivery needle, which is passed by means of a catheter through a working channel of the endoscope. Next, in step 3703, vapor is delivered into a submucosal space, predominantly ablating the submucosa and/or mucosa without irreversibly or significantly ablating the deep muscularis or the serosa. In one embodiment, the mucosa can be optionally resected with a snare or a needle knife for histological evaluation in step 3704. In some embodiments, the submucosa is pre-treated to create a submucosal lift with either a saline injection, glucose solution, glycerol, sodium hyaluronate (SH), colloids, hydroxypropyl methylcellulose, fibrinogen solution, autologous blood, or other alternatives or injection of other agents known in the art, such as Eleview™.

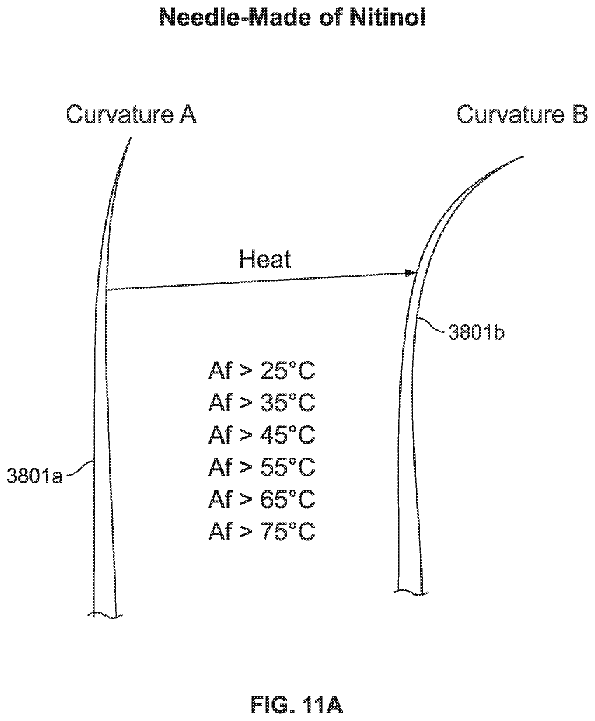

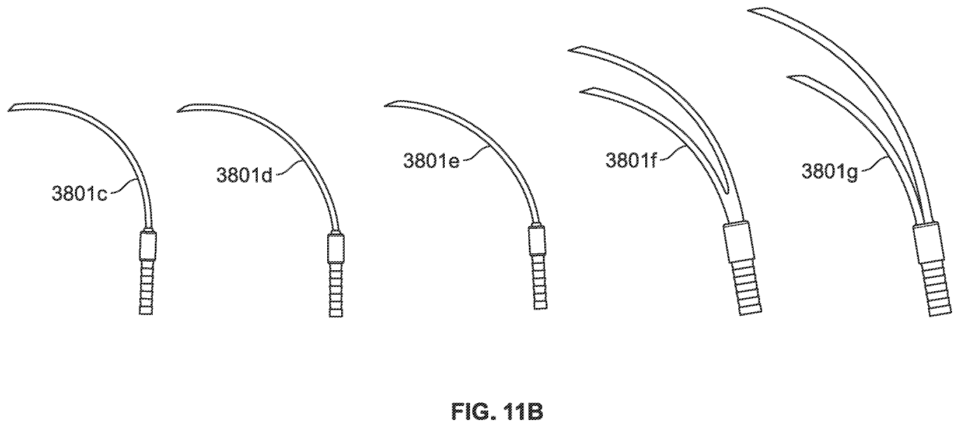

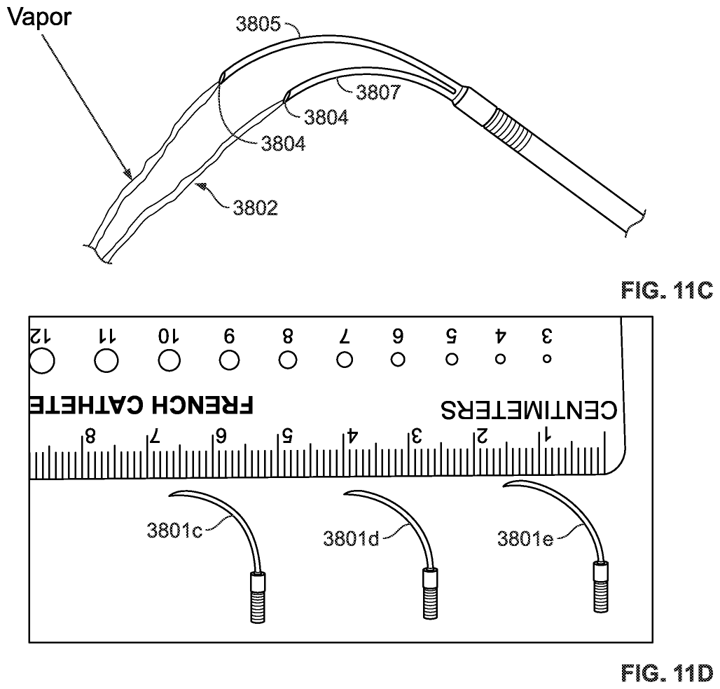

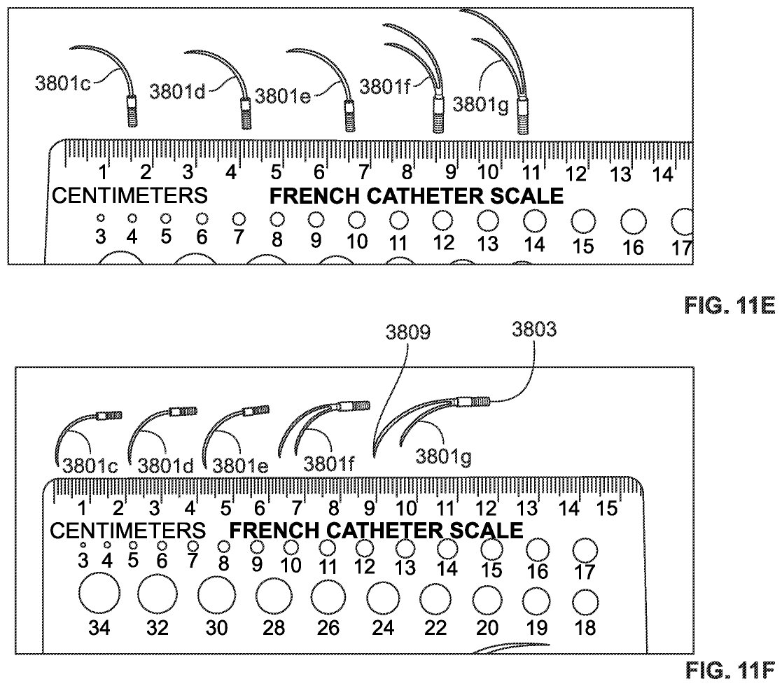

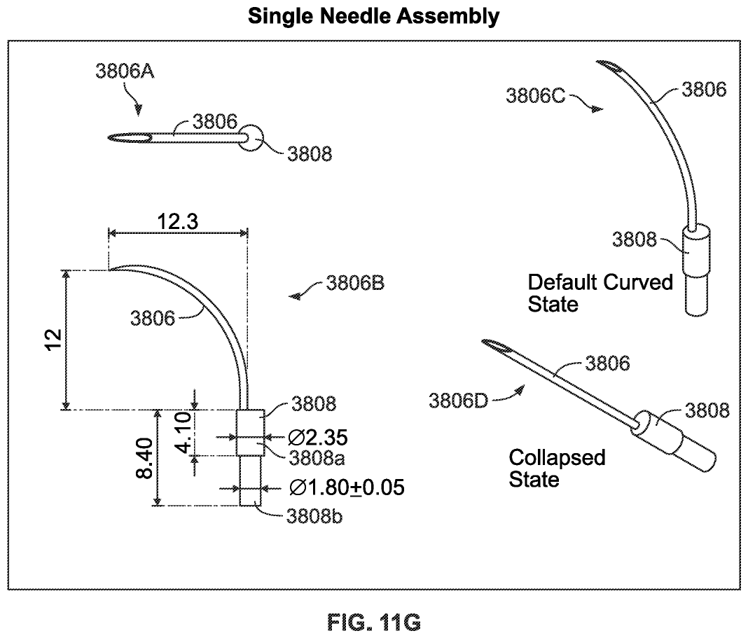

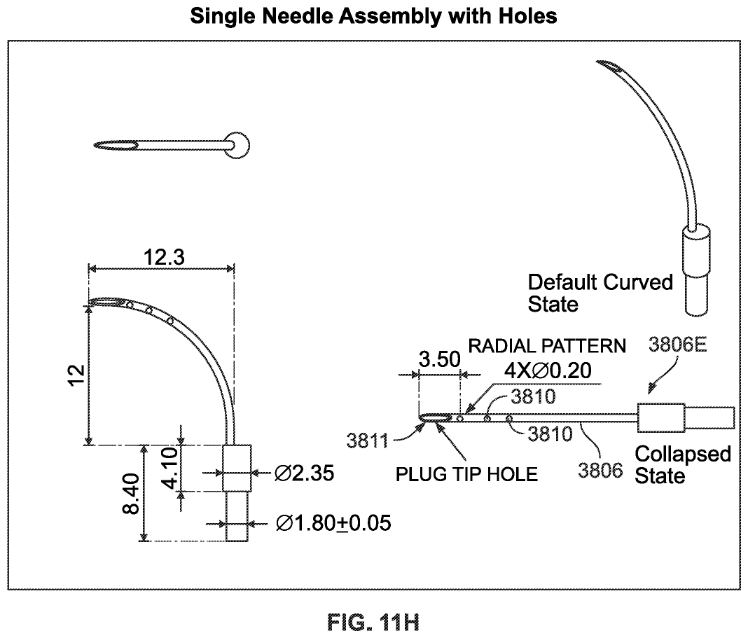

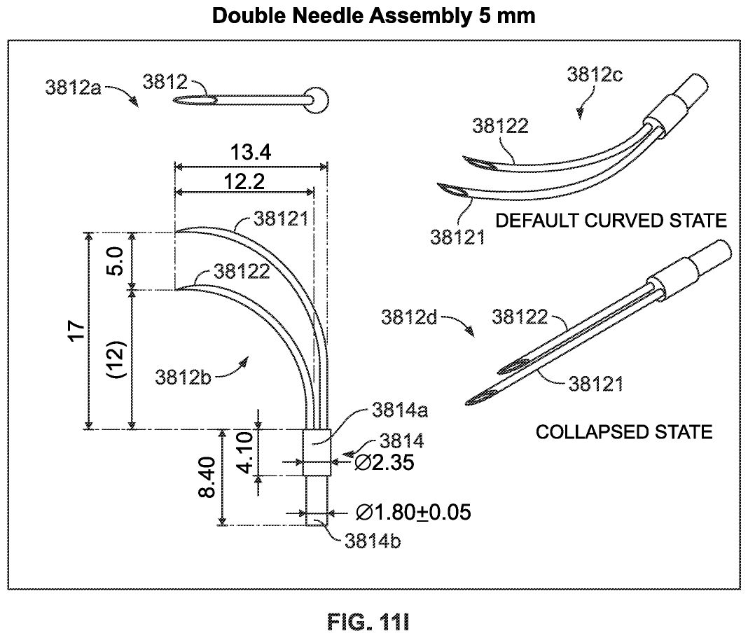

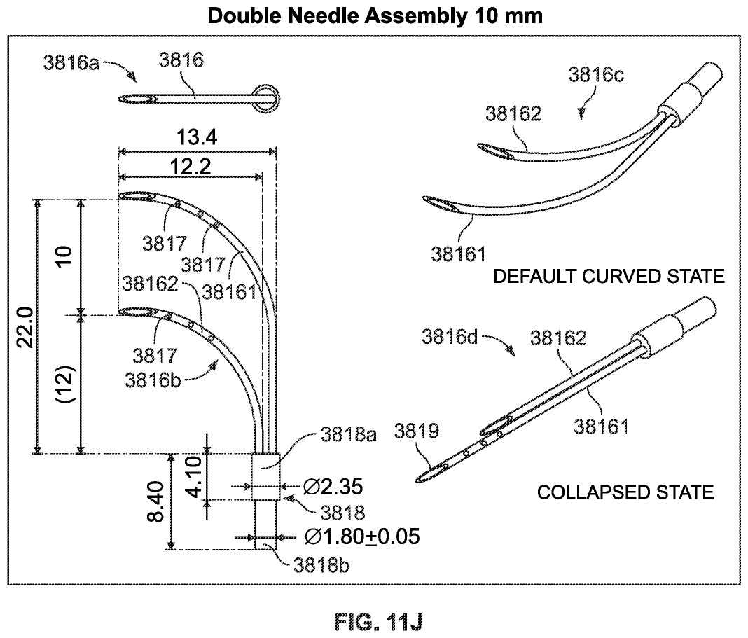

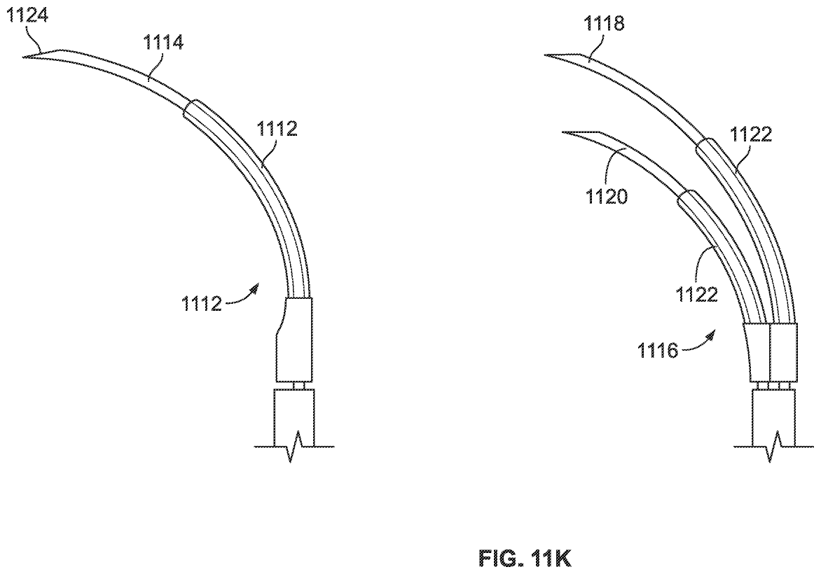

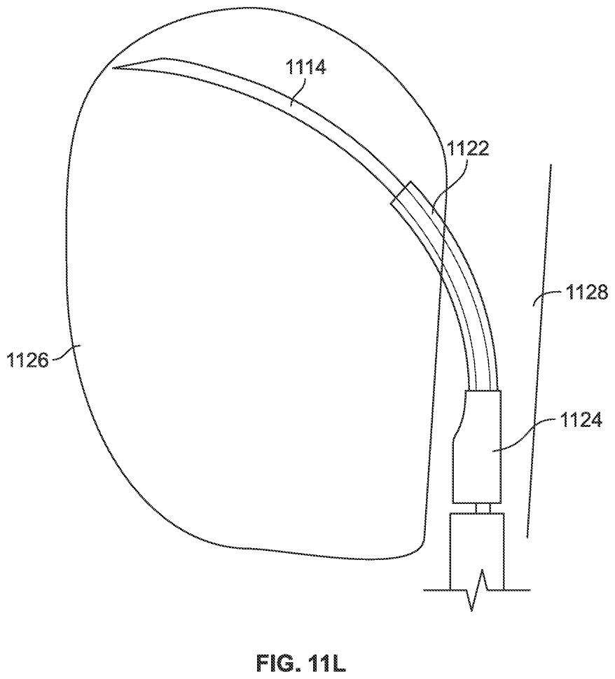

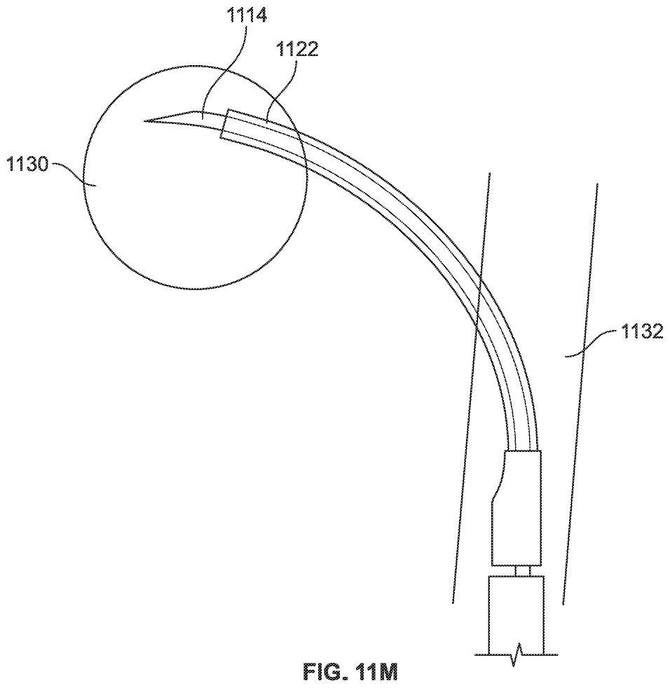

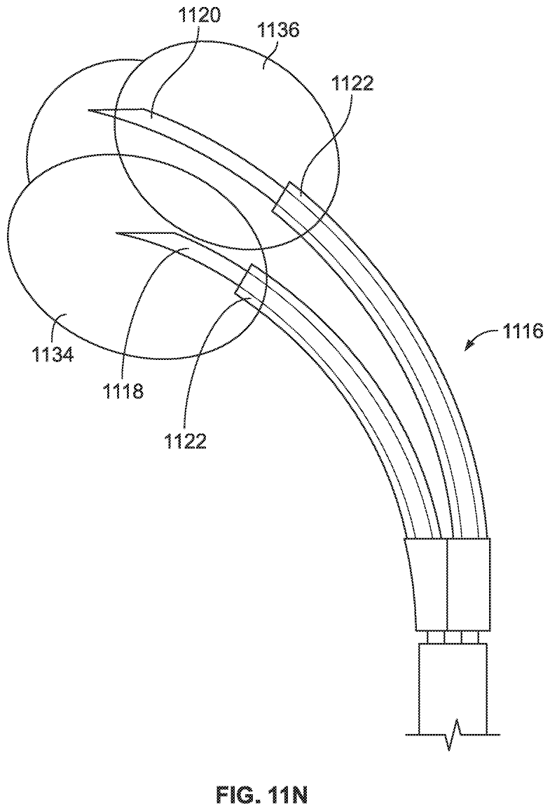

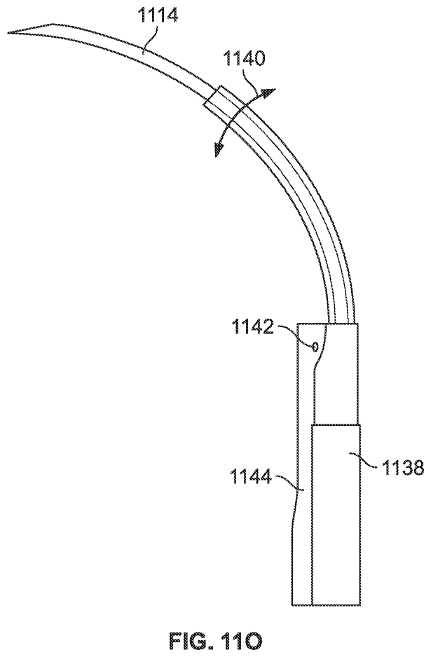

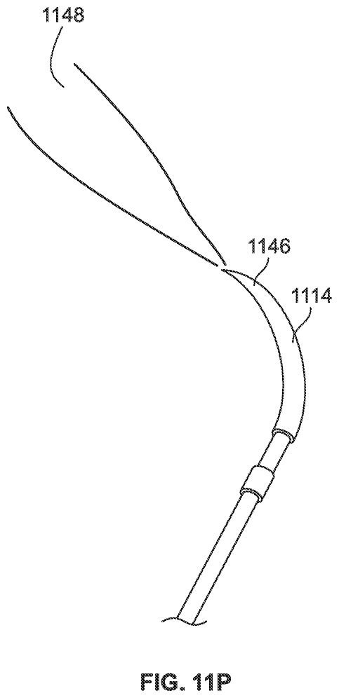

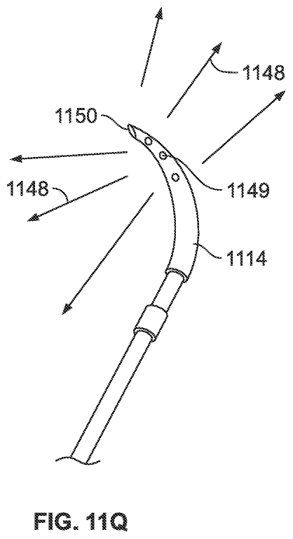

In another embodiment, the present specification discloses shape changing needles for ablation of prostatic tissue. FIG. 11A is an exemplary illustration of shape changing needles. Referring to FIG. 11A, needle 3801a is made up of a flexible material, such as nitinol, and has a curvature in the range of −30 to 120°. In some embodiments, the needle tip curves from 0 to 180°. In one embodiment, when heat is applied to the needle 3801a, its curvature increases, as shown by 3801b. In one embodiment, for an increase in temperature in the range of 25 degrees C. to 75 degrees C., the increase in the curvature of the needle ranges from −30 to 120°. In accordance with an aspect, the needle 3801a is hollow and includes at least one opening to allow delivery of an ablative agent, such as steam or vapor through the needle. In some embodiments, tension wires fixed to the needle can be pulled to change the shape of the needle or stabilize the needle to assist in puncture. In some embodiments, pulling on these tension wires can assist with making the puncture or help drive the needle deep into the prostatic tissue.

FIG. 11B illustrates different embodiments of needles, in accordance with the present specification. Referring to FIG. 11B, needles 3801c, 3801d, and 3801e, are single needles of different curvatures. Needles 3801f and 3801g are double needles of different sizes. In some embodiments, the needles 3801c, 3801d, 3801e, 3801f and 3801g are covered in an outer insulation layer, described subsequently in FIGS. 11K to 11Q. Needles 3801f and 3801g illustrate exemplary embodiments of two needles that are extended from a single port. In some embodiments, needles of FIG. 11B are made from 22 gauge stainless steel. FIG. 11C illustrates an exemplary process of delivery of an ablative agent 3802 from hollow openings 3804 at the edges of a pair of needles 3805, 3807 of a double needle, such as double needles 3801f or 3801g of FIG. 11B, in accordance with some embodiments of the present specification.

FIG. 11D illustrates exemplary depths or penetrating depths of needles 3801c, 3801d, and 3801e of different curvatures, in accordance with some embodiments of the present specification. The depth increases with the increase in the curvature. In some embodiments, the needles 3801c, 3801d, and 3801e have a curvature that varies between 0 and 150 degrees, with a diameter from 15 to 30 Gauge, and a length of each needle 3801c, 3801d, and 3801e ranging from 0.2 to 5 centimeters (cm). FIG. 11E illustrates exemplary depths or penetrating depths of needles 3801f and 3801g, relative to needles 3801c, 3801d, and 3801e of FIG. 11D, in accordance with some embodiments of the present specification. FIG. 11F illustrates exemplary lengths of needles 3801c, 3801d, 3801e, 3801f, and 3801g of FIG. 11E, extending in a straight line from a proximal port 3803 to the farthest distal point 3809 reached by the body of the needles, in accordance with some embodiments of the present specification.

FIG. 11G illustrates different views of a single needle assembly 3806 extending from a port 3808, in accordance with some embodiments of the present specification. In embodiments, the port 3808 includes two cylindrical portions, a first portion 3808a and a second portion 3808b, where the second portion 3808b is connected to an inner catheter (such as inner catheter 107m of FIG. 1M), while the first portion 3808a is attached to the second portion 3808b and a distal edge of first portion 3808a provides for an exit of one or more needles, such as needle 3806. Additionally, FIG. 11G illustrates a top view 3806A, a side view 3806B, and a front perspective view 3806C of the needle 3806 in its default curved state. A side perspective view 3806D of the needle 3806 in a linear, collapsed state is also illustrated. In an embodiment, a length of the needle 3806 extending in a straight line from the a distal edge of first portion 3808a to the farthest point of the needle 3806 is approximately 12 mm, and a depth from a sharp edge of the needle 3806 to the port, measured in a straight line, is approximately 12.3 mm. In some embodiments, the first portion 3808a of the port 3808 has a length of approximately 4.10 mm and a diameter of approximately 2.35 mm. In some embodiments, the second portion 3808b of the port 3808 has a length of approximately 4.30 mm and a diameter in a range of approximately 1.75 to 1.85 mm. FIG. 11H illustrates one or more holes 3810 at the sharp edge of the needle 3806 in another horizontal view of the needle 3806, in accordance with some embodiments of the present specification. In some embodiments, each of the holes 3810, used to deploy ablative vapor, extends for a length of about 3.50 mm at one side of tip of the hollow cylindrical needle 3806. The holes are positioned on a side along the length of the needle 3806, while a distal tip of the needle 3806 is occluded. In some embodiments, the distal tip may be occluded with a plug 3811 made from biocompatible material, such as for example stainless steel. In some embodiments, the distal tip is occluded and the vapor comes exits from the sides of the distal tip.

FIG. 11I illustrates different views of a double needle assembly 3812 extending from a port 3814, in accordance with some embodiments of the present specification. FIG. 11J illustrates different views of another double needle assembly 3816 extending from a port 3818, in accordance with some embodiments of the present specification. Referring simultaneously to FIGS. 11I and 11J, the port 3814, 3818 may include two cylindrical portions, a first portion 3814a, 3818a and a second portion 3814b, 3818b, where the second portion 3814b, 3818b is connected to an inner catheter (such as inner catheter 107m of FIG. 1M), while the first portion 3814a, 3818a is attached to the second portion 3814b, 3818b and a distal edge of first portion 3814a, 3818a provides for an exit of a double needle assembly 3812, 3816. The double needle assembly includes a first needle 38121, 38161 and a second needle 38122, 38162. FIGS. 11I and 11J illustrate a top view 3812a, 3816a, a side view 3812b, 3816b, and a top side perspective view 3812c, 3816c of the needles 3812, 3816 in their default curved states. A side perspective view 3812d, 3816d of the needles 3812, 3816 in a linear, collapsed state is also illustrated. Referring to FIG. 11I, a length of the needle 38121 extending in a straight line from a distal edge of port 3814 to the farthest point of the needle 38121 is approximately 17 mm, and a depth from a sharp edge of the needle 38121 to the port 3814 measured in a straight line, is approximately 13.4 mm. A length of the needle 38122 extending in a straight line from a distal edge of port 3814 to a farthest point of the needle 38122 is approximately 12 mm, and a length from a sharp edge of the needle 38122 to the port 3814 measured in a straight line, is approximately 12.2 mm. In embodiments, the port 3814 is configured similarly to port 3808. The distance between the sharp edges of needles 38121 and 38122 is approximately 5 mm. Referring to FIG. 11J, a length of the needle 38161 extending in a straight line from a distal edge of port 3818 to the farthest point of the needle 38161 is approximately 22 mm, and a length from a sharp edge of the needle 38161 to the port 3818 measured in a straight line, is approximately 13.4 mm. A length of the needle 38162 extending in a straight line from a distal edge of port 3818 to the farthest point of the needle 38162 is approximately 12 mm, and a length from a sharp edge of the needle to the port 3818 measured in a straight line, is approximately 12.2 mm. In embodiments the port 3818 is configured similarly to port 3808.

The distance between the sharp edges of needles 38161 and 38162 is approximately 10 mm. In some embodiments, one or both of needles 38161 and 38162 has one or more openings or holes 3817 on the sides, along their length, while distal tip of the one or both needles 38161 and 38162 that has the holes 3817 is occluded with a plug 3819. The holes 3817 provide an exit for ablation vapors.

FIG. 11K illustrates an insulation 1122 on a single needle configuration 1112 comprising a needle 1114, and a double needle configuration 1116 comprising needles 1118 and 1120. Each of needle 1114, 118, and 1120 may have one or more openings, such as an opening 1124 at the tip of the needle 1114, to enable an exit for vapor during ablation. The insulation 1122 insulates a portion of the needles' 1114, 118, and 1120 outer length. The insulation 1122 can be added, in some embodiments, as a shrink tube or as spray on. In different embodiments, insulation 1122 extends along any portion of a length of needles 1114, 118, and 1120, from their distal tip to their base, but do not cover any openings at the distal tip or along the length of the needles. An ablation area may be modified by changing distribution of insulation 1122 on the needles. This is illustrated with reference to FIGS. 11L, 11M, and 11N.

FIG. 11L illustrates a single needle configuration 1114 with insulation 1122 positioned inside a prostatic tissue 1126 in accordance with some embodiments of the present specification. The insulation 1122 covers the portion of the needle 1114 that extends from a catheter 1124 to the length of the needle 1114 before a tip of the needle, so that a portion of the insulation 1122 extends into the prostatic tissue from a urethra 1128, therefore protecting the urethra 1128. FIG. 11M illustrates a single needle configuration 1114 with insulation 1122 positioned inside a uterine fibroid 1130 in accordance with some embodiments of the present specification. The needle 1114 extends from a uterus 1132 into the fibroid 1130. The insulation 1122 covers a greater extent of the needle 1114, relative to the extent shown in FIG. 11L, so that the insulation 1122 extends into the fibroid 1130 along with a small portion of the tip of the needle 1114 and delivers ablation vapor only to the fibroid 1130, while protecting parts of the anatomy outside the fibroid. FIG. 11N illustrates a double needle configuration 1116 where the two needles 1118 and 1120 are inserted into separate prostate lobes 1134 and 1136, in accordance with some embodiments of the present specification. The insulation 1122 covering both needles 1118 and 1120 extends into the lobes 1134 and 1136 along with the non-insulated distal tips of the needles.

FIG. 11O illustrates an exemplary embodiment of a steerable catheter shaft 1138 in accordance with some embodiments of the present specification. Catheter shaft 1138 is configured to be flexible so that it may be steered by a user to direct a needle 1114 in a required direction. Referring to the figure, an arrow 1140 indicates the ability to steer the needle in different directions, using the catheter shaft 1138. In embodiments, a viewing device 1142 is configured at the tip of the catheter shaft 1138 at the base of the needle 1114 to help the user articulate direct visualization of the needle 1114. In embodiments, the viewing device 1142 includes a camera, lens, LEDs, or any other equipment to facilitate direct visualization of the needle's 1114 position and movement within the anatomy of a patient, thereby aiding the physician in steering the needle 1114. In embodiments, a channel 1144 in the catheter shaft 1138 provides for containing optical and electrical wires that connect the viewing device 1142 to a controller, such as controller 15q, for power and for displaying the captured images on a screen or split-screen for both viewing the ablation area and controlling the ablation delivery. In some other embodiments, the viewing device 1142 interfaces with a peripheral computing and/or imaging device, for example, an iPhone, to display the images captured by its camera. In embodiments, controls of the viewing device 1142 are provided in a handle of the catheter shaft 1138. In one embodiment, the needle is steered using a plurality of tension wires attached to the needle and pulling on those tension wires allow to manipulate the position or direction of the needle tip.

FIG. 11P illustrates a needle 1114 with an open tip 1146, in accordance with some embodiments of the present specification. The figure also shows steam 1148 that sprays out from the opening at the distal tip 1146. In practice, needle 1114 is first flushed with water to get any air out, prior to spraying ablation vapor or steam 1148. FIG. 11Q illustrates an alternative embodiment of a needle 1114 with a plug 1150 at its distal tip to occlude the tip and comprising holes or openings 1149 along an uninsulated length of the needle 1114, close to the tip, to provide a sprinkler-style spray of steam 1148, in accordance with the present specification.

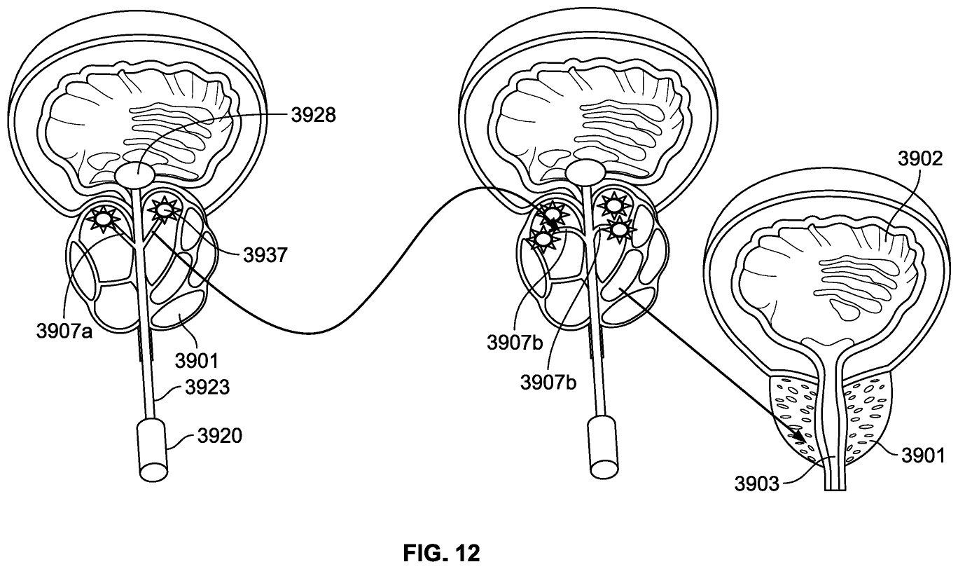

FIG. 12 is an illustration of transurethral prostate ablation being performed on an enlarged prostrate 3901 in a male urinary system using an ablation device, which makes use of shape changing needles, in accordance with one embodiment of the present specification. Also depicted in FIG. 12 are the urinary bladder 3902 and prostatic urethra 3903. An ablation catheter 3923 with a handle 3920 and a positioning element 3928 is inserted into the urethra 3903 and advanced into the bladder 3902. In one embodiment, the positioning element 3928 is inflated and pulled to the junction of the bladder with the urethra, thus positioning needles 3907a at a predetermined distance from the junction. Using a pusher (not shown) coupled to the handle 3920, the needles 3907a are then pushed out at an angle between 10 and 90 degrees from the catheter 3923 through the urethra 3903 into the prostate 3901. Vapor is administered through a port (not shown) that travels through the shaft of the catheter 3923 and exits from openings 3937 in the needles 3907a into the prostatic tissue, thus ablating the prostatic tissue. According to an embodiment, vapor delivery heats the needles and the needles change shape from substantially straight 3907a to curved in 3907b, while vapor is being delivered. On cessation of vapor delivery, the needles revert back to their original straight shape, which allows for easy retraction into the catheter. The mechanical shape change of needles allows for more effective distribution of the ablative energy within the prostatic tissue. In embodiments, the vapor is generated in the handle 3920 or the body 3923 of the catheter using inductive heating or resistive heating.

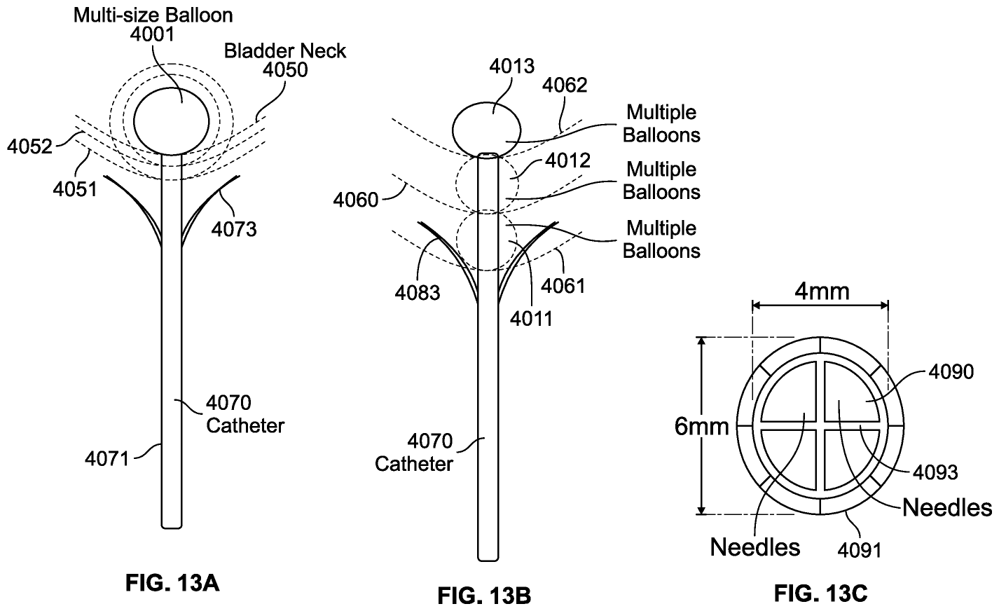

FIG. 13A is an illustration of one embodiment of a positioning element 4001 of an ablation catheter 4070 with needles 4073 attached to the catheter body. In various embodiments, the positioning element 4001 is an inflatable balloon. The positioning element, or balloon 4001, is inflated to a first volume, thus positioning needles 4073 at a predetermined distance from the bladder neck 4050 and bringing them into contact with the target tissue. In one embodiment, an ablative agent, such as steam or vapor, is delivered to the target tissue through the catheter 4070. Travelling through the shaft 4071 of the catheter, the vapor exits from openings (not shown) in the needles 4073 into the prostatic tissue, thus ablating the prostatic tissue. In one embodiment, the balloon 4001 is capable of being expanded to different sizes. This feature is used, in one embodiment, to progressively or sequentially inflate the balloon 4001 to different sizes, thereby positioning the needles at various fixed distances 4051, 4052 from the bladder neck 4050, allowing for treatment of discrete regions of the prostate tissue. In one embodiment, the predetermined distance at which the balloon may be used to place the needles ranges from 1 mm to 50 mm from the bladder neck. In one embodiment, the positioning element 4001 can be moved relative to the needle 4073, adjusting the range of the needles from 1 mm to 50 mm from the positioning element 4073. In another embodiment, the positioning element 4001 can engage with a length of the needle 4073, applying mechanical force helping the needle pierce the target tissue.

In another embodiment shown in FIG. 13B, a plurality of inflatable balloons 4011, 4012, 4013 are employed as positioning elements. These balloons may be used to position the needles 4083 at various fixed distances 4061, 4062 from the bladder neck 4060, allowing for treatment of discrete regions of the prostate tissue. It may be noted that any one of the plurality of balloons may be inflated, depending on the region of tissue to be ablated. The balloons may also be ablated in a sequential manner, to allow comprehensive coverage of target tissue. In one embodiment, the number of balloons ranges from one to five.

FIG. 13C illustrates a cross section of the distal tip of a catheter 4091, in accordance with an embodiment of the present specification. In one embodiment, for ablation of prostatic tissue, an inner diameter (ID) of the catheter employed is about 4 mm, and an outer diameter (OD) is about 6 mm. A plurality of thermally conductive elements 4090, such as needles, extend at an angle from the catheter 4091, wherein the angle ranges between 30 to 90 degrees. In one embodiment, the needles may be retracted into the catheter after ablation.

In one embodiment, the balloon is inflated prior to ablation. In another embodiment, the ablative agent, such as steam or vapor also transmits thermal energy and assists in balloon expansion. That is, thermal energy from the ablative agent is transferred from the lumen of the catheter into the air in the balloon, further expanding the volume of the balloon and pushing the needles further into the target tissue. In yet another embodiment, the balloon is inflated by filling it with a coolant that is supplied to the balloon through a coolant port at the proximal end of catheter. During use, the balloon is inflated with the coolant while vapor or steam is delivered through the plurality of needles. Since the needles pierce into the target tissue during use, the steam or vapor delivered through the pierced needles cause ablation of tissue located deep within the target tissue. The coolant filled inflated balloon contacts the surface of the target tissue and maintains the ambient temperature on the surface of the target tissue to a desired level, such as below 60 degrees C. in some embodiments. This enables the vapor to ablate deeper tissue without ablating the tissue at the surface.

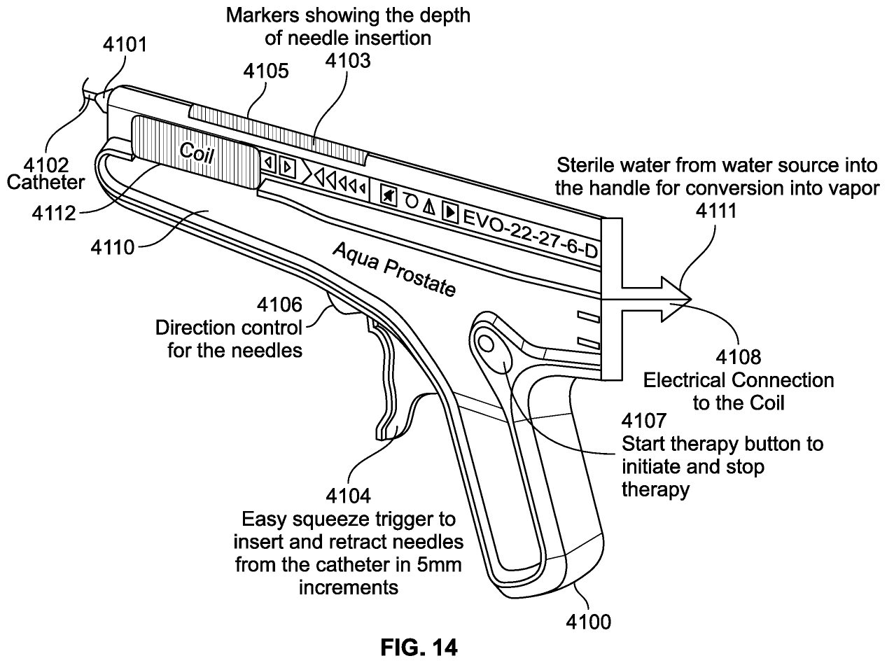

FIG. 14 illustrates one embodiment of a handle mechanism 4100 that may be used for deployment and retrieval of needles at variable depths of insertion, when ablating prostatic tissue. Referring to FIG. 14, in one embodiment, the handle 4100 is shaped like a handheld gun or pistol, which allows it to be conveniently operated by a physician for the treatment of prostatic tissue. The tip 4101 of the handle is equipped with a slot, into which an ablation catheter 4102 may be inserted for passing into the urethra of the patient. Ablation needles are coupled to the catheter, as explained in the embodiments above, and are used to deliver steam vapor to target tissue. On the top of the handle 4100, markers 4103 are placed, which indicate the depth of insertion of the needles. The markers may be placed by printing, etching, painting, engraving, or by using any other means known in the art suitable for the purpose. In one embodiment, the ablation needles may be inserted or retracted in increments of a fixed distance—such as 5 mm, and therefore markers are placed correspondingly to reflect the increments. A button 4105 is provided on the markers, which advances or retracts by a mark, each time the catheter and the needles are advanced or retracted by the preset distance. In one embodiment, a trigger 4104 is provided on the handle mechanism, which may be pressed to advance the needles for the preset increment of distance. In one embodiment, once the needles are advanced to the maximum distance by repeatedly pressing the trigger—as indicated by the button 4105 on the markers, further pressing of the trigger results in retraction of the needles, one increment of distance at a time. It may be noted that as explained in the embodiments above, the catheter is also equipped with a positioning element, such as a balloon, which does not allow the catheter and the needles to be advanced beyond a fixed distance in the urethra.

In one embodiment, a knob or a button 4106 is provided which may be turned or pressed to control the direction of movement of the catheter and the needles. That is, the knob 4106 may be used to determine whether the catheter and needles are moved forward (advanced) or backward (retracted), each time the trigger 4104 is pressed.

In one embodiment, the handle mechanism 4100 also comprises a heating chamber 4110, which is used to generate steam or vapor for supplying to the catheter 4102. The heating chamber 4110 comprises a metal coil 4112 wound about a ferromagnetic core. The chamber is filled with water via a water inlet port 4111 located at a proximal end of the handle mechanism 4100. In one embodiment, sterile water is supplied from a water source into the handle for conversion into vapor. The handle is also equipped with an electrical connection 4108 to supply the coil 4112 with electrical current from a current generator. Alternating current is provided to the coil 4112, thereby creating a magnetic field that induces electric current flow in the ferromagnetic core. This causes heating in the chamber 4110 and causes the water within to vaporize. The resulting steam or vapor, generated in the chamber 4110, is delivered through the needles placed at the appropriate location to ablate target tissue.

In an embodiment, a start/stop button 4107 is also provided on the handle 4100 to initiate or stop ablation therapy as required.

The same functionality can be achieved by other handle form-factors known in the art and also described in this application.

FIG. 15A is a flowchart illustrating a method of ablation of prostatic tissue in accordance with one embodiment of the present specification. Referring to FIG. 15A, the first step 4201 includes passing a catheter of an ablation device into a patient's urethra, wherein the catheter includes a hollow shaft through which an ablative agent can travel, at least one first positioning element, at least one second positioning element positioned distal to said at least one first positioning element, at least one input port for receiving an ablative agent, and a plurality of needles positioned on said catheter between said first and second positioning elements and configured to deliver ablative agent to a prostatic tissue. In an embodiment, the ablation device includes a controller comprising a microprocessor for controlling the delivery of the ablative agent. The catheter is passed through the urethra such that the first positioning element is positioned proximal to the prostatic tissue to be ablated and the second positioning element is positioned either in or distal to the prostatic tissue to be ablated. Next, in step 4202, the positioning elements are deployed such that they contact the urethra and the catheter is positioned within the urethra, proximate the prostatic tissue to be ablated. In the next step 4203, the plurality of needles is passed through the urethra into the prostatic tissue to be ablated. Finally, in step 4204, an ablative agent is delivered through the needles to ablate the prostatic tissue. Optionally, a sensor is used to measure a parameter of the prostate in step 4205 and the measurement is used to increase or decrease the flow of ablative agent being delivered in step 4206. Optionally, in an embodiment, a cystoscope is first inserted in the patient's urethra and the catheter is inserted through the cystoscope. In some embodiments, or more of the positioning elements is inflated with an insulative or a cooling fluid to insulate or cool the bladder neck or the prostatic urethra.

FIG. 15B is a flowchart illustrating a method of ablation of prostatic tissue in accordance with another embodiment of the present specification. Referring to FIG. 15B, the first step 4211 includes passing a catheter into a patient's urethra, wherein the catheter includes a hollow shaft through which an ablative agent can travel, at least one first positioning element, at least one second positioning element positioned distal to said at least one first positioning element, at least one input port for receiving an ablative agent, and a plurality of needles positioned on said catheter between said first and second positioning elements and configured to deliver ablative agent to a prostatic tissue. In an embodiment, the ablation device includes a controller comprising a microprocessor for controlling the delivery of the ablative agent. The catheter is passed through the urethra such that the first positioning element is positioned proximate the prostatic tissue to be ablated and the second positioning element is positioned within the bladder of the patient. Next, in step 4212, the second positioning element is deployed and the catheter is pulled back such that the second positioning element abuts a urethral opening at the neck of the bladder. The first positioning element is deployed such that the catheter is positioned within the urethra proximate to the prostatic tissue to be ablated in 4213. In the next step 4214, the plurality of needles is passed through the urethra into the prostatic tissue to be ablated. Finally, in step 4215, an ablative agent is delivered through the needles to ablate the prostatic tissue. Optionally, in an embodiment, a cystoscope is first inserted in the patient's urethra and the catheter is inserted through the cystoscope. In various embodiments, the order of deployment of the positioning elements can be reversed. In other embodiments, only one of the two positioning elements may be deployed to deliver the therapy.

FIGS. 15C to 15E illustrate an embodiment of using an expandable catheter 1500 to expand/widen a constricted prostatic urethra 1538, in accordance with some embodiments of the present specification. The prostatic urethra 1538 has been constricted by an enlarged prostate 1530. Referring to FIG. 15C, compressed catheter 1500 with an expandable element 1525 is advanced into a prostatic urethra 1538. In embodiments, the expandable element 1525 comprises an expandable balloon or a self-expanding balloon. In embodiments, the expandable element 1525 is covered, for example, by a semi-permeable sheath. In other embodiments, the expandable element 1525 is uncovered. In embodiments, the expandable catheter 1500 includes a center post 1537. The center post includes one or more rows 1533 each comprising a plurality of openings for the delivery of ablative agent. In embodiments, each plurality of openings has a pattern of openings which may vary in shape, diameter, and quantity of openings to regulate ablative agent distribution. Referring to FIG. 15D, the expandable element 1525 on catheter 1500 is expanded and presses on the urethral walls 1539 which presses on the prostate 1530. Ablative agent 1541, such as steam, is then delivered into the prostatic tissue from the plurality of openings. Referring to FIG. 15E, catheter 1500 is removed from urethra 1538, leaving a widened prostatic urethra 1538. FIG. 15F illustrates an expanded expandable element 1525 of a catheter 1500 and an exemplary use of one or more needles 1550 to allow delivery of an ablative agent 1541, such as steam or vapor, through a hollow exit at the edge of the needle 1550. The needles extend from the center post 1537 of the catheter 1500, through the urethral wall 1539 and into the prostate 1530 to deliver ablative agent 1541 to the prostatic tissue. While the illustration of FIG. 15F describes the placing of the element 1525 into the prostate, the same arrangement can be used for both BPH and urethral strictures. In embodiments, needles 1550 are one or the needles illustrated and described in context of FIGS. 11A to 11J. In some embodiments, the expandable element 1525 is a wire mesh stent which can be removed at a later date. In another embodiment, the expandable element 1525 is made of a bioresorbable material and resorbs after a predetermined time. In some embodiments, the expandable element 1525 has a constraining and/or removing mechanism attached to it for removal at a later date. In some embodiments, the constraining and or removing mechanism is a PTFE, ePTFE or silk suture. In some embodiments, the expandable element comprises extracellular matrix to help proper healing of the prostatic urethra post-ablation.

Median lobe hyperplasia is a benign condition in which the median lobe of the prostate become enlarged and presses into the base of the bladder, causing a ball valve type obstruction at the bladder neck. For ablation therapy, it is desirable to access the median lobe, especially the most affected part of the median lobe, trans-cystically rather than transurethrally. Accessing the median lobe of the prostate through the bladder, rather than through the urethra, has the advantage of not causing ablation damage to the urethra with subsequent urethral stricture. FIG. 15G illustrates an ablation catheter 1560 used to ablate prostatic tissue of a patient with median lobe hyperplasia via a trans-cystic approach, in accordance with one embodiment of the present specification. FIG. 15H illustrates an ablation catheter used to ablate prostatic tissue of a patient with median lobe hyperplasia via a trans-cystic approach, in accordance with another embodiment of the present specification. In the embodiment of FIG. 15G, the catheter 1560 includes at least one curved vapor delivery needle 1561 extending at its distal end. In the embodiment of FIG. 15H, the catheter 1565 includes at least one straight vapor delivery needle 1566 extending at its distal end. The one or more needles, and their composition and method of deployment, may be similar to the other needle embodiments discussed in the embodiments of the present specification. Referring to FIGS. 15G and 15H simultaneously, the catheter 1560, 1565 is depicted inserted into and through the patient's spongy or penile urethra 1571, through a prostatic urethra 1572, and into the patient's bladder 1573. In embodiments, the distal end of the catheter 1560, 1565 is advanced to be positioned just beyond a bladder neck 1574 and within the bladder 1573, just into the bladder 1573 past the internal urethral meatus 1576 (opening of the bladder into the prostatic urethra). At least one needle 1561, 1566 is extended from the distal end of the catheter 1560, 1565 into the bladder 1573 cavity, through the bladder wall 1577, into the medial lobe 1575. Ablative agent, in the form of vapor or steam, is delivered through the at least one needle 1561, 1566 to ablate the tissue of the median lobe 1575. In some embodiments, the catheter 1560, 1565 optionally includes at least one positioning element 1562, 1564 configured to position the catheter inside the bladder 1573 and to stabilize the needle 1561, 1566 to assist in the needle 1561, 1566 penetrating the median lobe 1575. In various embodiments, the positioning element 1562, 1564 comprises a shape memory material configurable between a first, collapsed configuration for delivery and a second, expanded configuration for positioning. In various embodiments, the positioning element 1562, 1564, in the second, expanded configuration, has a disc, cone, hood, ovoid, oval, square, rectangular, or flower shape. In various embodiments, tension wires attached to the needle may be used to manipulate the needle and assist in puncturing into the prostate.

FIG. 15I is a flowchart listing the steps in one method of using an ablation catheter to ablate prostatic tissue of a patient with median lobe hyperplasia via a trans-cystic approach, in accordance with one embodiment of the present specification. At step 1580, an ablation catheter comprising at least one needle is passed into a patient's spongy urethra and through a prostatic urethra such that a distal end of the catheter is positioned within the patient's bladder. Optionally, the ablation catheter further comprises at least one positioning element configured to position the catheter in the bladder and to stabilize the at least one needle for penetrating the median lobe. Optionally, at step 1581, the positioning element is deployed to position the catheter and stabilize the at least one needle. At step 1582, the at least one needle is extended from the distal end of the catheter and is passed through the bladder or bladder neck wall and into the median lobe of the prostate. At step 1583, an ablative agent is delivered through the at least one needle into the median lobe to ablate prostatic tissue. In embodiments, the ablation catheter is a part of an ablation system comprising a controller and a means for generating the ablative agent. At step 1584, the controller controls the delivery of ablative agent to maintain a pressure in the bladder and median lobe below 5 atm.

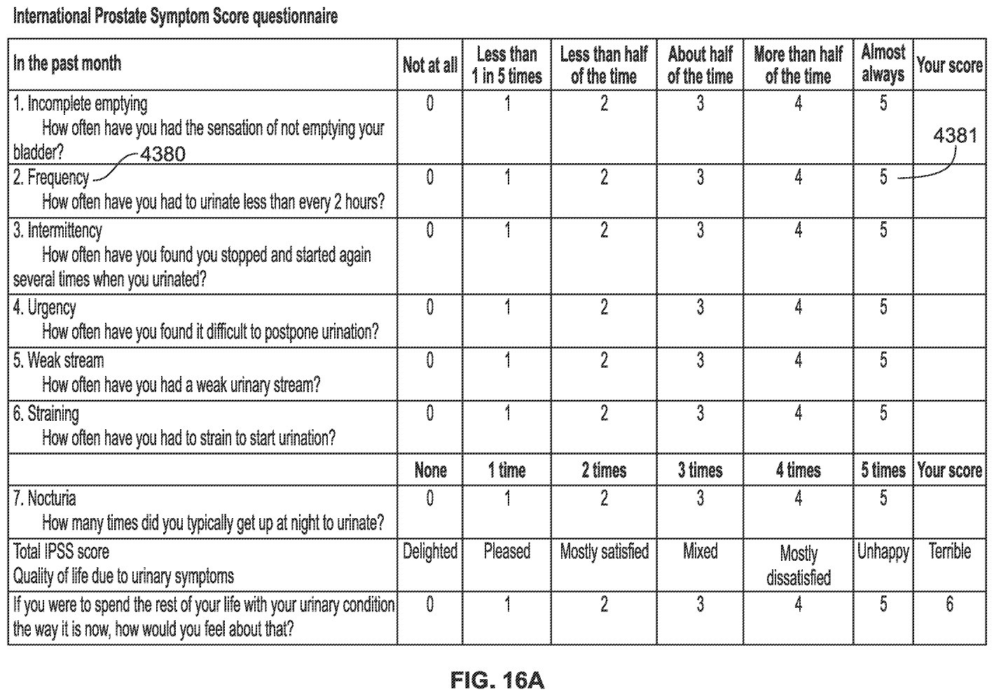

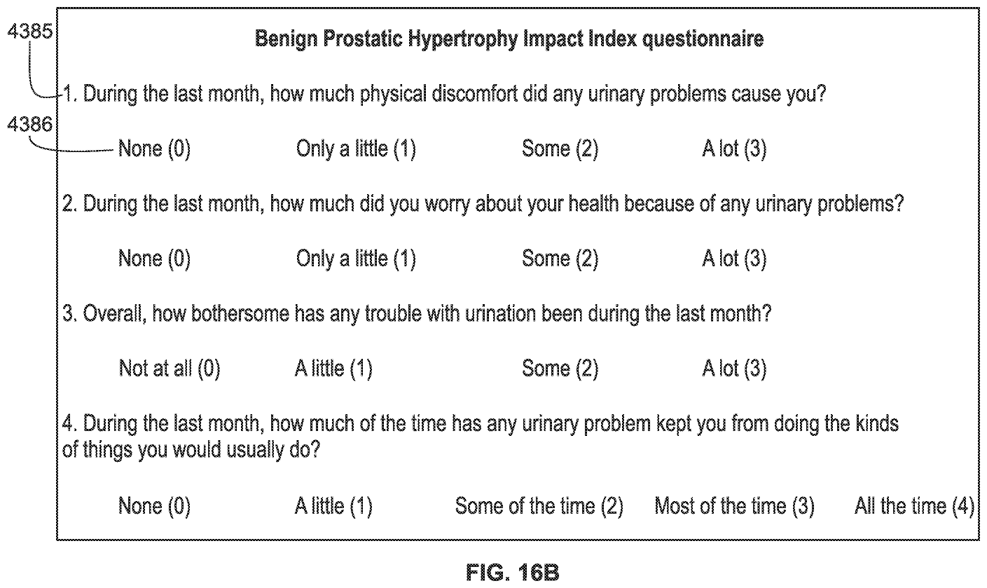

In various embodiments, ablation therapy provided by the vapor ablation systems of the present specification is delivered to achieve the following therapeutic endpoints for prostate ablation: maintain a tissue temperature at 100° C. or less; improve patient urine flow by at least 5% relative to pre-treatment urine flow at six-month follow-up from the treatment; decrease prostate volume by at least 5% relative to pre-treatment prostate volume at follow-up after six months from treatment; decrease in post-void residual by greater than 5% at the six-month follow-up; decrease in incidence of acute urinary retention by 5% at 12-month follow-up; decrease in prostate-specific antigen by 5% at six-month follow-up; improvement in the American Urological Association symptom index by more than 5% at the six-month follow-up; ablate the prostate tissue without circumferentially ablating a urethral tissue; improve International Prostate Symptom Score (IPSS) by at least 5% relative to a pre-treatment IPSS score, wherein the IPSS questionnaire, depicted in FIG. 16A, comprises a series of questions 4380 regarding a patient's urinary habits with numerical scores 4381 for each question; improve Benign Prostatic Hypertrophy Impact Index Questionnaire (BPHIIQ) score by at least 10% relative to a pre-treatment BPHIIQ score, wherein the BPHIIQ, depicted in FIG. 16B, comprises a series of questions 4385 regarding a patient's urinary problems with numerical scores 4386 for each question; and patient reported satisfaction with the ablation procedure of greater than 25%.

### Endometrial Ablation

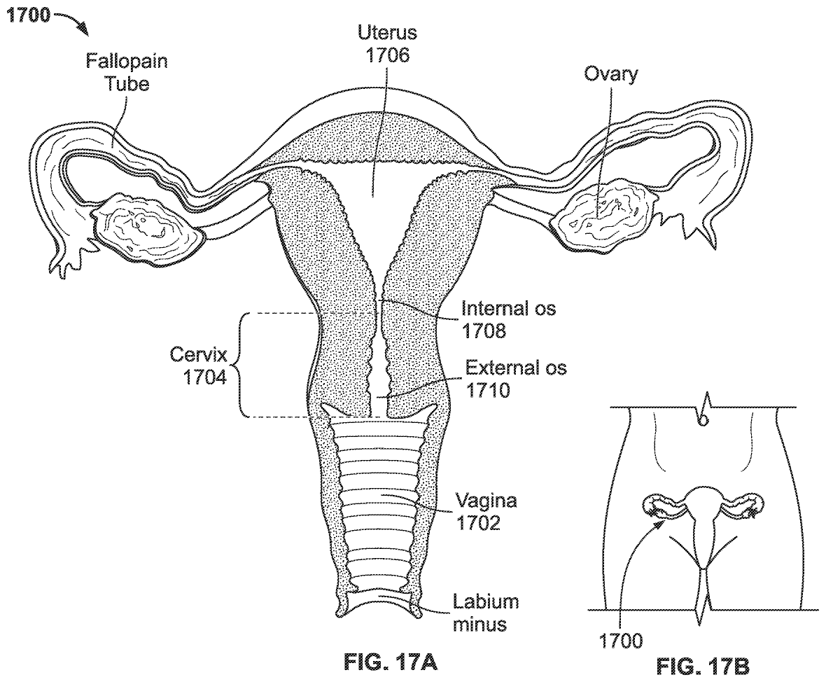

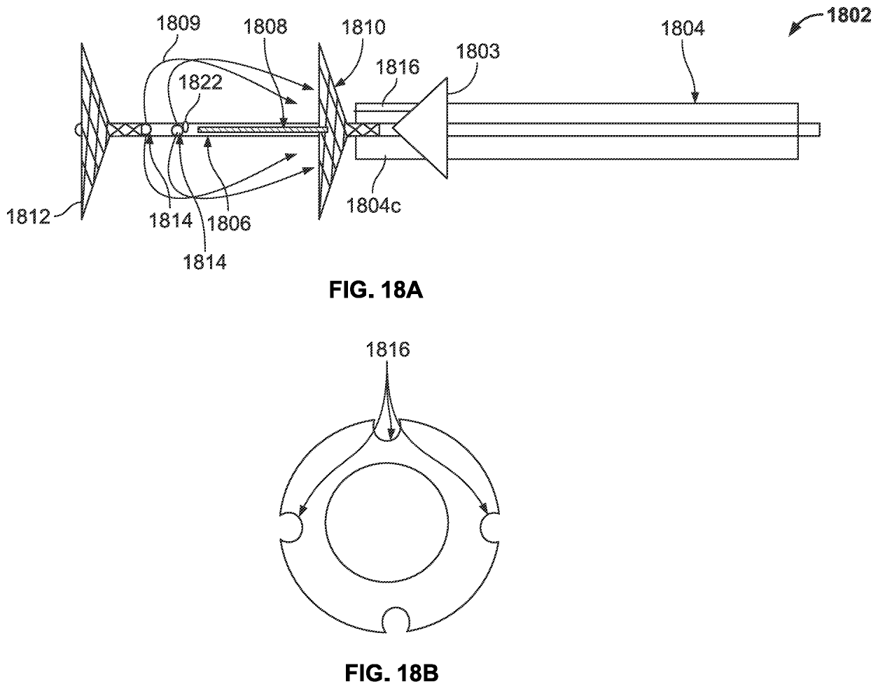

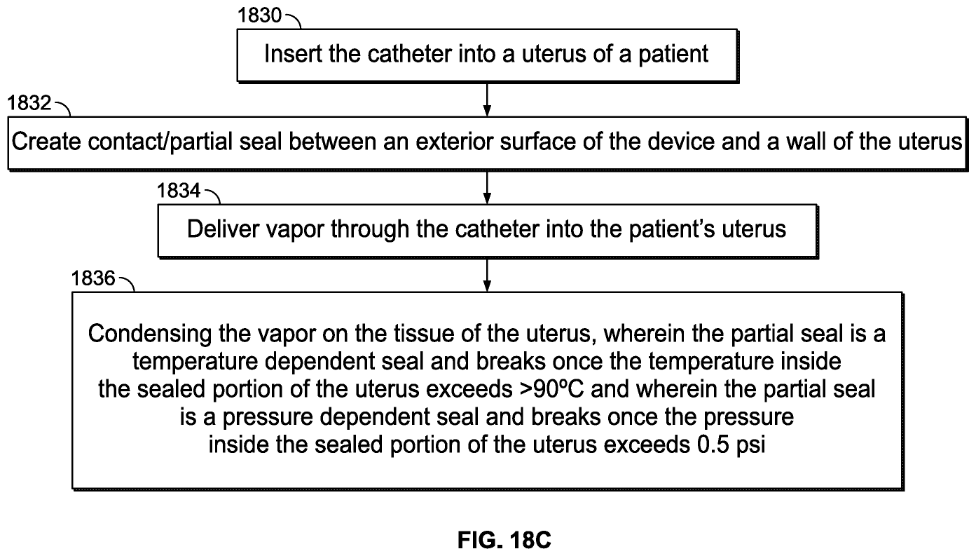

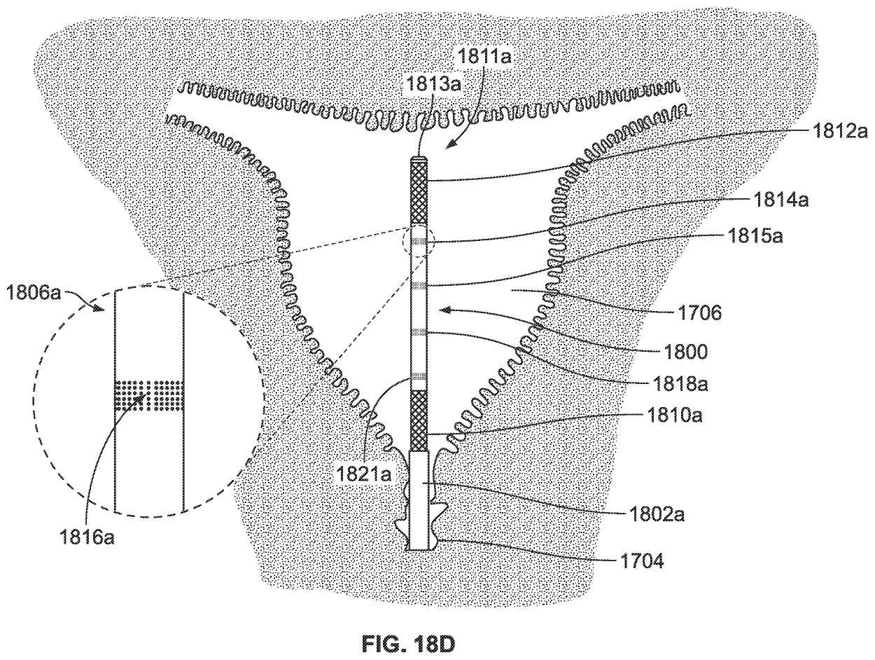

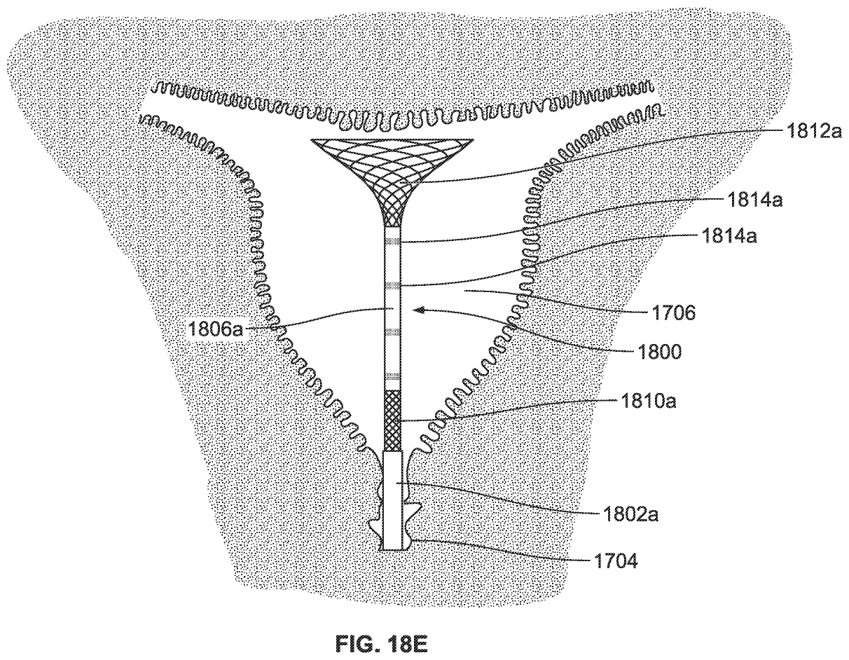

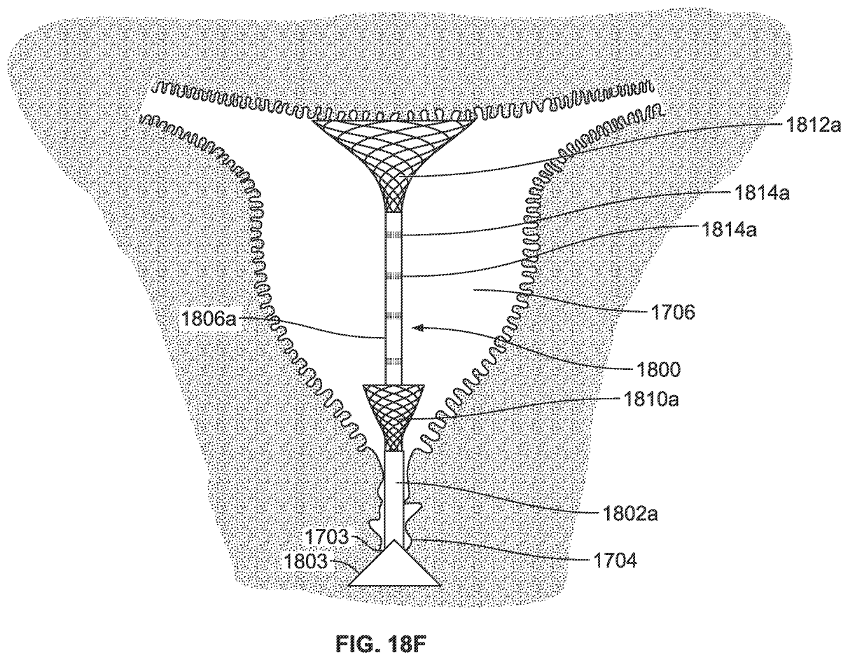

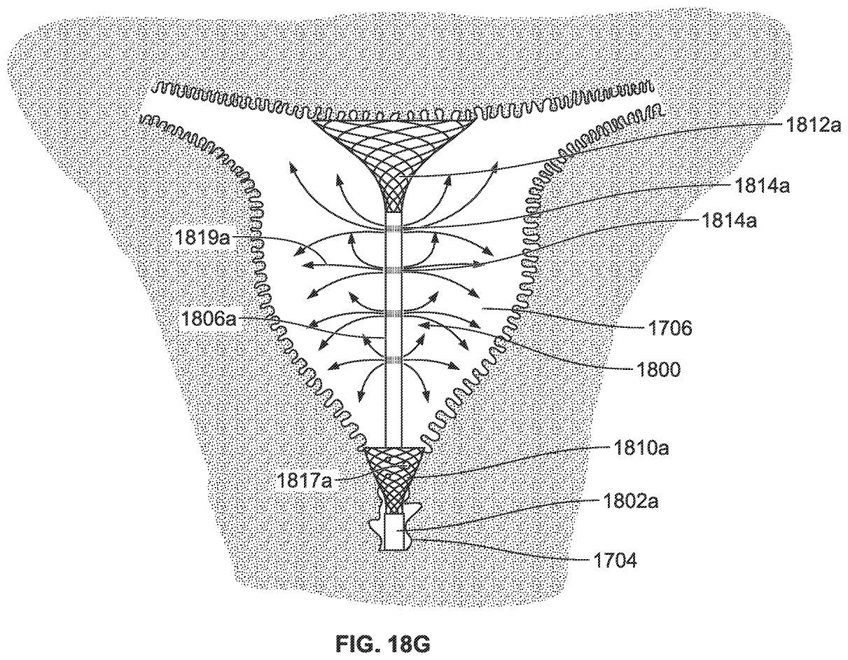

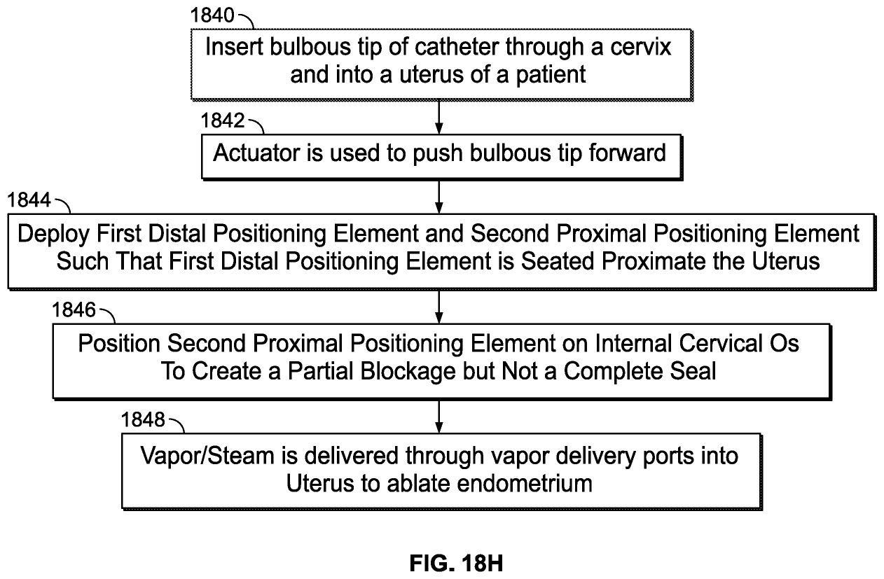

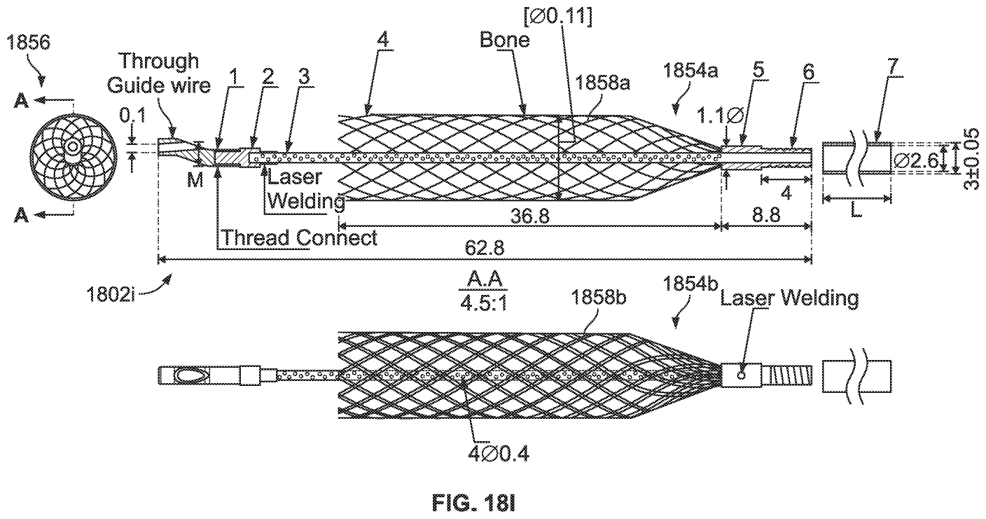

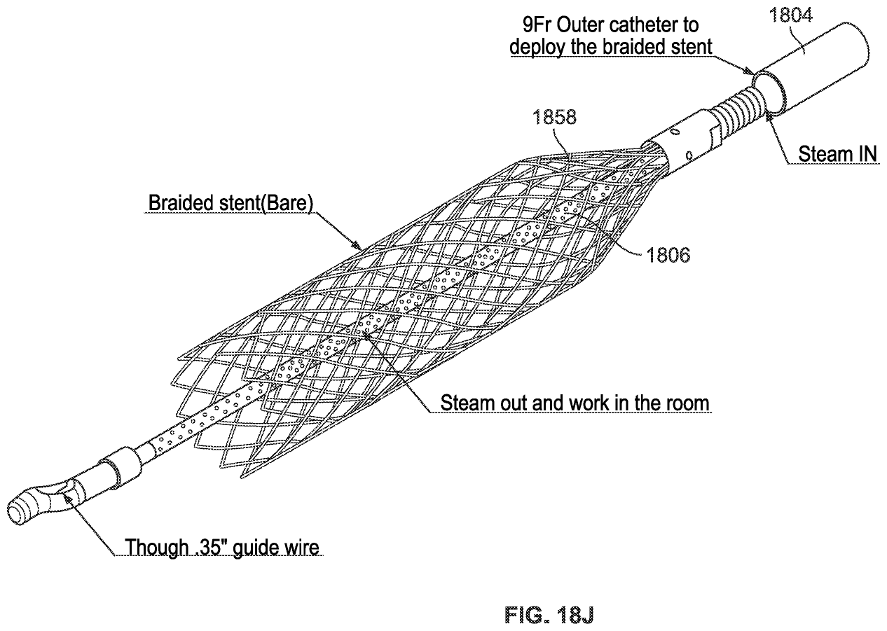

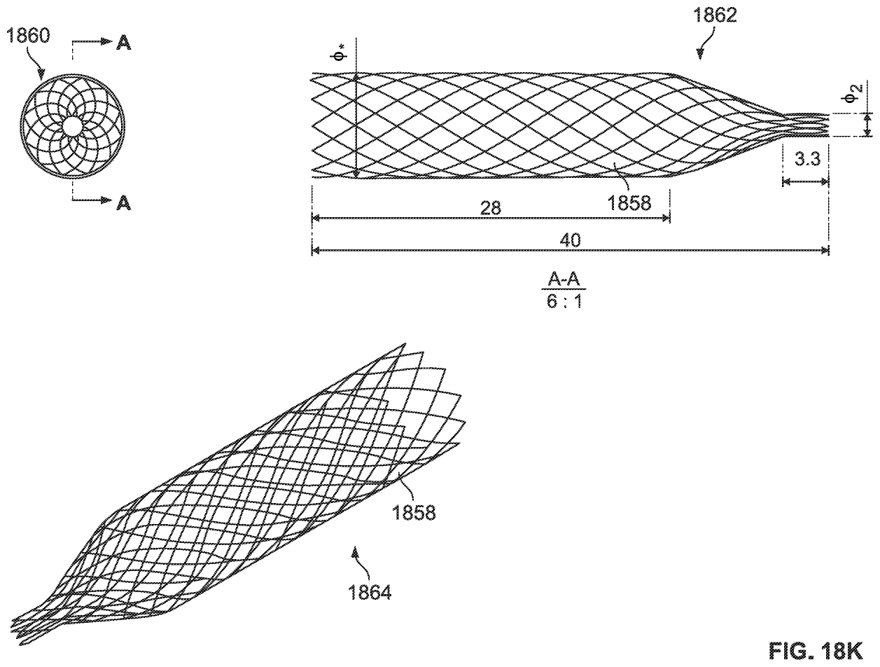

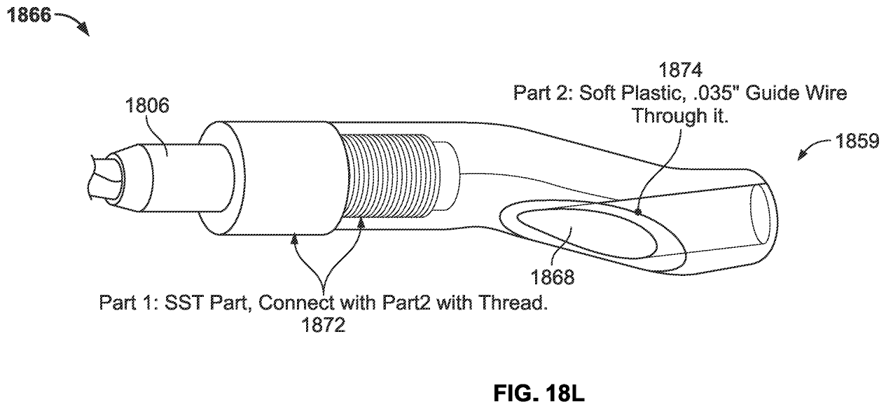

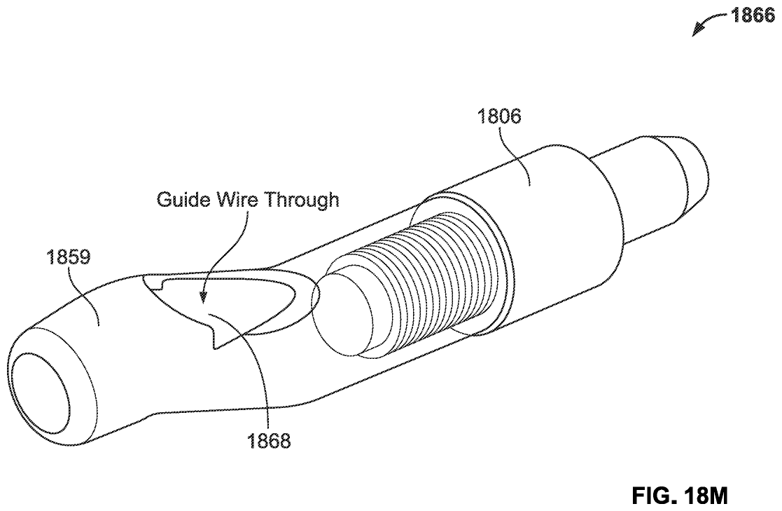

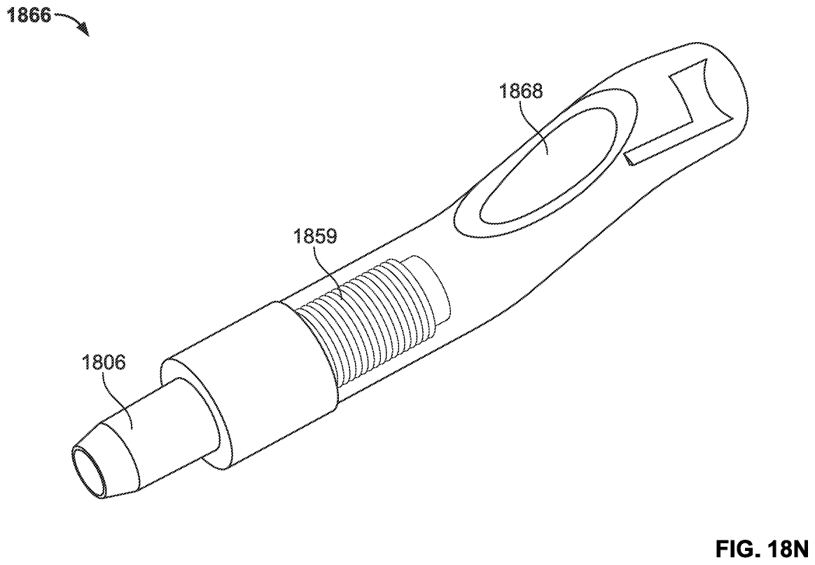

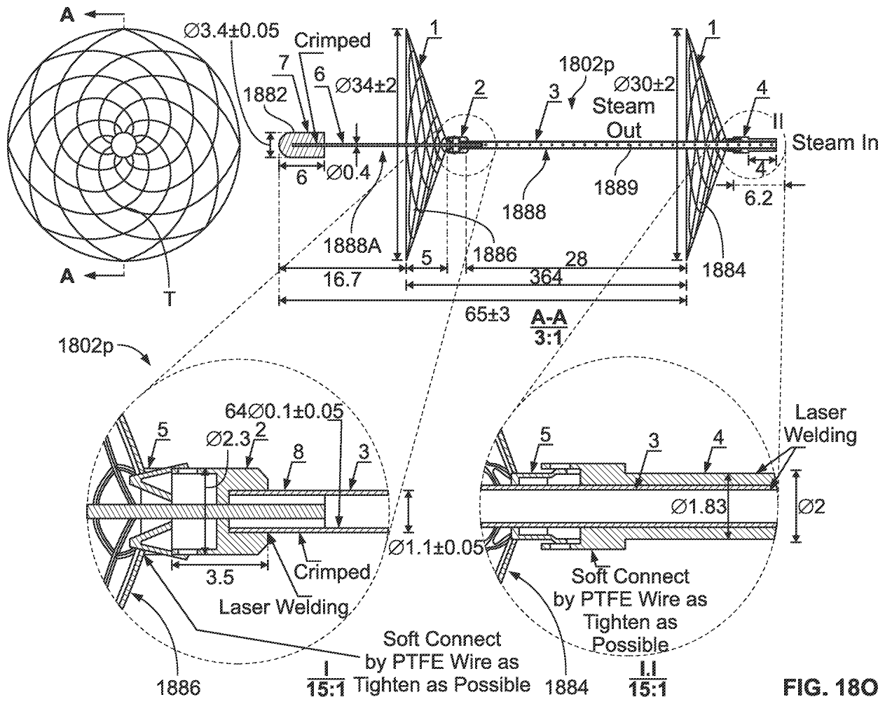

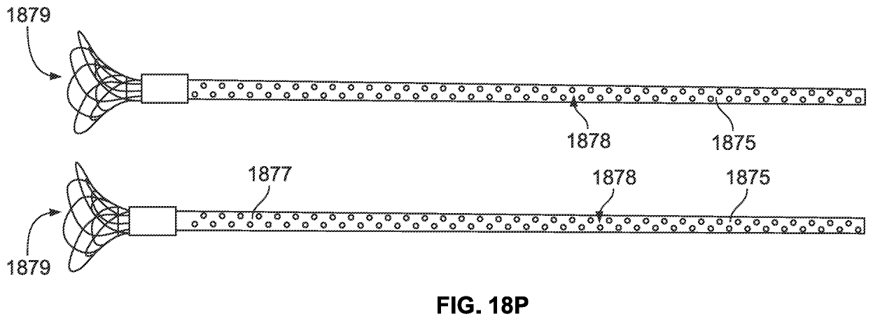

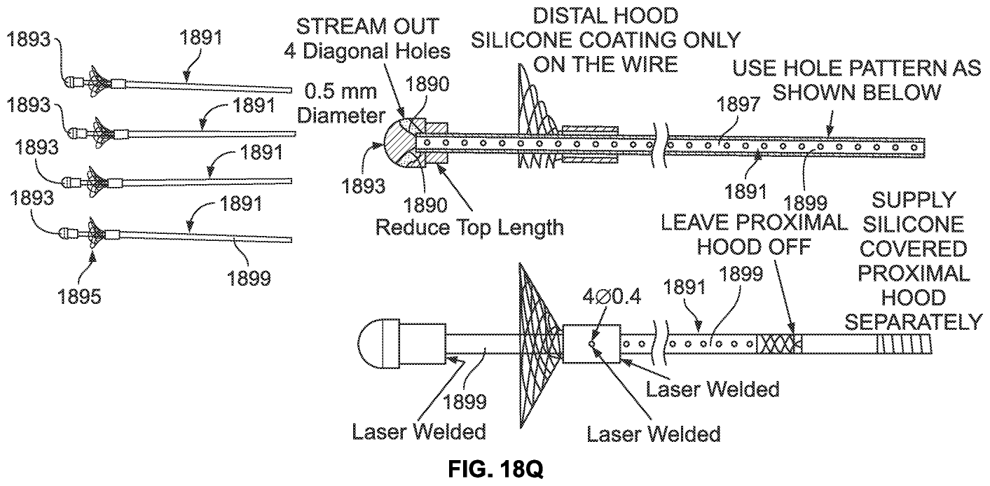

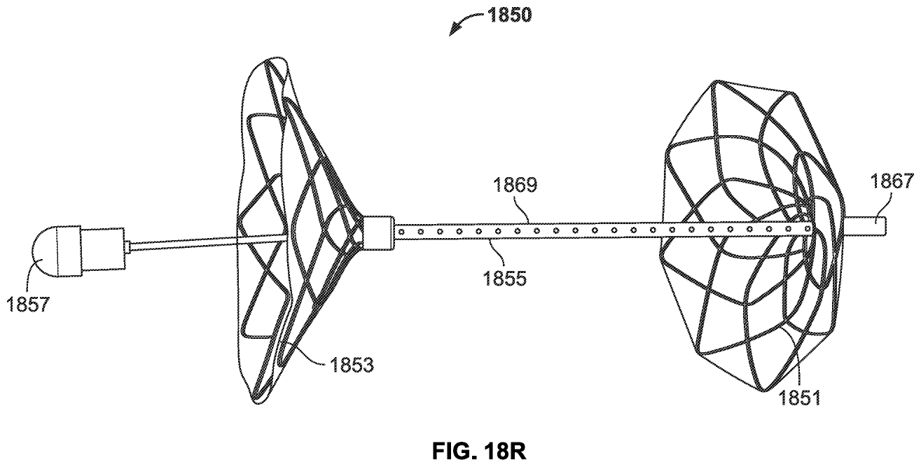

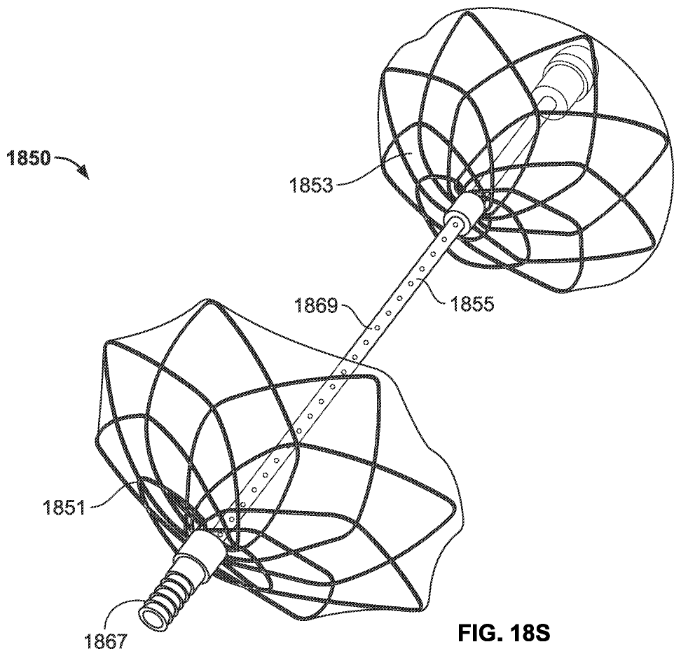

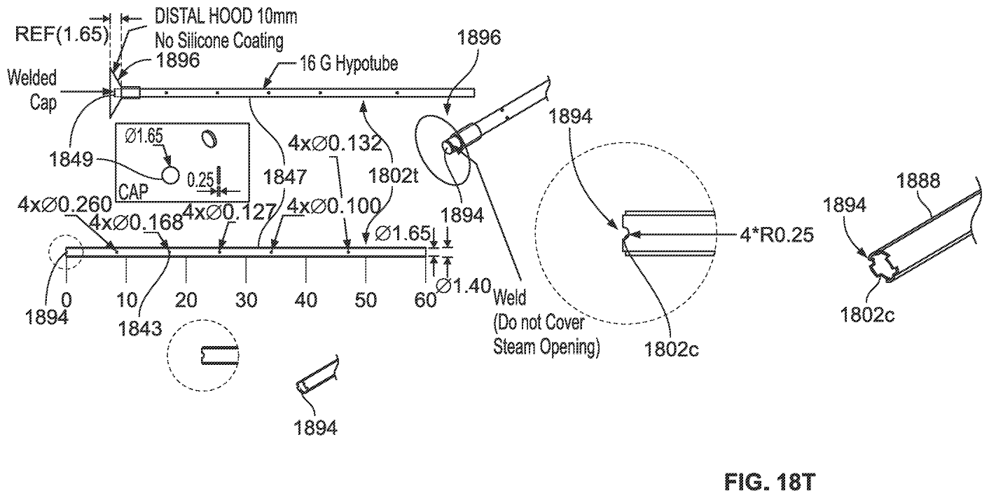

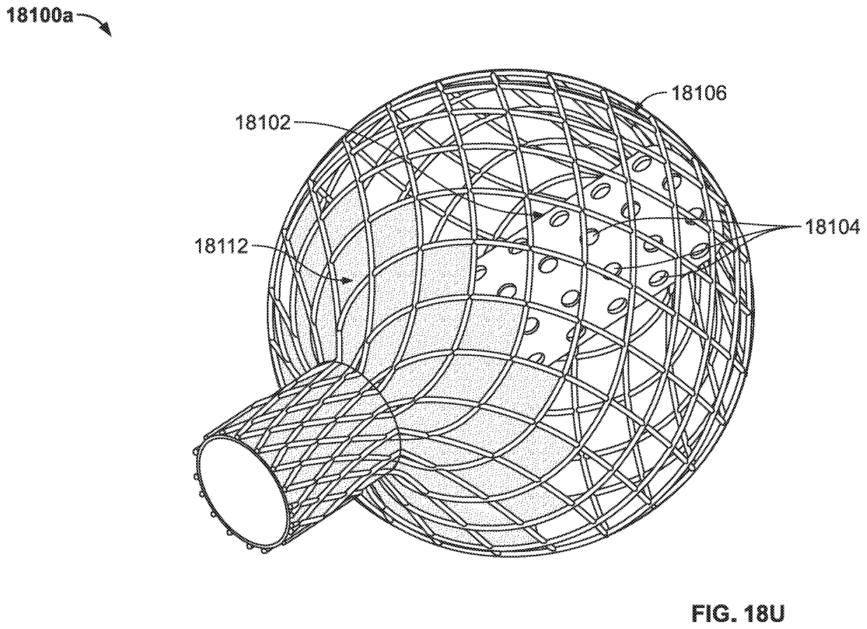

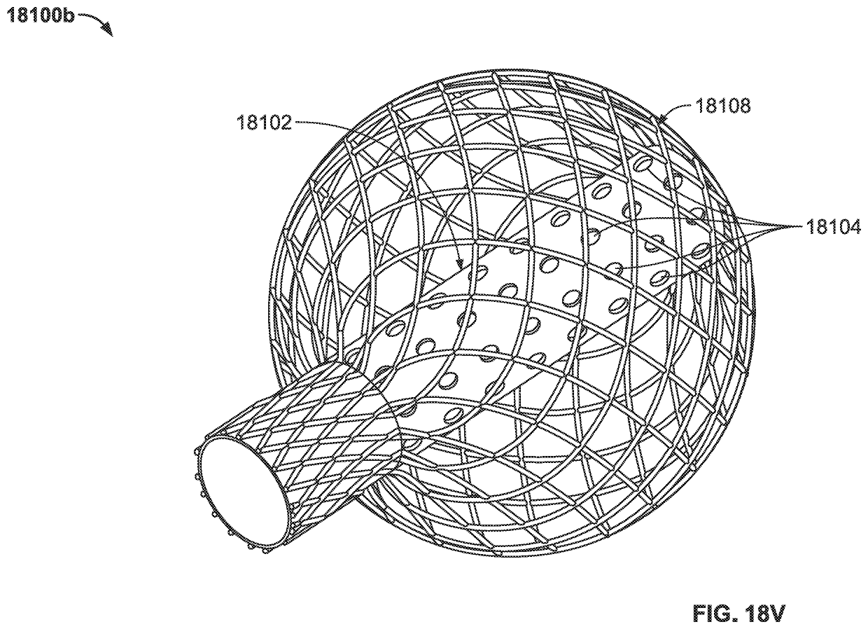

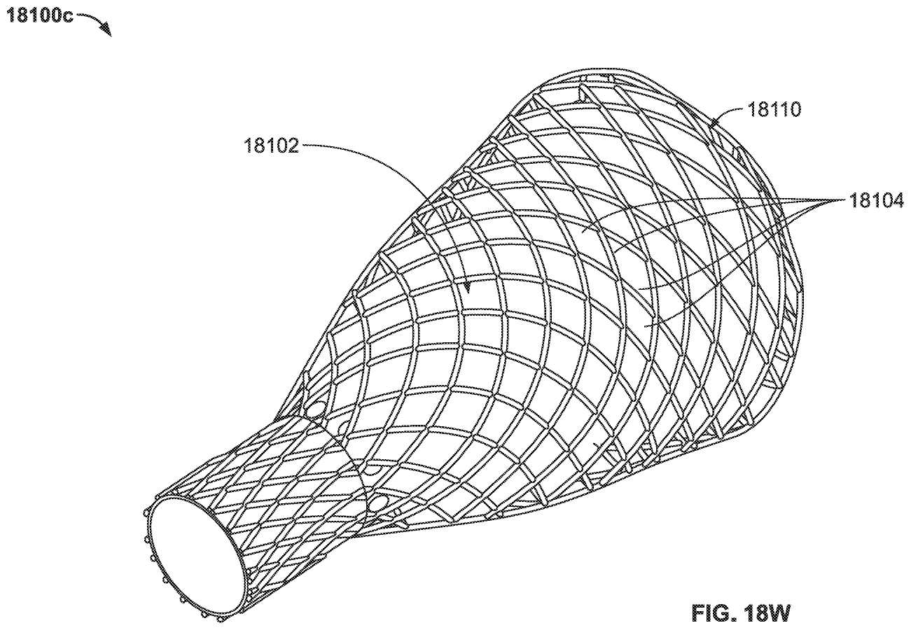

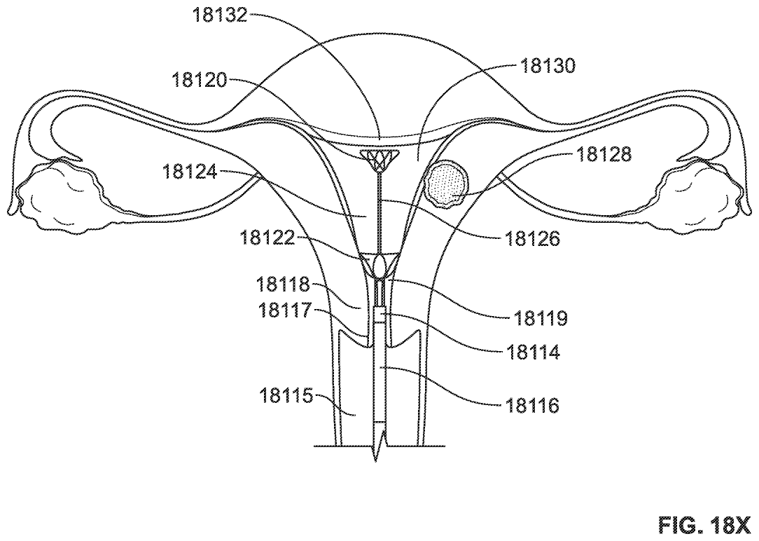

FIG. 17A illustrates a typical anatomy 1700 of the uterus 1706 and uterine tubes of a human female. FIG. 17B illustrates the location of the uterus and surrounding anatomical structures 1700 within a female body. FIG. 18A illustrates an exemplary ablation catheter 1802 arrangement for ablating the uterus 1706, in accordance with some embodiments of the present specification. Referring simultaneously to FIGS. 17A and 18A, in embodiments, a coaxial catheter 1802 is used to insert into vagina 1702 of a patient and advanced toward the cervix 1704. Catheter 1802 comprises an outer catheter 1804 and an inner catheter 1806. Inner catheter 1806 is concentric with and has a smaller radius than outer catheter 1804. An electrode 1808 for heating the catheter tip is located between the two positioning elements 1810, 1812. In some embodiments, the electrode 1808 is proximal to the proximal positioning element 1810. In some embodiments, the positioning elements are discs—a proximal disc 1810 and a distal disc 1812. For purposes of the present specification, discs 1810 and 1812 may also be referred to as hoods 1810 and 1812. In some embodiments, the distal hood 1812 has a smaller diameter than the proximal hood 1810. In some embodiments, the distal hood 1812 is approximately 5 mm smaller than the proximal hood 1810. In embodiments, the hoods 1810 and 1812 are made from wires with different wire stiffness. The distal hood 1812 is configured to contact fundus of the uterus 1706, and acts like a scaffolding to push to halves of uterus away from each other. The proximal hood 1810 is configured to occlude an internal cervical os 1708.

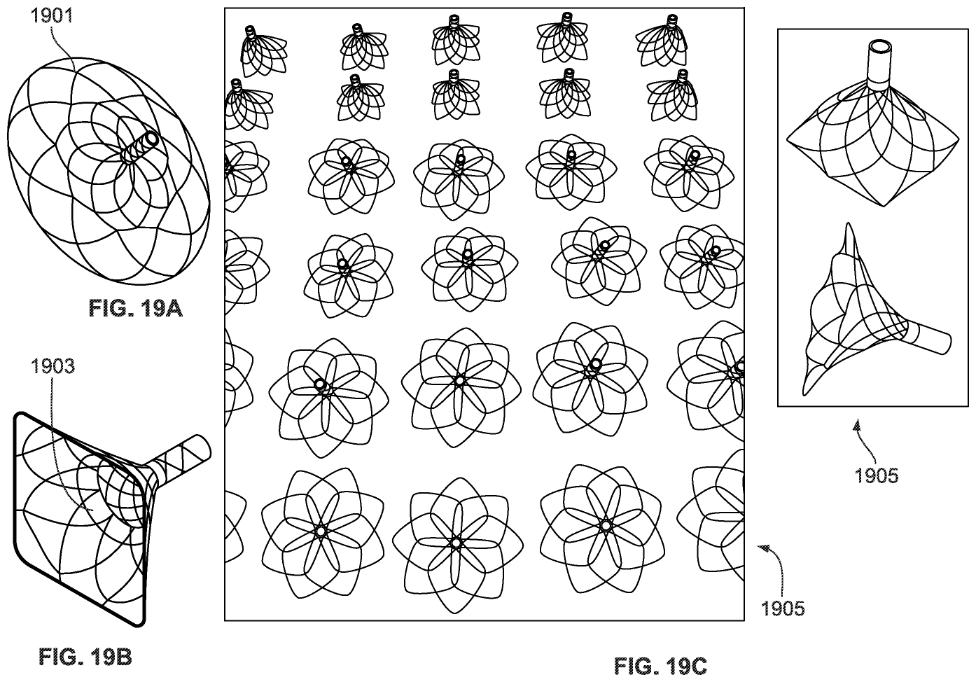

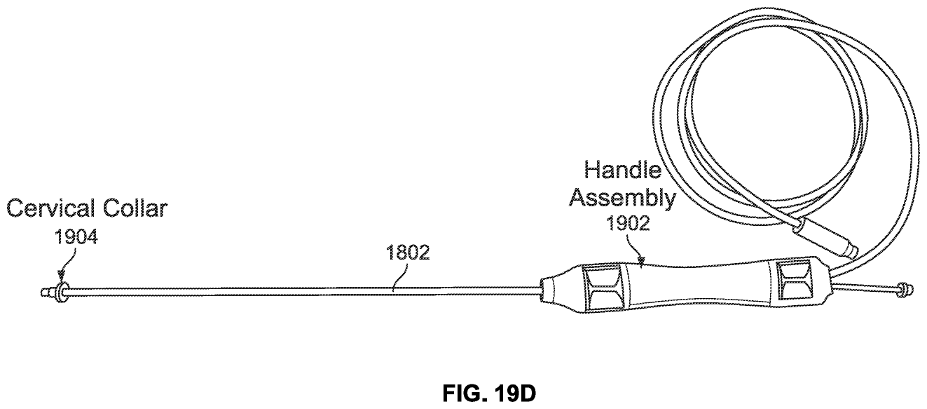

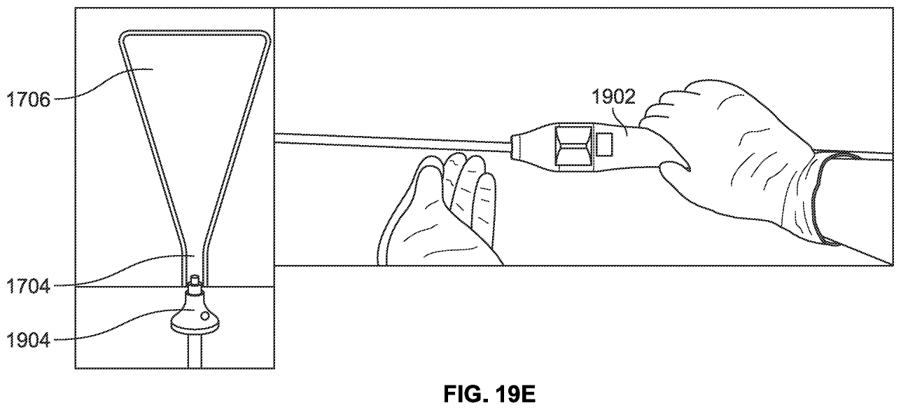

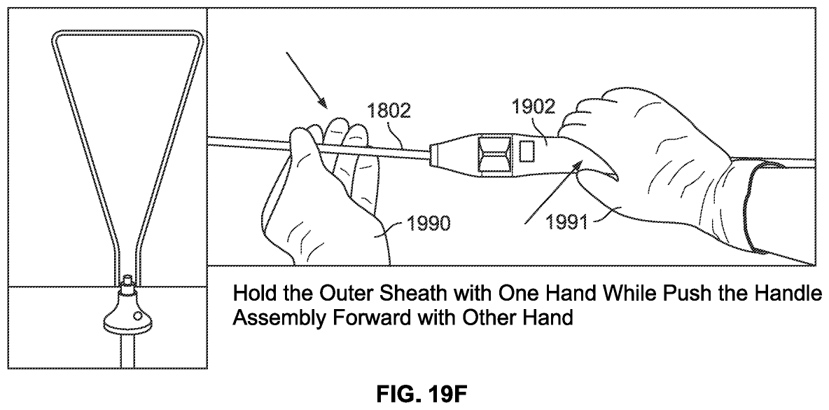

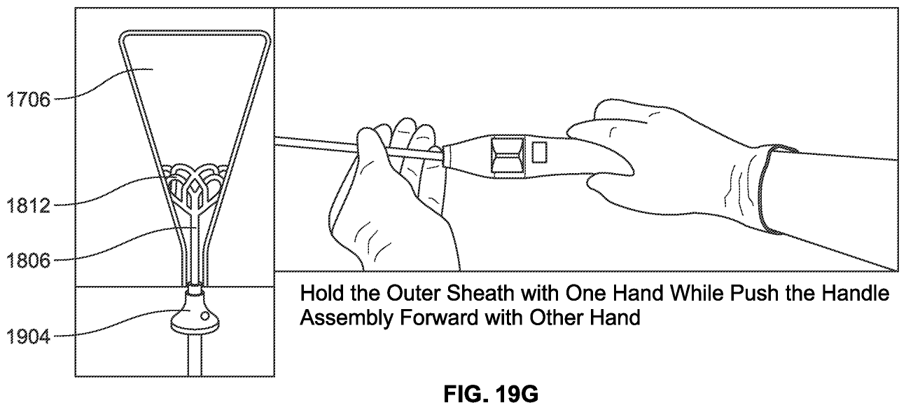

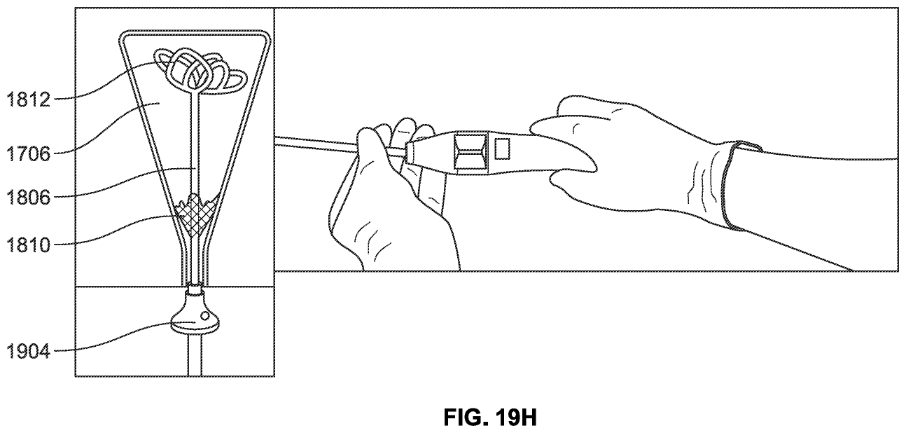

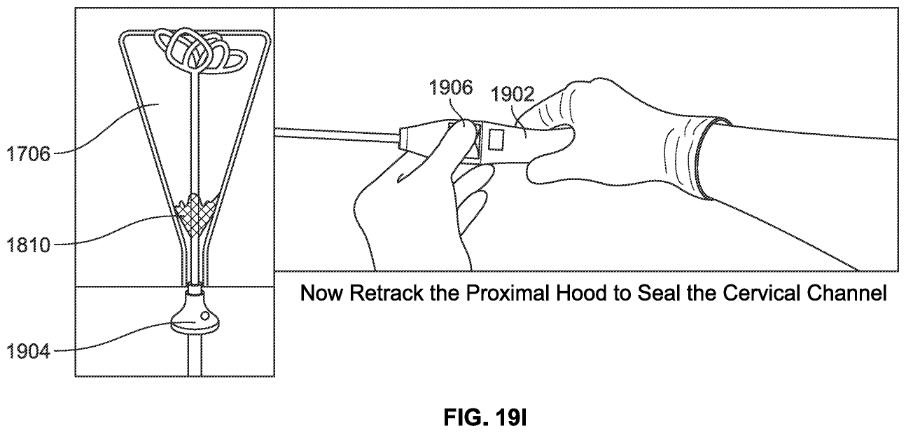

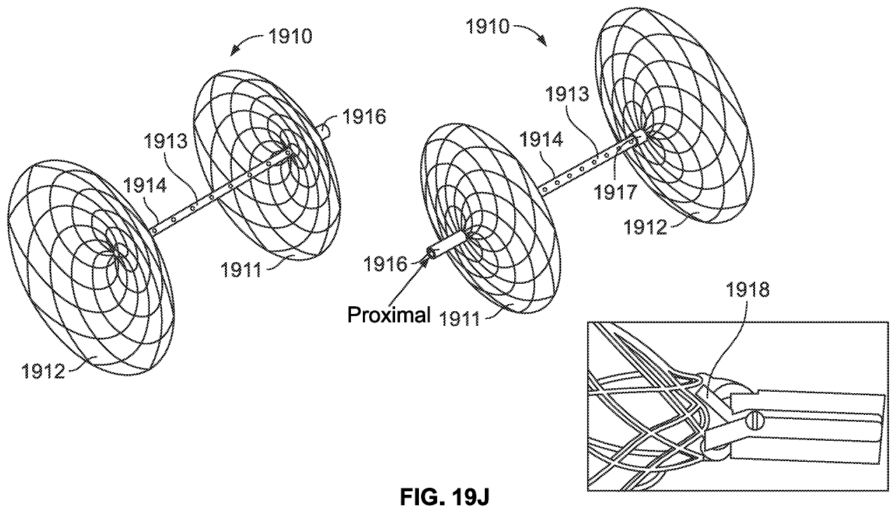

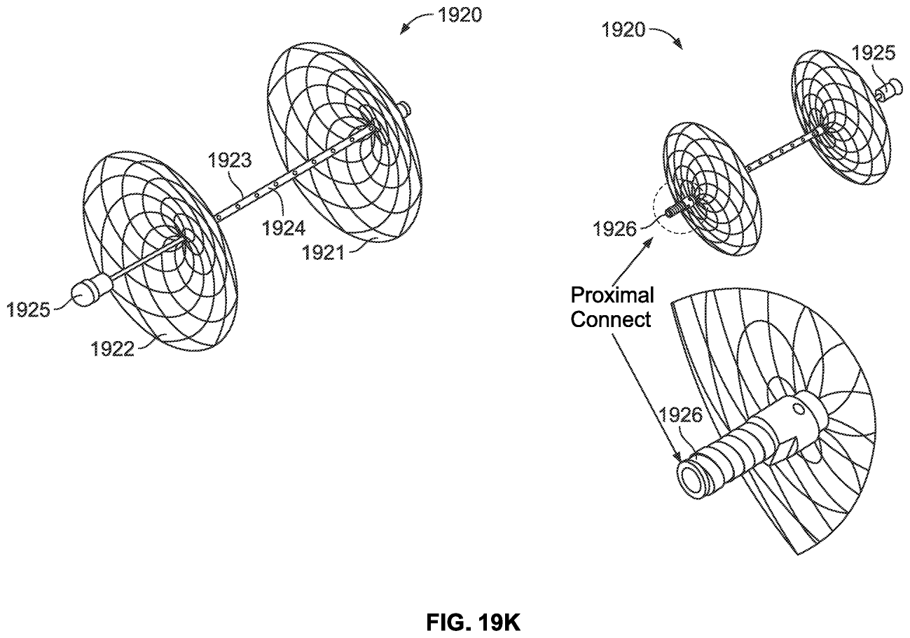

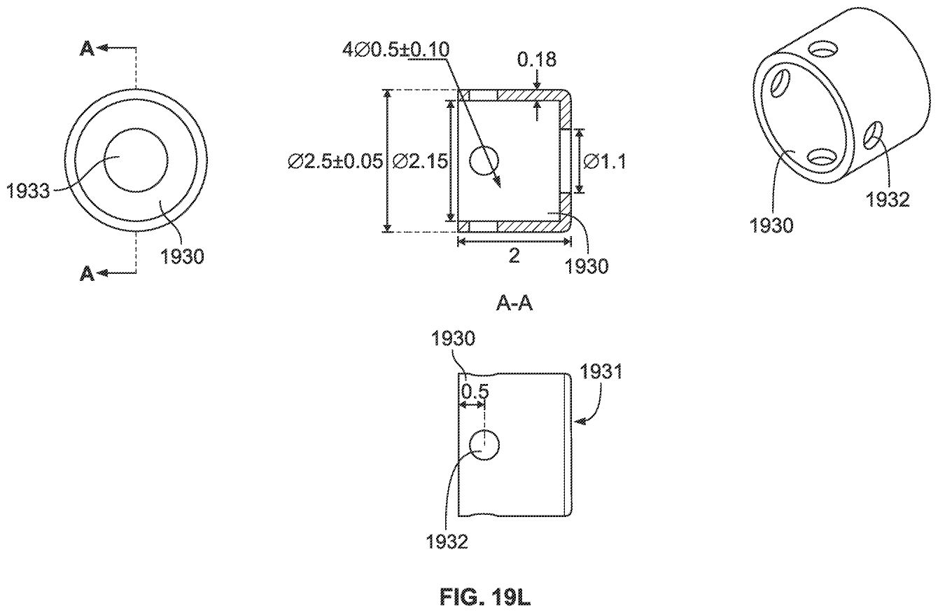

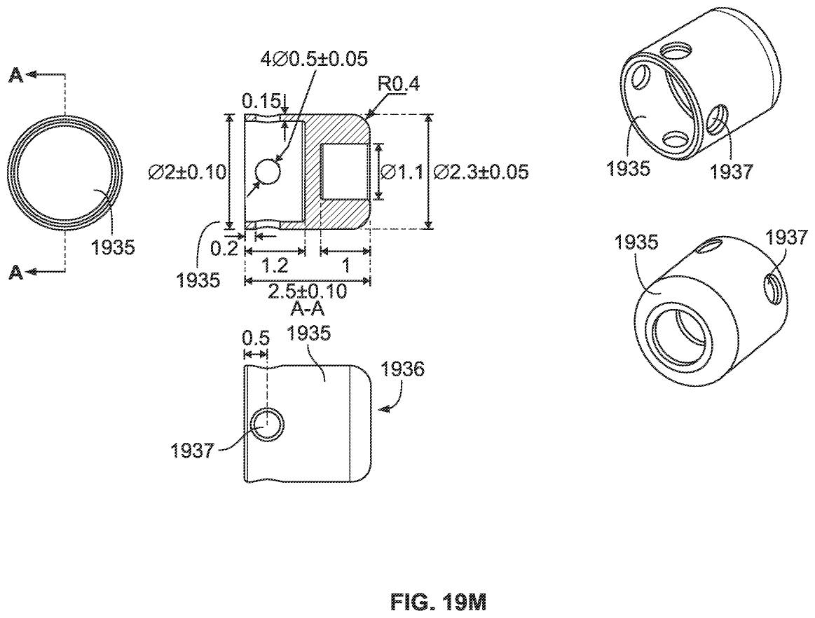

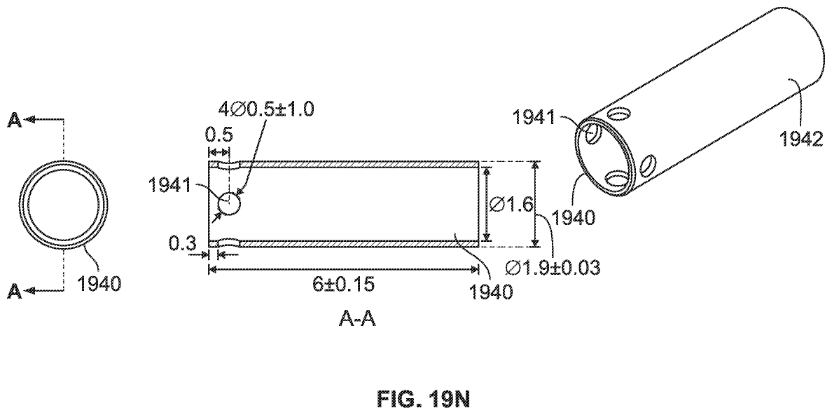

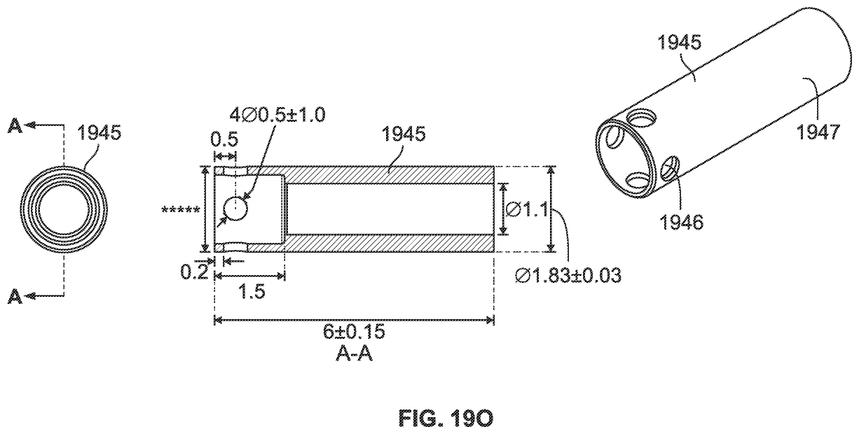

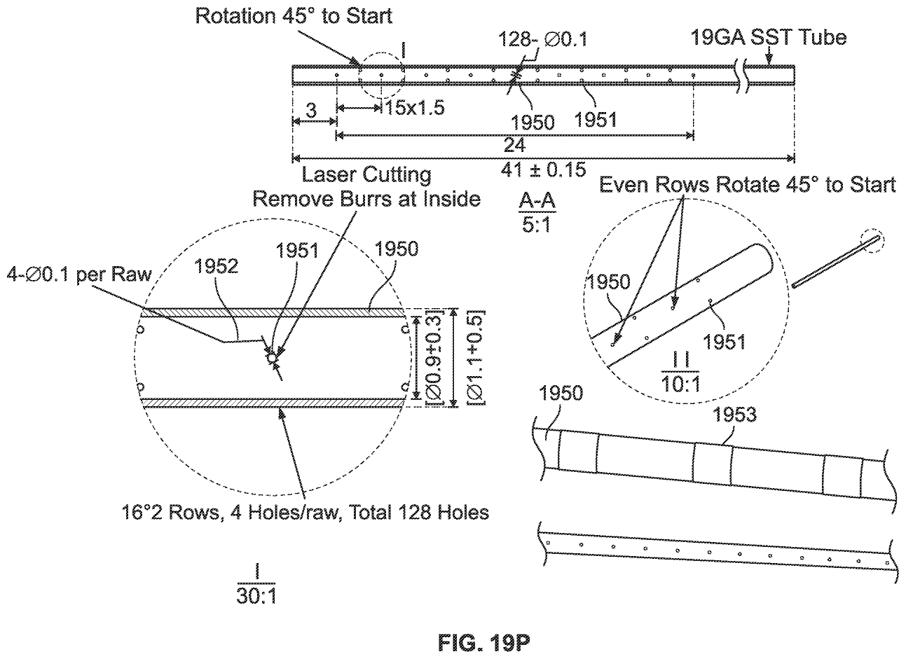

FIGS. 19A, 19B, and 19C illustrate different types of configurations 1901, 1903, 1905 of distal and proximal discs 1812, 1810, which may be used in accordance with the embodiments of the present specification. The discs may differ in stiffness and size and may be chosen by a physician based on the indication for treatment. In some embodiments, the discs are conical in shape with diameters varying from 5 mm to 50 mm. In some embodiments, the positioning elements are ovoid cones with a first proximal diameter of the cone less than a second distal diameter of the cone to approximate the shape or dimensions of a uterine cavity. In various embodiments, a first positioning element may have a different shape or size from a second positioning element. One or more positioning elements maybe used to accomplish the therapeutic purpose.

In some embodiments, the discs 1812, 1810 are formed with wire made from one or a combination of polymers and metal, such as including and not limited to Polyether ether ketone (PEEK) and Nickel Titanium (NiTi). In some embodiments, the wire are covered with elastomers such as PU and/or silicone in a variety of patterns. The various cells in the discs 1812, 1810 may be covered or uncovered based on hood functionality such as whether it is to be used for sealing, or for venting, or for any other purpose. In embodiments where the positioning elements 1812, 1810, are made from Nitinol wire meshes, the wires have a diameter in a range of 0.16 to 0.18 mm. In some embodiments, for the distal positioning element 1812, the wire mesh is coated with silicone but not the areas between wires in the mesh, therefore allowing steam to escape/vent from these spaces between the wires. In some embodiments, for proximal positioning elements 1810, wires and space between wires are covered with silicone.

In embodiments, the inner catheter 1806 is movable into and out of the outer catheter 1804 such that the outer catheter 1804 covers the inner catheter 1806 and restrains the positioning elements 1810, 1812 before insertion into a patient's uterus. The positioning elements 1810, 1812 are composed of a shape memory material such that they expand into a deployed configuration, as shown in FIG. 18A, once the inner catheter 1806 is extended beyond the distal end of the outer catheter 1804.

In embodiments, catheter 1802, with the inner catheter 1806 disposed within the outer catheter 1804 and the positioning elements 1810, 1812 in a first, restrained configuration, is inserted into the vagina 1702 so that the distal end of the outer catheter 1804 is positioned proximate the internal os 1708. The internal catheter is then advanced into the uterus 1706. The catheter 1802 is advanced until distal disc 1812 is within uterus 1706 and proximal disc 1810 occludes the uterus 1706 by positioning it proximate the internal os 1708. In embodiments, the catheter 1802 includes a cervical collar 1803 attached to the outer 1804 catheter. The cervical collar 1803 rests against an external os when the catheter 1802 is deployed in a patient's uterus and assists in maintaining the catheter 1802 in a correct position. A distal portion 1804c of the outer catheter 1804, which extends from the cervical collar 1803 to a point proximal to the proximal positioning element 1810 is positioned within the cervix or the cervical canal when the catheter 1802 is deployed. In embodiments, the distal 1812 and proximal 1810 positioning elements move independently, or expand and lock together. In embodiments, the inserted length of the internal catheter 1806 is used to measure the uterine depth and determine the amount of the vapor ablative to be used in order to keep the pressure inside the uterus 1706 below a predefined threshold. Vapor ports 1814 are positioned on the inner catheter 1806 between the distal disc 1812 and proximal disc 1810 to output vapor for ablation. The plurality of vapor ports are positioned in a circumferential pattern around a length of the catheter. Vapor ports may vary in size, shape or port density (number of ports/length of a catheter) to optimize vapor delivery into the uterine cavity. The vapor 1809 heats the endometrium proximate the distal disc 1812 and then travels in a direction toward the proximal disc 1810, while pushing the endometrial air out. In another embodiment, the vapor delivery ports are configured to heat the entire endometrial cavity simultaneously and uniformly. In embodiments, at least one of the inner catheter 1806 or outer catheter 1804 catheter include venting elements or grooves 1816 that allow venting of the uterus 1706, to allow escape of the endometrial air and preventing over-pressurization of the endometrial cavity. In some embodiments, the grooves may be present around a percentage of a total circumference of the inner catheter 1806 or outer catheter 1804. In some embodiments, the grooves are present around a total circumference of the inner catheter 1806 or the outer catheter 1804, and more preferably around 1 to 90% of a total circumference of the inner catheter 1806 or the outer catheter 1804. FIG. 18B illustrates an exemplary embodiment of grooves 1816 configured in the inner catheter 1806 walls, in accordance with some embodiments of the present specification. In some embodiments, openings in the proximal disc 1810 allow venting of the uterus 1706. In embodiments, the proximal disc 1810 is covered in an elastomer, such as PU or silicone, in a pattern having various cells or openings in the disc 1810 which are uncovered and allow for venting during ablation. In other embodiments, where a seal is desired, there are no uncovered cells or openings in the disc 1810 to allow for venting. In embodiments, a pressure sensor 1822 is used with catheter 1802 to check and subsequently maintain a pressure within the uterus 1706 below 50 mm Hg, preferably below 30 mm Hg, and more preferably below 15 mm Hg. In embodiments, the pressure is also maintained at no more than 10% above atmospheric pressure. As a result of the low pressure level that is maintained within the uterus, embodiments of the present specification are able to forego an integrity check, which is otherwise time consuming and involves risks, and is required in implementation of the prior art. In one embodiment, the endometrial cavity pressure is measured by a generator by measuring the back pressure on the saline being pushed through the inner catheter to the electrode and can be modulated by modulating the saline flow to maintain the endometrial cavity pressure at less than 5 atm. In some embodiments, the endometrial cavity pressure is maintained at less than 0.5 atm.

FIG. 18C is a flowchart of one method of using the catheter of FIG. 18A to ablate endometrial tissue, in accordance with some embodiments of the present specification. At step 1830, the catheter is inserted into a uterus of a patient. At step 1832, contact, or a partial seal is created between an exterior surface of the device and a wall of the uterus. Vapor is then delivered through the catheter into the patient's uterus at step 1834. At step 1836, the vapor condenses on the tissue of the uterus, wherein the partial seal is a temperature dependent seal and breaks once the temperature inside the sealed portion of the uterus exceeds >90° C. and wherein the partial seal is a pressure dependent seal and breaks once the pressure inside the sealed portion of the uterus exceeds 1.5 psi, preferably 1.0 psi, and more preferably 0.5 psi. In another embodiment, the partial seal breaks once the pressure inside the sealed portion of the uterus exceeds 2 psi or 10 mm Hg. In another embodiment, the partial seal breaks when the pressure exceeds 6 psi or 30 mm Hg. In some embodiments, the partial seal is a pressure dependent seal and breaks once the temperature inside the sealed portion of the uterus exceeds 101° C. and the pressure exceeds 0.5 psi. In some embodiments, the partial seal is a pressure dependent seal and breaks once the temperature inside the sealed portion of the uterus exceeds 102° C. and the pressure exceeds 1.0 psi. In some embodiments, the partial seal is a pressure dependent seal and breaks once the temperature inside the sealed portion of the uterus exceeds 103° C. and the pressure exceeds 1.5 psi.

FIGS. 18D to 18G illustrate an embodiment of an endometrial ablation catheter 1800 of the system of FIG. 1P, in accordance with the present specification. Referring to FIG. 18D, catheter 1800 has an outer catheter or sheath 1802a and an inner catheter 1806a. In some embodiments, an outer diameter of inner catheter 1806a is approximately 3.5 mm. In some embodiments, the distal end 1811a of the catheter 1800 has a bulbous tip 1813a to allow for atraumatic insertion into a patient's vagina, through the patient's cervical canal 1704, and into the uterus 1706, without the need of pre-dilation of the cervix. A plurality of rows 1814a, 1815a, 1818a, 1821a each having a plurality of vapor delivery ports 1816a, is positioned between a distal positioning element 1812a and a proximal positioning element 1810a. In different embodiments, the numbers of ports 1816a vary from 1 to 10,000. In some embodiments, the numbers of ports 1816a are within a range of 64 to 96 ports. In embodiments, size of the hole in each port 1816a is within a range of 0.01 to 1 mm. In an embodiment, size of the hole is 0.1 mm. In various embodiments, the vapor delivery ports 1816a are sized differently in the different rows 1814a, 1815a, 1818a, 1821a, creating a steam gradient along the catheter and within the organ volume. For example, in some embodiments, larger delivery ports are positioned at the distal row 1814a to maximize steam in a larger volume of cavity and smaller delivery ports are positioned at proximal row 1821a for a smaller volume of cavity. In embodiments, row 1815a includes ports smaller than those of row 1814a while row 1818a includes ports smaller than those of 1815a but larger than those of 1821a. In other embodiments, the ports in distal rows 1814a, 1815a, or distal half of the catheter 1800, have a total surface greater than a total surface area of the ports in the proximal rows 1818a, 1821a, or proximal half, of the catheter 1800. In another embodiment, port size remains consistent and the port density in the various rows or regions of the catheter varies.

Referring to FIG. 18E, catheter 1800 is advanced through a cervical canal 1704 and into a uterus 1706 such that the inner catheter 1806a is positioned within uterus 1706 and the outer sheath 1802a is positioned within cervical canal 1704. The distal positioning element 1812a is expanded. In embodiments, the positioning element 1812a may vary in size, shape, diameter, geometry, or any other structural feature, so as to regulate steam distribution in a desired manner. Referring to FIG. 18F, distal positioning element 1812a, having, in one embodiment, a funnel shape, is expanded and catheter 1800 is further advanced into the uterus 1706 so that the distal end of the outer catheter 1804a is positioned proximate the internal os 1708. Proximal positioning element 1810a, having, in one embodiment, a funnel shape with or without venting, is also expanded. Additionally, an external cervical stabilizing element, or cervical collar 1803, is positioned at an external cervical os 1703. Referring to FIG. 18G, vapor 1819a is delivered through the plurality of ports 1816a within rows 1814a. In some embodiments, areas on a surface of the proximal positioning element 1810a provide for venting of vapor or steam. In some embodiments, the proximal positioning element 1810a comprises a plurality of openings 1817a to allow for venting. In various embodiments, the proximal positioning element 1810a is covered by a gas permeable membrane or porous membrane to allow for venting. In some embodiments, for the distal positioning element 1812, the wire mesh is coated with silicone but not the areas between wires in the mesh, therefore allowing steam to escape/vent from these spaces between the wires. In some embodiments, for proximal positioning elements 1810, wires and space between wires are covered with silicone.

In some embodiments, the proximal positioning element may be attached to a middle catheter and allow for venting between the middle catheter and the inner catheter. In another embodiment, the proximal positioning element may be attached to the outer catheter and allow for venting between the outer catheter and the inner catheter.

FIG. 18H is a flow chart illustrating the steps involved in using an ablation catheter to ablate an endometrium of a patient, in accordance with embodiments of the present specification. In various embodiments, the catheter is similar to those described with reference to FIGS. 18D-18G. In some embodiments, the method does not require pre-dilation of the cervix before ablation. At step 1840, a physician inserts a bulbous tip (such as bulbous tip 1813a of catheter 1800 in FIG. 18D) into and through a patient's cervix and advances the catheter into the patient's uterus. The bulbous tip helps to guide the device through the cervix and allows for atraumatic insertion. In some embodiments, the bulbous tip includes an olive-shaped attachment 1882, described in context of FIG. 18O, for atraumatic insertion. In some embodiments, at step 1842, an actuator (such as actuator 191p on handle 190p in FIG. 1P) is used to push forward the bulbous tip. For example, referring to FIG. 1P, on a dorsal side of handle, an actuator 191p in the form of a slide, is moved forward to activate/push forward the bulbous tip. Once the catheter is advanced into the uterus, at step 1844, the first and second positioning elements are deployed and the distal positioning element is seated proximate the uterus. In some embodiments, the positioning elements are deployed using actuators as described with reference to FIG. 1P. At step 1846, the second proximal positioning element is positioned on the internal cervical os to create a partial blockage but not a complete seal. As described with reference to FIG. 18G, areas on surface of the disc will provide for venting of pressurized air or steam. In one embodiment, the venting occurs through a neck of the positioning element. In another embodiment, the venting occurs between an inner and middle or an inner and outer catheter. At step 1848, vapor or steam is delivered through the plurality of vapor delivery ports on the catheter into the uterus to ablate the endometrium.

FIGS. 19D to 19I illustrate an endometrial ablation catheter at different stages of an exemplary method of deployment of the catheter 1802, in accordance with some embodiments of the present specification. FIG. 19D illustrates an assembly of catheter 1802 with a handle 1902, and a cervical collar 1904, in accordance with some embodiments of the present specification. FIG. 19E illustrates a position of the cervical collar 1904 as it sits at an external os, outside the uterus 1706 and cervix 1704, before deployment of the catheter 1802. In the figure, the uterus 1706, cervix 1704, and cervical collar 1904 are shown on the left while specific hand movements on the handle 1902 are shown on the right to demonstrate deployment of the catheter 1802. FIG. 19F illustrates an exemplary position of hands 1990, 1991 to hold the catheter 1802 and handle 1902 for deploying the proximal positioning element 1810, in accordance with some embodiments of the present specification. A user holds the outer sheath of catheter 1802 with one hand 1990 while pushing the handle 1902 forward with the other hand 1991. FIG. 19G illustrates expanding of the distal positioning element 1812 while the user pushes the handle 1902 of the catheter 1802 to extend the inner catheter 1806 within the uterus 1706. FIG. 19H illustrates fully deploying the distal positioning element 1812, which may be uncoated or selectively coated with silicone, and deploying of the proximal positioning element 1810, in accordance with some embodiments of the present specification. So far, in the process of deploying the catheter 1802, nothing is seated yet. The user may decide to stop here or adjust the position of the catheter 1802 till a distance is achieved that is just short of the length of the uterus 1706 to prevent perforation. In some embodiments, the user may decide to push the catheter 1802 until its distal positioning element 1812 abuts a fundus of the uterus, indicating resistance at the fundus. The user may, in some embodiments, turn a dial, provided on the handle 1902, clockwise to retract the proximal positioning element 1810 and extend further the distal positioning element 1812. In some embodiments, when the proximal positioning element 1810 is expanded, it moves in a direction toward the cervical collar 1904 while the cervical collar 1904 moves in an opposite direction, toward the proximal positioning element 1810 (similar to a Chinese finger puzzle) FIG. 19I illustrates turn of a dial 1906 to further retract first positioning element 1810 to partially seal a cervical os, so as to isolate the uterus 1706. In some embodiments, the partial seal is not perfect (escape vents are provided in openings or holes of the proximal positioning element or venting elements/grooves are provided in one or both of the inner catheter or outer catheter/sheath) to allow for release of vapor out of the uterus, maintaining a low pressure. In some embodiments, the user ablates the uterus by delivering steam through the catheter 1802 for about a 40 second cycle. In embodiments, proximal positioning element 1810 has selective coating and it provides for a drain to collect water produced as the vapor condenses during and after ablation.

FIGS. 18I to 18N illustrate exemplary embodiments of distal end of an endometrial ablation catheter having a single positioning element, in accordance with the present specification. FIG. 18I illustrates a cross-section side view 1854a, side view 1854b, and distal end front-on view 1856, of the endometrial ablation catheter 1802i, in accordance with some embodiments of the present specification. The catheter 1802i is shown with a braided stent 1858. The stent 1858 functions as a positioning element described with reference to the endometrial ablation catheters of the present specification. In embodiments, the braided stent 1858 is made from Nitinol wire mesh, or any other shape memory material such that the stent 1858 expands into a deployed configuration, as shown in FIG. 18I. In embodiments, the stent 1858 is made from a single wire mesh 1858a. In some embodiments, the stent 1858 is made from a double wire mesh 1858b. FIG. 18J illustrates a perspective side view of the catheter of FIG. 18I with the stent 1858 extending over the inner catheter 1806, and extending out from the outer catheter 1804. Steam from within the inner catheter 1806 is deployed while the braided stent 1858 is in an expanded state and deployed within the uterus. The catheter includes an atraumatic distal tip 1859 with guide wire lumen, as described with reference to FIGS. 18L through 18N. The guidewire lumen could be large enough to accommodate a uterine sound. FIG. 18K illustrates a cross section view 1862, a perspective side view 1864, and a distal end front-on view 1860 of the braided stent 1858, in accordance with some embodiments of the present specification. The proximal conical end of the positioning element is either partially or completely covered by an insulating membrane made of silicone or PTFE.

FIG. 18L illustrates a side perspective view of an atraumatic tip 1859 for attaching to a distal end 1866 of an inner catheter 1806 of an endometrial ablation catheter, in accordance with some embodiments of the present specification. FIG. 18M illustrates a side front perspective view of the atraumatic tip 1859 attached to the distal end 1866 of an inner catheter of an endometrial ablation catheter, in accordance with some embodiments of the present specification. FIG. 18N illustrates a top perspective view of the atraumatic tip 1859 attached to the distal end 1866 of an inner catheter of an endometrial ablation catheter, in accordance with some embodiments of the present specification. Referring simultaneously to FIGS. 18L, 18M, and 18N, the atraumatic tip 1859 includes an opening 1868 for a passage of a guide wire. In embodiments, the opening 1868 is configured to receive a 0.035 inch guide wire. The atraumatic tip 1859 is connected to the inner catheter 1806 at its distal end 1866. In some embodiments, the atraumatic tip 1859 is connected to the distal end 1866 of the inner catheter 1806 via a threaded screw 1872. The atraumatic tip 1859 is made from a soft plastic material and includes grooves to receive and lock with the threaded screw 1872 to connect with the inner catheter 1806.

FIG. 18O illustrates different views of a double-positioning element ablation catheter 1802p with an atraumatic olive tip end 1882, in accordance with another embodiment of the present specification. The olive tip end 1882 ensures that the uterus is not punctured and provides for an atraumatic insertion of the catheter 1802p. In some embodiments, olive tip end attachment 1882 may include a hollow channel within its body, the channel opening at the distal edge of attachment 1882, to enable delivery of steam through the channel. In some embodiments, one or more holes in tip of the attachment 1882 enable delivery of steam. All the holes may be of similar or varying diameters. Two positioning elements—a proximal positioning element 1884 and a distal positioning element 1886, are provided with catheter 1802p. Positioning elements 1884 and 1886 are in the form of hoods, where distal hood 1886 may have a diameter ranging from 25 to 34 mm+/−2 mm, and the proximal hood 1884 may have a diameter ranging from 25 to 30 mm+/−2 mm. A distance between the two hoods 1884 and 1886 may be in a range of 28 to 36.4 mm. Each hood 1884/1886 may have a depth of approximately 5 mm along the length of the catheter 1802p. In embodiments, each hood 1884/1886 is attached to the shaft 1888 using a soft connect mechanism with a PTFE wire. A distance between distal end of the distal hood 1886 and a distal tip of the olive tip end 1882 may be approximately 16.7 mm. The shaft portion 1888a extending between the distal hood 1886 and the olive tip attachment 1882 can also include one or more holes for distributing steam during ablation. In some embodiments, holes may also be present before the distal hood 1886, between the distal hood 1886 and the proximal hood 1884, for disseminating steam.

Length of the olive tip end 1882 may extend for approximately 6 mm. A diameter of the distal tip of the olive tip end 1882 may be in a range of 3.4+/−0.05 mm. Steam enters a catheter 1802p shaft 1888, and exits through openings 1889 along the shaft 1882 during ablation. The shaft 1888 between the two hoods 1884 and 1886 may have a diameter of approximately 1.1+/−0.05 mm. In embodiments, there are additional openings in the olive tip end 1882 and catheter shaft 1888a distal to the distal hood 1886. The shaft 1888a extending from the distal end of the distal hood 1886 to the olive tip end 1882 may be made from Nitinol and has a diameter of approximately 0.4 mm.

FIG. 18P illustrates distal ends of ablation catheters 1878 having distal positioning elements 1879 and a plurality of ports 1877 along a length of the catheter shaft 1875, in accordance with some embodiments of the present specification. FIG. 18Q illustrates distal ends of ablation catheters 1891 having distal olive tips 1893, positioning elements 1895, and a plurality of ports 1897 along a length of the catheter shaft 1899, in accordance with some embodiments of the present specification. The olive tip 1893 is rounded and bulbous and configured to by atraumatic to body tissues. A cross-sectional view of the olive tip end 1893 shows diagonal openings or holes 1890 inside the tip end 1893. In embodiments, the olive tip end 1893 has four identical and symmetrically configured openings within its distal spherical tip. Each opening 1890 is connected to and extends outwards from the hollow catheter shaft 1899 extending beyond the distal hood 1886. The openings 1890 provide an exit for steam out distal to the positioning element 1895 during ablation. FIG. 18R illustrates a side view a distal end of an ablation catheter 1850 having a distal olive tip 1857, a distal positioning element 1853, a proximal positioning element 1851, and a plurality of ports 1855 along a length of the catheter shaft 1869, in accordance with some embodiments of the present specification. FIG. 18S illustrates a rear perspective view of the catheter 1850 of FIG. 18R. The ablation catheter 1850 includes a connector 1867 at its proximal end for connecting to a proximal catheter portion.

FIG. 18T illustrates a distal end of an ablation catheter 1802t with half-circle openings 1802c at the distal end and a distal positioning element 1896, in accordance with some embodiments of the present specification. Although the figure illustrates half-circle opening 1802c, the openings could be of other shapes such as but not limited to, half-rectangular. In some embodiments, the positioning element 1896 is deformable, flattening out as it is pushed against the fundus of a uterus. The distal end 1894 of catheter 1802t may be open, or covered, but in either case includes half-circle openings 1802c. In some embodiments, the circular distal end of the shaft 1888 is configured to include at least three equidistant half-circle openings 1802c. In some embodiments, the distal end 1894 is closed with a cap 1849. In some embodiments, the cap 1849 has a diameter of approximately 1.65 mm. In some embodiments, the cap 1849 is welded to the distal end 1894. The cap 1849 serves to close the open distal end of catheter 1802t, while the half circles openings 1802c still allow an opening for the steam to exit during ablation and reach the fundus of the uterus. In some embodiments, the ablation catheter 1802t does not include a cap 1849. In embodiments, the shaft 1847 of the catheter 1802t includes a plurality of ports 1843 for the delivery of vapor to other portions of the uterus.

FIG. 18U illustrates a distal end of an ablation catheter 18100a having a spherical shaped distal positioning element 18106 and a cover 18112 extending over the entirety or a portion of the positioning element 18106, in accordance with an exemplary embodiment of the present specification. FIG. 18V illustrates a distal end of an ablation catheter 18100b having a spherical shaped distal positioning element 18108, in accordance with another exemplary embodiment of the present specification. FIG. 18W illustrates a distal end of an ablation catheter 18100c having a conical shaped distal positioning element 18110, in accordance with yet another exemplary embodiment of the present specification. Embodiments of FIGS. 18U, 18V, and 18W, may be used in catheter devices for endometrial ablation as well as for ablation of urinary bladder as described in subsequent figures. Referring simultaneously to FIGS. 18U, 18V, and 18W, a distal tip 18102 of the catheter shaft extends into the positioning elements 18106, 18108, 18110. The distal tip 18102 is an extension of the catheter shaft and is configured to have a smooth rounded tip at its most distal end. In some alternative embodiments, the distal tip 18102 is soft and is configured to have half circles, similar to embodiment of FIG. 18T. A portion of the distal tip 18102 has at least one or a plurality of openings 18104 to provide an exit for steam during ablation. In some embodiments, the openings 18104 are circular, slotted, semi-circular, or of any other shape. In some embodiments, 1 to 1000 openings 18104 are distributed over a length of 3 to 7 cm across the length and surface of the distal tip 18102, where each opening has a length or a diameter in a range of 0.1 to 1 mm. In some embodiments, 64 to 96 openings are distributed over the distal tip 18102. In embodiments, the distal tip 18102 of the catheter is encompassed within the positioning element, such as a spherical element 18106 of FIG. 18U, a spherical element 18108 of FIG. 18V, or an inverted 3-dimensional (3D) conical shaped wire mesh 18110 of FIG. 18WX. In embodiments, positioning elements 18106, 18108, and 18110 are configured to compress or deform when they contact the fundus of the uterus or bladder. A tip of each positioning element 18106, 18108, and 18110 is free floating and the positioning elements 18106, 18108, and 18110 are attached to the respective catheter at a proximal neck of the distal tip 18102. Therefore, the positioning elements 18106, 18108, and 18110 act as a ‘bumper’ and are atraumatic to the fundus, while also allowing for the distribution of steam at the fundus. Each positioning element 18106, 18108, and 18110 is configured from a wire mesh so that there is sufficient space between the wires of the mesh for steam to exit. Referring to FIG. 18U, a cover 18112 is provided to partially cover the openings through the wire mesh on a proximal (bottom) side of the spherical positioning element 18106 to prevent steam from flowing in this direction. In some embodiments, cover 18112 is silicone. FIG. 18V illustrates an alternative embodiment of the spherical positioning element 18106, in the form of the spherical positioning element 18108 that does not include a cover 18112. FIG. 18W illustrates use of a conical positioning element 18110, which is similar to an upside down Erlenmeyer Flask and is configured to approximate a shape of a uterus.

FIG. 18X illustrates an atraumatic soft tip 18114 of a catheter shaft 18116 that is used for insertion into a cervix 18118, in accordance with some embodiments of the present specification. In some embodiments of the present specification, a catheter shaft 18116 is inserted through a patient's vagina canal 18115 and into and through a portion of the patient's cervix. During delivery, a distal hood 18120, inner catheter shaft 18126, and proximal hood 18122 are all disposed within catheter shaft 18120 such that soft tip 18114 comprises the distal end of the catheter. Soft tip 18114 is configured to be soft and atraumatic to the vaginal canal 18115, external cervical os 18117, and cervix 18118 during positioning. During deployment, inner catheter shaft 18126 is extended from catheter shaft 18116, through the cervix 18118 and into a uterus 18124, such that inner catheter shaft 18126 is positioned within the uterus 18124 proximate a fibroid/tumor/lesion 18128 that is required to be treated with ablation. Distal hood 18120 is deployed proximate a fundus 18132 of the uterus 18124 and proximal hood 18122 is deployed proximate an internal cervical os 18119 to firmly position the inner catheter shaft 18126 within the uterus. Openings in the inner catheter shaft 18126 are then used to deliver steam or vapor 18130 to ablate the target area. In some embodiments, a 40 second cycle of vapor ablation is delivered to the uterus. During the ablation, the distal hood 18120 may be pulled back slightly to ensure complete coverage of the target area, including the fundus 18132 of the uterus. The atraumatic soft tip 18114 ensures that the body tissues of the patient are protected during insertion of the catheter and pulling back of the distal hood 18120.

FIG. 19J illustrates a distal end of an ablation catheter 1910 having a proximal positioning element 1911, distal positioning element 1912, and a plurality of ports 1913 along a length of the catheter shaft 1914, in accordance with some embodiments of the present specification. In embodiments, the catheter 1910 includes a proximal connector 1916 for connecting the proximal positioning element 1911 and for connecting the catheter 1910 to a proximal catheter portion, and a distal connector 1917 for connecting the distal positioning element 1912. In some embodiments, the positioning elements 1911, 1912 have conical or circular shapes. In some embodiments, the positioning elements are connected via sutures or wires 1918.

FIG. 19K illustrates a distal end of an ablation catheter 1920 having a proximal positioning element 1921, a distal positioning element 1922, a distal olive tip 1925, and a plurality of ports 1923 along a length of the catheter shaft 1924, in accordance with some embodiments of the present specification. In some embodiments, the catheter 1920 includes a proximal connector 1926 having a screw thread for connecting to a proximal catheter portion.

FIG. 19L illustrates a connector 1930 for connecting a distal positioning element to a distal end of an ablation catheter, in accordance with some embodiments of the present specification. In embodiments, the connector 1930 has a flat distal end 1931, is configured to fit coaxially over a distal portion of an ablation catheter, and includes a plurality of openings 1932 for passage of suture or wire for securing a distal positioning element. In embodiments, the connector 1933 includes an opening at its distal end to allow vapor to escape and reach a fundus of a uterus.

FIG. 19M illustrates another connector 1935 for connecting a distal positioning element to a distal end of an ablation catheter, in accordance with other embodiments of the present specification. In embodiments, the connector 1935 has a rounded distal end 1936 configured to be atraumatic to body tissues, is configured to fit coaxially over a distal portion of an ablation catheter, and includes a plurality of openings 1937 for passage of suture or wire for securing a distal positioning element.

FIG. 19N illustrates a connector 1940 for connecting a proximal positioning element to a distal end of an ablation catheter, in accordance with some embodiments of the present specification. In embodiments, a distal end of the connector 1940 includes a plurality of openings 1941 for passage of suture or wire for securing a proximal positioning element and a proximal end 1942 of the connector is configured to connect to a proximal catheter portion.

FIG. 19O illustrates another connector 1945 for connecting a proximal positioning element to a distal end of an ablation catheter, in accordance with other embodiments of the present specification. In embodiments, a distal end of the connector 1945 includes a plurality of openings 1946 for passage of suture or wire for securing a proximal positioning element and a proximal end 1947 of the connector is configured to connect to a proximal catheter portion.

FIG. 19P illustrates a shaft 1950 of an ablation catheter depicting a plurality of ports 1951, in accordance with some embodiments of the present specification. The ports 1951 are configured to allow the release of vapor from the shaft lumen 1952 into a uterus. In some embodiments, the ports 1951 are arranged in rows 1953.

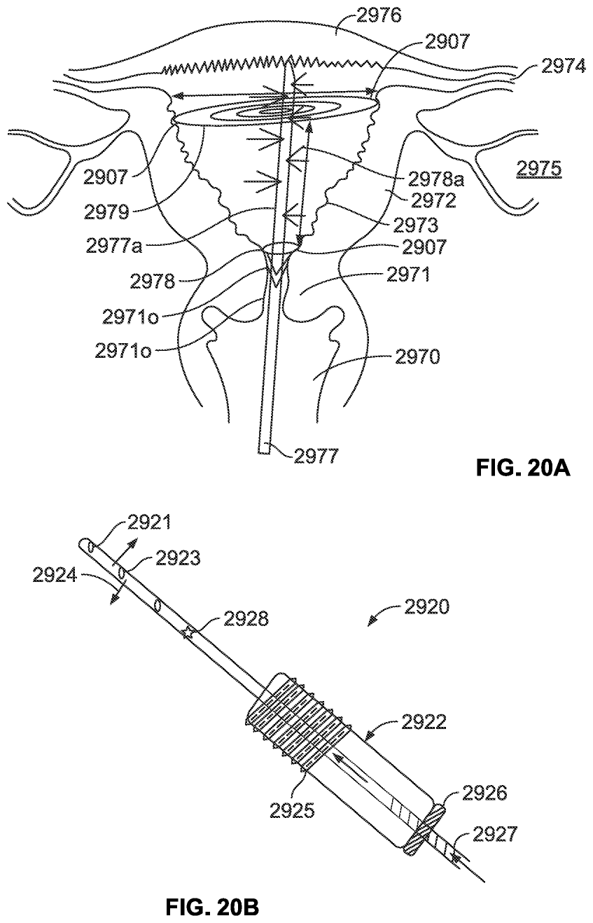

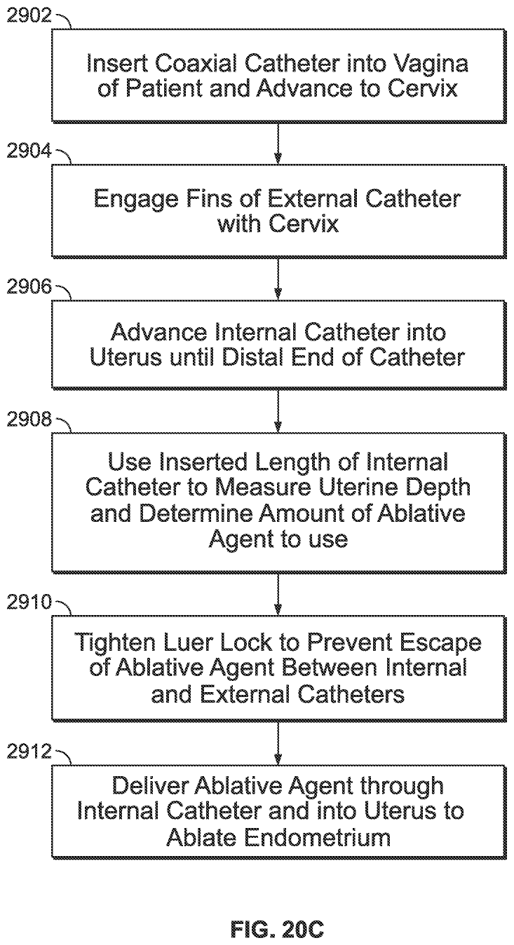

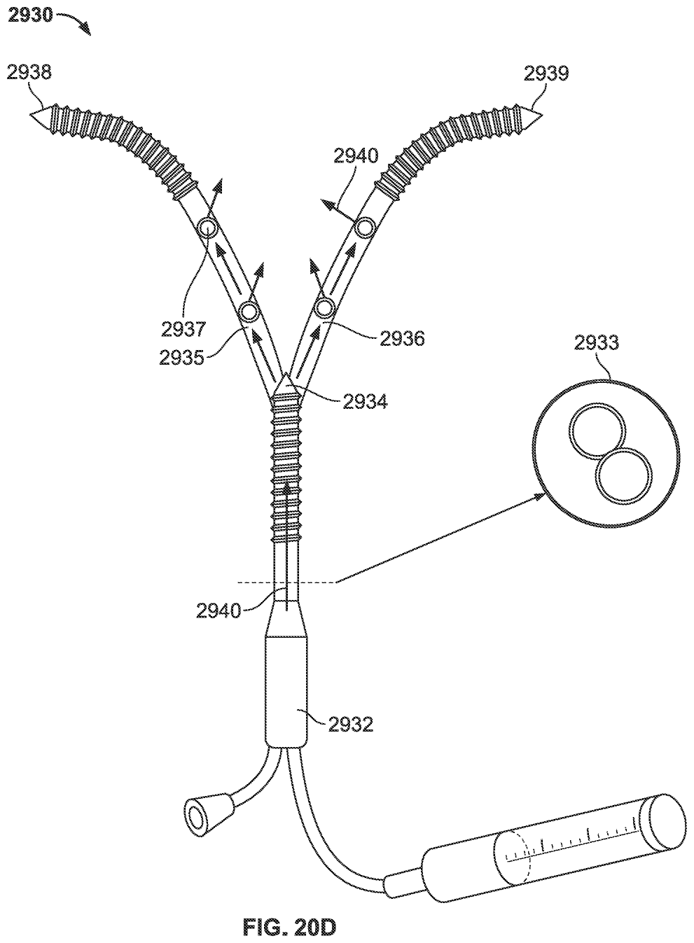

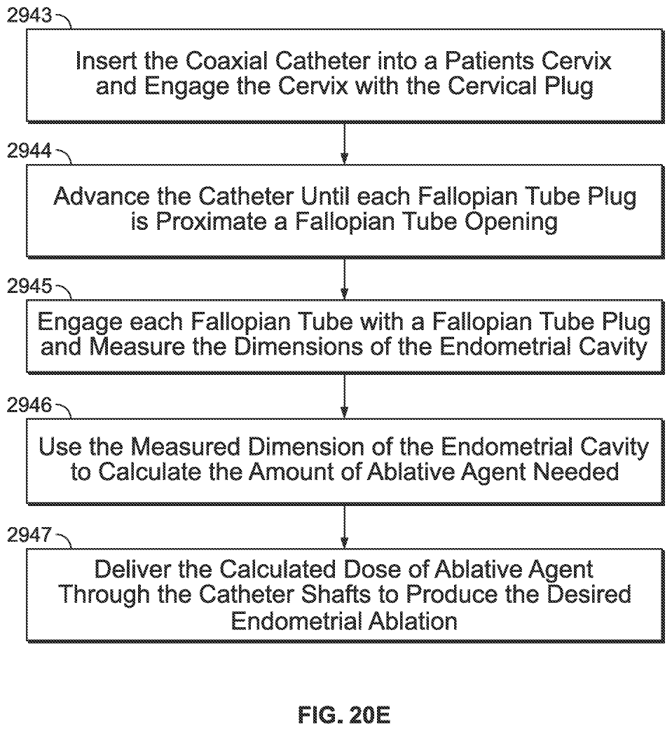

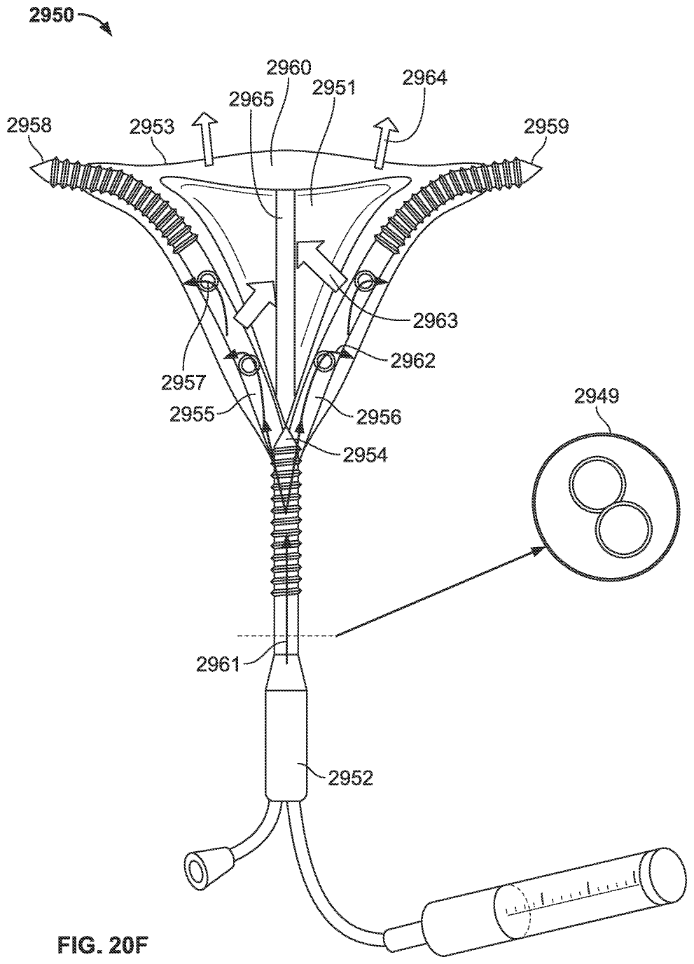

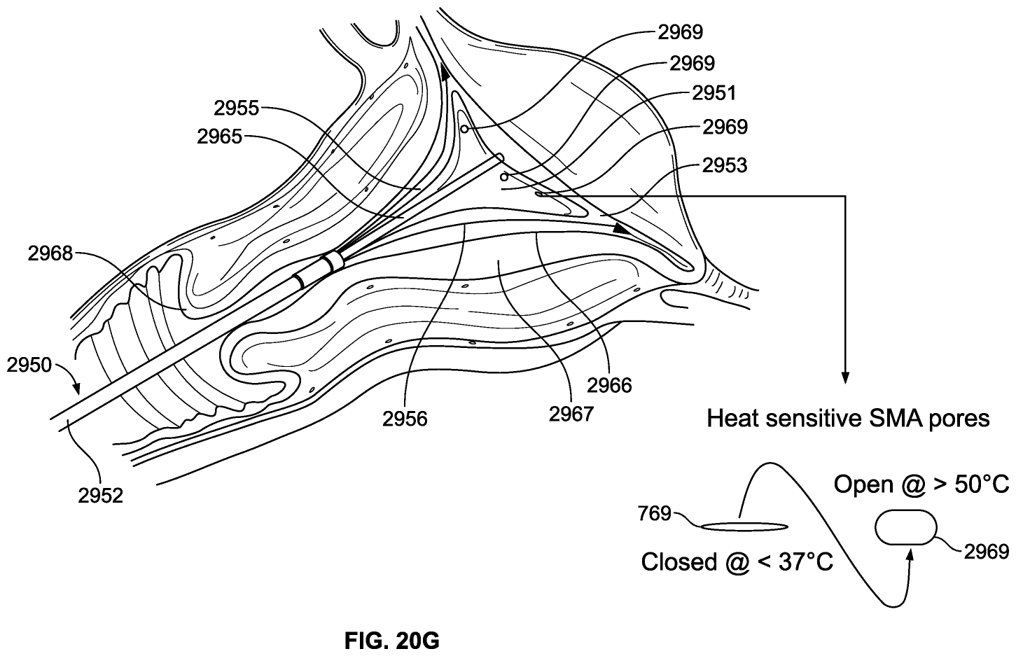

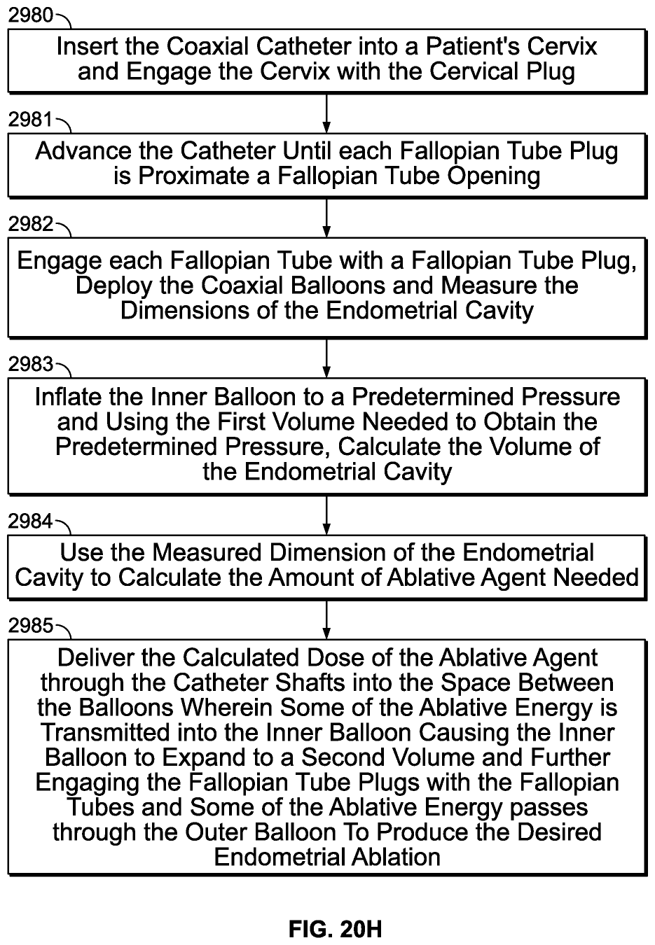

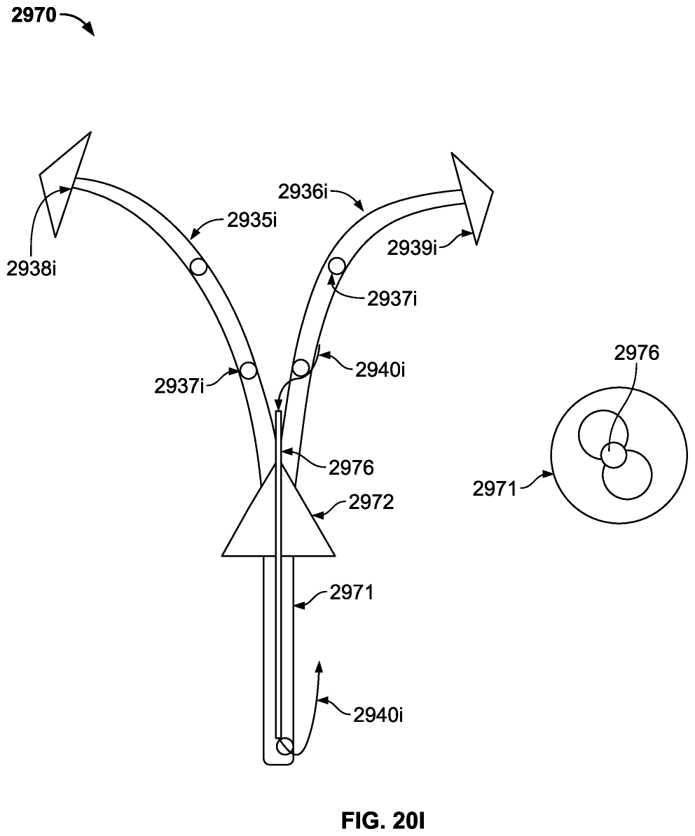

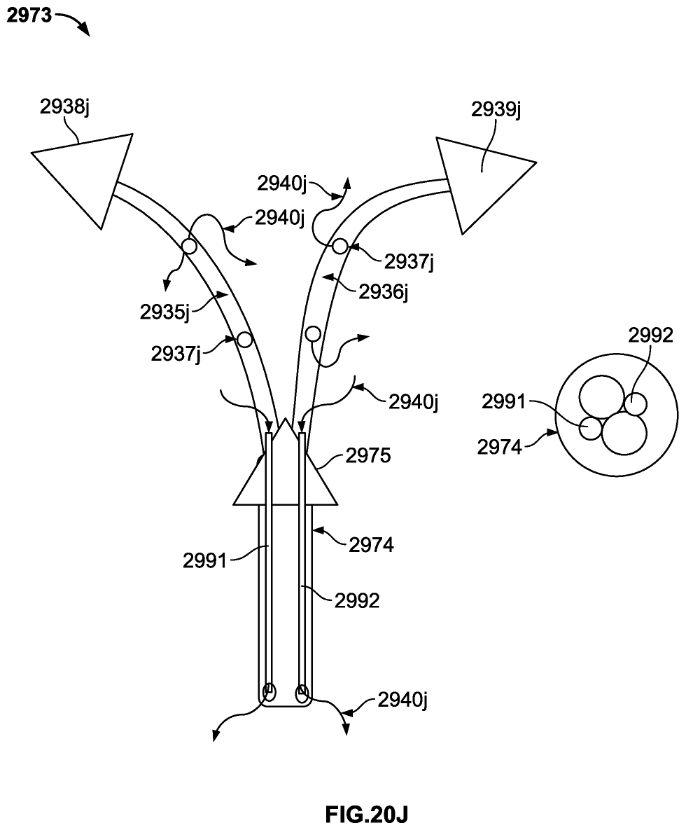

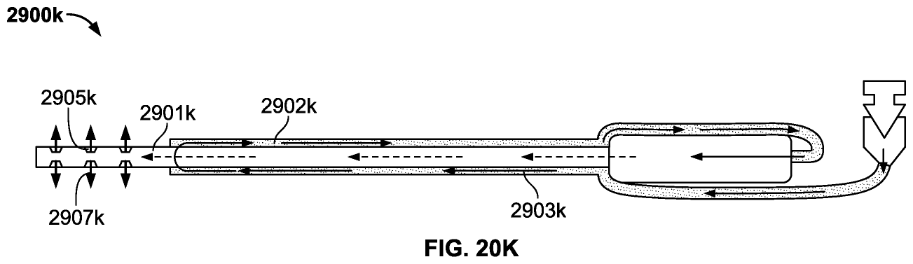

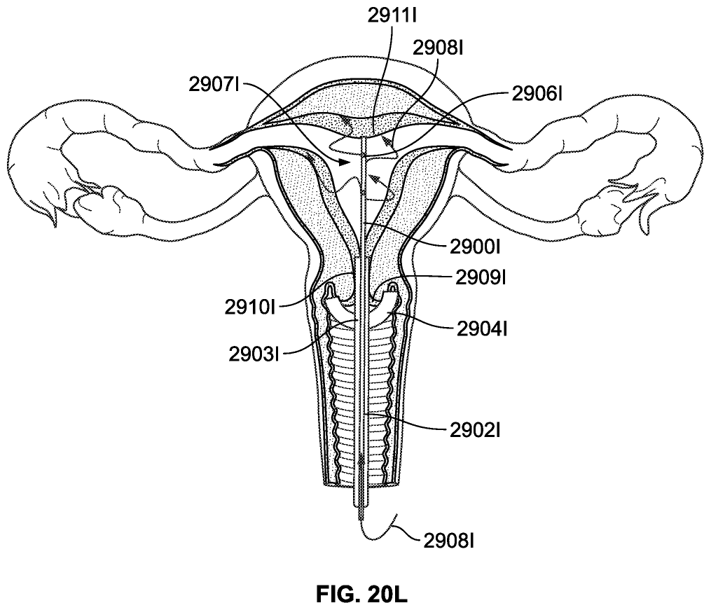

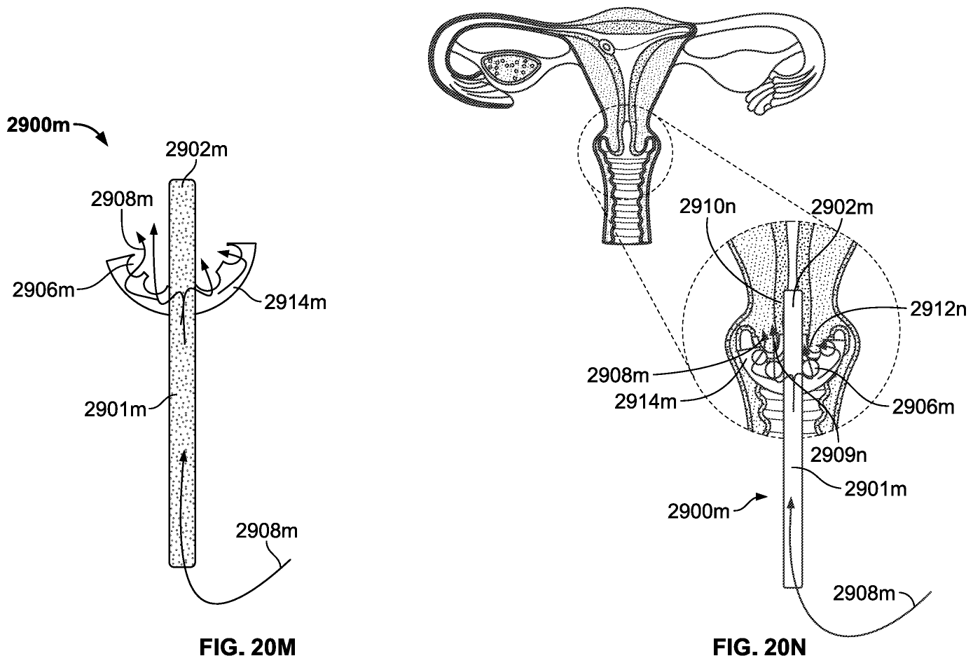

FIG. 20A illustrates endometrial ablation being performed in a female uterus by using the ablation device, in accordance with an embodiment of the present specification. A cross-section of the female genital tract comprising a vagina 2970, a cervix 2971, a uterus 2972, an endometrium 2973, fallopian tubes 2974, ovaries 2975 and the fundus of the uterus 2976 is illustrated. A catheter 2977 of the ablation device is inserted into the uterus 2972 through the cervix 2971 at the cervical os. In an embodiment, the catheter 2977 has two positioning elements, a conical positioning element 2978 and a disc shaped positioning element 2979. The positioning element 2978 is conical with an insulated membrane partially or completely covering the conical positioning element 2978. The conical element 2978 positions the catheter 2977 in the center of the cervix 2971 and the insulated membrane prevents the escape of thermal energy or ablative agent out the cervix 2971 through the os 2971o. The second disc shaped positioning element 2979 is deployed close to the fundus of the uterus 2976 positioning the catheter 2977 in the middle of the cavity. An ablative agent 2978a is passed through infusion ports 2977a for uniform delivery of the ablative agent 2977a into the uterine cavity. Predetermined length ‘l’ of the ablative segment of the catheter and diameter ‘d’ of the positioning element 2979 allows for estimation of the cavity size and is used to calculate the amount of thermal energy needed to ablate the endometrial lining. In one embodiment, the positioning elements 2978, 2979 also act to move the endometrial tissue away from the infusion ports 2977a on the catheter 2977 to allow for the delivery of ablative agent. Optional temperature sensors 2907 deployed close to the endometrial surface are used to control the delivery of the ablative agent 2978a. Optional topographic mapping using multiple infrared, electromagnetic, acoustic or radiofrequency energy emitters and sensors can be used to define cavity size and shape in patients with an irregular or deformed uterine cavity due to conditions such as fibroids. Additionally, data from diagnostic testing can be used to ascertain the uterine cavity size, shape, or other characteristics. In one embodiment, the distal positioning element 2979 is also conical and is either partially or completely covered with an insulating membrane. Various shapes of positioning elements described in this application can be used in various combinations to achieve the desired therapeutic goals.

In an embodiment, the ablative agent is vapor or steam which contracts on cooling. Steam/vapor turns to water which has a lower volume as compared to a cryogen that will expand or a hot fluid used in hydrothermal ablation whose volume stays constant upon contacting the tissue. With both cryogens and hot fluids, increasing energy delivery is associated with increasing volume of the ablative agent which, in turn, requires mechanisms for removing the agent, otherwise the medical provider will run into complications, such as perforation. However, steam, on cooling, turns into water which occupies significantly less volume; therefore, increasing energy delivery is not associated with an increase in volume of the residual ablative agent, thereby eliminating the need for continued removal. This further decreases the risk of leakage of the thermal energy via the fallopian tubes 2974 or the cervix 2971, thus reducing any risk of thermal injury to adjacent healthy tissue.

In one embodiment, the positioning attachment must be separated from the ablation region by a distance of greater than 0.1 mm, preferably 1 mm and more preferably 1 cm. In another embodiment, the positioning attachment can be in the ablated region as long as it does not cover a significant surface area. For endometrial ablation, 100% of the tissue does not need to be ablated to achieve the desired therapeutic effect. Hence, in some embodiments, the positioning element can contact and cover 5% or less of the endometrial surface area.

In one embodiment, the preferred distal positioning attachment is an uncovered wire mesh that is positioned proximate to the mid body region. In one embodiment, the preferred proximal positioning device is a covered wire mesh that is pulled into the cervix, centers the device, and occludes the cervix and or the internal os. FIGS. 19A, 19B, and 19C illustrates some of the various embodiments of the positioning devices. One or more such positioning devices may be helpful to compensate for the anatomical variations in the uterus. The distal positioning device is preferably oval, with a long axis between 0.1 mm and 10 cm (preferably 1 cm to 5 cm) and a short axis between 0.1 mm and 5 cm (preferably 0.5 cm to 1 cm). The proximal positioning device is preferably circular with a diameter between 0.1 mm and 10 cm, preferably 1 cm to 5 cm.

In another embodiment, the catheter is a coaxial catheter comprising an external catheter and an internal catheter wherein, upon insertion, the distal end of the external catheter engages and stops at the cervix while the internal extends into the uterus until its distal end contacts the fundus of the uterus. FIG. 18A illustrates an exemplary embodiment of the catheter configuration in accordance with the present specification. The length of the internal catheter that has passed into the uterus is then used to measure the depth of the uterine cavity and determines the amount of ablative agent to use. Ablative agent is then delivered to the uterine cavity via at least one port on the internal catheter. In one embodiment, during treatment, intracavitary pressure within the uterus is kept below 100 mm Hg, and preferably below 30 mm Hg (no more than 10% above atmospheric pressure). In one embodiment, the coaxial catheter further includes a pressure sensor to measure intracavitary pressure. In one embodiment, the coaxial catheter further includes a temperature sensor to measure intracavitary temperature. In one embodiment, the ablative agent is steam and the steam is released from the catheter at a pressure of less than 100 mm Hg, and preferably below 30 mm Hg. In one embodiment, the steam is delivered with a temperature between 90 and 100° C. In another embodiment the steam is delivered between the temperature of 100-110° C.

FIG. 20B is an illustration of a coaxial catheter 2920 used in endometrial tissue ablation, in accordance with one embodiment of the present specification. The coaxial catheter 2920 comprises an inner catheter 2921 and outer catheter 2922. In one embodiment, the inner catheter 2921 has one or more ports 2923 for the delivery of an ablative agent 2924. In one embodiment, the ablative agent is steam. In one embodiment, the outer catheter 2922 has multiple fins 2925 to engage the cervix to prevent the escape of vapor out of the uterus and into the vagina. In one embodiment, the fins are composed of silicone. The fins 2925 ensure that the cervix is not completely sealed. In embodiments, multiple holes are configured in the fins 2925, which direct the vapor escaping the uterus into a lumen of the outer catheter 2922. In one embodiment, the outer catheter 2922 includes a luer lock 2926 to prevent the escape of vapor between the inner catheter 2921 and outer catheter 2922. In one embodiment, the inner catheter 2921 includes measurement markings 2927 to measure the depth of insertion of the inner catheter 2921 beyond the tip of the outer catheter 2922. Optionally, in various embodiments, one or more sensors 2928 are incorporated into the inner catheter 2921 to measure intracavitary pressure or temperature.

FIG. 20C is a flow chart listing the steps involved in an endometrial tissue ablation process using a coaxial ablation catheter, in accordance with one embodiment of the present specification. At step 2902, the coaxial catheter is inserted into the patient's vagina and advanced to the cervix. Then, at step 2904, the coaxial catheter is advanced such that the fins of the outer catheter engage the cervix, effectively stopping advancement of the outer catheter at that point. The inner catheter is then advanced, at step 2906, until the distal end of the internal catheter contacts the fundus of the uterus. The depth of insertion is then measured using the measurement markers on the internal catheter at step 2908, thereby determining the amount of ablative agent to use in the procedure. At step 2910, the luer lock is tightened to prevent any escape of vapor between the two catheters. The vapor is then delivered, at step 2912, through the lumen of the inner catheter and into the uterus via the delivery ports on the internal catheter to ablate the endometrial tissue.

FIG. 20D is an illustration of a bifurcating coaxial catheter 2930 used in endometrial tissue ablation, in accordance with one embodiment of the present specification. The catheter 2930 includes a first elongate shaft 2932 having a proximal end, a distal end and a first lumen within. The first lumen splits in the distal end to create a coaxial shaft 2933. The distal end of the first shaft 2932 also includes a first positioning element, or cervical plug 2934, that occludes a patient's cervix. The catheter 2930 bifurcates as it extends distally from the cervical plug 2934 to form a second catheter shaft 2935 and a third catheter shaft 2936. The second and third catheter shafts 2935, 2936 each include a proximal end, a distal end, and a shaft body having one or more vapor delivery ports 2937. The second and third catheter shafts 2935, 2936 include second and third lumens respectively, for the delivery of ablative agent. The distal ends of the second and third catheter shafts 2935, 2936 include second and third positioning elements, or fallopian tube plugs 2938, 2939 respectively, designed to engage a patient's fallopian tubes during an ablation therapy procedure and prevent the escape of ablative energy. The fallopian tube plugs 2938, 2939 also serve to position the second and third shafts 2935, 2936 respectively, in an intramural portion or isthmus of a patient's fallopian tube. The second and third catheter shafts 2935, 2936 are independently coaxially extendable and the length of each shaft 2935, 2936 is used to determine the dimension of a patient's endometrial cavity. An ablative agent 2940 travels through the first catheter shaft 2932, through both second and third catheter shaft 2935, 2936, and out the vapor delivery ports 2937 and into the endometrial cavity to ablate endometrial tissue.

FIG. 20E is a flowchart listing the steps of a method of using the ablation catheter of FIG. 20D to ablate endometrial tissue, in accordance with one embodiment of the present specification. At step 2943, the coaxial catheter is inserted into a patient's cervix and the cervix is engaged with the cervical plug. The catheter is then advanced until each fallopian tube plug is proximate a fallopian tube opening at step 2944. Each fallopian tube is then engaged with a fallopian tube plug at step 2945 and the dimensions of the endometrial cavity are measured. The measurements are based on the length of each catheter shaft that has been advanced. At step 2946, the measured dimensions are used to calculate the amount of ablative agent needed to carry out the ablation. The calculated dose of ablative agent is then delivered through the catheter shafts and into the endometrial cavity to produce the desired endometrial ablation at step 2947.

FIG. 20F is an illustration of a bifurcating coaxial catheter 2950 with expandable elements 2951, 2953 used in endometrial tissue ablation, in accordance with one embodiment of the present specification. Similar to the catheter 2930 of FIG. 20D, the catheter 2950 depicted in FIG. 20F includes a first elongate coaxial shaft 2952 having a proximal end, a distal end and a first lumen within. The first lumen splits in the distal end to create a coaxial shaft 2949. The distal end of the first shaft 2952 also includes a first positioning element, or cervical plug 2954, that occludes a patient's cervix. The catheter 2950 bifurcates as it extends distally from the cervical plug 2954 to form a second catheter shaft 2955 and a third catheter shaft 2956. The second and third catheter shafts 2955, 2956 each include a proximal end, a distal end, and a catheter shaft body having one or more vapor delivery ports 2957. The second and third catheter shafts 2955, 2956 include second and third lumens respectively, for the delivery of ablative agent. The distal ends of the second and third catheter shafts 2955, 2956 include second and third positioning elements, or fallopian tube plugs 2958, 2959 respectively, designed to engage a patient's fallopian tubes during an ablation therapy procedure and prevent the escape of ablative energy. The fallopian tube plugs 2958, 2959 also serve to position the second and third shafts 2955, 2956 respectively, in an intramural portion or isthmus of a patient's fallopian tube. The second and third catheter shafts 2955, 2956 are independently coaxially extendable and the length of each catheter shaft 2955, 2956 is used to determine the dimension of a patient's endometrial cavity.

The catheter 2950 further includes a first expandable member or balloon 2951 and a second expandable member or balloon 2953 comprising a coaxial balloon structure. In one embodiment, the first balloon 2951 is a compliant balloon structure and the second balloon 2953 is a non-compliant balloon structure shaped to approximate the uterine cavity shape, size or volume. In another embodiment, the second balloon 2953 is partially compliant. In another embodiment, the compliance of the two balloons 2951, 2953 is substantially equivalent. The balloons 2951, 2953 are attached to the second and third catheter shafts 2955, 2956 along an inner surface of each shaft 2955, 2956. The first, inner balloon 2951 is positioned within the second, outer balloon 2953. The inner balloon 2951 is designed to be inflated with air and a first volume of the inner balloon 2951 is used to measure a dimension of a patient's endometrial cavity. An ablative agent 2961 is introduced into the catheter 2950 at its proximal end and travels through the first catheter shaft 2952 and into the second and third catheter shafts 2955, 2956. The second and third catheter shafts 2955, 2956 are designed to release ablative energy 2962 through delivery ports 2957 and into a space 2960 between the two balloons 2951, 2953. Some of the ablative energy 2963 is transferred to the air in the inner balloon 2951, expanding its volume from said first volume to a second volume, resulting in further expansion of said inner balloon 2951 to further occlude the patient's endometrial cavity for ideal vapor delivery. In one embodiment, the second volume is less than 25% greater than the first volume. The expansion also forces the fallopian tube plugs 2958, 2959 to further engage the openings of the fallopian tubes. A portion of the ablative agent or ablative energy 2964 diffuses out of the thermally permeable outer balloon 2953 and into the endometrial cavity, ablating the endometrial tissue. In various embodiments, the thermal heating of the air in the balloon occurs either through the walls of the inner balloon, through the length of the catheter, or through both. In one embodiment, the catheter 2950 includes an optional fourth catheter shaft 2965 extending from the first catheter shaft 2952 and between the second and third catheter shaft 2955, 2956 within the inner balloon 2951. Thermal energy from within the fourth catheter shaft 2965 is used to further expand the inner balloon 2951 and assist with ablation.

In one embodiment, the volume of the inner balloon 2951 is used to control the pressure exerted by the outer balloon 2953 on the wall of the uterus. The pressure in the inner balloon 2951 is monitored and air is added to or removed from the inner balloon 2951 to maintain a desirable therapeutic pressure in the outer balloon 2953.

FIG. 20G is an illustration of the catheter 2950 of FIG. 20F inserted into a patient's uterine cavity 2966 for endometrial tissue 2967 ablation, in accordance with one embodiment of the present specification. The catheter 2950 has been inserted with the first shaft 2952 extending through the patient's cervix 2968 such that the second shaft 2955 is positioned along a first side of the patient's uterine cavity 2966 and the third shaft 2956 is positioned along a second side opposite said first side. This positioning deploys the inner balloon 2951 and outer balloon 2953 between the second and third shafts 2955, 2956. In the pictured embodiment, the catheter 2950 includes an optional fourth shaft 2965 to further expand the inner balloon 2951 with thermal energy and assist with ablation of endometrial tissue 2967. In one embodiment, the inner balloon 2951 is optional and the outer balloon 2953 performs the function of both sizing and delivery of the ablative agent. In one embodiment, the outer balloon includes heat sensitive pores 2969 which are closed at room temperature and open at a temperature higher than the body temperature. In one embodiment, the pores are composed of a shape memory alloy (SMA). In one embodiment, the SMA is Nitinol. In one embodiment, the austenite finish (Af) temperature, or temperature at which the transformation from martensite to austenite finishes on heating (alloy undergoes a shape change to become an open pore 2969), of the SMA is greater than 37° C. In other embodiments, the Af temperature of the SMA is greater than 50° C. but less than 100° C.

FIG. 20H is a flowchart listing the steps of a method of using the ablation catheter of FIG. 20F to ablate endometrial tissue, in accordance with one embodiment of the present specification. At step 2980, the coaxial catheter is inserted into a patient's cervix and the cervix is engaged with the cervical plug. The catheter is then advanced until each fallopian tube plug is proximate a fallopian tube opening at step 2981. Each fallopian tube is then engaged with a fallopian tube plug at step 2982, which also deploys the coaxial balloons in the endometrial cavity, and the dimensions of the endometrial cavity are measured. The measurements are based on the length of each catheter shaft that has been advanced and a first volume needed to expand the inner balloon to a predetermined pressure. At step 2983, the inner balloon is inflated to said predetermined pressure and a first volume of the inner balloon at said pressure is used to calculate the volume of the endometrial cavity. The measured dimensions are then used at step 2984 to calculate the amount of ablative agent needed to carry out the ablation. The calculated dose of ablative agent is then delivered through the catheter shafts and into the space between the coaxial balloons at step 2985. Some of the ablative energy is transmitted into the inner balloon to expand the inner balloon to a second volume which further expands the endometrial cavity and, optionally, further pushes the fallopian tube plugs into the fallopian tube openings to prevent the escape of thermal energy. Another portion of the ablative energy passes through the thermally permeable outer balloon to produce the desired endometrial ablation.

In another embodiment, a vapor ablation device for ablation of endometrial tissue comprises a catheter designed to be inserted through a cervical os and into an endometrial cavity, wherein the catheter is connected to a vapor generator for generation of vapor and includes at least one port positioned in the endometrial cavity to deliver the vapor into the endometrial cavity. The vapor is delivered through the port and heats and expands the air in the endometrial cavity to maintain the endometrial cavity pressure below 200 mm Hg and ideally below 50 mm of Hg. In one embodiment, an optional pressure sensor measures the pressure and maintains the intracavitary pressure at the desired therapeutic level, wherein the endometrial cavity is optimally expanded to allow for uniform distribution of ablative energy without the risk of significant leakage of the ablative energy beyond the endometrial cavity and damage to the adjacent normal tissue.

FIG. 20I is an illustration of a bifurcating coaxial catheter 2970 used in endometrial tissue ablation, in accordance with another embodiment of the present specification. Forming a seal at the cervix is undesirable as it creates a closed cavity, resulting in a rise of pressure when vapor is delivered into the uterus. This increases the temperature of the intrauterine air, causing a thermal expansion and further rise of intracavitary pressure. This rise in pressure may force the vapor or hot air to escape out of the fallopian tubes, causing thermal injury to the abdominal viscera. This requires for continuous measurement of intracavitary pressure and active removal of the ablative agent to prevent leakage of thermal energy outside the cavity. Referring to FIG. 20I, the catheter 2970 includes a coaxial handle 2971, a first positioning element 2972, a first bifurcated catheter arm 2935i with a second positing element 2938i at its distal end, a second bifurcated catheter arm 2936i with a third positioning element 2939i at its distal end, and a plurality of infusion ports 2937i along each bifurcated catheter arm 2935i, 2936i. The catheter 2970 also includes a venting tube 2976 which extends through the coaxial handle 2971 and through the first positioning element 2972 such that the lumen of a patient's uterus is in fluid communication with the outside of the patient's body when the first positioning element 2972 is in place positioned against a cervix. This prevents formation of a tight seal when the catheter 2970 is inserted into the cervix. Since the cervix is normally in a closed position, insertion of any device will inadvertently result in formation of an undesirable seal. The venting tube allows for heated air or extra vapor 2940i to vent out as it expands with delivery of vapor and the intracavitary pressure rises. In some embodiments, the venting tube includes a valve for unidirectional flow of air.

FIG. 20J is an illustration of a bifurcating coaxial catheter 2973 used in endometrial tissue ablation, in accordance with yet another embodiment of the present specification. The catheter 2973 includes a coaxial handle 2974, a first positioning element 2975, a first bifurcated catheter arm 2935j with a second positing element 2938j at its distal end, a second bifurcated catheter arm 2936j with a third positioning element 2939j at its distal end, and a plurality of infusion ports 2937j along each bifurcated catheter arm 2935j, 2936j. The catheter 2973 also includes two venting tubes 2991, 2992 which extend through the coaxial handle 2974 and through the first positioning element 2975 such that the lumen of a patient's uterus is in fluid communication with the outside of the patient's body when the first positioning element 2975 is in place positioned against a cervix. This prevents formation of a tight or complete seal when the catheter 2973 is inserted into the cervix. The venting tubes 2991, 2992 allow for heated air or extra vapor 2940j to vent out as it expands with delivery of vapor and the intracavitary pressure rises. In some embodiments, the venting tubes 2991, 2992 include a valve for unidirectional flow of air.

FIG. 20K is an illustration of a water cooled catheter 2900k used in endometrial tissue ablation, in accordance with one embodiment of the present specification. The catheter 2900k comprises an elongate body 2901k having a proximal end and a distal end. The distal end includes a plurality of ports 2905k for the delivery of vapor 2907k for tissue ablation. A sheath 2902k extends along the body 2901k of the catheter 2900k to a point proximal to the ports 2905k. During use, water 2903k is circulated through the sheath 2902k to cool the catheter 2900k. Vapor 2907k for ablation and water 2903k for cooling are supplied to the catheter 2900k at its proximal end.

FIG. 20L is an illustration of a water cooled catheter 2900l used in endometrial tissue ablation and positioned in a uterus 2907l of a patient, in accordance with another embodiment of the present specification. The catheter 2900l comprises an elongate body 2901l, a proximal end, distal end, and a sheath 2902l covering a proximal portion of the body 2901l. Extending from, and in fluid communication with, the sheath 2902l is a cervical cup 2904l. The catheter 2900l further includes a plurality of ports 2906l at its distal end configured to deliver ablative vapor 2908l to the uterus 2907l. Vapor 2908l is supplied to the proximal end of the catheter 2900l. The ports 2906l are positioned on the catheter body 2901l distal to the sheath 2902l. The cervical cup 2904l is configured to cover the cervix 2909l and a distal end of the sheath 2902l extends into the cervical canal 2910l. Water 2903l is circulated through the sheath 2902l and cervical cup 2904l to cool the cervical canal 2910l and/or cervix 2909l while vapor 2908l is delivered through the vapor delivery ports 2906l to ablate the endometrial lining 2911l.

In various embodiments, ablation therapy provided by the vapor ablation systems of the present specification is delivered to achieve the following therapeutic endpoints for uterine ablation: maintain a tissue temperature at 100° C. or less; increase patient's hemoglobin by at least 5% or at least 1 gm % relative to pre-treatment hemoglobin; decrease menstrual blood flow by at least 5% as measured by menstrual pad weight relative to pre-treatment menstrual blood flow; ablation of endometrial tissue in a range of 10% to 99%; decrease in duration of menstrual flow by at least 5% relative to pre-treatment menstrual flow; decrease in amenorrhea rate by at least 10% relative to pre-treatment amenorrhea rate; and patient reported satisfaction with uterine ablation procedure of greater than 25%.

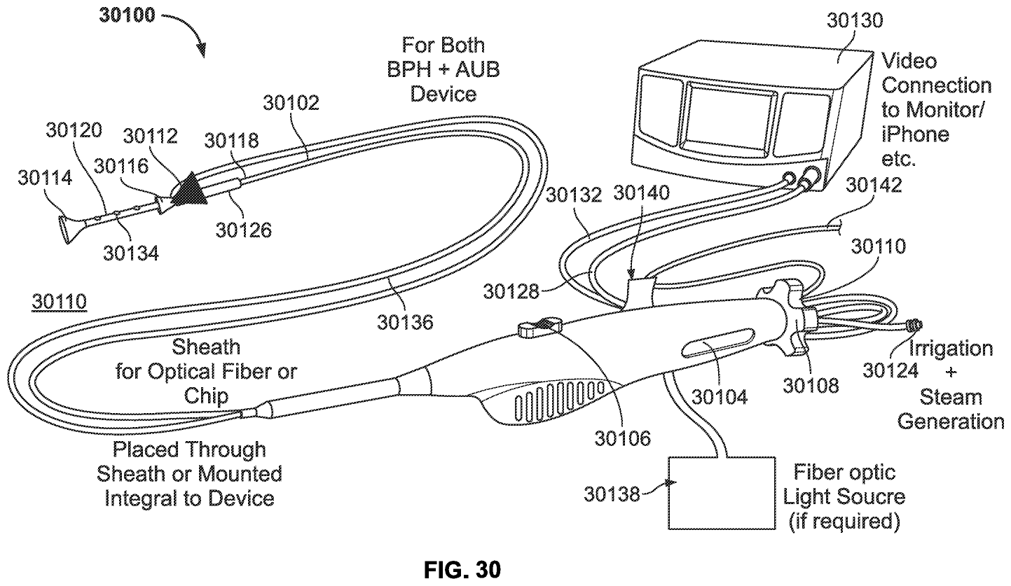

FIG. 20M is an illustration of a water cooled catheter 2900m used in cervical ablation, in accordance with one embodiment of the present specification, and FIG. 20N is an illustration of the catheter 2900m of FIG. 20M positioned in a cervix 2909n of a patient. Referring to FIGS. 29M and 29N simultaneously, the catheter 2900m comprises an elongate body 2901m, a proximal end, a distal end, and a water cooled tip 2902m at its distal end. A cervical cup 2914m is attached to the catheter body 2901m and includes a plurality of ports 2906m which are in fluid communication with the proximal end of the catheter 2900m. Vapor 2908m is provided at the proximal end of the catheter 2900m and is delivered to the cervix 2909n via ports 2906m. In an embodiment, the vapor 2908m ablates the transformation zone 2912n at the cervix 2909n. The water cooled tip 2902m of the catheter 2900m cools the cervical canal 2910n during ablation. In various embodiments, cooling methods known in the art can be used to cool the catheter tip.

FIG. 20O is a flowchart listing the steps involved in cervical ablation performed using the catheter of FIGS. 29M. At step 2902o the distal tip of the catheter is inserted into the cervical canal until the cervical cup of the catheter encircles the cervix. Water is circulated through the water cooled tip to cool the cervical canal at step 2904o. At step 2906o vapor is passed through the vapor delivery ports in the cervical cup to ablate the cervix.

In various embodiments, ablation therapy provided by the vapor ablation systems of the present specification is delivered to achieve the following therapeutic endpoints for cervical ablation: maintain a tissue temperature at 100° C. or less; ablate a cervical mucosa without significant injury to the cervical canal; ablate at least 50% of a surface area of a targeted abnormal cervical mucosa such that, upon healing, said abnormal cervical mucosa is replaced by normal cervical mucosa; elimination of more than 25% of abnormal cervical mucosa as assessed by colposcopy; and ablate more than 25% of abnormal cervical mucosa and less than 25% of a total length of a cervical canal.

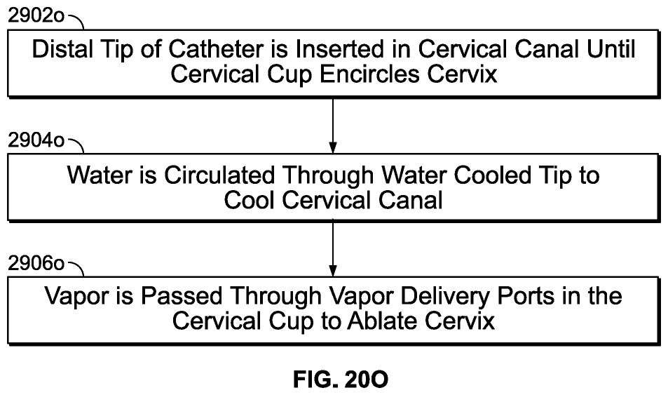

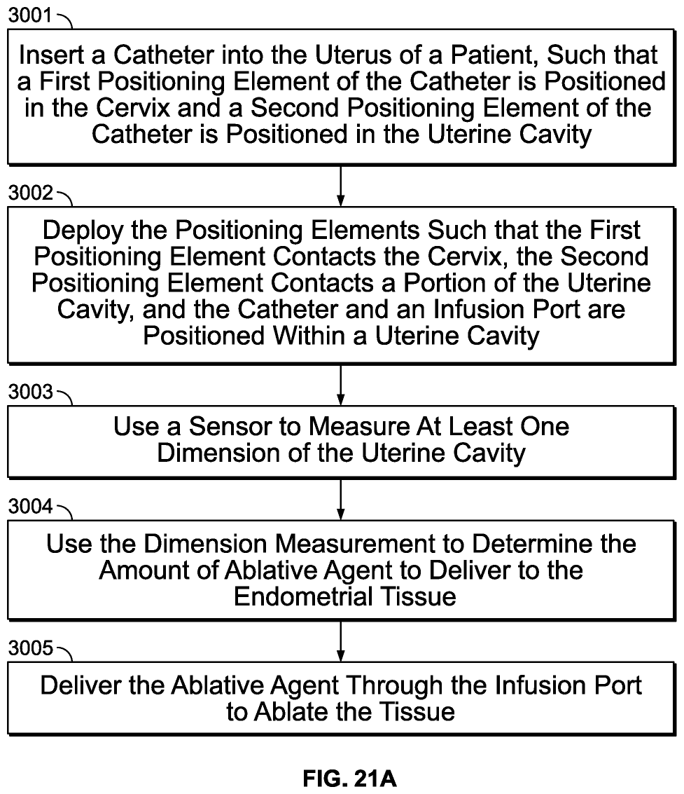

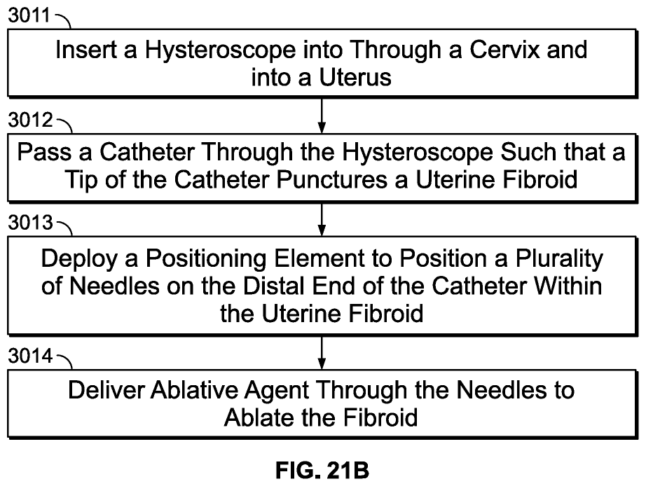

FIG. 21A is a flowchart illustrating a method of ablation of endometrial tissue in accordance with one embodiment of the present specification. Referring to FIG. 21A, the first step 3001 includes inserting a catheter of an ablation device through a cervix and into a uterus of a patient, wherein the catheter includes a hollow shaft through which an ablative agent can travel, at least one first positioning element, at least one second positioning element positioned distal to the at least one first positioning element, and at least one infusion port for delivering the ablative agent. In an embodiment, the ablation device includes a controller comprising a microprocessor for controlling the delivery of the ablative agent. The catheter is passed through the cervix such that a first positioning element is positioned in the cervix and a second positioning element is positioned in the uterine cavity. In one embodiment, the second positioning element is positioned proximate the fundus of the uterus. The two positioning elements are deployed such that the first positioning element contacts the cervix, the second positioning element contacts a portion of the uterine cavity, and the catheter and an infusion port are positioned within a uterine cavity of the patient in step 3002. Finally, in step 3005, an ablative agent is delivered through the infusion port to ablate the endometrial tissue. Optionally, a sensor is used to measure at least one dimension of the uterine cavity in step 3003 and the measurement is used to determine the amount of ablative agent to be delivered in step 3004.

FIG. 21B is a flowchart illustrating a method of ablating a uterine fibroid. Referring to FIG. 21B, the first step 3011 includes inserting a hysteroscope through the cervix and into a uterus of a patient. Next, in step 3012 a catheter of an ablation device is passed through the hysteroscope, wherein the catheter includes a hollow shaft through which an ablative agent can travel, a puncturing tip at its distal end, at least one positioning element, and a plurality of needles on a distal end of the catheter and configured to deliver ablative agent to said uterine fibroid. In an embodiment, the ablation device includes a controller comprising a microprocessor for controlling the delivery of the ablative agent. The catheter is passed through said hysteroscope such that the puncturing tip of the catheter punctures the uterine fibroid. In the next step 3013, the at least one positioning element is deployed to position the catheter within the uterine fibroid such that the plurality of needles on the distal end of said catheter are positioned within the uterine fibroid. Finally, in step 3014, an ablative agent is delivered through the needles to ablate the fibroid. In some embodiments, the positioning element positions the catheter in the fibroid at approximate ½ the average transverse dimension of the fibroid. In other embodiments, the positioning element positions the catheter in the fibroid at approximate 25% to 75% of an average transverse dimension of the fibroid.

### Urinary Bladder Cancer Ablation and Treating OAB

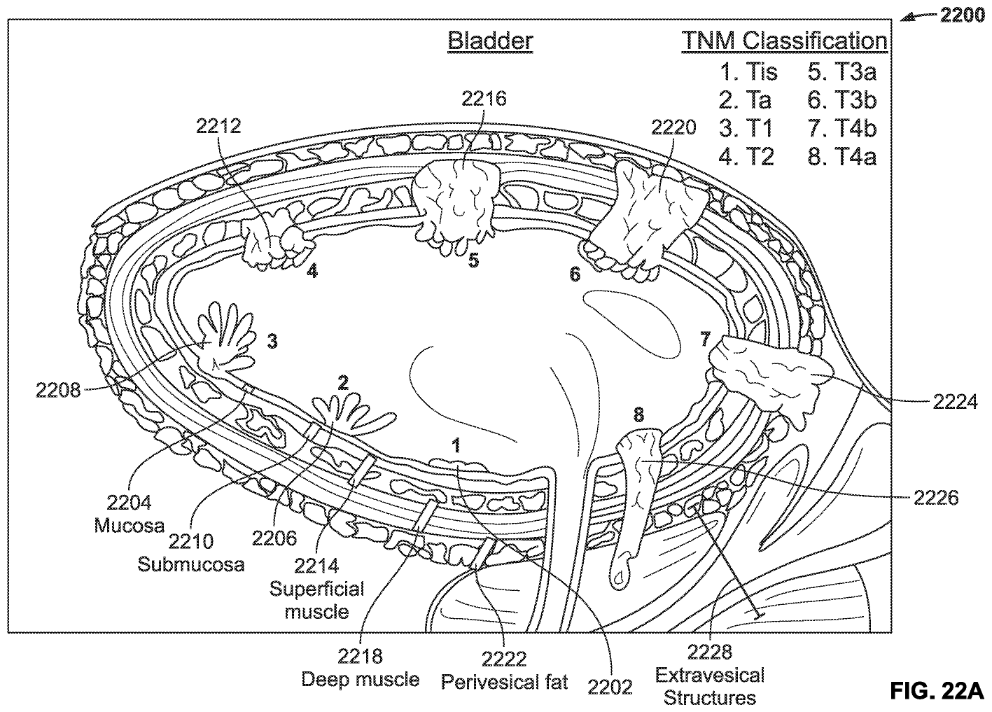

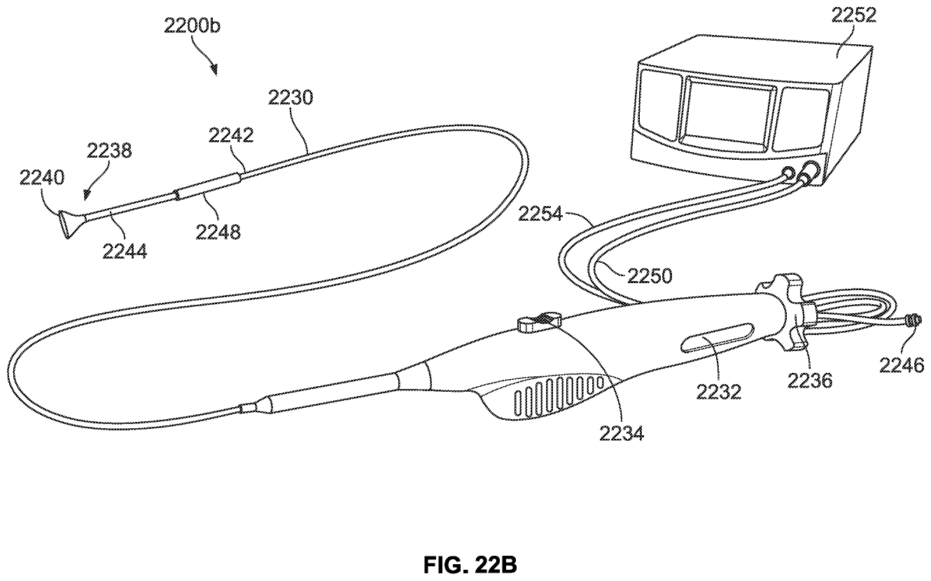

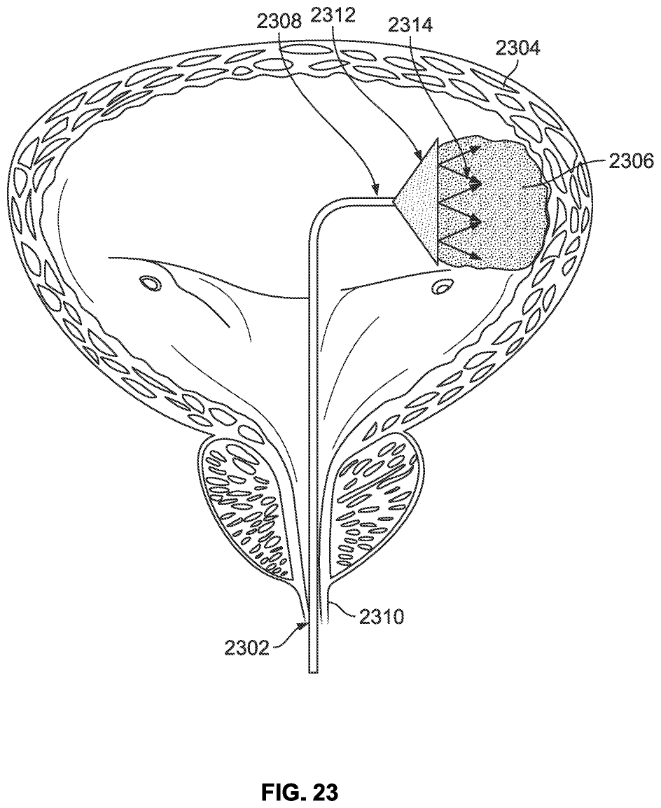

FIG. 22B illustrates a system 2200b for use in the ablation of bladder tissue, in accordance with an embodiment of the present specification. The system 2200b comprises a catheter 2230 which, in some embodiments, includes a handle 2232 having actuators 2234, 2236 for pushing forward a distal tip 2238 of the catheter 2230 and for deploying a distal positioning element 2240 at the distal end of the catheter 2230. In embodiments, the catheter 2230 comprises an outer sheath 2242 and an inner catheter 2244. In embodiments, the distal positioning element 2240 is expandable, positioned at the distal end of the inner catheter 2244, and may be compressed within the outer sheath 2242 for delivery. In some embodiments, actuators 2234 and 2236 comprise knobs. In some embodiments, actuator/knob 2236 is used to deploy the distal positioning element 2240. For example, in embodiments, actuator/knob 2236 is turned one quarter turn to deploy the distal positioning element 2240. In some embodiments, other combinations of actuators/knobs are used to the positioning element 2240. In some embodiments, the catheter 2230 includes a port 2246 for the delivery of fluid, for example cooling fluid, during ablation. In some embodiments, port 2246 is also configured to provide for fluid collection, provide vacuum, and provide CO₂ for an integrity test. In some embodiments, the port 2246 is positioned on the handle 2232. In some embodiments, at least one electrode 2248 is positioned at a distal end of the catheter 2230. The electrode 2248 is configured to receive electrical current, supplied by a connecting wire 2250 extending from a controller 2252 to the catheter 2230, to heat and convert a fluid, such as saline supplied via a tubing 2254 extending from the controller 2252 to the catheter 2230. Heated fluid or saline is converted to vapor or steam to be delivered by ports for ablation. In some embodiments, the catheter 2230 is made of or covered with an insulated material to prevent the escape of ablative energy from the catheter body. A plurality of small delivery ports are positioned on the inner catheter 2244 between the distal positioning element 2240 and the electrode 2248. Ports are used for the infusion of an ablative agent, such as steam. Delivery of the ablative agent is controlled by the controller 2252 and treatment is controlled by a treating physician via the controller 2252. In embodiments, the system 2200b of FIG. 22B is configured to be used for ablation of the urinary bladder, and may be used with catheters, positioning elements, and needles, described subsequently in context of FIGS. 23 to 28.

FIG. 23 illustrates an exemplary catheter 2302 for insertion into a urinary bladder 2304 for ablating bladder cancer 2306, in accordance with some embodiments of the present specification. Exemplary embodiments of distal ends of catheter 2302 are illustrated in context of FIGS. 18V, 18W, and 18X. A distal end 2308 of the catheter 2302 is advanced through a urethra 2310 and into a urinary bladder 2304. A cystoscope may be used for advancement of the catheter or, in some embodiments, visualization capability is provided in the catheter to navigate the catheter. A positioning element 2312 attached to the distal end 2308 of the catheter 2302 is used to position the ablation catheter 2302 inside the bladder 2304. In some embodiments, positioning element 2312 comprises a plurality of wires woven into a pattern, for example a spiral pattern. In embodiments, the wires are composed of a shape memory material to allow for compression of the positioning element 2312 during delivery. In some embodiments, the shape memory material is Nitinol. In various embodiments, the positioning element 2312 has a disc, cone, funnel, bell, spherical, oval, ovoid, or acorn shape and is substantially cylindrical when compressed. The positioning element 2312, when deployed, abuts and rests in the bladder 2304 encircling a portion of the tissue to be ablated.

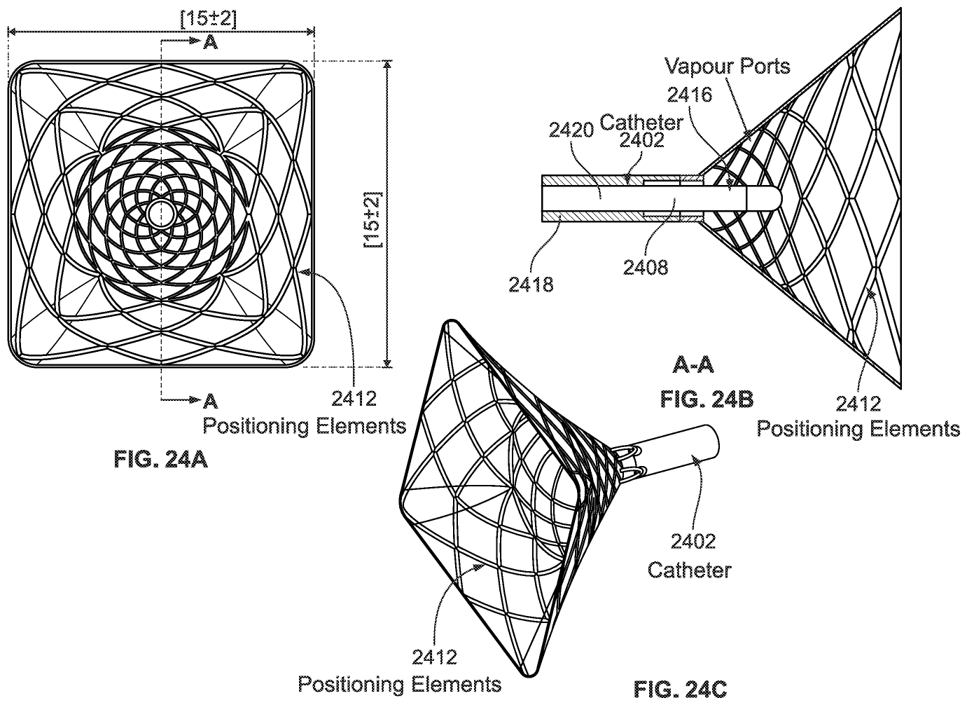

FIGS. 24A, 24B, and 24C illustrate different views of an exemplary configuration of a distal end of a catheter 2402 with a positioning element 2412, in accordance with some embodiments of the present specification. FIG. 24A illustrates a front end view of the positioning element 2412. FIG. 24B illustrates a side view of the catheter 2402 and positioning element 2412. FIG. 24C illustrates a front side perspective view of the catheter 2402 and positioning element 2412. Referring simultaneously to FIGS. 24A, 24B, and 24C, positioning element 2412 is in a shape of a pyramid, with four sides, providing an open square form at its distal end. In some embodiments, the length and width of the positioning element 2412 at its open, distal end is in a range of 13 to 17 mm. Catheter 2402 is attached at its distal end 2408 to the positioning element 2412. Catheter 2402 includes an outer catheter 2418 and an inner catheter 2420. In embodiments, the positioning element 2402 is attached with a connecting mechanism to the distal end 2408 of the outer catheter 2418. The inner catheter 2420 is positioned inside and coaxially with the outer catheter 2418. Vapor ports 2416 are configured on the inner catheter 2420, which provide an outlet for vapor 2314 (FIG. 23) during ablation.

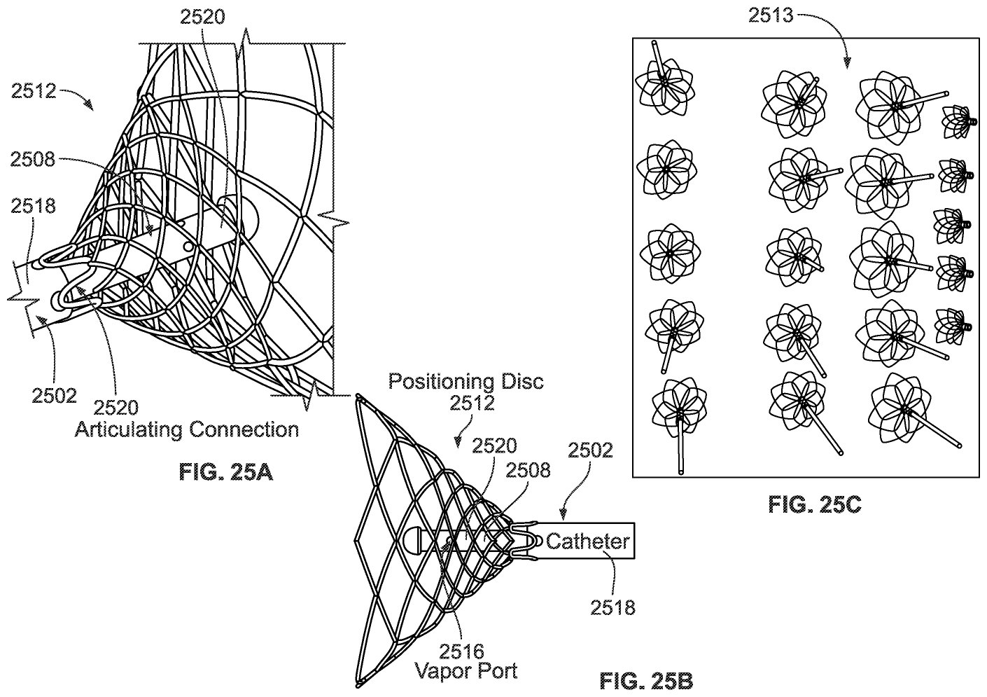

FIGS. 25A, 25B, and 25C illustrate designs of a positioning element 2512, in accordance with some embodiments of the present specification. FIG. 25A illustrates a close-up view of a connection 2520 between positioning element 2512 and a catheter 2502, in accordance with some embodiments of the present specification. In alternative embodiments, the positioning element 2512 is fused with the catheter 2502, is free floating with metal or polymer sutures, is hinged with laser welded Nitinol, where the hinge is cut with a laser, or is attached with a Nitinol sleeve that is welded to it. In some embodiments, the connection 2520 is an over-sleeve or part of a distal end 2508 of the catheter 2502. FIG. 25B illustrates a side view of positioning element 2512 attached to distal end 2508 of the catheter 2502. One or more vapor ports 2516 are configured on an inner catheter 2520 within an outer catheter 2518, at the distal end of the catheter 2502, where the distal portion of the catheter 2502 is located within the funnel-shaped volume of the positioning element 2512. In embodiments, the inner catheter 2520 is movable into and out of the outer catheter 2518 such that the outer catheter 2518 covers the inner catheter 2520 and restrains the positioning element 2512 before insertion into a patient's urethra. The positioning element 2512 is composed of a shape memory material such that it expands into a deployed configuration, as shown in FIG. 25A, once the inner catheter 2520 is extended beyond the distal end of the outer catheter 2518. FIG. 25C illustrates different types of configurations of positioning elements 2513 which may be used in accordance with the embodiments of the present specification. In some embodiments, the positioning elements are conical in shape with diameters varying from 5 mm to 50 mm. In some embodiments, the positioning elements are ovoid cones with a first proximal diameter of the cone less than a second distal diameter of the cone to approximate the shape or dimensions of a urethra. In various embodiments with multiple positioning elements, a first positioning element may have a different shape or size from a second positioning element. One or more positioning elements maybe used to accomplish the therapeutic purpose.

In some embodiments, the positioning elements 2512 are formed with wire made from one, or a combination, of polymers and metal, such as including and not limited to Polyether ether ketone (PEEK) and Nickel Titanium (NiTi). In some embodiments, the wire is covered with elastomers such as PTFE, ePTFE, PU and/or silicone in a variety of patterns. The various cells in the positioning elements 2513 may be covered or uncovered based on hood functionality such as whether it is to be used for sealing, or for venting, or for any other purpose. In embodiments where the positioning elements 2513 are made from Nitinol wire meshes, the wires have a diameter in a range of 0.16 to 0.18 mm. In some embodiments, for the positioning element 2513, the wire mesh is coated with silicone but not the areas between wires in the mesh, therefore allowing steam to escape/vent from these spaces between the wires. In some embodiments, wires and space between wires are covered with silicone.

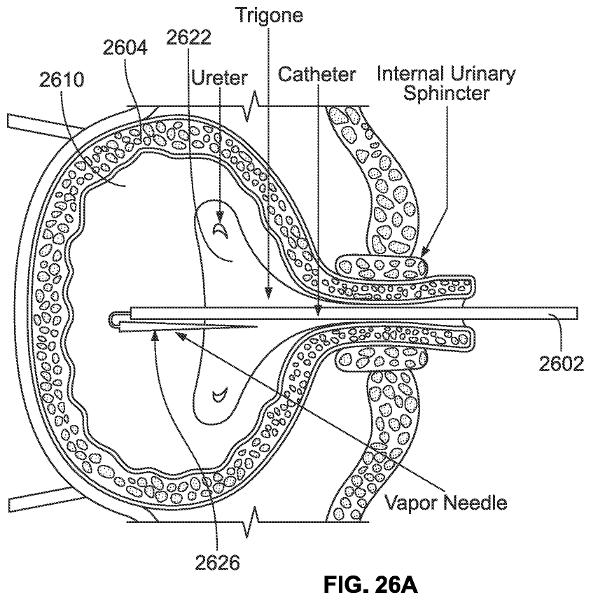

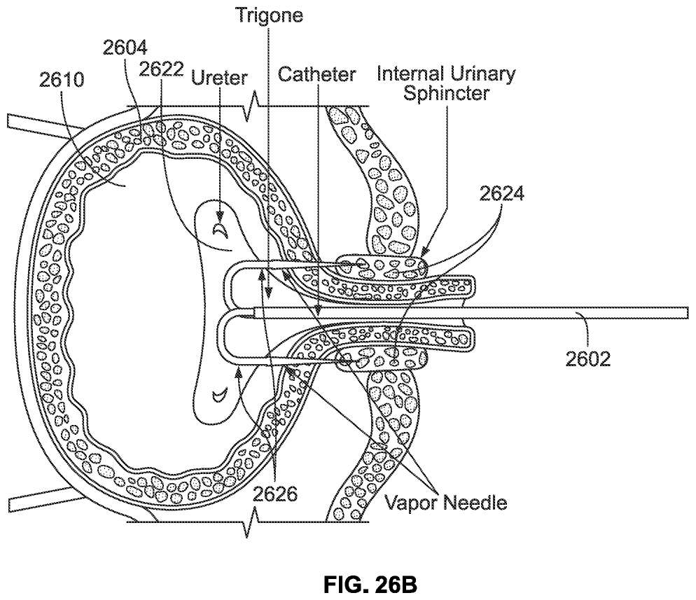

Embodiments of the present specification may also be used in the ablation of bladder neck tissue and/or an internal bladder sphincter for treatment of an OAB, as described with reference to the embodiments of subsequent FIGS. 26A and 26B. An OAB is related to a sudden, uncontrolled need or urge to urinate. OAB is different from stress urinary incontinence (SUI), where people leak urine while sneezing, laughing, or doing other physical activities. OAB may result from improper coordination of the nerve signals between the bladder and the brain. The signals might tell a patient to empty the bladder, even when the bladder is not full. OAB can also be caused when muscles in the bladder are too active. In this case, the bladder muscles contract to pass urine before the bladder is full, causing a sudden urge to urinate. Treating the bladder neck and/or an internal bladder sphincter with the ablation methods of the present specification provide a method of treating OAB. Accordingly, vapor is delivered selectively to ablate nerve-rich layers of deep detrusor and adventitial space beneath trigone. Alternatively, vapor is delivered selectively with the help of the RF generator to ablate the bladder neck, internal urinary sphincter (IUS) and nerves supplying the IUS. The RF generator provides power to electrodes in a heating chamber within the catheter. When fluid flows through spaces in the heating chamber and power is applied to the electrodes causing the electrodes to charge which is conducted through the saline, resistively heating the saline and vaporizing the water in the saline. The thermal energy remodels the tissue, resulting in improved barrier function and fewer random relaxations that cause incontinence due to OAB.

FIG. 26A illustrates positioning of a needle ablation catheter 2602 for delivering vapor to selectively ablate nerve-rich layers of deep detrusor and adventitial space beneath trigone 2622, in accordance with embodiments of the present specification. FIG. 26B illustrates positioning of a needle ablation device for delivering vapor to selectively ablate the bladder neck, IUS and nerves supplying the IUS 2624 and bladder neck, in accordance with embodiments of the present specification. Referring simultaneously to FIGS. 26A and 26B, one or more needles 2626 are used to deliver vapours to a target area 2622 or 2624. In embodiments, sensor probes are used to measure one or more parameters and thereby control the ablation. In one embodiment, a sensor probe may be positioned at the distal end of the heating chambers within the catheter. During vapor generation, the sensor probe communicates a signal to the controller. The controller may use the signal to determine if the fluid has fully developed into vapor before exiting the distal end of the heating chamber. Sensing whether the saline has been fully converted into vapor may be particularly useful for many surgical applications, such as in the ablation of various tissues, where delivering high quality (low water content) steam results in more effective treatment.

The ablation system of FIGS. 26A and 26B comprise a catheter 2602 having an internal heating chamber, disposed within a lumen of the catheter and configured to heat a fluid provided to the catheter 2602 to change the fluid to a vapor for ablation therapy. In some embodiments, the catheter 2602 is made of or covered with an insulated material to prevent the escape of ablative energy from the catheter body. A plurality of openings are located proximate the distal end of the catheter 2602 for enabling a plurality of associated thermally conductive elements, such as needles 2626, to be extended (at an angle from the catheter 2602, wherein the angle ranges between 30 to 180 degrees) and deployed or retracted through the plurality of openings. In accordance with an aspect, the plurality of retractable needles 2626 are hollow and include at least one infusion port to allow delivery of an ablative agent, such as steam or vapor, through the needles 2626 when the needles 2626 are extended and deployed through the plurality of openings on the elongated body of the catheter 2602. In some embodiments, the infusion ports are positioned along a length of the needles 2626. In some embodiments, the infusion ports are positioned at a distal tip of the needles 2626. In various embodiments, a tension wire attached to the needle is used to control the shape and position of the needle to assist in puncturing the bladder wall. In some embodiments, the vapor is applied through the needle to the trigone of the bladder ablating the nerves in trigone of the bladder to prevent or treat OAB.

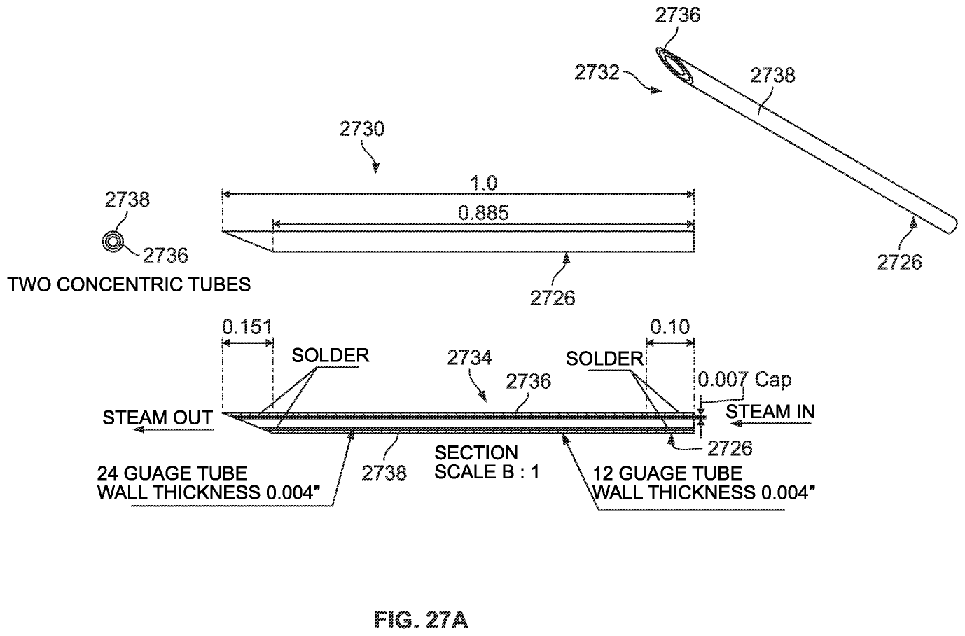

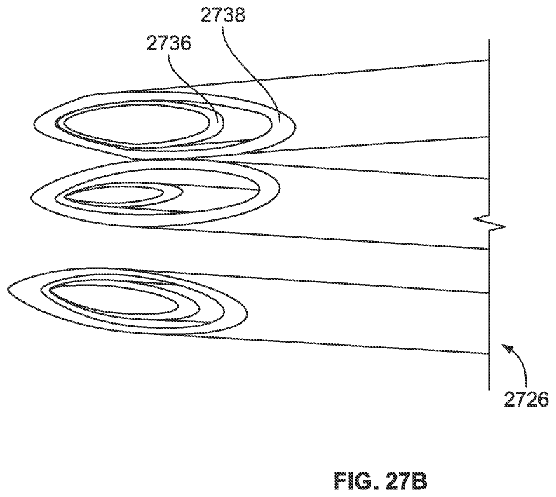

FIG. 27A illustrates different views of a coaxial needle 2726 that may be used for ablation for treatment of OAB, in accordance with some embodiments of the present specification. The figure shows a side view 2730, a front-end side perspective view 2732, and a cross-section of the side view 2734, of the needle 2726. In some embodiments, the needle 2726 comprises two concentric tubes with lumens—an inner tube 2736 within an outer tube 2738. The needle 2726 is diagonally sectioned at its sharp distal end, so that in one embodiment, a length of the needle extending to its sharp pointed distal end is about 1 mm, and a length extending to its proximal distal end is about 0.885 mm. The inner tube 2736 comprises a first lumen to provide for a channel to exit vapours for ablation. In one embodiment, a gap between the inner and outer tubes 2736 and 2738 is of about 0.007 mm. The inner and outer tubes 2736, 2738, are soldered together for about 0.151 mm at the distal end and for about 0.10 mm at the proximal end of the needle 2726. The vapours generated in the catheter travel through one or more openings where the one or more needles 2726 are connected to the catheter, enter the needles 2726 from the hollow of inner tube 2736 at the proximal side of the needle 2726 and exit from the distal side of the needle 2726. FIG. 27B illustrates the distal ends of coaxial needles 2726 comprising inner tubes 2736 with lumens and outer tubes 2738 with lumens, in accordance with some embodiments of the present specification. In some embodiments, a gap between the inner tubes 2736 with lumens and outer tubes 2738 with lumens is filled with air or a fluid for insulation. The gap may be flushed and may be used for aspiration in some embodiments.

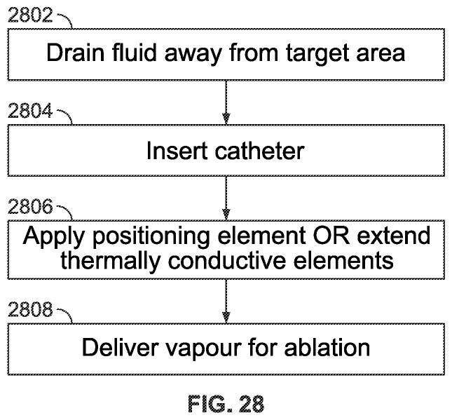

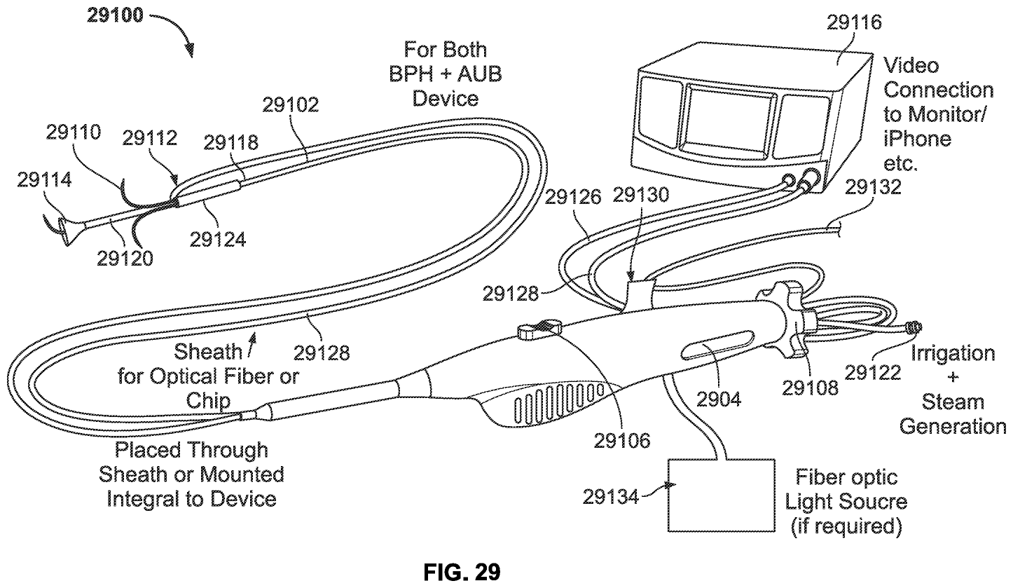

FIG. 28 is a flow chart illustrating an exemplary process of ablating the urinary bladder and/or its peripheral areas, in accordance with some embodiments of the present specification. An ablation system described in context of the various figures above is used for ablating a target area within or proximate the urinary bladder of a patient. The target area may include a tissue, cyst, tumour of stages 1 to 8, within the urinary bladder, so as to treat cancerous growth. The target area may also include nerve-rich layers of the deep detrusor and adventitial space beneath the trigone, and the bladder neck, IUS, and nerves supplying the IUS and bladder neck. In accordance with the present specification, at step 2802, liquid (urine) from the urinary bladder is drained. The bladder is drained to empty the bladder so that there is no anticipation of the urine wetting the target area or pooling on or around the target area. Draining the target area is performed to ensure the removal of bulk urine for effective ablation. In some embodiments, the urine is removed from the bladder. In some embodiments, additionally, air or CO₂ is insufflated into the bladder to expand the bladder. The air is used to dry the internal surface of the bladder before ablation. In some embodiments, additionally, the patient is positioned to drain any residual urine away from a targeted part of the bladder using gravity, by making the targeted portion of the bladder non-dependent. At step 2804, a catheter of the ablation system is inserted into the urinary bladder. At step 2806, a positioning element, such as positioning element 2312 of FIG. 23, is deployed proximate the target area, so as to enclose a portion or whole of the target area to be ablated. Alternatively, a thermally conductive element, such as one or more needles 2626 of FIGS. 26A and 26B, are deployed to access peripheral areas of the urinary bladder, including and not limited to, the area beneath the trigone and the IUS of the patient or a prostate of the patient. At step 2808, vapor is delivered to the target area to ablate the target area. In embodiments, pressure within the bladder is maintained to a level below 5 atm during the ablation.

### Imaging Capabilities

Imaging capabilities may be added to the ablation systems used for benign prostatic hyperplasia (BPH), abnormal uterine bleeding (AUB), over-active bladder (OAB), and for any other tissue ablation processes described in the embodiments of the present specification. In embodiments, the imaging capabilities are provide in the form of an integrated optical chip with the ablation system or as a coaxial fiber optical wire with the sheath of the catheter of the ablation system.

FIG. 29 illustrates a system 29100 for use in the ablation and imaging of prostatic tissue, in accordance with an embodiment of the present specification. The system 29100 comprises a catheter 29102 which, in some embodiments, includes a handle 29104 having actuators 29106, 29108 for extending at least one needle or a plurality of needles 29110 from a distal end of the catheter 29112 and expanding a positioning element 29114 at the distal end of the catheter 29112. In some embodiments, actuators 29106 and 29108 may be one of a knob or a slide or any other type of switch or button to enable extending of at least one needle from the plurality of needles 29110. Delivery of vapor via the catheter 29102 is controlled by a controller 29116. In embodiments, the catheter 29102 comprises an outer sheath 29118 and an inner catheter 29120. The needles 29110 extend from the inner catheter 29120 at the distal end of the sheath 29118 or, in some embodiments, through openings proximate the distal end of the sheath 29118. In embodiments, the positioning element 29114 is expandable, positioned at the distal end of the inner catheter 29120, and may be compressed within the outer sheath 29118 for delivery. In some embodiments, actuator 29108 comprises a knob which is turned by a first extent, for example, by a quarter turn, to pull back the outer sheath 29118. As the outer sheath 29118 retracts, the positioning element 29114 is revealed. In embodiments, the positioning element 29114 comprises a disc or cone configured as a bladder anchor. In embodiments, actuator/knob 29108 is turned by a second extend, for example, by a second quarter turn, to pull back the outer sheath 29118 further to deploy the needles 29110. In some embodiments, referring to FIGS. 29, 4C and 4E simultaneously, the needles 29110, 3116a are deployed out of an internal lumen of the inner catheter 29120, 3111a through slots or openings 3115a in the outer sheath 29118, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

Referring again to FIG. 29, in some embodiments, the catheter 29102 includes a port 29122 for the delivery of fluid, for example cooling fluid, during ablation. In some embodiments, port 29122 is also configured to provide for fluid collection, provide vacuum, and provide CO₂ for an integrity test. In some embodiments, port 29122 is used for fluid irrigation as well as for steam generation and aspiration. In some embodiments, the port 29122 is positioned on the handle 29104. In some embodiments, at least one electrode 29124 is positioned at a distal end of the catheter 29102 proximal to the needles 29110. The electrode 29124 is configured to receive electrical current, supplied by a connecting wire 29128 extending from the controller 29116 to the catheter 29102, to heat and convert a fluid, such as saline supplied via tubing 29126 extending from the controller 29116 to the catheter 29102. Heated fluid or saline is converted to vapor or steam to be delivered by the needles 29110 for ablation.

In embodiments, a capability for imaging is integrated with the system 29100. In some embodiments, sheath 29118 includes an optical fiber connected to a fiber optic light source 29134 to illuminate the passage of the distal end of catheter 29102. In some embodiments, a sheath 29128 is provided parallel to outer sheath 29118, where the sheath 29128 includes the optical fiber, or includes an optical chip. In some embodiments, the sheath 29128 is coaxial with the outer sheath 29118, parallel to an inner sheath 29120. In some embodiments, the catheter 29102 is a multi-lumen catheter, with one lumen for the camera and electronics (sheath 29128). The sheath 29128 may be made from materials such as polyurethane or thermoplastic polymers. In some embodiments, the system 29100 includes an integrated optical circuit (IC), which is mounted within the system 29100. FIG. 11O illustrates and describes detailed of embodiments of a viewing device that may be integrated with the catheter 29102, in accordance with some embodiments. The IC may be a part of the catheter 29102, or in the generator 29116, or in a third party computing device that is in communication with the system 29100. An eyepiece 29130 is integrated within the handle 29104. The eyepiece 29130 enables a user, such as the physician, to view the passage of the catheter 29102, captured by the optical system (optical fiber, integrated optical circuit). In some embodiments, a video of the images captured by the optical system is transmitted using a video correction cable 29132, to a display, such as a screen of a computer or a phone. A button or an interactive interface or trigger is provided in the generator 29116 or with a third party computing device in communication with the system 29100, which enable controlling the capture of still and video images.

FIG. 30 illustrates a system 30100 for use in the ablation of endometrial tissue, in accordance with an embodiment of the present specification. The ablation system 30100 comprises a catheter 30102 which, in some embodiments, includes a handle 30104 having actuators 30106, 30108, 30110 for pushing forward a distal bulbous tip of the catheter 30102 and for deploying a first distal positioning element 30114 and a second proximal positioning element 30116 at the distal end of the catheter 30102. In embodiments, the catheter 30102 comprises an outer sheath 30118 and an inner catheter 30120. In embodiments, the catheter 30102 includes a cervical collar 30122 configured to rest against an external os once the catheter 30102 has been inserted into a uterus of a patient. In embodiments, the distal first positioning element 30114 and proximal second positioning element 30116 are expandable, positioned at the distal end of the inner catheter 30120, and may be compressed within the outer sheath 30118 for delivery. In some embodiments, actuators 30108 and 30110 comprise knobs. In some embodiments, actuator/knob 30108 is used to deploy the distal first positioning element 30114. For example, in embodiments, actuator/knob 30108 is turned one quarter turn to deploy the distal first positioning element 30114. In some embodiments, actuator/knob 30110 is used to deploy the proximal second positioning element 30116. For example, in embodiments, actuator/knob 30110 is turned one quarter turn to deploy the proximal second positioning element 30116. In some embodiments, the handle 30104 includes only one actuator/knob 30108 which is turned a first quarter turn to deploy the first distal positioning element 30114 and then a second quarter turn to deploy the second proximal positioning element 30116. In other embodiments, other combinations of actuators/knobs are used to deploy one or both of the first distal positioning element 30114 and second proximal positioning element 30116. In some embodiments, the catheter 30102 includes a port 30124 for the delivery of fluid, for example cooling fluid, during ablation. In some embodiments, port 30124 is also configured to provide for fluid collection, provide vacuum, and provide CO₂ for an integrity test. In some embodiments, port 30124 is used for fluid irrigation as well as for steam generation or aspiration. In some embodiments, the port 30124 is positioned on the handle 30104. In some embodiments, at least one electrode 30126 is positioned at a distal end of the catheter 30102 proximal to the proximal second positioning element 30116. The electrode 30126 is configured to receive electrical current, supplied by a connecting wire 30128 extending from a controller 30130 to the catheter 30102, to heat and convert a fluid, such as saline supplied via a tubing 30132 extending from the controller 30130 to the catheter 30102. Heated fluid or saline is converted to vapor or steam to be delivered by ports 30134 for ablation. In some embodiments, the catheter 30102 is made of or covered with an insulated material to prevent the escape of ablative energy from the catheter body. A plurality of small delivery ports 30134 is positioned on the inner catheter 30120 between the distal first positioning element 30114 and the second proximal positioning element 30116. Ports 30134 are used for the infusion of an ablative agent, such as steam. Delivery of the ablative agent is controlled by the controller 30130 and treatment is controlled by a treating physician via the controller 30130.

In embodiments, a capability for imaging is integrated with the system 30100. In some embodiments, sheath 30118 includes an optical fiber connected to a fiber optic light source 30138, to illuminate the passage of the distal end of catheter 30102. In some embodiments, a sheath 30136 is provided parallel to outer sheath 30118, where the sheath 30136 includes the optical fiber, or includes an optical chip. In some embodiments the system 30100 includes an integrated optical circuit, which is mounted within the system 30100. FIG. 11O illustrates and describes detailed of embodiments of a viewing device that may be integrated with the catheter 29102, in accordance with some embodiments. An eyepiece 30140 is integrated within the handle 30104. The eyepiece 30140 enables a user, such as the physician, to view the passage of the catheter 30102, captured by the optical system (optical fiber, integrated optical circuit). In some embodiments, a video of the images captured by the optical system is transmitted using a video correction cable 30142, to a display, such as a screen of a computer or a phone.

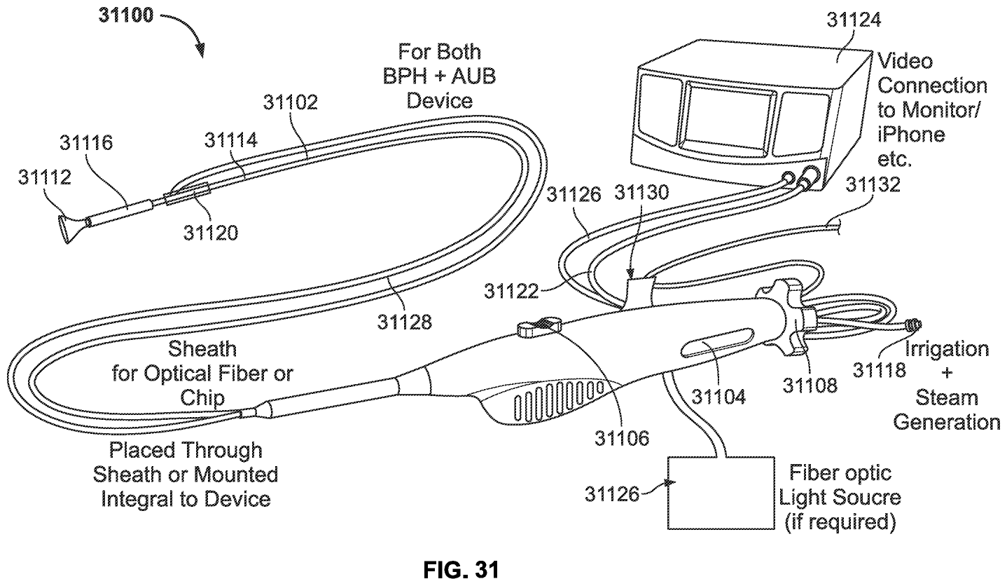

FIG. 31 illustrates a system 31100 for use in the ablation of bladder tissue, in accordance with an embodiment of the present specification. The ablation system 31100 comprises a catheter 31102 which, in some embodiments, includes a handle 31104 having actuators 31106, 31108 for pushing forward a distal tip of the catheter 31102 and for deploying a distal positioning element 31112 at the distal end of the catheter 31102. In embodiments, the catheter 31102 comprises an outer sheath 31114 and an inner catheter 31116. In embodiments, the distal positioning element 31112 is expandable, positioned at the distal end of the inner catheter 31116, and may be compressed within the outer sheath 31114 for delivery. In embodiments, the positioning element 31112 comprises a disc or cone configured as a bladder anchor. In some embodiments, actuators 31106 and 31108 comprise knobs. In some embodiments, actuator/knob 31108 is used to deploy the distal positioning element 31112. For example, in embodiments, actuator/knob 31108 is turned one quarter turn to deploy the distal positioning element 31112. In other embodiments, other combinations of actuators/knobs are used to deploy the first positioning element 31112. In some embodiments, the catheter 31102 includes a port 31118 for the delivery of fluid, for example cooling fluid, during ablation. In some embodiments, port 31118 is also configured to provide for fluid collection, provide vacuum, and provide CO2 for an integrity test. In some embodiments, port 31118 is used for fluid irrigation as well as for steam generation. In some embodiments, the port 31118 is positioned on the handle 31104. In some embodiments, at least one electrode 31120 is positioned at a distal end of the catheter 31102. The electrode 31120 is configured to receive electrical current, supplied by a connecting wire 31122 extending from a controller 31124 to the catheter 31102, to heat and convert a fluid, such as saline supplied via a tubing 31126 extending from the controller 31124 to the catheter 31102. Heated fluid or saline is converted to vapor or steam to be delivered by ports and/or needles for ablation. In some embodiments, the catheter 31102 is made of or covered with an insulated material to prevent the escape of ablative energy from the catheter body. Delivery of the ablative agent is controlled by the controller 31124 and treatment is controlled by a treating physician via the controller 31124.

In embodiments, a capability for imaging is integrated with the system 31100. In some embodiments, sheath 31114 includes an optical fiber connected to a fiber optic light source 31126, to illuminate the passage of the distal end of catheter 31102. In some embodiments, a sheath 31128 is provided parallel to outer sheath 31114, where the sheath 31128 includes the optical fiber, or includes an optical chip. FIG. 11O illustrates and describes detailed of embodiments of a viewing device that may be integrated with the catheter 29102, in accordance with some embodiments. In some embodiments the system 31100 includes an integrated optical circuit, which is mounted within the system 31100. An eyepiece 31130 is integrated within the handle 31104. The eyepiece 31130 enables a user, such as the physician, to view the passage of the catheter 31102, captured by the optical system (optical fiber, integrated optical circuit). In some embodiments, a video of the images captured by the optical system is transmitted using a video correction cable 31132, to a display, such as a screen of a computer or a phone.

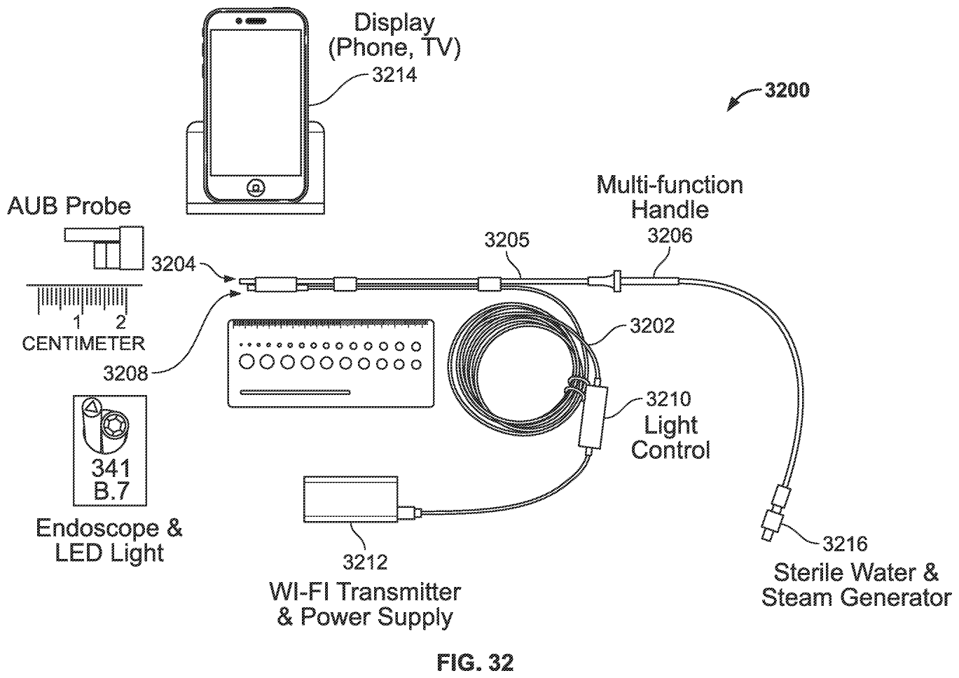

FIG. 32 illustrates various components of an optical/viewing system 3200 for direct visualization that can be used in accordance with the embodiments of the present specification. An optical/electrical catheter 3202 is placed alongside, or within a channel or a sheath of, an ablation catheter 3204, for example, an abnormal uterine bleeding (AUB) probe used to ablate a portion of a uterus. The ablation catheter 3204 is steered by a physician with controls that are provided on a multi-function handle 3206 that is included on the catheter shaft 3205. The multi-function handle 3206 also connects the ablation catheter 3204 with a fluid source via a connection tube 3216 that provides the water or saline to be converted to vapor for ablation. The distal end of the optical/electrical catheter 3202 comprises a viewing device 3208, such as a camera with a light source, such as an LED light. In embodiments, the optical/electrical catheter 3202 includes a button, a switch, or any other type of interface 3210, which enables the user to control an intensity of the light emitted at the viewing device 3208 by the light source. In some embodiments, a wireless transmitter and a power supply 3212 at a proximal end of the optical/electrical catheter 3202 provide for powering the viewing device 3208 and communicating the images taken by the viewing device 3208 to a peripheral device 3214 for display. In some embodiments, the peripheral device 3214 is a television or a computer screen, a mobile or portable display device, or a mobile phone. In some embodiments, communication between the transmitter and power supply is wired.

FIG. 33 illustrates components of a distal end 3350 of an ablation system that may be used in treatment of abnormal uterine bleeding (AUB), for use in accordance with the embodiments of the present specification. The distal end 3350 comprises a distal hood or positioning element 3352, inner catheter shaft 3353, and a proximal hood or positioning element 3354 extending from an ablation catheter 3355, and a viewing device 3356 with a light source and a camera of an optical/electrical catheter 3342. The viewing device 3356 is positioned so that the proximal hood 3354 is located distal to the viewing device along the length of the distal end 3350. In operation, a physician may view, using the viewing device 3356, the distal end of the catheter 3355, along with the distal hood 3352, inner catheter shaft 3353, and proximal hood, 3352, to ensure proper positioning of these elements prior to the initiation of vapor delivery. In embodiments, the size, stiffness, and position of each hood 3352 and 3354 is adjustable (see FIGS. 18S, 18T, 19A-C, for details). In embodiments, a length of the distal end 3350 that is between the distal 3352 and proximal 3354 hoods is also adjustable. The length, once adjusted, may be locked to position and hold the hoods 3352 and 3354 in place.

FIG. 34 illustrates an image 3450 viewed on a display device 3452, such as an iPhone, in accordance with some embodiments of the present specification. The exemplary image shows a distal hood 3454 (similar to distal hood 3352) reaching a surface that could be a fundus 3456 of a uterus during an ablation procedure. The image 3450 is captured by a viewing device such as the device 3356 of FIG. 33.

FIG. 35A depicts a cross-sectional view of an embodiment of a combination catheter 3500a comprising lumens 3502a for an optical/electrical catheter alongside a lumen 3504a for an ablation catheter, in accordance with some embodiments of the present specification. Referring simultaneously to FIG. 33, the ablation catheter lumen 3504a is configured to receive the ablation catheter shaft 3355. In some embodiments, the ablation catheter lumen 3504a has a diameter of approximately 3.5 mm. Similarly, the lumens 3502a for the optical/electrical catheter are configured to receive the optical/electrical catheter 3342 components that may include the viewing device 3356 with a light source and a camera. The lumens 3502a for the optical/electrical catheter include a camera lumen 3506a for the electronics for the viewing device 3356. The camera lumen 3506a may be square in shape, with a diagonal distance extending to 1.5 mm and sides of 1.1 mm, in an embodiment. In some embodiments, the camera lumen 3506a is configured to receive the electronics for an OV6946 camera with a resolution of 160,000 (400×400). Additionally, lumens 3502a for the optical/electrical catheter include lumens 3508a above and below lumen 3506a for holding the electronics for the light sources. The lumens 3508a may be configured to receive the electronics for LEDs with an illuminance of approximately 700 Lux. In some embodiments, the lumens 3508a are rectangular in shape. The combination catheter 3500a may be circular with a diameter of approximately 5 mm, so as to accommodate the optical/electrical catheter 3342 alongside the ablation catheter 3355.

FIG. 35B depicts a cross-sectional view of another embodiment of a combination catheter 3500b comprising lumens 3502b an optical/electrical catheter alongside a lumen 3504b for an ablation catheter, in accordance with some embodiments of the present specification. Referring simultaneously to FIG. 33, the ablation catheter lumen 3504b is configured to receive the ablation catheter shaft 3355. In some embodiments, the ablation catheter lumen 3504b has a diameter in a range of approximately 2.8 to 3.0 mm. Similarly, the lumens 3502b for the optical/electrical catheter are configured to receive the optical/electrical catheter 3342 components that may include the viewing device 3356 with a light source and a camera. In some embodiments, the lumens 3502b for the optical/electrical catheter comprise an area of the combination catheter 3500b having a diameter that may range from 1.7 mm to 3.9 mm. In an embodiment, the diameter of the area of the combination catheter housing the lumens 3502b for the optical/electrical catheter is approximately 2.0 mm. The lumens 3502b for the optical/electrical catheter include a camera lumen 3506b for the electronics for the viewing device 3356. The camera lumen 3506b may be square in shape, with a diagonal distance extending to 1.5 mm and sides of 1.1 mm, in an embodiment. In some embodiments, the camera lumen 3506b is configured to hold an OV6946 camera with a resolution of 160,000 (400×400). Additionally, lumens 3502b for the optical/electrical catheter include lumens 3508b above and below lumen 3506b for holding the electronics for the light sources. The lumens 3508b may be configured to receive the electronics for LEDs with an illuminance of 700 Lux. In some embodiments, the lumens 3508b are rectangular in shape. The combination catheter 3500b may be circular with a diameter of approximately 5.3 mm, so as to accommodate the optical/electrical catheter 3342 alongside the ablation catheter 3355.

FIG. 35C depicts a cross-sectional view of yet another embodiment of a combination catheter 3500c comprising a lumen 3502c for an optical/electrical catheter alongside a lumen 3504c for an ablation catheter 3504c, in accordance with some embodiments of the present specification. In embodiments, the catheter 3500c may have a diameter of approximately 8 mm. In embodiments, the optical/electrical catheter lumen 3502c has a diameter 3.9 mm and is configured to receive the optical/electrical catheter 3342 of FIG. 33, including the electronics for both the camera and LEDs. In embodiments, the ablation catheter lumen 3504c has a diameter 3.5 mm and is configured to receive the ablation catheter 3355 of FIG. 33. Therefore, in different embodiments, different sizes of combination catheter, optical/electrical catheter, and ablation catheter are possible.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A vapor ablation system for ablating prostate tissue of a patient, wherein the system comprises:
at least one pump;
a catheter having a length extending between a proximal end and a distal tip, wherein the catheter comprises:
a connection port positioned on the proximal end of the catheter, wherein, through the connection port, the catheter is in fluid communication with the at least one pump;
a first lumen in fluid communication with the connection port and configured to receive, via the connection port, saline from the at least one pump;
at least one electrode positioned within the first lumen; and
at least one thermally conductive, elongated element having a lumen and configured to be coupled to the distal tip of the catheter such that a proximal end of the at least one thermally conductive, elongated element is positioned at least 0.1 mm and no more than 60 mm from a distal most electrode of the at least one electrode and such that the lumen of the at least one thermally conductive, elongated element is in fluid communication with the first lumen; and
a controller having at least one processor in data communication with the at least one pump, wherein, upon activating, the controller is configured to:
control a delivery of saline into the first lumen; and
control a delivery of an electrical current to the at least one electrode positioned within the first lumen.

2. The vapor ablation system of claim 1, wherein the at least one thermally conductive, elongated element comprises a needle and a needle attachment component.

3. The vapor ablation system of claim 2, wherein the needle has a tapered distal tip.

4. The vapor ablation system of claim 2, wherein the needle and the needle attachment component are made of the same material.

5. The vapor ablation system of claim 4, wherein the same material is stainless steel.

6. The vapor ablation system of claim 2, wherein a proximal portion of the needle is configured to be threaded onto a distal end of the needle attachment component

7. The vapor ablation system of claim 1, further comprising a needle chamber coupled to the distal tip of the catheter and configured to be retractable along a length of the catheter.

8. The vapor ablation system of claim 7, wherein the needle chamber has an exterior surface and an internal lumen that defines an internal surface, wherein the exterior surface comprises a first material, wherein the internal surface comprises a second material, and wherein the first material is different from the second material.

9. The vapor ablation system of claim 8, wherein the first material is a polymer and the second material is metal.

10. The vapor ablation system of claim 7, wherein the needle chamber has an internal lumen that defines an internal surface, wherein the internal lumen is curved to receive a curved needle.

11. The vapor ablation system of claim 7, wherein the at least one thermally conductive, elongated element comprises a needle, wherein, in a pre-deployment state, the needle chamber is configured to be positioned over the needle and wherein, in a post-deployment state, the needle chamber is configured to be retracted toward a proximal end of the catheter and the needle is positioned outside the needle chamber.

12. The vapor ablation system of claim 11, wherein the needle is further adapted to have a pre-needle chamber state, wherein, in the pre-needle chamber state, the needle has a first degree of curvature, wherein, in the pre-deployment state, the needle has a second degree of curvature, wherein, in the post-deployment state, the needle has a third degree of curvature, wherein the first degree of curvature is different from both the second degree of curvature and third degree of curvature, and wherein the second degree of curvature is different from the third degree of curvature.

13. The vapor ablation system of claim 11, wherein the needle is further adapted to have a pre-needle chamber state, wherein, in the pre-needle chamber state, the needle has a first degree of curvature, wherein, in the pre-deployment state, the needle has a second degree of curvature, wherein, in the post-deployment state, the needle has a third degree of curvature, wherein the first degree of curvature is greater than both the second degree of curvature and third degree of curvature, and wherein the third degree of curvature is greater than the second degree of curvature.

14. The vapor ablation system of claim 11, wherein, in a post-deployment state, the needle is configured to extend outward at an angle between 30° and 90° from an external surface of the catheter.

15. The vapor ablation system of claim 1, wherein the at least one thermally conductive, elongated element comprises a needle and a needle attachment component and wherein the needle comprises an internal channel in fluid communication with the first lumen and a port to allow a passage of vapor to an external environment from the internal channel.

16. The vapor ablation system of claim 1, wherein the at least one thermally conductive, elongated element comprises more than one needle.

17. The vapor ablation system of claim 1, wherein the at least one thermally conductive, elongated element comprises a needle having a length extending from a proximal end to a tapered, distal end and further comprises insulation positioned over the length of needle.

18. The vapor ablation system of claim 17, wherein the insulation is adapted to cover at least 5% of the length of the needle, beginning from the proximal end and wherein the insulation is adapted to no more than 90% of the length of the needle, beginning from the proximal end.

19. The vapor ablation system of claim 1, wherein the controller is adapted to control the delivery of saline into the first lumen and control the delivery of the electrical current to the at least one electrode such that greater than 0% and less than 75% of a contiguous circumference of a prostatic urethra of the patient is ablated.

20. The vapor ablation system of claim 1, wherein the controller is adapted to control the delivery of saline into the first lumen and control the delivery of the electrical current to the at least one electrode such that greater than 0% and less than 75% of a contiguous circumference of an ejaculatory duct of the patient is ablated.

21. The vapor ablation system of claim 1, wherein the controller is adapted to control the delivery of saline into the first lumen and control the delivery of the electrical current to the at least one electrode such that greater than 0% and less than 75% of a thickness of the rectal wall is ablated.

22. The vapor ablation system of claim 1, wherein the controller is adapted to control the delivery of saline into the first lumen and control the delivery of the electrical current to the at least one electrode such that greater than 0% and less than 75% of one of a contiguous circumference of an ejaculatory duct and a central zone of the prostate is ablated.

23. The vapor ablation system of claim 1, wherein the controller is adapted to control the delivery of saline into the first lumen and control the delivery of the electrical current to the at least one electrode such that a transitional zone of a prostate of the patient is ablated and greater than 0% and less than 75% of an anterior fibromuscular strauma of the patient is ablated.
